(19) United States
(12) Patent Application Publication
(10) Pub. No.: US 2026/0176341 A1
(43) Pub. Date: Jun. 25, 2026

(54) ANTI-THYMOCYTE GLOBULIN COMPOSITIONS AND METHODS

(71) Applicant: SAB, LLC, Sioux Falls, SD (US)

(72) Inventors: Eddie J. SULLIVAN, Sioux Falls, SD (US); Thomas LUKE, Sioux Falls, SD (US); Kristi A. EGLAND, Sioux Falls, SD (US); Hua WU, Sioux Falls, SD (US); Christoph L. BAUSCH, Sioux Falls, SD (US); Alexandra Kropotova, Newtown Square, PA (US)

(21) Appl. No.: 19/427,007

(22) Filed: Dec. 19, 2025

Related U.S. Application Data
(60) Provisional application No. 63/737,514, filed on Dec. 20, 2024.
(60) Provisional application No. 63/737,542, filed on Dec. 20, 2024.
(60) Provisional application No. 63/875,143, filed on Sep. 3, 2025.
(60) Provisional application No. 63/882,245, filed on Sep. 15, 2025.
(60) Provisional application No. 63/882,247, filed on Sep. 15, 2025.

(51) Int. Cl. C07K 16/18; A61K 39/00; A61K 47/18; A61K 47/26; A61P 3/10

(52) U.S. Cl. CPC C07K 16/18; A61K 47/183; A61K 47/26; A61P 3/10; A61K 2039/505; A61K 2039/545; C07K 2317/14; C07K 2317/31

(57) ABSTRACT

Provided are human anti-thymocyte globulin (ATG) products, and methods of making and using the same. In particular, the disclosure provides an ungulate-derived polyclonal immunoglobulin, comprising a population of fully human or substantially human immunoglobulins. The population of fully human or substantially human immunoglobulins specifically binds human thymocytes, T cells, B cells, and/or monocytes. Such compositions may be made by immunization of transgenic animals having a human Ig locus with human thymocytes. This method generates polyclonal immunoglobulin with yield, purity, and antigen specificity that enable the use of this product in medical applications.

### CROSS-REFERENCE TO RELATED APPLICATIONS

This Application claims priority to U.S. Provisional Application No. 63/737,514 filed on Dec. 20, 2024; U.S. Provisional Application No. 63/737,542, filed on Dec. 20, 2024, U.S. Provisional Application No. 63/875,143 filed on Sep. 3, 2025; U.S. Provisional Application No. 63/882,245, filed on Sep. 15, 2025, and U.S. Provisional Application No. 63/882,247 filed on Sep. 15, 2025, each of which is incorporated herein by reference in its entirety.

### STATEMENT REGARDING GOVERNMENT FUNDING

This invention was made with government support under Grant Number 5R44AI142905-03, awarded by the National Institute of Allergy and Infectious Diseases. The government has certain rights in the invention.

### TECHNICAL FIELD

The invention relates generally to methods of treating type 1 diabetes with fully human polyclonal anti-thymocyte immunoglobulin compositions derived from transchromosomic ungulates.

### BACKGROUND

ATG (anti-thymocyte globulin) is a polyclonal immunoglobulin that is FDA-approved for use in organ transplantation. ATG is also used in clinical trials for treatment of graft-versus-host disease and type 1 diabetes (T1D). ATG is used as monotherapy to preserve β cell function in TiD and as an immunosuppressive for nonmyeloablative hematopoietic stem cell transplantation.

Current ATG products are produced by immunization of rabbits or horses to generate polyclonal xenobiotic immunoglobulin. The use of xenobiotic ATG therapy puts patients at risk for serum sickness as the recipient's immune system reacts to the xenobiotic immunoglobulin in the ATG. The immune response to ATG also renders redosing problematic. Furthermore, serum sickness in T1D is managed with glucocorticoids which impairs the function of the β cells of the patient and leads to transient loss of glycemic control.

Thus, there remains a need in the art for improved methods of producing ATG, with associated compositions and methods of use.

### SUMMARY

The present disclosure provides ungulate derived polyclonal immunoglobulin compositions. Also provided herein are methods of making the compositions and methods ofusing the compositions.

In an aspect, the current disclosure describes a method for producing a plurality of human antibodies or fragments thereof against human thymocytes, comprising administering a plurality of human thymocytes to a transchromosomic ungulate, wherein a viability of the plurality of human thymocytes is at least about 70%; collecting plasma from the transchromosomic ungulate; and purifying the plurality of human antibodies from the plasma, wherein the viability can be measured by dividing a number of viable cells over a number of total cells, wherein the method produces a purified human immunoglobulin (IgG) product.

In some embodiments, the method further comprises administering to the transchromosomic ungulate an adjuvant. In some embodiments, the plurality of human thymocytes comprises at least 75%, 80%, 85%, 90%, or 95% intact cells. In some embodiments, the viability is measured by counting intact cells. In some embodiments, the administering the plurality of human thymocytes is administered via a subcutaneous injection. In some embodiments, the subcutaneous injection is administered at one or more different locations of the transchromosomic ungulate. In some embodiments, the administering step is followed by collecting a plasma sample from the transchromosomic ungulate. In some embodiments, the plasma sample is about 4 L to about 10 L for a 400 kg transchromosomic ungulate per collection. In some embodiments, the plasma sample comprises about 1% to 3% of a body weight of the transchromosomic ungulate. In some embodiments, a plasma sample is collected from each of two or more transchromosomic ungulates.

In some embodiments, the method comprises pooling the plasma sample collected from each of the two or more transchromosomic ungulates to produce a pooled plasma. In some embodiments, the method comprises purifying the pooled plasma. In some embodiments, the method comprises subjecting a purified human IgG product to at least one of ultrafiltration, diafiltration, bioburden reduction, sterile filtration, or a combination thereof, to generate a storage formulation.

In some embodiments, the storage formulation comprises a) about 1 to 100 mM L-glutamic acid monosodium salt; b) about 50 to 500 mM D-sorbitol; c) about 0.01 to 2 mg/mL Tween 80; and d) about 21 to 31 mg/mL human IgG. In some embodiments, a total human IgG content in the pooled plasma is greater than 2.0 mg/mL. In some embodiments, the ungulate is a bovine.

In another aspect, the disclosure provides a pharmaceutical composition, comprising a plurality of fully human or substantially human antibodies or fragments thereof against human thymocytes, wherein the plurality of fully human or substantially human antibodies comprises an immunoglobulin (IgG) profile that is about 80% IgG1; and one or more pharmaceutically acceptable excipients.

In another aspect, the disclosure provides a pharmaceutical composition, comprising a plurality of fully human or substantially human antibodies or fragment thereof against human thymocytes, wherein the plurality of fully human or substantially human antibodies comprises a glycosylation profile having detectable alpha-galactose in released N-linked glycans and the released N-linked glycans can be measured by mass spectrometry; and one or more pharmaceutically acceptable excipients.

In some embodiments, the plurality of fully human or substantially human antibodies or fragments thereof are multi-specific. In some embodiments, the plurality of fully human or substantially human antibodies or fragments thereof, has an avidity to an immune cell surface protein of at least 1000 seconds k_off residence time.

In another aspect, the current human ATG product is produced by immunizing a transchromosomic ungulate with a plurality of human thymocytes, wherein a viability of the plurality of human thymocytes is at least about 70%, and wherein the composition comprises a plurality of fully human or substantially human antibodies or fragments thereof. In some embodiments, wherein the viability can be measured by dividing a number of viable cells over a number of total cells.

In some embodiments, the plurality of fully human or substantially human antibodies or fragments thereof is a purified human IgG product. In some embodiments, the viability of the plurality of human thymocytes is measured by counting intact cells. In some embodiments, the immunizing comprises administering a second immunization to the transchromosomic ungulate 1, 2, 3, 4, 5, 6, 7, or 8 times after a first immunization.

In another aspect, the current disclosure provides a plurality of fully human or substantially human antibodies or fragments thereof against human thymocytes, produced by pooling plasma collected from two or more transchromosomic ungulates subsequent to immunizing the two or more transchromosomic ungulates with a plurality of human thymocytes.

In some embodiments, the pooled plasma contains human anti-thymocyte immunoglobulin (ATG), and the pooled plasma is further purified to produce a purified human IgG product. In some embodiments, any anti-red blood cell antibody is substantially removed in a first purified plasma sample; any viruses, pathogens, and/or non-IgG bovine plasma proteins are removed in a second purified plasma sample; transchromosomic IgG is isolated from the pooled plasma by capturing a kappa Fab fragment in a third purified plasma sample; transchromosomic IgG is further captured in a fourth purified plasma sample; IgA, IgM, host cell proteins, DNA, endotoxin, or a combination thereof are substantially removed in a fifth purified plasma sample; viruses are substantially removed; or the human ATG is concentrated, diafiltrated, or a combination thereof as a purified human IgG product. In some embodiments, the human IgG product is formulated with an excipient buffer via diafiltration to form a storage formulation. In some embodiments, the excipient buffer comprises 50 mM L-glutamic acid monosodium salt; 262 mM D-sorbitol; 0.05 mg/ml Tween 80; and pH 5.5±0.1. In some embodiments, the storage formulation is further purified via an 0.1 to 0.3 μm filter, a sterile filter, or a combination thereof to produce a final distribution formulation.

In another aspect, the disclosure provides a method for treating Type 1 Diabetes (T1D) in a subject in need thereof, comprising administering to the subject an effective amount of the composition of claims 21-29, wherein the T1D in the subject is treated.

In some embodiments, the subject has Stage 3 New Onset of T1D (NOT1D). In some embodiments, the effective amount for patient body weight comprises between about 0.5 mg/kg to 4.5 mg/kg of a purified human IgG product formulated with a buffer. In some embodiments, the administering comprises: a first dose of about 0.5 mg/kg; and a second dose of about 1 mg/kg or 2 mg/kg, wherein the second dose is administered the day after the first dose.

In some embodiments, the composition is administered about every 6 months after a first single dose or a split dose. In some embodiments, the treatment of the T1D in the subject produces an effect of partial clinical remission or stabilization of type 1 diabetes. In some embodiments, the treatment of the T1D in the subject produces substantially no or reduced adverse events compared to administering of a rabbit anti-thymocyte globulin.

### BRIEF DESCRIPTION OF THE DRAWINGS

FIG. 1A shows a flow of the isHAC and isKcHACA vector construction. The bovinizing vector pCC1BAC—is HAC is a BAC-based one (backbone is pCC1BAC vector), consisting of 10.5 kb and 2 kb of genomic DNA as a long and short arm, respectively, 9.7 kb of the bovine genomic DNA covering the bovine I_{γ1}-S_{γ1} and its surrounding region to replace the human corresponding 6.8 kb of I_{γ1}-S_{γ1} region, the chicken j-actin promoter-driven neo gene flanked by FRT sequence and DT-A gene. After the targeted bovinization, the neo cassette is removed by FLP introduction.

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from X, phage genomic library constructed from CHO cells containing the κHAC by screening using a probe around the human I_{γ1}-S_{γ1} region. The 9.7 kb fragment (5′ end through Bsu36 I) was obtained from clone b42 derived from the λ phage bovine genomic library.

FIG. 1C shows genotyping of the bovinized I_{γ1}-S_{γ1} region. Five sets of genomic PCR were implemented, as indicated. iscont1-F1/R1 is a positive PCR specific to the homologous recombination. iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Bam HI+Pvu II and Age I, Sma I or Pvu II, respectively. bNeo 5′-RxbIgG1-5′-seq-R6 is to confirm the presence of FRT sequence.

FIG. 1D shows genotyping after the FLP-FRT deletion of the neo cassette.

FIG. 1E shows extensive genomic PCR for genotyping of the isHAC vector. Location of each genomic PCR primer pair is depicted in relation to the isHAC vector structure.

FIG. 1F shows CGH analysis among three different CHO clones containing the isHAC vector. DNA from isC1-133 was used as a reference. There was no apparent structural difference of the isHAC among the three cell lines.

FIG. 1G shows extensive genomic PCR for genotyping of the isKcHACA vector. Location of each genomic PCR primer pair is depicted in relation to the isKcHACA vector structure.

FIG. 1H shows CGH analysis among three different CHO clones containing the is KcHACΔ vector. DNA from isKCDCl5-8 was used as a reference. There was no apparent structural difference of the is KcHACΔ among the three cell lines.

FIG. 2A is a graph showing SAB-142 and rATG binding to CD3 cells. Error bars show SEM, N=6.

FIG. 2B is a graph showing SAB-142 and rATG binding to CD4 cells. Error bars show SEM, N=6.

FIG. 2C is a graph showing SAB-142 and rATG binding to CD8 cells. Error bars show SEM, N=6.

FIG. 2D is a graph showing SAB-142 and rATG binding to CD2 cells. Error bars show SEM, N=6.

FIG. 2E is a graph showing SAB-142 and rATG binding to CD45 cells. Error bars show SEM, N=6.

FIG. 2F is a graph showing SAB-142 and rATG binding to HLA-DR cells. Error bars show SEM, N=6.

FIG. 3 is a graph showing resonance units of SAB=142 against T cell CD markers.

FIG. 4A is a graph showing avidity of polyclonal rATGs against T cell CD markers compared to SAB-142.

FIG. 4B is a graph showing avidity of plasma samples V3, V4, V5, V6 and V7 against CD markers CD2, CD3, CD4 and CD8 collected after vaccination time points.

FIG. 5 is a chart showing cytokine secretion for IFN-γ, TNF, IL-10, IL-6, IL-4, and IL-2 with standard deviation measurements.

FIG. 6 is a graph showing real-time measurements of apoptosis and necrosis in human CD4+ T cells, including measurements of phosphatidylserine (PS) exposure, cell death, staurosporine, naive control, and SAB-142.

FIG. 7A is a chart showing caspase 3/7 activity over time from the extrinsic apoptosis pathway.

FIG. 7B is a graph showing caspase 8 activity over time from the intrinsic apoptosis pathway.

FIG. 8A is a graph showing competitive inhibition of FcRn binding for SAB-142 compared to rATG.

FIG. 8B is a graph showing competitive inhibition of FcγRI binding for SAB-142 compared to rATG.

FIG. 8C is a graph showing competitive inhibition of FcγRIIIa(V158) binding.

FIG. 8D is a graph showing antibody-dependent cellular cytotoxicity (ADCC) activation data on Jurkat cells.

FIG. 9A is a graph showing unstained lymphocytes, used as a control.

FIG. 9B is a graph showing naive isotype IgG used as a control that has no target specificity to measure any non-specific binding and fluorophore-related background signal.

FIG. 9C is a graph showing SAB-142 binding to lymphocytes isolated from lymph nodes from non-human primates following SAB-142 infusion at 50 mg/kg. OI refers to cells incubated only with the detection agent; NC IgG is the negative control IgG; NHP SAB-142 is non-human primate SAB-142.

FIG. 9D is a graph showing SAB-142 binding to lymphocytes isolated from lymph nodes from non-human primates following SAB-142 infusion at 25 mg/kg.

FIG. 9E is a graph showing SAB-142 binding to lymphocytes isolated from lymph nodes from non-human primates following SAB-142 infusion at 10 mg/kg.

FIG. 10 is a chart showing relative absolute lymphocytes for placebo, Cohort 3 (0.5 mg/kg), Cohort 4 (1.5 mg/kg), and Cohort 5 (2.5 mg/kg).

FIG. 11A is a chart showing relative absolute red blood cells for placebo, Cohort 3 (0.5 mg/kg), Cohort 4 (1.5 mg/kg), and Cohort 5 (2.5 mg/kg).

FIG. 11B is a chart showing relative absolute platelets for placebo, Cohort 3 (0.5 mg/kg), Cohort 4 (1.5 mg/kg), and Cohort 5 (2.5 mg/kg).

FIG. 11C is a chart showing relative absolute neutrophils for placebo, Cohort 3 (0.5 mg/kg), Cohort 4 (1.5 mg/kg), and Cohort 5 (2.5 mg/kg).

FIG. 11D is a chart showing relative CD3+CD4+ T cells for placebo, Cohort 3 (0.5 mg/kg), Cohort 4 (1.5 mg/kg), and Cohort 5 (2.5 mg/kg).

FIG. 11E is a chart showing relative CD3+CD8+ T cells for placebo, Cohort 3 (0.5 mg/kg), Cohort 4 (1.5 mg/kg), and Cohort 5 (2.5 mg/kg).

FIG. 11F is a chart showing relative B cells for placebo, Cohort 3 (0.5 mg/kg), Cohort 4 (1.5 mg/kg), and Cohort 5 (2.5 mg/kg).

FIG. 11G is a chart showing relative NK cells for placebo, Cohort 3 (0.5 mg/kg), Cohort 4 (1.5 mg/kg), and Cohort 5 (2.5 mg/kg).

FIG. 11H is a chart showing relative PD-1+ Tconv cells for placebo, Cohort 3 (0.5 mg/kg), Cohort 4 (1.5 mg/kg), and Cohort 5 (2.5 mg/kg).

FIG. 11I is a chart showing relative TIGIT+ Tconv cells for placebo, Cohort 3 (0.5 mg/kg), Cohort 4 (1.5 mg/kg), and Cohort 5 (2.5 mg/kg).

FIG. 11J is a heatmap showing Tconv median percent change from pre-infusion at day 30, day 45, day 90, and day 120 post-administration for PD-1/KLRG1, PD-1/TIGIT, and KLRG1/TIGIT compared between placebo and SAB-142 groups.

FIG. 12 is a graph showing TIGIT+ Treg cells in mean subject-normalized plots from Treg populations among treated subjects.

FIG. 13A is a graph showing Tconv cells (CD3+CD4+CD127 hi/loCD25lo) measured as a percent of pre-infusion levels for SAB-142 compared to placebo.

FIG. 13B is a graph showing Treg cells (CD3+CD4+CD127loCD25+FoxP3+) measured as a percent of pre-infusion levels for SAB-142 compared to placebo.

FIG. 13C is a graph showing CD3+CD8+ T cells measured as a percent of pre-infusion levels for SAB-142 compared to placebo.

FIG. 13D is a chart showing mean frequencies for naive, CM, EM, and TEMRA cells, demonstrating phenotypic shifts.

FIG. 14A is a graph showing mean IL-2 measurements in pg/mL from day 0 to day 30 for placebo, Cohort 3 (0.5 mg/kg), Cohort 4 (1.5 mg/kg), and Cohort 5 (2.5 mg/kg).

FIG. 14B is a graph showing mean IL-6 measurements in pg/mL from day 0 to day 30 for placebo, Cohort 3 (0.5 mg/kg), Cohort 4 (1.5 mg/kg), and Cohort 5 (2.5 mg/kg).

FIG. 14C is a graph showing mean IL-8 measurements in pg/mL from day 0 to day 30 for placebo, Cohort 3 (0.5 mg/kg), Cohort 4 (1.5 mg/kg), and Cohort 5 (2.5 mg/kg).

FIG. 14D is a graph showing mean IL-10 measurements in pg/mL from day 0 to day 30 for placebo, Cohort 3 (0.5 mg/kg), Cohort 4 (1.5 mg/kg), and Cohort 5 (2.5 mg/kg).

FIG. 14E is a graph showing mean TNF-α measurements in pg/mL from day 0 to day 30 for placebo, Cohort 3 (0.5 mg/kg), Cohort 4 (1.5 mg/kg), and Cohort 5 (2.5 mg/kg).

FIG. 14F is a graph showing mean IFNγ measurements in pg/mL from day 0 to day 30 for placebo, Cohort 3 (0.5 mg/kg), Cohort 4 (1.5 mg/kg), and Cohort 5 (2.5 mg/kg).

FIG. 15 is a schematic diagram illustrating steps for detecting anti-drug antibodies, including 1) obtaining a Meso Scale Discovery (MSD) plate coated with streptavidin; 2) obtaining SAB-142 labeled with Biotin or SULFO-TAG®; 3) incubate patient serum with acidic buffer to dissociate any existing drug+anti-drug complexes; 4) add dissociated SAB-142 treated patient serum to MSD plate; 5) record results of analysis including 5a) no anti-drug antibodies present in patient serum and therefore no bridge is formed and no signal is detected, and 5b) anti-drug antibodies are present in the patient serum and a bridge is formed between the two labeled SAB-142 antibodies and a signal is detected.

FIG. 16 is a graph showing SAB-142 concentration versus timepoint measurements for placebo, Cohort 3 (0.5 mg/kg), Cohort 4 (1.5 mg/kg), and Cohort 5 (2.5 mg/kg).

FIG. 17 is a schematic diagram illustrating steps for detecting SAB-142 in the participant serum, including 1) incubating SAB-142 participant serum samples with pooled human PBMCs; 2) after washing, incubating the cells with biotin-labeled Fab anti-human IgG; 3) finally incubating cells with streptavidin-PE; 4) Fluorescently-labeled PBMCs are gated for lymphocytes and analyzed via flow cytometry to detect SAB-142 bound to lymphocytes.

FIG. 18 is a chart showing the standard curve between SAB-142 concentration and fluorescence intensity of flow cytometry in human serum.

FIG. 19 is a diagram that describes a manufacturing method of producing human antibodies against human thymocytes from transchromosomic ungulates.

### DETAILED DESCRIPTION

The present inventors have developed a human ATG product that overcomes limitations of animal ATGs. Transgenic animals with the endogenous Ig locus replaced by a human artificial chromosome encoding a human Ig locus express fully human polyclonal antibodies. Immunization of such a transgenic animal with human thymocytes generates polyclonal immunoglobulin with yield, purity, and antigen specificity that enable use of this product in medical applications. Various embodiments of the invention are provided in the description that follows.

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a” “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, camel, llama, alpaca, buffalo, bison, and antelope.

The term “transgenic” means the cells of the ungulate comprise one or more exogenous polynucleotides encoding one or more heterologous genes (e.g. an immunoglobulin locus). The term “transgenic” is used interchangeably with “transchromosomal” herein. The exogenous polynucleotide may be maintained episomally as a portion of an artificial chromosome, integrated into the host genome, or a combination thereof. Alternatively, or in addition to an artificial chromosome, one or more polynucleotides encoding a exogenous gene may be integrated into the genome of the cells of the ungulate.

The term “polyclonal” refer to a population of immunoglobulins including multiple distinct antibody species with diverse variable regions that recognize various epitopes while sharing common regions. A polyclonal immunoglobulin population is derived from multiple B cell clones, each producing antibodies with unique antigen-binding specificities. The term polyclonal does not, however, exclude immunoglobulins derived from a single B cell precursor or single recombination event, as may be the case when a dominant immune response is generated. The term “polyclonal serum” refers to blood serum containing a polyclonal population of immunoglobulins The term “polyclonal plasma” refers to blood plasma containing a polyclonal population of immunoglobulins. The term “polyclonal immunoglobulin” refers to a population of immunoglobulins having shared constant regions but diverse variable regions, whether present in serum, plasma, or in purified form. A polyclonal serum or plasma may contain multiple soluble forms of immunoglobulins, including IgG, IgM, IgA, and other immunoglobulin classes. The term “purified polyclonal immunoglobulin” refers to polyclonal immunoglobulin that has been isolated or purified from serum or plasma. Methods of purifying polyclonal immunoglobulin include, without limitation, protein A or protein G affinity chromatography, caprylic acid fractionation, ammonium sulfate precipitation, cold ethanol fractionation, and adsorption with red blood cells (RBCs) to separate antibodies from plasma.

The present disclosure provides a human polyclonal anti-thymocyte immunoglobulin produced from the plasma of transchromosomic (Tc) Bovines™ immunized with purified human thymocytes from pediatric donors. The terms “SAB-142”, “human IgG product”, “purified human IgG product”, “SAB-142 immunoglobulins”, and “purified human ATG” can be used interchangeably, and can refer to a highly purified human polyclonal anti-thymocyte immunoglobulin (ATG) that has not undergone at least one of diafiltration, sterile filtration, bioburden reduction, or final formulation with a buffer for administration to a subject. In some embodiments, the term “SAB-142” can also refer to a pharmaceutical composition including highly purified human polyclonal anti-thymocyte immunoglobulins produced from the plasma of transchromosomic (Tc) Bovines™ immunized with purified human thymocytes from donors (eg., pediatric donors), formulated with pharmaceutically acceptable excipients. In some embodiments, the terms “SAB-142 formulation”, “human ATG formulation”, “final formulation”, “storage formulation,” and “final distribution formulation” can be used interchangeably and refer to the human IgG product sterilized and formulated for administration to a subject. In some embodiments, the term “final formulation”, “storage formulation,” or “final distribution formulation” refers to a pharmaceutical formulation including a concentration of purified human IgG product that has undergone at least one of diafiltration, sterile filtration, bioburden reduction, and final formulation with a buffer for administration to a subject.

In some embodiments, a pharmaceutical composition including an amount of a purified human IgG product (e.g., the purified human IgG product formulated with a buffer has a concentration of about 20.8 mg/kg to 31.2 mg/mL human IgG protein) includes an amount of highly purified human polyclonal anti-thymocyte immunoglobulins present in the pharmaceutical composition. The highly purified human polyclonal anti-thymocyte immunoglobulins may be present in an amount of, for example, 2 mg/mL to 200 mg/mL in a pharmaceutical composition, a formulation, or a storage/final distribution formulation including the highly purified human polyclonal anti-thymocyte immunoglobulins and at least one pharmaceutically acceptable excipient.

A “population” of immunoglobulins refers to immunoglobulins having diverse sequences, as opposed to a sample having multiple copies of a single immunoglobulin species having identical or substantially identical heavy and light chain sequences. The term population excludes immunoglobulins secreted from a single B cell, a single plasma cell, or a hybridoma in culture, or from a host cell transduced or transformed with recombinant polynucleotide(s) encoding a single pair of heavy and light chain immunoglobulin sequences. A population of immunoglobulins may include immunoglobulins of different classes (e.g., IgG, IgM, IgA) and subclasses (e.g., IgG1, IgG2, IgG3, IgG4).

The terms “immunoglobulin” and “antibody” are used interchangeably herein. “Immunoglobulin” refers to the protein molecule itself, while “antibody” emphasizes its function in binding to antigens. “Immunoglobulin” and “antibody” refer to a protein complex including at least two heavy chains and at least two light chains in a 1:1 ratio, interconnected by disulfide bonds. Immunoglobulins belong to one of five classes: IgM, IgG, IgA, IgD, or IgE, and may further belong to various subclasses (e.g., IgG1, IgG2, IgG3, IgG4, IgAQ1, IgA2). Each heavy chain includes a heavy chain variable region (abbreviated herein as VH) and a heavy chain constant region (CH). The heavy chain constant region includes three domains, CH1, CH2, and CH3. Each light chain includes a light chain variable region (abbreviated herein as VL) and a light chain constant region including one domain (CL). The VH and VL regions can be further subdivided into regions of hypervariability, termed complementarity-determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the immunoglobulin can mediate the binding of the immunoglobulin to host tissues or factors, including various cells of the immune system (e.g., effector cells).

The term “antibody,” as used herein, refers to complete immunoglobulin molecules as well as antigen-binding fragments capable of competing with intact antibodies for binding to specific targets. Such fragments may be produced through enzymatic digestion or chemical processing of complete antibodies. Representative binding fragments include Fv, Fab, Fab′, and F(ab′)2 structures. A Fab fragment includes an entire light chain paired with the VH and CH1 domains of a single heavy chain, where the heavy chain portion cannot form disulfide bonds with another heavy chain. A Fab′ fragment includes a light chain and a heavy chain portion that extends into the constant region between CH1 and CH2, permitting disulfide bond formation between two heavy chains. When two Fab′ fragments join through inter-heavy-chain disulfide bonds, the resulting structure is termed F(ab′)2. The Fv fragment consists solely of the variable domains from both heavy and light chains without constant regions. Unless context suggests otherwise, the term “antibody” as used herein includes “antibody peptide(s).”

The term “polyclonal antibody” refers to a diverse collection of antibody molecules obtained from biological sources such as serum, plasma, colostrum, milk, or other fluids from animals that have been exposed to an immunogen or antigenic derivative. These antibodies are generally collected in bulk from the biological fluids of immunized animals and include a heterogeneous mixture of immunoglobulin molecules directed against one or more antigens, with specificity for multiple distinct epitopes. Polyclonal antibodies may include any isotype, such as IgA, IgD, IgE, IgG, or IgM, and may be bispecific, multifunctional, or include fragments that maintain binding capability to target antigens or pathogenic components.

The term “antigen-binding portion” of an antibody refers to fragments that preserve the capacity to bind specifically to a target antigen (such as human thymocytes), representing functional antibody fragments. Antigen recognition can be accomplished by various fragments derived from complete antibodies. “Specific binding” in the context of polyclonal antibodies describes the preferential recognition of a target antigen within a mixture containing multiple different antigens. In certain embodiments, specific binding distinguishes between target and non-target antigens present in a sample. In some embodiments, polyclonal antibodies may recognize epitopes common to multiple cell types or surface markers while discriminating against unrelated epitopes.

An antibody is deemed to substantially bind to a T-cell surface marker when, in the presence of an excess of antibody, the amount of T-cell surface marker bound to an anti-T-cell surface marker antibody is reduced by at least about 20%, 40%, 60%, 80%, 85%, or more, as determined in an in vitro competitive binding assay.

The term “epitope”, as used herein, refers to any antigenic determinant on a polypeptide that is capable of specific binding with an immunoglobulin or a T-cell receptor. Epitope determinants may include chemically active surface features such as amino-acid side chains, carbohydrate moieties, phosphoryl or sulfonyl groups, and may exhibit defined three-dimensional conformations and/or charge characteristics. An epitope is the portion of an antigen contacted by an antibody or functional fragment thereof, or by an antigen-binding portion or fragment thereof. An antibody is considered to specifically bind an antigen when it preferentially recognizes its target antigen within a heterogenous mixture of proteins and/or macromolecules; in certain embodiments, the overall strength of binding between a population of antibodies and their corresponding antigens is referred to as “avidity”, which can be measured using residence time (1/k_off). A strong avidity value is, for example, greater than 1×10⁻⁵ at a concentration of 0.1 mg/mL. Avidity can also be measured with ELISA based assays, such as chaotropic avidity-ELISA based assays.

In variations, the immunoglobulin is engineered in any of various ways known in the art or prospectively discovered, including, without limitation, mutations to change glycosylation patterns and/or to increase or decrease complement dependent cytotoxicity. An immunoglobulin is “fully human” or “substantially human” when the amino acid sequence of the immunoglobulin exhibits sufficient structural and functional similarity to the sequence of a native human immunoglobulin such that, when administered to a human subject, the immunoglobulin does not generate a significantly greater anti-immunoglobulin immune response than would be generated by a corresponding native human immunoglobulin of the same isotype and specificity. The determination of whether an immunoglobulin qualifies as fully human or substantially human may be assessed through immunogenicity assays, including but not limited to anti-drug antibody (ADA) detection assays as the fully human protein sequences of a polyclonal antibody should not elicit an immune response from human recipients. A fully human immunoglobulin may comprise one or more amino acid substitutions, insertions, or deletions in the variable regions that arise from the processes of V(D)J recombination, somatic hypermutation, class switch recombination, clonal selection, and affinity maturation that occur during normal B-cell development and immune responses. Such modifications are considered within the scope of fully human immunoglobulins provided they do not introduce non-human sequences or significantly alter the overall human characteristics of the molecule. The constant regions of fully human or substantially human immunoglobulins typically retain the native human sequence to ensure proper effector function and minimize immunogenicity risk.

A fully human or substantially human immunoglobulin may be engineered in any of various ways known in the art or prospectively discovered, including, without limitation, alterations to glycosylation patterns (such as modifications to N-glycolylneuraminic acid (NGNA)-bearing glycans or fucosylated glycans), modifications to modulate Fc receptor binding (including FcRn, FcγRI, FcγRIIa, and FcγRIIIa binding), mutations to alter IgG subclass distribution (such as achieving at least about 70% IgG1, less than about 30% IgG2, or less than 4% of IgG3 and IgG4) and/or mutations to increase or decrease complement-dependent cytotoxicity (CDC) or antibody-dependent cellular cytotoxicity (ADCC).

The terms “thymocytes”, “T cells”, “B cells”, and “monocytes” are given their ordinary meaning in the art. Thymocytes are immature T-lineage cells in the thymus derived from hematopoietic progenitor cells that undergo differentiation and selection to become mature T lymphocytes. Thymocytes are distinct from mature T cells in that they are undergoing developmental processes, including positive and negative selection, within the thymic microenvironment. A thymocyte may express at least one receptor such as CD4, CD8, CD127, CD2, CD3, and others, and may include double-negative (CD4-CD8-), double-positive (CD4+CD8+), and single-positive (CD4+ or CD8+) developmental stages. For purposes of this invention, thymocytes specifically refer to cells capable of eliciting an immune response that generates antibodies with therapeutic immunomodulatory properties when used to immunize transchromosomic ungulates as described herein. In the methods of the disclosure, administering (human) thymocytes may refer, in some embodiments, to administering a mixed population of cells that includes thymocytes, provided thymocytes are present in sufficient quantity and purity to generate an anti-thymocyte immune response in the transchromosomic ungulate. In variations of the methods of the disclosure, non-human thymocytes are used, such as, for example, thymocytes of a non-human primate. In some embodiments, the human thymocytes are fresh human thymocytes. Fresh human thymocytes are preferably administered within 24 hours of isolation, more preferably within 12 hours of isolation, and most preferably within 6 hours of isolation

The term “regulatory T cells” or “Tregs” refers to a subset of CD4+ T cells characterized by expression of CD25, FoxP3, and low expression of CD127 (CD3+CD4+CD127^{lo}CD25+FoxP3+). Tregs function to suppress immune responses, maintain self-tolerance, and prevent autoimmunity.

The term “effector function” refers to the biological activities mediated by the Fc region of an immunoglobulin following antigen binding. Effector functions include, but are not limited to, antibody-dependent cellular cytotoxicity (ADCC), complement-dependent cytotoxicity (CDC), antibody-dependent cellular phagocytosis (ADCP), and binding to Fc receptors (such as FcRn, FcγRI, FcγRIIa, and FcγRIIIa).

The term “immunomodulation” or “immunomodulatory” refers to the alteration or regulation of immune system function, including but not limited to modulating T cell activation, inducing T cell exhaustion, preserving regulatory T cell function, altering cytokine profiles, or shifting T cell phenotypes. Immunomodulation does not necessarily require sustained lymphodepletion or cytotoxicity.

The term “lymphodepletion” refers to a sustained reduction in the number of lymphocytes in the peripheral blood or lymphoid tissues. Lymphodepletion is distinguished from transient lymphopenia, which involves temporary reduction in circulating lymphocytes due to margination or redistribution without sustained cell death.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

The terms “immunization” and “immunizing” refer to administering a composition to a subject (e.g., a transchromosomic ungulate) in an amount sufficient to elicit, after one or more administering steps, a desired immune response (e.g., a polyclonal immunoglobulin response specific to thymocytes). Administration may be by subcutaneous injection, intramuscular injection, intravenous injection, intraperitoneal injection, or any other suitable route. Immunization may comprise between one and twenty, or more administrations (e.g. injections) of the composition, such as 1, 2, 3, 4, 5, 6, 7, 8, 9, 10, 11, 12, 13, 14, 15, 16, 17, 18, 19, 20, or more administrations. The first administration may elicit no detectable immune response, and each subsequence administration can boost the immune response generated by prior administrations.

The term “target antigen” refers to any antigen used to elicit a desired immune response in a transchromosomic ungulate for the production of polyclonal immunoglobulins. The target antigen used to generate an ATG product may be fresh human thymocyte cells, cells sharing one or more endogenous protein markers with thymocytes, cells recombinantly expressing one or more thymocyte proteins, recombinant thymocyte proteins, or nucleic acids that encode thymocyte proteins (e.g. RNA, linear DNA, or plasmid DNA). In some embodiments, the target antigen comprises fresh human thymocytes administered subcutaneously in small volumes at various sites, optionally with an adjuvant administered separately, to generate fully human or substantially human immunoglobulins that specifically bind to human T cells, B cells, and/or monocytes.

The term “glycosylation” refers to the enzymatic process of adding glycan (sugar) moieties to proteins. Immunoglobulin glycosylation patterns, particularly N-glycans attached to the Fc region, can influence antibody effector functions, stability, and immunogenicity.

The term “purify” refers to separating a target cell or molecule (e.g. a population of immunoglobulins, thymocytes, polyclonal antibodies, specific binding molecules) from other substances present in a composition. Immunoglobulins may be purified by fractionation of plasma or serum, by affinity chromatography (e.g. protein A or protein G binding, or antigen-specific binding, or other capture molecules), by charge separation (e.g. ion-exchange chromatography, by size (e.g. size exclusion chromatography, gel filtration), by precipitation methods, or otherwise.

Purifying a population of immunoglobulins may comprise treating a composition comprising the population of immunoglobulins with one or more of acids, bases, salts, enzymes, coagulation factors, detergents, organic solvents, or other suitable agents. Purifying may further include adsorption of a composition comprising a target cell or molecule and an impurity onto non-target cells or molecules (e.g., red blood cells or other adsorbent materials) to partially or completely remove the impurity. Purifying may further include pre-treatment of serum or plasma, e.g., caprylic acid fractionation, filtration, centrifugation, or other preparatory steps. The purification process may involve single-step or multi-step procedures, including combinations of the aforementioned methods, to achieve the desired level of purity and concentration suitable for therapeutic applications.

The term “substantially removed, as used herein, refers to reducing a specified component (e.g., an impurity, process reagent, host-derived protein, nucleic acid, virus, endotoxin, or particulate) to a level that is functionally negligible for the intended use of the composition or process. In certain embodiments, “substantially removed” means the component is reduced by at least about 90%, 95%, 97%, 98%, or 99% relative to its level before the removal step. In other embodiments, it means the residual level is below a defined specification, the limit of detection (LOD) or limit of quantitation (LOQ) of the applicable analytical method, or below a prespecified absolute threshold (for example, ≤100 ppm, 10 ppm, ≤1 ppm, or non-detectable). Unless otherwise indicated, the applicable benchmark (percent reduction, LOD/LOQ, or absolute threshold) will be apparent from context or stated in the relevant section.

The term “subject”, the term “participant”, and the term “patient” are used interchangeably, and refer to a mammal, including but not limited to humans, non-human primates, ungulates (such as bovine, equine, porcine, ovine, or caprine), rodents, canines, and felines. In some embodiments, the subject is a human.

The terms “treating” and “treatment” refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition. In the context of conditions such as type 1 diabetes, “treating” includes preserving beta cell function, maintaining or improving C-peptide levels, reducing HbA1c levels, preventing or delaying disease progression, modulating immune responses, inducing immunological tolerance, reducing inflammation, preventing complications associated with the condition, and improving quality of life measures. Treatment may involve immunomodulatory interventions that target specific immune cell populations while preserving regulatory immune functions.

The term “type 1 diabetes” or “T1D” refers to an autoimmune disease characterized by the destruction of insulin-producing beta cells in the pancreatic islets of Langerhans by autoreactive immune cells. Type 1 diabetes may be classified into stages based on disease progression. “Stage 1 type 1 diabetes” refers to the presence of two or more islet autoantibodies with normoglycemia and no clinical symptoms. “Stage 2 type 1 diabetes” refers to the presence of two or more islet autoantibodies with dysglycemia but without overt clinical symptoms. “Stage 3 type 1 diabetes” refers to clinical diabetes with symptomatic hyperglycemia requiring exogenous insulin therapy.

The term “new onset type 1 diabetes” or “NOT1D” or “Stage 3 NOT1D” refers to type 1 diabetes diagnosed within approximately 100 days of clinical presentation. The term “recent onset type 1 diabetes” or “ROT1D” refers to type 1 diabetes diagnosed greater than 100 days but less than one year from initial diagnosis. The term “established type 1 diabetes” or “EOT1D” refers to type 1 diabetes diagnosed greater than one year but less than two years from initial diagnosis. In some contexts, established type 1 diabetes may also refer to disease duration extending beyond two years. The term “type 2 diabetes” or “T2D” refers to a metabolic disorder characterized by insulin resistance and relative insulin deficiency, which is distinct from the autoimmune etiology of type 1 diabetes. The compositions and methods described herein are directed primarily toward type 1 diabetes and its various stages, though immunomodulatory approaches may have applications in other diabetes-related conditions where immune dysfunction contributes to disease pathology.

For purposes of this disclosure, “clinical remission” in T1D refers to a sustained period during which endogenous insulin secretion and glycemic control improve to the extent that exogenous insulin requirements are meaningfully reduced while maintaining target glycemic metrics. In certain embodiments, clinical remission is evidenced by one or more of the following over a predefined interval (e.g., >3 months): a reduction in total daily insulin dose of at least about 50% from baseline or absolute insulin use <0.3 units/kg/day; preservation or increase of stimulated C-peptide (e.g., area under the curve [AUC] on mixed-meal tolerance testing) relative to baseline; HbA1c maintained at or below approximately 7.0-7.5% without excessive hypoglycemia; and continuous glucose monitoring (CGM) time-in-range (70-180 mg/dL) of at least about 60-70% with low time below range (e.g., <4%<70 mg/dL), indicating recovery or preservation of beta-cell function and improved glycemic stability. “Stabilization” of T1D refers to halting or slowing the trajectory of beta-cell decline and glycemic deterioration, such that key clinical and biochemical measures remain stable within prespecified bounds over time. In certain embodiments, stabilization is demonstrated by maintenance of C-peptide levels within a non-inferiority margin versus baseline (e.g., decline less than a rate observed in a control arm or historical controls), stable or reduced total daily insulin dose without worsening hypoglycemia, HbA1c maintained within target (e.g., ≤7.5-8.0%), and CGM metrics showing preserved time-in-range and acceptable glycemic variability. Unless otherwise specified, determinations of remission or stabilization are made using standardized testing (e.g., mixed-meal tolerance testing for C-peptide), validated continuous glucose monitoring-derived metrics, and contemporaneous insulin-dosing records, with thresholds set prospectively in the protocol to ensure clinical relevance and reproducibility.

The term “adverse event” means any untoward medical occurrence in a clinical study participant that either emerges during the study, or if present pre-dose, worsens during the study, and which does not necessarily have to have a causal relationship with the study treatment. An AE can, therefore, be any unfavorable and unintended sign (including an abnormal laboratory finding, for example), symptom, or disease associated with study participation, whether or not considered related to the study treatment. Surgical procedures themselves are not AEs; they are therapeutic measures for conditions that require surgery. The condition for which the surgery is required is an AE if it occurs or is detected following the first dose of study treatment through follow-up visit.

Conditions leading to planned surgical procedures are not AEs if the condition(s) was (were) known before study treatment. In the latter case, the condition should be reported as medical history. Pregnancy in itself is not an AE; however, an untoward unplanned negative outcome of pregnancy is an AE. A treatment-related adverse event (TEAE) is an AE that occurs from the start of the first dose of study drug administration (Day 1) through the end of a clinical study or through the time up to early termination.

The term “serious adverse event” refers to any adverse event that at any dose results in death or is life-threatening, requires inpatient hospitalization or prolongation of an existing hospitalization, results in persistent or significant disability or incapacity, or is a congenital anomaly or birth defect that occurs in the offspring of a participant exposed to the therapeutic agent. The term “life-threatening” in the definition of “serious” refers to an event in which the participant was at risk of death at the time of the event; it does not refer to an event which hypothetically might have caused death, if it were more severe. Only hospitalizations that are longer than expected based on Investigator judgement will be considered prolonged hospitalizations. Hospitalizations for elective procedures will not be considered as an SAE, unless the hospitalization is prolonged based on Investigator judgement. An event will have resulted in persistent or significant disability or incapacity if it results in a substantial disruption of a person's ability to conduct normal life functions. Other situations may be considered serious even if they are not immediately life threatening or result in death or hospitalization but might jeopardize the participant or may require medical or surgical intervention to prevent one of the other outcomes listed in the above definition. Examples of such events are intensive treatment in an emergency room or at home for allergic bronchospasm, blood dyscrasias or convulsions that do not result in hospitalization.

The term “suspected unexpected serious adverse reaction” means an adverse event that is serious, for which there is at least a reasonable possibility of a causal relationship with the therapeutic composition, and that is considered unexpected. An event is “unexpected” if it is not listed in a clinical trial list of possible events, or if it is not listed at the specificity or severity that has been observed; the term also encompasses adverse events that are described in a clinical trial protocol as occurring with a class of drugs or as anticipated based on the pharmacological properties of the therapeutic agent but are not specifically identified as occurring with the particular therapeutic agent under investigation. Abnormal laboratory findings (for example, serum chemistry, hematology, and urinalysis) or other abnormal assessments (for example, vital signs and physical examination findings) that an Investigator judges to be clinically significant will be recorded as adverse events or serious adverse events if they meet the foregoing definitions. Clinically significant abnormal laboratory findings or other abnormal assessments detected after the first administration of the study drug, or present at baseline and significantly worsened following administration of the study drug, will be reported as adverse events or serious adverse events. An Investigator can exercise medical and scientific judgment in determining whether an abnormal laboratory finding or other abnormal assessment is clinically significant.

The term “adverse event of special interest” includes events such as Grade 4 systemic infusion-related reactions (IRRs), including cytokine release syndrome (CRS); Grade 4 allergic reactions, including severe allergic reactions such as anaphylaxis; Grade 3 or higher infections, including all opportunistic infections of viral, fungal, or bacterial origin; malignancies, including lymphomas; and Grade 3 or higher liver function abnormalities involving AST, ALT, or bilirubin, defined as an aspartate aminotransferase (AST) or alanine aminotransferase (ALT) value greater than 5.0×the upper limit of normal (ULN) or a bilirubin value greater than 3.0×ULN persisting for more than 14 days. The term “immediately reportable event” refers to any adverse event in a clinical trial that requires prompt notification within a specified timeframe due to its potential impact on participant safety, study conduct, or regulatory compliance. Immediately reportable events may include serious adverse events, suspected unexpected serious adverse reactions, adverse events of special interest, events that result in study drug discontinuation, protocol deviations that may significantly affect participant safety or data integrity, pregnancies occurring in study participants or partners of study participants, and any other events that the Investigator determines warrant immediate reporting.

The term “clinically significant diabetic ketoacidosis” is defined as current or very recent hyperglycemia, for example, a blood glucose level of greater than 250 milligrams per deciliter (13.9 millimoles per liter), combined with acidemia, for example arterial pH less than or equal to 7.3, bicarbonate level of less than or equal to 18 milliequivalents per liter adjusted for albumin gap of 10-12.3, and ketonemia or ketonuria, for example serum or urine ketones elevated beyond the upper limit of normal, and requiring medical attention such as unplanned outpatient care, emergency room care, or hospitalization.

The term “anticipated event” is any adverse event (serious or non-serious) that commonly occurs as a consequence of the underlying disease and/or the background treatment regimen. Disease-specific events such as hypoglycemia and hyperglycemia are anticipated in TID and are related to insulin intake, and they are even more anticipated in patients with New Onset Type 1 Diabetes (NOT1D) who are still learning to manage their newly diagnosed condition.

The term “pharmaceutically acceptable” means biologically or pharmacologically compatible for in vivo use in animals or humans and can mean approved by a regulatory agency of the Federal or a state government or listed in the U.S. Pharmacopeia or other generally recognized pharmacopeia for use in animals, and more particularly in humans. Pharmaceutically acceptable carriers, excipients, and formulations are those that are suitable for administration to subjects without causing unacceptable toxicity, irritation, allergic response, or other adverse effects. Such carriers and excipients include, but are not limited to, sterile water, saline solutions, phosphate buffered saline, dextrose solutions, glycerol, ethanol, and combinations thereof. Pharmaceutically acceptable salts include acid addition salts and base addition salts that retain the biological effectiveness and properties of the parent compound while providing improved solubility, stability, or other desirable characteristics. The selection of appropriate pharmaceutically acceptable carriers and excipients will depend on the specific route of administration, the nature of the active ingredient, and the intended therapeutic application.

The term “C-peptide” refers to a peptide that connects the A-chain and B-chain of proinsulin and is cleaved off during insulin processing. C-peptide levels serve as a biomarker for endogenous insulin production and beta-cell function in subjects with diabetes.

The term “T cell exhaustion” refers to a state of T cell dysfunction characterized by sustained expression of inhibitory receptors (such as PD-1, TIGIT, and KLRG1), reduced effector function, and altered metabolic activity. T cell exhaustion may occur during chronic antigen exposure and may be therapeutically induced to reduce autoimmune responses.

The term “hyperimmunized” refers to an immunization regimen that generates an immune response in the transchromosomic ungulate greater than required to produce a desired antibody titer (e.g., a binding titer) after dilution of the polyclonal immunoglobulin produced by the transchromosomic ungulate. For example, if a desired titer is 1:100, one may hyperimmunize a transchromosomic ungulate by a prime immunization with human thymocytes followed by one, two, three or more boost immunizations to produce a 1:1,000 titer, or greater titer, in the transchromosomic ungulate, so that the polyclonal immunoglobulin produced by the transchromosomic ungulate may be diluted in the production of a biotherapeutic composition in order to achieve a desired titer in the biotherapeutic composition.

An immunoglobulin is “specific to” or “specifically binds” (used interchangeably herein) to a target (e.g., thymocytes or a thymocyte antigen) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular cell or substance if it binds with greater affinity, avidity, more readily, and/or with greater duration than it binds to an alternative particular cell or substance. For example, an immunoglobulin that specifically or preferentially binds to thymocytes is an immunoglobulin that binds thymocytes with greater affinity, avidity, more readily, and/or with greater duration than it binds to other cells. An immunoglobulin that specifically binds to a first cell or substance may or may not specifically or preferentially bind to a second cell or substance. As such, “specific binding” does not necessarily require (although it can include) exclusive binding. Generally, but not necessarily, reference to binding means specific binding. The binding strength of an antibody to a monovalent epitope is referred to as affinity. Polyclonal antibodies may interact with multiple epitopes on one or more antigens. As used herein, “avidity” refers to the overall binding strength between a polyclonal antibody population and multivalent antigens, which encompasses the combined effects of individual antibody-epitope affinities, the number of binding sites on the antigens, and the spatial arrangement of the antibody-antigen complexes. The strength of a polyclonal antibody population is therefore defined by measuring avidity. For a polyclonal immunoglobulin composition, binding strength can be characterized by avidity, which encompasses the collecting binding interactions of multiple antibody species with multiple epitopes on target antigens. Avidity may be measured by surface plasmon resonance (SPR) with the resonance units (RU) expressed as off-rate constants or residence time (the inverse of the off-rate constant). Higher avidity is indicated by slower off-rate constants and longer residence times. In some embodiments, avidity measurements demonstrate that polyclonal immunoglobulin compositions exhibit enhanced binding strength compared to individual antibody species, due in part to the cooperative binding effects of multiple antibodies engaging multiple epitopes simultaneously. In some embodiments, avidity measurements include chaotropic avidity assays, showing the binding of polyclonal antibodies to epitopes on human thymocytes.

The term “HAC vector” or “human artificial chromosome vector” means a vector which comprises at least a human chromosome-derived centromere sequence, a telomere sequence, and a replication origin, and may contain any other sequences as desired for a given application, including genes encoding human immunoglobulin heavy and light chains. When present in a host cell, the HAC vector exists independently from a host cell chromosome in the nucleus as an episomal element. The HAC vector enables stable maintenance and expression of large DNA inserts, such as entire immunoglobulin loci, in transgenic animals. Any suitable methods can be used to prepare HAC vectors and to insert nucleic acids of interest into the HAC, including but not limited to those described in the examples that follow, and in U.S. Pat. No. 9,902,970, the disclosure of which is incorporated by reference herein. The HAC vector is a double stranded DNA vector, as is known to those of skill in the art.

The term “resonance unit” (RU) as used herein refers to a unit used to measure changes in mass on a gold-coated sensor chip in surface plasmon resonance (SPR). A shift of 104 degrees in the resonance angle is equal to 1 RU, which corresponds to an increase of approximately 1 μg per square millimeter of surface area. RU is a quantitative measure that can be used to determine the relative binding capacity and amount of different polyclonal antibody products that bind to a particular antigen of interest, providing a standardized method for comparing binding interactions across different compositions.

### Type 1 Diabetes and Transchromosomic Bovine Anti-Thymocyte Globulin

Type 1 diabetes is an autoimmune disease that targets and destroys the insulin-secreting R cells located in the islets of Langerhans in the pancreas. Deprived of the insulin-secreting pancreatic β cells, the body is unable to sense glucose levels and produce insulin. Without therapy, this leads to severe metabolic dysregulation, uncontrolled blood glucose (BG) elevation (hyperglycemia), rapid wasting, and potentially death.

Type 1 diabetes is a lifelong, life-threatening disorder afflicting millions of people worldwide. It often presents in children and adolescents and is one of the three most prevalent severe chronic diseases of childhood, along with asthma and cancer. In 2021 there were approximately 8.4 million people worldwide with T1D, and this is predicted to increase rapidly to 13.5-17.4 million cases by 2040. Due to common and significant medical complications, T1D is both a daily and life-long burden for individuals and their families.

For those diagnosed with Stage 3 T1D, exogenous insulin is required almost immediately following the diagnosis and for the duration of their lifetime. Although remarkable progress has been made in the development of insulin therapies and glucose monitoring technologies, they remain unable to fully mimic the body's innate capacity for precise metabolic regulation. Glycemic management for T1D is a daily challenge with a constant balance between BG readings, caloric intake and composition, type and amount of injected insulin, injection site locations, time of the day, health status and activity level to determine each dose of insulin. Dosing errors can result in hypoglycemia, seizures and death. Underdosing can cause hyperglycemia, dehydration, and diabetic ketoacidosis (DKA), which can result in cerebral oedema and death. Insulin overdosing can cause hypoglycemia, an acute life-threatening condition that requires immediate medical intervention. If left untreated, hypoglycemia can lead to seizures, coma, and death. Less severe hypoglycemia can manifest as dizziness and weakness, which can lead to fall injuries and motor vehicle accidents. Despite best efforts to achieve glycemic management, those with T1D are at risk for long-term complications, including severe renal, cardiac, neurologic and micro- and macro-vascular disease resulting in blindness, myocardial infarctions, strokes/cerebral vascular accident, extremity amputation, erosive non-healing foot ulcers, chronic local and systemic infections, and renal failure requiring dialysis or even kidney transplantation. Those who develop T1D during childhood or adolescence are at increased risk for significant neurocognitive disorders including behavioral issues, lower intellectual performance and motor dysfunction, and associated structural changes in the brain.

The underlying pathophysiology of TiD disease progression is currently debated, however it is generally understood that T-cells play a central role in pathogenesis. Autoreactive T-cells, CD4+, which recognize fragments of β cell proteins, interact with antigen presenting cells, such as dendritic cells, to activate CD8+ T-cells and direct B-cells to produce high affinity antibodies. These cells traffic to the pancreas and secrete proinflammatory cytokines, which subsequently stimulate other immune cells, including macrophages, which further adds to the destruct

tion of β cells. Over time (which may be many years) this cascade of cellular and humoral immune mediators destroys p cells until a critical mass is lost and an individual becomes symptomatic from hyperglycemia. Once diagnosed with T1D, if the functional and seemingly non-functional remaining β cells can be spared from autoimmune destruction, this may result in substantial clinical benefit. A number of immunomodulatory therapies, such as T and B-cell targeting biologics and small molecules are being investigated with the aim to preserve residual p cell mass in T1D patients.

One immunomodulatory modality that has shown promise in T1D recently is an anti-thymocyte globulin (ATG), Thymoglobulin®. Anti-thymocyte antibodies such as Thymoglobulin® are generated by inoculating animals (rabbits) with normal human thymus cells. The resultant polyclonal antibodies have multiple antigen specificities for lymphocytes. It is well documented that the efficacy of Thymoglobulin® at high dose levels approved for transplant rejection indications is primarily due to its ability to act as T-cell depleting antibodies. Two ATG antibody therapeutics have been approved by the Food and Drug Administration (FDA), the European Medicines Agency (EMA) and the Therapeutic Goods Administration (TGA) for transplant rejection. The approved therapeutics are Thymoglobulin® (Sanofi) derived from rabbits and ATGAM® (Pfizer) derived from horses. Of these, Thymoglobulin® has been successfully studied in several clinical trials in patients with new onset Stage 3 T1D.

Thymoglobulin®, a rabbit-derived ATG, has demonstrated efficacy in delaying the progression of T1D in clinical trials. There is substantial clinical evidence to suggest that a low dose 2.5 mg/kg of Thymoglobulin®, administered as a single course to Stage 3 T1D patients, shows disease-modifying therapeutic potential in preservation of C-peptide and better glucose control as measured by HbA1c. However, a clinical trial with higher dose of Thymoglobulin® at 6.5 mg/kg failed to achieve β cell functional preservation measured by C-peptide. Reported AEs associated with Thymoglobulin® (2.5 mg/kg) treatment in participants with new onset T1D (age 12-45 years) have included immune system disorders such as cytokine release syndrome (CRS—with symptoms of fever, headaches, nausea), lymphopaenia and serum sickness, the latter is a consequence of immunogenicity to the rabbit antibodies.

SAB-142 is a fully human ATG therapy designed to achieve robust efficacy while minimizing the AEs typically associated with administering heterologous animal-derived immunoglobulins to humans.

### Transchromosomic Bovine Anti-Thymocyte Globulin “SAB-142”

SAB-142 is a fully human multi-specific, polyclonal, anti-thymocyte immunoglobulin produced using advanced genetic engineering and antibody science. It is a disease-modifying therapeutic agent to delay the onset and progression of type 1 diabetes. Current immunoglobulin treatments are known to cause serum sickness and anaphylaxis because they trigger an immune response to the treatment antibodies. SAB-142 has been demonstrated in a Phase I clinical trial, SAB-142-101,to have a low-immunogenic profile when administered intravenously with no anti-drug antibodies (ADAs) for target dose levels. Furthermore, due to the fully human nature of the antibodies, SAB-142 does not cause serum sickness, a common problem with immunoglobulin treatments. The Phase I safety and immunogenicity data are consistent with the evidence of the low-immunogenic properties of SAB-142.

SAB-142, therefore, has greatly improved safety and dosing attributes for patients with type 1 diabetes. In preclinical studies, SAB-142 showed binding of peripheral blood mononuclear cells (PBMCs), analogous to that of both rabbit- (Thymoglobulin®) and equine- (ATGAM®) derived ATG products. This was further corroborated by in vivo immune-profiling conducted during a toxicology study in non-human primates and in a Phase I clinical trial in healthy volunteers. Moreover, SAB-142 demonstrated similar in vitro and in vivo T-cell exhaustion profile in humans as rabbit-ATG, along with T-regulatory cell preservation and T-conventional cell survival.

### Pharmaceutical Compositions Including SAB-142

SAB-142 drug product is essentially fully human IgG or fragment thereof raised against human thymocytes, formulated as sterile liquid human immunoglobulin product intended for intravenous (IV) delivery. For IV administration, the SAB-142 drug product is diluted in sodium chloride 0.900 (w/v) for infusion. The product is comprised of the disclosed concentrated and purified transchromosomic bovine anti-thymocyte globulin, formulated at a target concentration of 20.8-31.2 mg/mL in a formulation buffer comprising 10 mM L-glutamic acid monosodium salt, 262 mM D-sorbitol, <0.5 mg/mL Tween 80, pH 5.5 and aseptically filled into pre-sterilized Type I borosilicate clear glass vials, sealed with a chlorobutyl rubber stopper and an aluminum crimp cap, and stored at 2-8° C. TABLE 1 provides additional details of the SAB-142 pharmaceutical composition.

TABLE 1
describes the pharmaceutical compositions including SAB-142.

| Attribute | Current target/specification | |
|---|---|---|
| Buffer composition | 10 mM L-glutamic acid monosodium salt; | |
| | 262 mM D-sorbitol; 0.05 mg/mL Tween 80; | |
| | pH 5.5 ± 0.1 | |
| Final protein concentration (release spec) | 20.8-31.2 | mg/mL |
| pH (release spec) | 5.2-5.7 | |
| Osmolality (release spec) | 240-360 | mOsmol/kg |
| Visual Appearance | Meets USP <790> and Ph. Eur. 2.9.20-Essentially free | |
| | of particles (Pass/Fail) | |
| | Colorless | |
| Residual Bovine IgG | <100 | ppm |
| Residual Bovine Plasma Protein | <100 | ppm |
| Residual Bovine Serum Albumin | <100 | ppm |
| Heme | ≤8.0 | mg/dL |
| Bioburden | ≤1 CFU/10 mL TSA | |
| | ≤1 CFU/10 mL SDA | |
| Endotoxin (LAL) | ≤2.0 | EU/mg |

| | |
|---|---|
| | (EU/mL) |
| Size Exclusion Chromatography High | >95.0% Monomer plus Dimer |
| Performance | |
| Liquid Chromatography | ≤3.0% IgG Aggregates |
| (SEC HPLC) | ≤5.0% IgG Aggregates plus Fragments |
| CE-SDS¹ | Heavy Chain 50-125 kDa |
| | Light Chain 25-28 kDa |
| | ≥90% Purity |

¹Testing not routinely performed for bulk process intermediate release unless the lot is placed on stability.

Preliminary formulation screens compared multiple buffers for stability (potency, aggregation, degradation) across temperatures (2-8° C., 18-30° C. and 35-39° C.) and pH. To monitor the stability of the human IgG under different conditions, samples were tested on days 30, 60, 90, and 120 by SDS-PAGE and SEC-HPLC for degradation and aggregation, by potency and by visual inspection for particulates. The following formulations were tested: Formulation 1: 0.2 M glycine, 0.01% v/v Tween 80, 0.05 M NaCl, pH 6.2-6.5
Formulation 2: 0.2 M glycine, pH 4.5
Formulation 3: 0.25 M L-proline, pH 4.8
Formulation 4 (selected): 10 mM L-glutamic acid monosodium salt, 262 mM D-sorbitol, 0.05 mg/mL Tween 80, pH 5.5.

The formulation buffer used for the SAB-142 pharmaceutical composition was developed and tested in a study that tested stability of a hyperimmune anti-Anthrax human polyclonal antibody IgG (HT-468), produced in transchromosomic bovines, when formulated at 20 and 40 mg/mL in a buffer that contain 10 mM of glutamic acid monosodium salt, 262 mM of sorbitol and 0.05 mg/mL Tween 80. The stability was assessed at different temperatures (−20° C., 4° C., 22° C. and 37° C.) and different pH (5,0, 5.5, 6.0) over 12 months. At different stability time points (30, 90, 180, 270 and 365 days), product's appearance, potency, aggregations and degradation levels have been evaluated using adequate analytical methods (titer neutralization [TNA], size exclusion chromatography high performance liquid chromatography [SEC-HPLC], sodium dodecyl-sulfate polyacrylamide gel electrophoresis [SDS-PAGE], protein concentration [A280]). Results of this study have shown that the anti-anthrax hyperimmune specific immunoglobulins derived from Tc Bovine plasma is stable at all test conditions detailed above. No significant increase in IgG aggregates, dimers and fragments was observed during the 12 months period at temperatures tested. With regards to pH, the products proved to be stable in study pH range, 5.0 to 6.0.

The formulation buffer was added into purified transchromosomic bovine globulin by way of tangential flow filtration and exchanging the buffer used in previous steps with the SAB established formulation buffer. The final formulation used a 7-10 diavolume exchange which results in a 99.4-99.9% small molecule removal rate. Results of this study showed that the hyperimmune specific immunoglobulins derived from transchromosomic bovine plasma is stable at all test conditions detailed above. No significant increase in IgG aggregates, dimers, and fragments was observed during the 12-months period at temperatures tested. Regarding pH, the products proved to be stable within the study pH range of 5.0 to 6.0.

The formulation buffer was added to the purified immunoglobulins via tangential flow filtration, exchanging the buffer used in previous steps with the SAB-established formulation buffer. The final formulation uses a 7-10 diavolume exchange which results in a 99.4-99.9% small molecule removal rate. Solution 20 (10 mM L-glutamic acid monosodium salt; 262 mM D-sorbitol; 0.05 mg/mL Tween 80; pH 5.5) is explicitly identified as the Final Formulation solution used in manufacturing (non-clinical and clinical scale).

The formulation buffer was also used as blanks/controls in certain analytical methods, and the CE-SDS method description notes specificity with “no peaks observed in the formulation buffer sample” beyond expected system peaks, supporting method specificity in the presence of excipients.

Specifications connected to the formulated state include protein concentration 20.8-31.2 mg/mL, pH 5.2-5.7, and osmolality 240-360 mOsmol/kg, alongside identity (human IgG western blot), purity/aggregation (SEC-HPLC), and visible particulates standards, which, taken together, bound the formulation's acceptable quality attributes for the bulk SAB-142 composition. The stability program for the drug substance includes pH, protein concentration, bioburden, anti-human PBMC-CDC activity; and quantitative particulate testing.

The disclosure provides pharmaceutical compositions, comprising a population of fully human or substantially human immunoglobulins, and one or more pharmaceutically acceptable excipients. In some embodiments, the population of fully human or substantially human immunoglobulins specifically binds human thymocytes, T cells, B cells, and/or monocytes.

In some embodiments, the pharmaceutical composition comprises at least about 1 mg/mL, at least about 50 mg/mL, at least about 100 mg/mL, or at least about 1,000 mg/mL of fully human or substantially human immunoglobulin. In some embodiments, the pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin. Residual non-human proteins are tightly controlled: residual bovine IgG, bovine plasma proteins, and bovine serum albumin each have a limit of less than 100 ppm. The human IgG content of SAB-142 is >99.99% by mass relative to bovine proteins in the SAB-142 immunoglobulins. Chimeric antibodies are less than 2% of total IgG. These parameters support the “substantially human IgG” characterization for SAB-142.

Each immunoglobulin G (IgG) molecule contains two heavy chains and two light chains. The heavy chain has a molecular weight of approximately 50 kiloDaltons (kDa), while the light chain has a molecular weight of approximately 25 kDa. Overall, the IgG molecule has a molecular weight of approximately 150 kDa. The drug product contains all human IgG subclasses with human IgG1 as the primary antibody subclass. The chimeric antibodies (containing human heavy chain and bovine kappa light chain) are usually less than 2% of total IgG.

A test method is designed to capture human Fc and to detect human Fc to quantitatively measure the presence of fully human IgG. A separate test is used to capture and detect and chimeric IgG containing the human heavy chain and bovine kappa light chain present in test samples. Transchromosomic bovine-derived plasma may contain fully human, fully bovine, chimeric, and trans-bovine (t-bovine) IgG molecules. Since the transchromosomic bovine-derived human IgG product mainly consists of human kappa light chain, it is expected that some human kappa light chains may have similar amino acid sequences to bovine kappa light chains, and thus form hybrid Ig molecule types consisting of chimeric and t-bovine IgG. For this reason, test samples derived from Tc Bovine plasma have the possibility of containing chimeric and t-bovine IgG in addition to fully human IgG. This test method specifically detects all IgG molecules containing human Fc, which includes both fully human IgG and chimeric IgG.

The pharmaceutical composition of SAB-142 includes a newly developed buffer to increase stability of the composition in addition to the purified SAB-142 transchromosomic bovine anti-thymocyte globulins. The pharmaceutical compositions also include pharmaceutically acceptable excipients. Pharmaceutically acceptable excipients can include a buffer, a disintegrant, a preservative, a stabilizer, a surfactant, a solvent, and a combination thereof.

Buffers may be used to maintain the pH of the formulation within a desired range, such as pH 4.5 to 7.0, pH 5.0 to 6.5, or pH 5.5±0.1. Suitable buffers include, but are not limited to, L-glutamic acid monosodium salt, histidine, histidine hydrochloride, sodium phosphate, potassium phosphate, sodium citrate, citric acid, acetic acid, sodium acetate, glycine, arginine, arginine hydrochloride, tris(hydroxymethyl)aminomethane (Tris), and combinations thereof. Buffer concentrations may range from about 1 mM to about 100 mM, from about 5 mM to about 50 mM, or from about 10 mM to about 25 mM.

Stabilizers may be used to prevent protein aggregation, degradation, or denaturation during manufacturing, storage, and administration. Suitable stabilizers include, but are not limited to, sugars such as sucrose, trehalose, glucose, fructose, lactose, and maltose; sugar alcohols such as D-sorbitol, mannitol, xylitol, and erythritol; amino acids such as glycine, L-proline, L-arginine, L-histidine, L-methionine, and L-glutamic acid; polyols such as propylene glycol and polyethylene glycol; and combinations thereof. Stabilizer concentrations may range from about 10 mM to about 500 mM, from about 50 mM to about 400 mM, from about 100 mM to about 350 mM, or from about 200 mM to about 300 mM. In some embodiments, the stabilizer is present at a concentration of about 262 mM.

Surfactants may be used to prevent protein aggregation, reduce surface adsorption, and protect against interfacial stress during manufacturing and handling. Suitable surfactants include, but are not limited to, polysorbates such as polysorbate 20 (Tween 20), polysorbate 40 (Tween 40), polysorbate 60 (Tween 60), and polysorbate 80 (Tween 80); poloxamers such as poloxamer 188 (Pluronic F-68) and poloxamer 407; and non-ionic surfactants such as Brij surfactants and Triton X-100. Surfactant concentrations may range from about 0.001 mg/mL to about 2 mg/mL, from about 0.01 mg/mL to about 1 mg/mL, from about 0.02 mg/mL to about 0.5 mg/mL, or from about 0.05 mg/mL to about 0.2 mg/mL In some embodiments, the surfactant is present at a concentration of less than about 0.5 mg/mL, less than about 0.2 mg/mL, or about 0.05 mg/mL.

Preservatives may be included to prevent microbial growth during storage, particularly in multi-dose formulations. Suitable preservatives include, but are not limited to, benzyl alcohol, phenol, m-cresol, chlorobutanol, methylparaben, propylparaben, benzalkonium chloride, and combinations thereof. Preservative concentrations may range from about 0.1% to about 2% (w/v), from about 0.5% to about 1.5% (w/v), or from about 0.9% to about 1.0% (w/v). In some embodiments, the formulation is preservative-free and intended for single-use administration.

Solvents provide the aqueous vehicle for the formulation. Suitable solvents include, but are not limited to, Water for Injection (WFI), sterile water, bacteriostatic water, and combinations thereof. The solvent is typically present in an amount sufficient to bring the formulation to a final volume of 1 mL per unit dose.

Tonicity agents may be used to adjust the osmolality of the formulation to be isotonic or near-isotonic with physiological fluids. Suitable tonicity agents include, but are not limited to, sodium chloride, potassium chloride, glycerin, D-sorbitol, mannitol, sucrose, trehalose, and combinations thereof. Tonicity agent concentrations may be adjusted to achieve an osmolality of about 250 mOsm/kg to about 360 mOsm/kg, about 270 mOsm/kg to about 330 mOsm/kg, or about 280 mOsm/kg to about 310 mOsm/kg.

Antioxidants may be included to prevent oxidative degradation of the immunoglobulin. Suitable antioxidants include, but are not limited to, methionine, ascorbic acid, sodium ascorbate, cysteine, N-acetylcysteine, glutathione, and combinations thereof. Antioxidant concentrations may range from about 0.1 mM to about 50 mM, from about 1 mM to about 25 mM, or from about 5 mM to about 15 mM.

Chelating agents may be included to bind metal ions that could catalyze oxidation or other degradation reactions. Suitable chelating agents include, but are not limited to, ethylenediaminetetraacetic acid (EDTA), diethylenetriaminepentaacetic acid (DTPA), citric acid, and combinations thereof. Chelating agent concentrations may range from about 0.01 mM to about 10 mM, from about 0.1 mM to about 5 mM, or from about 0.5 mM to about 2 mM.

In some embodiments, the SAB-142 formulation comprises 10-80 mg/mL SAB-142 immunoglobulin, 1 mM to 100 mM buffer, 10 mM to 500 mM stabilizer, and 0.001 mg/mL to 2 mg/mL surfactant at pH 4.5 to 7.0. In some embodiments, the SAB-142 formulation comprises about 15 to 35 mg/mL SAB-142 immunoglobulin, 5 mM to 50 mM buffer, 50 mM to 400 mM stabilizer, and 0.01 mg/mL to 1 mg/mL surfactant at pH 5.0 to 6.5. In some embodiments, the buffer is selected from L-glutamic acid monosodium salt, histidine, histidine hydrochloride, sodium phosphate, potassium phosphate, sodium citrate, citric acid, acetic acid, sodium acetate, and combinations thereof. In some embodiments, the stabilizer is selected from D-sorbitol, mannitol, sucrose, trehalose, glycine, L-proline, L-arginine, and combinations thereof. In some embodiments, the surfactant is selected from polysorbate 20, polysorbate 40, polysorbate 60, polysorbate 80, poloxamer 188, poloxamer 407, and combinations thereof. In some embodiments, the SAB-142 formulation comprises 21-31 mg/mL SAB-142 immunoglobulin, 50 mM L-glutamic acid monosodium salt, 262 mM D-sorbitol, and 0.05 mg/mL Tween 80 at pH 5.5±0.1. SAB-142 formulation comprises about 20 to 32 mg/mL SAB-142 immunoglobulin, about 8 mM to 15 mM buffer, about 200 mM to 300 mM stabilizer, and about 0.03 mg/mL to 0.1 mg/mL surfactant at pH about 5.3 to 5.7. In some embodiments, the SAB-142 formulation comprises about 25 to 30 mg/mL SAB-142 immunoglobulin, about 10 mM to 12 mM buffer, about 250 mM to 275 mM stabilizer, and about 0.04 mg/mL to 0.06 mg/mL surfactant at pH about 5.4 to 5.6. In some embodiments, the SAB-142 formulation comprises about 40 to 60 mg/mL SAB-142 immunoglobulin, about 20 mM to 40 mM buffer, about 150 mM to 350 mM stabilizer, and about 0.02 mg/mL to 0.5 mg/mL surfactant at pH about 5.0 to 6.0. In some embodiments, the SAB-142 formulation comprises about 65 to 80 mg/mL SAB-142 immunoglobulin, about 30 mM to 60 mM buffer, about 100 mM to 200 mM stabilizer, and about 0.05 mg/mL to 0.8 mg/mL surfactant at pH about 5.2 to 5.8. In some embodiments, the SAB-142 formulation comprises about 21-31 mg/mL SAB-142 immunoglobulin, about 50 mM L-glutamic acid monosodium salt, about 262 mM D-sorbitol, and about 0.05 mg/mL Tween 80 at pH about 5.5±0.1. In some embodiments, the SAB-142 formulation comprises about 20.8-31.2 mg/mL SAB-142 immunoglobulin, about 10 mM L-glutamic acid monosodium salt, about 262 mM D-sorbitol, and less than about 0.5 mg/mL Tween 80 at pH about 5.5. In some embodiments, the SAB-142 formulation comprises about 25-75 mg/mL SAB-142 immunoglobulin, about 5 mM to 75 mM L-glutamic acid monosodium salt, about 100 mM to 400 mM D-sorbitol, and about 0.01 mg/mL to 0.5 mg/mL polysorbate 80 at pH about 5.2 to 5.7. In some embodiments, the SAB-142 formulation comprises about 15-50 mg/mL SAB-142 immunoglobulin, about 10 mM to 30 mM histidine, about 200 mM to 300 mM sucrose, and about 0.02 mg/mL to 0.1 mg/mL polysorbate 80 at pH about 5.5 to 6.5. In some embodiments, the SAB-142 formulation comprises about 20-40 mg/mL SAB-142 immunoglobulin, about 15 mM to 25 mM sodium phosphate, about 220 mM to 280 mM trehalose, and about 0.03 mg/mL to 0.08 mg/mL poloxamer 188 at pH about 6.0 to 6.5.

### Dose-Dependent Plasma and Serum Concentrations

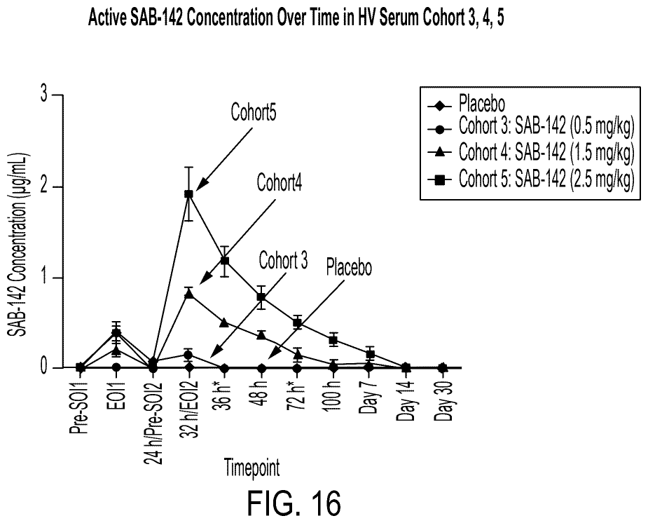

FcRn controls antibody recycling in the endocytic compartment following antibody internalization. Increased binding leads to higher antibody recycling and increased antibody concentrations in the plasma and tissues. SAB-142 demonstrates dose-dependent pharmacokinetic behavior with peak serum concentrations achieved within 24-48 hours following intravenous administration. In a Phase I study (SAB-142-101) the Cₘₐₓ and AUCₗₐₛₜ (area under the curve from time zero to the last time point with measurable concentration) values of SAB-142 in human serum increased in a dose proportional manner across 0.5, 1.5, and 2.5 mg/kg doses (FIG. 16). Although systemic exposure to SAB-142 was limited for the 0.5 mg/kg dose, the Cₘₐₓ values ranged from 0.679 to 1.01, 1.04 to 3.00 and 0.99 to 4.32 pg/mL for the 1.5 mg/kg dose and the 2.5 mg/kg dose, respectively, and AUCₗₐₛₜ values ranged from 9.15 to 36.0 and 31.7 to 105 h*μg/mL at 1.5 mg/kg and 2.5 mg/kg, respectively (FIG. 16). The half-life from the 2.5 mg/kg dose was between 26.4 and 61.6 hours.

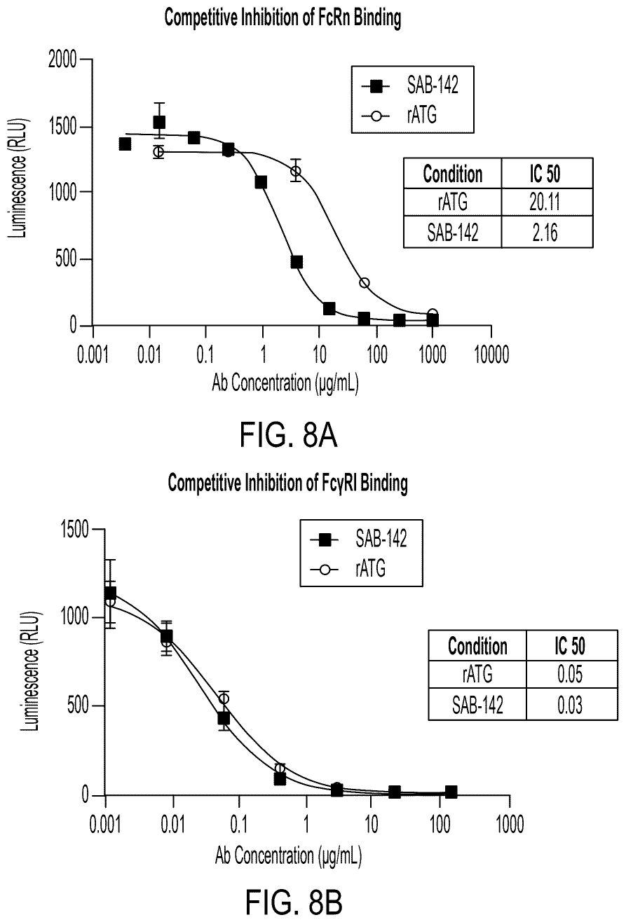

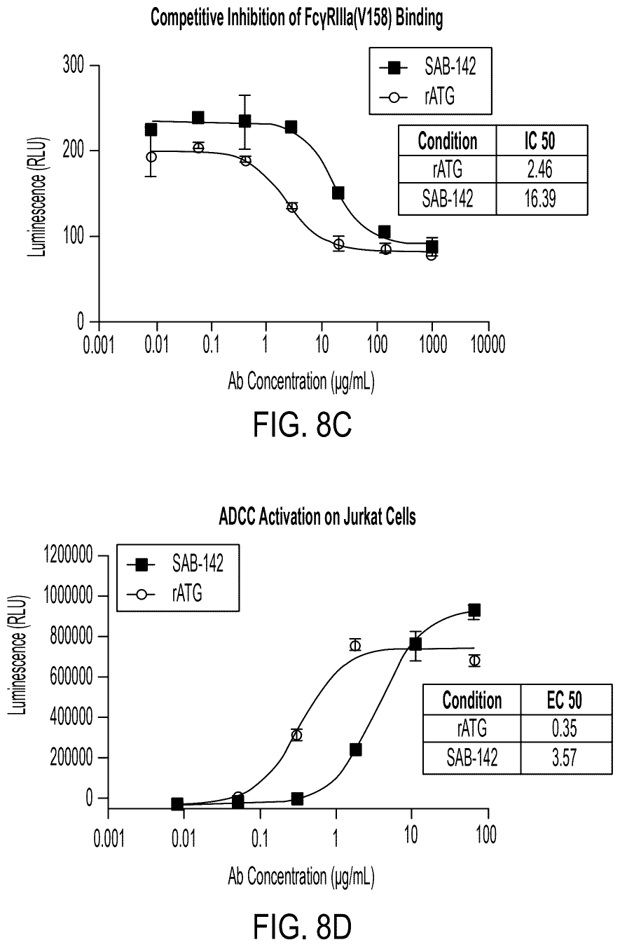

The extended serum half-life and improved pharmacokinetic properties compared to rabbit anti-thymocyte globulin may be due in part to enhanced binding of SAB-142 to human neonatal Fc receptor (FcRn) compared to rabbit anti-thymocyte globulin, with an IC50 of 2.16 for SAB-142 versus 20.11 for rATG (FIG. 8A). The enhanced FcRn binding affinity of SAB-142 is consistent with its fully human origin and may result in improved tissue distribution and prolonged circulation time compared to heterologous rabbit-derived anti-thymocyte globulin preparations.

### IgG Subclass Distribution and Characterization

SAB-142 was developed to be strongly biased to human IgG1, and includes at least 85% IgG1, with less than about 13% IgG2, and less than about 0.1% of IgG3 and IgG4 combined (see TABLE 2). Any chimeric antibodies including a human heavy chain and a bovine kappa light chain are less than about 2% of total IgG, showing that the IgG in SAB-142 is substantially human in composition. The IgG subclass distribution is optimized to provide appropriate effector functions while maintaining therapeutic efficacy.

TABLE 2
shows the IgG subclass present in normal
human IgG compared to SAB-142.

| IgG | Normal Human | Human IgG Subclass |
|---|---|---|
| Species | IgG Subclass % | % in SAB-142 |
| IgG1 | 60.3-71.5 | 87.7-88.3 |
| IgG2 | 19.4-31.0 | 11.7-12.2 |
| IgG3 | 5.0-8.4 | <0.01 |
| IgG4 | 0.7-4.2 | 0.07 |

In some embodiments, the ungulate-derived polyclonal human immunoglobulin compositions comprise at least about 70%, 75%, 80%, 85%, 90%, or 95% IgG1. In some embodiments, the human polyclonal immunoglobulins comprise less than about 30%, 25%, 20%, 15%, or 10% IgG2. In some embodiments, the human polyclonal immunoglobulins comprise less than about 4%, 3%, 2%, 1%, 0.5%, or 0.1% IgG3 and/or IgG4.

In some embodiments, the ungulate-derived polyclonal human immunoglobulin compositions comprise about 75% to about 95% IgG1, about 5% to about 19% IgG2, and about 0.01% to about 3% IgG3 and/or IgG4. In some embodiments, the ungulate-derived polyclonal human immunoglobulin compositions comprise about 76% to about 94% IgG1, about 6% to about 19% IgG2, and about 0.02% to about 2.5% IgG3 and/or IgG4. In some embodiments, the ungulate-derived polyclonal human immunoglobulin compositions comprise about 77% to about 93% IgG1, about 7% to about 18% IgG2, and about 0.03% to about 2% IgG3 and/or IgG4. In some embodiments, the ungulate-derived polyclonal human immunoglobulin compositions comprise about 78% to about 92% IgG1, about 8% to about 17% IgG2, and about 0.04% to about 1.8% IgG3 and/or IgG4. In some embodiments, the ungulate-derived polyclonal human immunoglobulin compositions comprise about 79% to about 91% IgG1, about 9% to about 16% IgG2, and about 0.05% to about 1.5% IgG3 and/or IgG4. In some embodiments, the ungulate-derived polyclonal human immunoglobulin compositions comprise about 80% to about 90% IgG1, about 10% to about 15% IgG2, and about 0.01% to about 2% IgG3 and/or IgG4. In some embodiments, the ungulate-derived polyclonal human immunoglobulin compositions comprise about 81% to about 89% IgG1, about 11% to about 14% IgG2, and about 0.05% to about 1.5% IgG3 and/or IgG4. In some embodiments, the ungulate-derived polyclonal human immunoglobulin compositions comprise about 82% to about 88% IgG1, about 12% to about 14% IgG2, and about 0.08% to about 1% IgG3 and/or IgG4. In some embodiments, the ungulate-derived polyclonal human immunoglobulin compositions comprise about 87% to about 89% IgG1, about 11% to about 12% IgG2, and about 0.01% to about 0.2% IgG3 and/or IgG4. In some embodiments, the ungulate-derived polyclonal human immunoglobulin compositions comprise about 85% to about 90% IgG1, about 10% to about 13% IgG2, and about 0.1% to about 0.5% IgG3 and/or IgG4.

In some embodiments, the ungulate-derived polyclonal human immunoglobulin compositions comprise about 90% IgG1, about 12% IgG2, and less than 0.1% IgG3 and/or IgG4. In some embodiments, ungulate-derived polyclonal human immunoglobulin compositions comprise at least about 70% IgG1. In some embodiments, human polyclonal immunoglobulins comprise less than about 30% IgG2. In some embodiments, human polyclonal immunoglobulins comprise less than about 4% IgG3 and/or IgG4.

In some embodiments, ungulate-derived polyclonal human immunoglobulin compositions comprise about 90% IgG1, about 10% IgG2 and less than 10% (e.g., less than 9, 8, 7, 6, 5, 4, 3, 2 1%) of IgG3 and/or IgG4.

### Glycosylation Profiles of SAB-142 Immunoglobulins

Glycosylation is the enzymatic process of adding carbohydrate moieties (glycans) to proteins, which influences antibody structure and function. SAB-142 comprises N-glycolylneuraminic acid (NGNA)-bearing glycans and fucosylated glycans characteristic of ungulate-derived antibodies. N-glycans are found in both human and bovine IgG, and they are carbohydrates that attach to a nitrogen atom of an asparagine amino acid in the immunoglobulin. These post-translational modifications primarily occur on the Fc region of immunoglobulins and are species-specific, with ungulate glycosylation patterns differing from those of both human and rabbit systems. Glycosylation patterns may influence antibody effector functions, including complement-dependent cytotoxicity and antibody-dependent cellular cytotoxicity, as well as antibody stability, pharmacokinetics, and immunogenicity profiles. Glycosylation of immunoglobulins can influence protein folding, structural stability, functional activity, localization, intracellular trafficking, and overall lifespan.

The glycosylation characteristics of SAB-142 were analyzed to determine the specific glycan structures present on the immunoglobulin molecules. N-glycans were enzymatically released from SAB-142 samples and compared to human intravenous immunoglobulin (hIVIG) as a reference standard. The released glycans were labeled and analyzed using hydrophilic interaction liquid chromatography with fluorescence detection and mass spectrometry (HILIC FLR-MS). Glycan assignments were made by comparing observed glucose unit (GU) values with library values, with confirmation by mass spectrometry. TABLE 3 and TABLE 4 summarize the glycosylation features and sialic acid content of SAB-142 compared to hIVIG. The hIVIG used was Gammagard, an immunoglobulin therapy used to treat primary immunodeficiency diseases, containing antibodies made from human plasma.

N-linked glycans were successfully released and labeled with RapiFluor-MS prior to analysis with HILIC FLR-MS. Glycans were assigned by comparing observed GU values with library values, which were then confirmed by mass spectrometry. Where mass spectral evidence was not consistent with any GU-based assignments, potential compositions have been provided based on the observed masses.

TABLE 3
describes different glycosylation features
compared between hIVIG and SAB-142.
Glycan Release

| Glycosylation Feature | hIVIG | % SAB-142 |
|---|---|---|
| Fucosylation | 95.2 | 93.6 |
| Bisecting GlcNAc | 16.8 | 8.4 |
| Alpha-gal | ND | 5.7 |
| Mono Sialylation | 15.1 | 11.4 |
| Di-Dialylation | 4.9 | 6.2 |
| Tri-Sialylation | ND | ND |
| Tetra-Sialylation | ND | ND |
| Total Sialylation | 20.0 | 13.9 |

ND: Not Detectable

Released N-linked glycans observed in FLR chromatograms were analyzed (data not shown). For GAMMAGARD F(6)A2G(4)1 was the most abundant glycan at 29.6%. F(6)A2, F(6)A2B, A2G(4)1, F(6)A2BG(4)1, A2G(4)2, F(6)A2G(4)2,F(6)A2BG(4)2, F(6)A2G(4)1S(6)1, F(6)A2G(4)2S(6)1, F(6)A2BG(4)1S(6)1, F(6)A2G(4)2S(6,6)2 and F(6)A2BG(4)2S(6,6)2 were also observed above 1%. Summary calculations for the sample show the majority of the glycans observed were fucosylated (95.2%). 16.8% of glycans contained bisecting GlcNAc. 20.0% of the observed glycans were Sialylated. No glycans containing NeuGc were observed. For SAB-142, F(6)A2G(4)1 was the most abundant glycan at 33.3%. F(6)A2, F(6)A2B, A2G(4)1, F(6)A2BG(4)1, A2G(4)2, F(6)A2G(4)2, F(6)A2BG(4)2, F(6)A2G(4)2Sg(6)1 were observed above 1%, as well as multiple glycans that were identified with potential compositions based on mass spectral evidence, or coeluting glycans. Summary calculations for SAB-142 glycan release show the majority of the glycans observed were fucosylated (93.6%). 8.4% of glycans contained bisecting GlcNAc. 13.9% of the observed glycans were sialylated and contained primarily NeuGc (1.056 μg/mg protein) with minor quantities of NeuAc (0.021 μg/mg protein).

The glycosylation analysis further included quantitative assessment of sialic acid content to characterize the specific sialic acid species present in SAB-142. Sialic acids are terminal monosaccharides that cap glycan structures and may influence antibody properties including serum half-life, immunogenicity, and effector function. Two primary forms of sialic acids were evaluated: N-acetylneuraminic acid (Neu5Ac), which is the predominant form in human immunoglobulins, and N-glycolylneuraminic acid (Neu5Gc), which is characteristic of non-human mammalian species including ungulates. TABLE 4 presents the sialic acid quantitation results for SAB-142 compared to hIVIG.

Sialic Acid Quantitation: Neu5Ac and Neu5Gc were successfully released from samples. Subsequently, glycans were labeled with and quantified by HPLC-FLR using Agilent's AdvanceBio Sialic Acid Profiling and Quantitation kit. Combined Neu5Ac and Neu5Gc standards were prepared to 0.025, 0.1, 0.4, 1, 1.6, 3.2, 3.6 and 5.0 μM for peak area calibration. A sialic acid reference panel (SARP) was prepared for identification of sialic acids based on retention time. A theoretical molecular weight of 150 kDa was used for conversion between molar and mass-based values.

TABLE 4
describes salic acid differences between hIVIG and SAB-142.
Sialic Acid Quantitation

| | Neu5Gc | | | Neu5Ac | | |
|---|---|---|---|---|---|---|
| | Concen- | | | Concen- | | |
| | tration | mmol/mol | ug/mg | tration | mmol/mol | ug/mg |
| | (uM) | protein | protein | (uM) | protein | protein |
| hIVIG | ND | ND | ND | 4.16 | 416.0 | 0.858 |
| SAB-142 | 4.87 | 487.2 | 1.056 | 0.10 | 10.3 | 0.021 |

ND: Not Detectable

Glycan release data demonstrate many similarities between GAMMAGARD (hIVIG) and SAB-142. Total sialylation levels of GAMMAGARD (20%) and SAB-142 (13.9%) are both low with the majority of glycans being mono sialylated and the remainer di-sialylated. In both SAB-142 and GAMMAGARD, no tri-sialylation or tetra-sialylation was detected. High levels of SAB-142 fucosylation (93.6%) were also similar to GAMMAGARD (95%). SAB-142 bisecting GlcNAc was measured at 8.4%, within the range of native human IgG (8-15%) and similar to the measured value of GAMMAGARD at 16.8%. In contrast to the similarities, SAB-142 contains two notable unique differences in glycan structure. Alpha-galactose content of SAB-142 was 5.7% verses undetectable levels in GAMMAGARD. The alpha-galactose content in SAB-142 is consistent with non-primate mammals normal glycosylation patterns due to the specific expression of α-1,3-galactosyltransferase (GGTA1), which adds galactose residues in an α-1,3 linkage to glycoproteins and glycolipids. GGTA1 expression was lost due to a frameshift mutation in Old World Primates resulting in it becoming a pseudogene in humans. Likewise, the terminal sialic acid content of SAB-142 contains some NGNA in addition to minor levels of NANA, whereas GAMMAGARD maintains undetectable levels of NGNA and all neuraminic acids are NANA. The presence of NGNA is due to differences in animal sialic acid biosynthesis. In non-human mammals, cytidine monophosphate-N-acetylneuraminic acid hydroxylase (CMAH) converts Neu5Ac to Neu5Gc by addition of a hydroxyl group to the N-acetyl group. Humans have lost CMAH activity due to gene inactivation. These data show a unique composition of SAB-142.

In some embodiments, the population of ungulate-derived polyclonal human immunoglobulin compositions comprises glycans covalently linked to the human immunoglobulins. In some embodiments, the glycans can be N-Glycolylneuraminic acid (NGNA) and/or N-Acetylneuraminic acid (NANA) moieties. Naturally occurring human immunoglobulin G isolated from humans, comprises N-Acetylneuraminic acid (NANA) moieties only. Ungulate-derived polyclonal human immunoglobulin compositions, in contrast, can comprise both NANA-bearing glycan moieties and N-Glycolylneuraminic acid (NGNA)-bearing glycan moieties. In some embodiments, the percentage of glycans that are N-Acetylneuraminic acid (NANA) moieties in ungulate-derived polyclonal human immunoglobulin compositions is about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more. In some embodiments, the percentage of N-Glycolylneuraminic acid (NGNA)-bearing glycans is about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, an ungulate-derived polyclonal human immunoglobulin composition comprises at least about 10%, 20%, 30%, 40%, 50%, 60%, 70%, at least about 80%, or at least about 90% N-Glycolylneuraminic acid (NGNA)-bearing glycans. In some embodiments, an ungulate-derived polyclonal human immunoglobulin composition comprises at least about 10%, 20%, 30%, 40%, 50%, 60%, 70%, at least about 80%, or at least about 90% NANA-bearing glycans. In some embodiments, an ungulate-derived polyclonal human immunoglobulin composition comprises less than 100%, less than 90%, less than 80%, less than 70%, less than 60%, less than 50%, less than 40%, less than 30%, less than 20%, less than 10%, less than 5%, or less than 1%.

### SAB-142 Composition Purity and Characterization

In some embodiments, the population of fully human or substantially human immunoglobulins are purified from the serum of the transchromosomic ungulate after immunization.

Residual non-human proteins are tightly controlled through comprehensive analytical testing and stringent manufacturing controls. The purification process is specifically designed to remove bovine-derived contaminants while preserving the fully human immunoglobulin content. Residual bovine IgG, residual bovine plasma proteins, and residual bovine serum albumin each have a stringent specification limit of less than 100 parts per million (ppm), ensuring minimal contamination from the source material. Clinical lots consistently demonstrated levels below the stringent purification criteria for all major bovine protein categories, indicating exceptional purity of the final drug substance. This rigorous control of bovine protein contaminants practically ensures that human IgG content is essentially human IgG1, demonstrating the effectiveness of the purification process in producing a substantially human immunoglobulin composition.

The major impurities in adsorbed transchromosomic bovine plasma are hemoglobin, bovine serum albumin (DSA), and other bovine plasma proteins (BPP). Removal of these impurities is important in the purification process. The disclosure includes the description of a series of steps that leads to a surprisingly high purity human immunoglobulin, SAB-142. The final composition has very few impurities, as shown in TABLE 5.

TABLE 5 shows the purity of the SAB-142 immunoglobulins.

| Impurity | Impurity | Molecule | | Mechanism of | Detection | SAB-142 |
|---|---|---|---|---|---|---|
| Name | Description | Description | Source | Removal | Method | Specification |
| Bovine | Residual | Large | Bovine | Precipitation by | Residual | <100 ppm |
| Serum | BSA | molecular | | caprylic acid | BSA ELISA | |
| Albumin | | weight | | fractionation, | | |
| (BSA) | | bovine | | Kappa affinity | | |
| | | albumin | | chromatography | | |
| Bovine | Residual | Variable | Bovine | Precipitation by | Residual | <100 ppm |
| Plasma | BPP | protein | | caprylic acid | BPP ELISA | |
| Protein | | structures | | fractionation, | | |
| (BPP) | | | | Kappa affinity | | |
| | | | | chromatography | | |
| | | | | and Q | | |
| | | | | Sepharose ® | | |
| | | | | chromatography | | |
| Bovine | Residual | Bovine | Bovine + | HC15 | Residual | <100 ppm |
| IgG | bovine IgG | heavy chain | Human | chromatography | Bovine IgG | |
| | | constant | Artificial | step | ELISA | |
| | | domain and | Chromosome | | | |
| | | human | (HAC) | | | |
| | | variable | | | | |
| | | domain | | | | |

SAB-142 is a fully or substantially fully human polyclonal anti-thymocyte immunoglobulin produced from the plasma of transchromosomic bovines immunized with purified human thymocytes from pediatric donors. The general characteristics of human immunoglobulins include the elucidation of relevant structure attributes, potency, and binding properties which are described below.

Examples of methods for detecting and measuring SAB-142 in a composition include a binding assay by an enzyme-linked immunosorbent assay, and the like. The binding amount of a human immunoglobulin may be measured by incubating the composition comprising the human immunoglobulin with cells (e.g., thymocytes, T cells, B cells and/or monocytes, or recombinant protein antigen(s)),and then using an antibody specifically recognizing human immunoglobulin.

Size exclusion chromatography, high performance liquid chromatography and sodium dodecyl-sulfate polyacrylamide gel electrophoresis showed that SAB-142 is of high purification, with over 95% monomer plus dimer, no more than 3% IgG aggregates, and no more than 5% IgG aggregates plus fragments. A first tested lot showed 99.47% monomer and 0.54% dimer, for a total of 100% monomer/dimer. There were 0% IgG aggregates and zero fragments. A second tested lot showed 99.46% monomer and 0.54% dimer for a total of 100% monomer/dimer, and there were 0% IgG aggregates or fragments. In addition, there were no more than 1 CFR/10 mL of viable microorganisms such as aerobic bacteria, anaerobic bacteria, fungi or spores. Endotoxins were limited to n more than 0.2 EU/mg (EU/mL). This high degree of purity and structural integrity is important for maintaining the therapeutic efficacy and safety profile of the anti-thymocyte globulin product, as lower-quality antibodies could potentially lead to adverse immunogenic responses or reduced therapeutic activity.

The transchromosomic bovine plasma contains several forms of immunoglobulins, including human IgG, bovine IgG, chimeric, and trans-bovine (t-bovine) IgG. Bovine IgG, chimeric, and t-bovine IgG are removed during the manufacturing process. Residual bovine IgG is quantified using the sandwich enzyme-linked immunosorbent assay (ELISA). Bovine IgG concentrations in drug substances are determined to ensure these impurities have been adequately removed. The bovine IgG ELISA is similar to the one discussed for residual bovine serum albumin (BSA). The anti-bovine IgG and anti-bovine IgG conjugated with horseradish peroxidase (HRP) are used as a capture and a secondary antibody, respectively. The lower limit of quantitation (LLOQ) for this method is 15.6 ng/mL.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transchromosomic ungulate with thymocytes; (2) collecting antibody-producing cells from the transchromosomic ungulate (i.e. from lymph nodes); (3) fusing the antibody-producing cells with myeloma cells; (4) selecting hybridomas that produce a monoclonal antibody specific to thymocytes from the fused cells obtained in the above step; and optionally (5) selecting a hybridoma that produces a monoclonal antibody specific to thymocytes from the selected hybridomas.

In embodiments of the methods of producing human anti-thymocyte globulin (ATG) of the disclosure, the transchromosomic ungulate produces human anti-thymocyte globulin (ATG). The method may comprise collecting the polyclonal serum and/or polyclonal plasma from the transchromosomic ungulate. In some embodiments, the ungulate is a bovine. In some embodiments, the polyclonal immunoglobulin composition comprises a population of fully human immunoglobulins, or of substantially human immunoglobulins.

Some embodiments of the methods of the disclosure, and related compositions, have the surprising advantage that the thymocyte-specific immunoglobulins are produced in high yield, in high purity, and/or as a high percentage of total immunoglobulin present in the serum or plasma of the transchromosomic ungulate. In some embodiments, the ungulate is a bovine.

In some embodiments of the methods and compositions of the disclosure, the polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-0.5%, 0.5-1%, 1-1.5%, 1.5-2%, 2-2.5%, 20.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9- 9.5%, 90.5-10% or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, the polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, the polyclonal immunoglobulin comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, the polyclonal immunoglobulin comprises 0-1%, 1-2%, 2-3%, 3-4%, 4-5%,5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, the polyclonal immunoglobulin comprises at least 5% fully human immunoglobulin by mass of total immunoglobulin in the polyclonal immunoglobulin.

In some embodiments of the methods and compositions of the disclosure, the polyclonal immunoglobulin comprises 2% to 5% fully human immunoglobulin by mass of total immunoglobulin in the polyclonal immunoglobulin.

In some embodiments, the ungulate-derived polyclonal immunoglobulin comprises “chimeric” human immunoglobulin having a human heavy chain and an ungulate kappa light chain (termed “cIgG”). In some embodiments, the polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

### Stability Profile and Storage Characteristics

SAB-142 is stored at 5±3° C. and maintains stability throughout the manufacturing process and storage conditions. The drug substance demonstrates robust stability profiles under controlled refrigerated conditions, with validated shelf-life specifications ensuring maintenance of potency, purity, and safety parameters. The composition undergoes terminal sterilization and fill processes under aseptic conditions to ensure sterility without compromising the integrity of the polyclonal immunoglobulin composition. The terminal sterilization process is validated to achieve appropriate sterility assurance levels while preserving the biological activity and structural integrity of the fully human anti-thymocyte globulin. Visual appearance at 1 month, 3 months, and 6 months was essentially free of particles; the composition was colorless, with low to no turbidity. The pH was stable at 5.6-5.7, with a protein concentration at To of 70.74 compared to a concentration at 6 months of 70.93, showing the high stability of the composition. In addition, at 6 months, the monomer was detected at 99.23%, the dimer at 0.68%, with no IgG aggregates or fragments. Heavy chain bands on an SDS gel was consistently visible at 62 kDa and 79 kDa, with a light chain band at 23 kDa (TO), 24 kDa (1 month), 24 kDa (3 months), and 24 kDa (6 months). An additional heavy chain band was visible at 108 kDa by month 6. Anti-human PBMC CDC showed an LT₂₀ of 6.68 μg/mL and LT₅₀ of 9.21 μg/mL at T₀, an LT₂₀ of 6.4 μg/mL and LT₅₀ of 7.93 μg/mL at 1 month, an LT₂₀ of 6.32 μg/mL and LT₅₀ of 9.18 μg/mL at 3 months, an LT₂₀ of 4.87 μg/mL and LT₅₀ of 7.46 μg/mL at 6 months. The bioburden was consistently 0 CFR/5 mL throughout the testing period (up to 6 months). Additional testing is verifying stability out to 24 months.

At accelerated storage conditions of 25±2° C., 60±5% relative humidity, visual appearance was essentially free of particles by 3 months, the composition was colorless, and turbidity was less than the reference suspension. The visible particles were less than the reference solution, and the pH was consistently at 5.6. Protein concentration was 70.74 at T₀, 73.84 at 3 months, and 76.09 at 6 months. In addition, the monomer was detected at 98.32% by 6 months compared to 99.46% at TO, the dimer was at 1.05% at 6 months compared to 0.54% at T₀, and fragments were detected at the low level of 0.63% by 6 months (no IgG aggregates), compared to no IgG aggregates or fragments at To. Purity as measured by CE SDS was at 92% at To and 91% at 6 months. Though the temperatures were relatively high and humidity was high as well, anti-human PBMC were detected at an LT₂₀ of 6.68 μg/mL and LT₅₀ of 9.21 μg/mL at T₀, and detected at an LT₂₀ of 5.58 μg/mL and LT₅₀ of 9.8 μg/mL at 6 months. Throughout the test period, CFR was 0/5 mL.

### Polyclonal Immunoglobulin Target Recognition

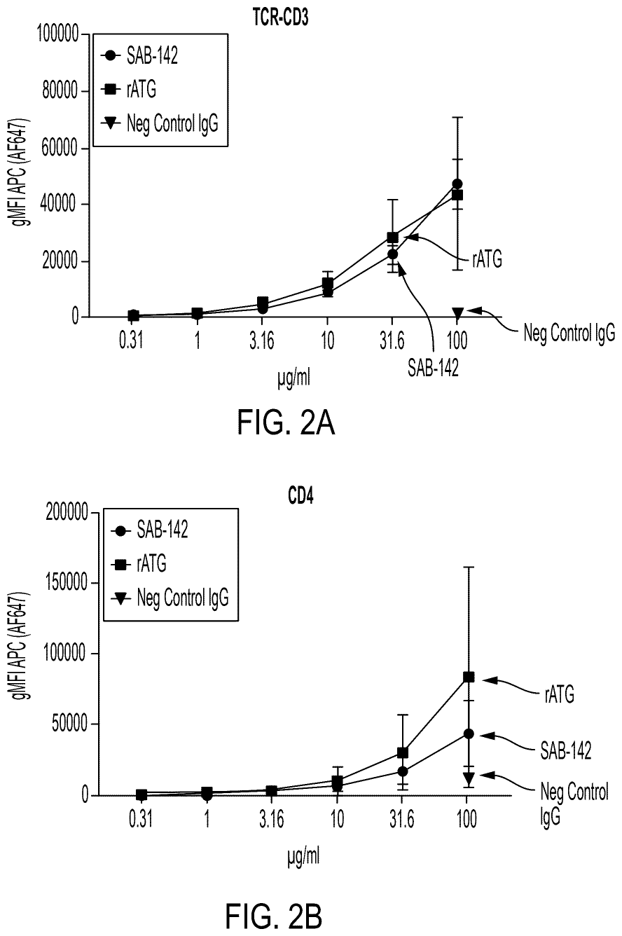

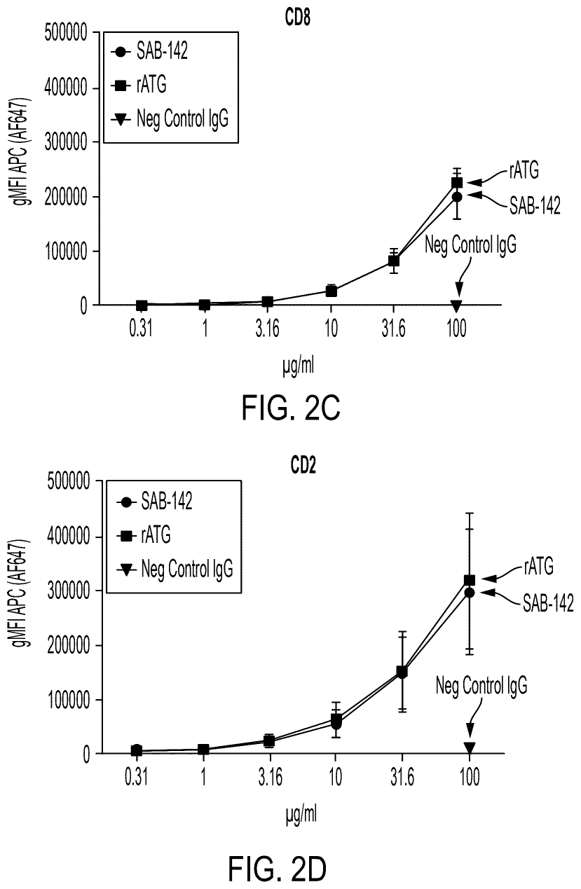

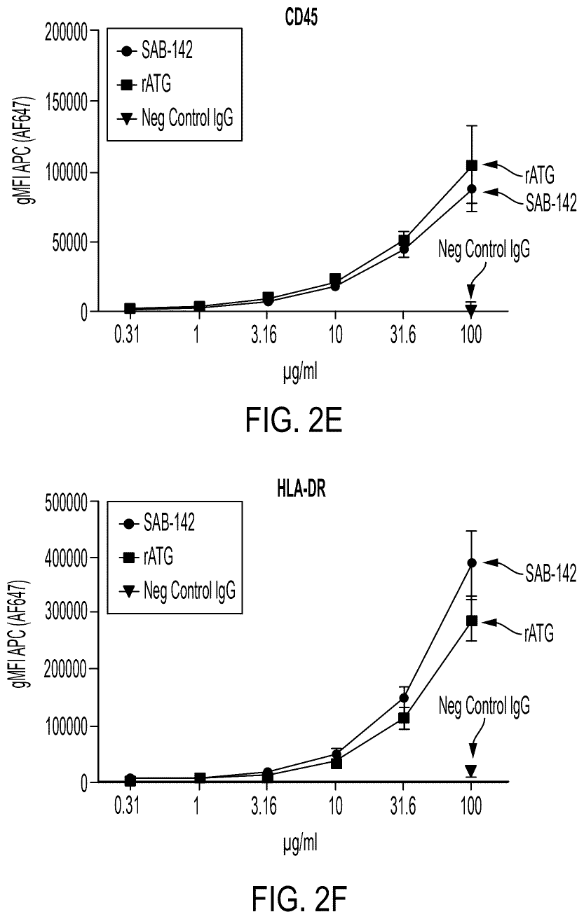

Surface plasmon resonance analysis demonstrates that SAB-142 exhibits comparable antigen binding characteristics to rabbit anti-thymocyte globulin across multiple CD markers. The polyclonal immunoglobulins of SAB-142 provide diverse binding specificities with varying avidities, contributing to its therapeutic efficacy. The polyclonal nature of SAB-142 may enable enhanced binding through multiple simultaneous interactions with different epitopes on target antigens, resulting in increased avidity and improved functional binding compared to monoclonal antibodies that recognize only single epitopes. This multivalent binding capability allows the polyclonal immunoglobulins to engage target cells through multiple binding sites with diverse affinities, potentially providing more robust and sustained immune cell engagement. SAB-142 demonstrates binding to human thymocytes, T cells, B cells, and monocytes, providing broad immunomodulatory activity against target immune cell populations involved in autoimmune processes. Binding studies show dose-dependent increases in fluorescence intensity, with SAB-142 demonstrating strong binding to target antigens across a range of concentrations from 0.31 to 100 μg/mL. Flow cytometry analysis reveals that SAB-142 binds to CD45+ cells, CD2+ cells, CD3+ cells, CD4+ cells, and CD8+ cells with binding profiles similar to those observed with rabbit anti-thymocyte globulin (FIG. 2B- FIG. 2E). The binding characteristics of SAB-142 to these immune cell surface proteins support its mechanism of action as an immunomodulatory agent for the treatment of autoimmune conditions such as type 1 diabetes.

The antigen binding characteristics of SAB-142 are defined by both affinity and specificity parameters that contribute to its therapeutic profile. Affinity refers to the strength of interaction between individual antibody molecules and their target epitopes, while specificity describes the selectivity of antibody binding to particular antigens over others. In the context of polyclonal immunoglobulin compositions such as SAB-142, the overall binding strength is characterized by avidity, which encompasses the collective binding interactions of multiple antibody species with multiple epitopes on target antigens. Avidity measurements may be determined using surface plasmon resonance (SPR) techniques with resonance units (RU) expressed as off-rate constants or residence time (the inverse of the off-rate constant). Avidity measurements may be also determined using a chaotropic ELISA assay. Higher avidity is indicated by slower off-rate constants, longer residence times, and increased resonance units. The polyclonal nature of SAB-142 provides enhanced binding strength compared to individual antibody species, likely due in part to the cooperative binding effects of multiple antibodies engaging multiple epitopes simultaneously. This multivalent binding capability allows SAB-142 to recognize diverse epitopes on thymocytes, T cells, B cells, and monocytes, providing broad immunomodulatory activity while maintaining specificity for target immune cell populations involved in autoimmune processes.

Surface plasmon resonance analysis was performed to characterize the avidity of SAB-142 for key T cell surface markers. The binding kinetics were evaluated by measuring resonance units (RU) as a function of antibody concentration and time, with data collected across multiple CD markers including CD2, CD3, CD4, and CD8. The experimental measurements demonstrated that SAB-142 exhibits measurable avidity to these target antigens, with residence times calculated from the dissociation phase of the binding curves. For CD2, SAB-142. By SPR, SAB-142 exhibits measurable resonance units against CD2, CD3, CD4, and CD8, with RU magnitudes within the disclosed range at 0.1 mg/mL and higher relative binding to CD8 than CD4; lot-to-lot differences reflect pooling strategy. The off-rate constants (k_{off}) were determined from the dissociation phase of the sensorgrams, with residence times (1/k_{off}) calculated to quantify the duration of antibody-antigen complex stability. These measurements provide a quantitative assessment of SAB-142's binding characteristics to multiple T cell surface markers, demonstrating the polyclonal composition's capacity for multivalent engagement with target immune cell populations.

Example 2 describes the results of testing the binding profile of SAB-142, which showed the highest binding for CD8 compared to the other cell surface markers tested. SAB-142 has been shown to bind to CD2, TCR-CD3, CD4, CD8, HLA-DR and CD45 surface proteins in a manner similar to rATG (FIG. 2A- FIG. 2E)

In some embodiments, SAB-142 exhibits differential binding affinities to specific T cell surface markers compared to rATG. In further embodiments, SAB-142 exhibits binding to CD8 that is within 5%, 10%, 15%, or 20% of the binding affinity of rATG to CD8. In additional embodiments, SAB-142 exhibits binding to CD4 that is within 5%, 10%, 15%, or 20% of the binding affinity of rATG to CD4. In additional embodiments, SAB-142 exhibits binding to HLA-DR that is within 5%, 10%, 15%, or 20% of the binding affinity of rATG to HLA-DR. The binding titers of SAB-142 to CD3, CD4, and CD8 were quantified by measuring its interaction with purified human ATG. The data below in TABLE 6 demonstrates that SAB-142 binds to CD3, CD4, and CD8. Further, as summarized in TABLE 6, these data may corroborate prior assay results by demonstrating that SAB-142 binds CD3 with markedly higher binding titer activity than binding CD4 or CD8. Together, the findings indicate a pronounced preference of SAB-142 for CD3 relative to the other T-cell surface markers, with TABLE 6 providing the comparative binding profile across the three targets.

TABLE 6
shows comparative binding values for CD3,
CD4, and CD8 in U/mg for SAB-142.

| CD | SAB-142 (U/mg) |
|---|---|
| CD3 | 37150 |
| CD4 | 5180 |
| CD8 | 6245 |

In some embodiments, SAB-142 demonstrates binding to CD2 that is within 5%, 10%, 15%, or 20% of the binding affinity of rATG to CD2. In other embodiments, SAB-142 exhibits binding to CD3 that is within 5%, 10%, 15%, or 20% of the binding affinity of rATG to CD3. In alternative embodiments, SAB-142 demonstrates binding to CD45 that is within 5%, 10%, 15%, or 20% of the binding affinity of rATG to CD45.

Avidity of SAB-142 and rATG to recombinant human CD3, CD4, and CD8 was assessed by a chaotropic ELISA to compare the binding characteristics of the two anti-thymocyte globulin products. High-binding plates were coated with purified human thymocytes, blocked, and incubated with serial dilutions of SAB-142 or rATG under standardized conditions. After equilibrium binding, plates were washed and challenged with a chaotropic agent (e.g., sodium thiocyanate or urea) at either a predefined discriminating concentration or across a graded series, followed by a wash and detection of residual bound SAB-142 or rATG using an HRP-conjugated secondary specific to the antibody isotype, TMB development, and OD450 measurement. The avidity index was calculated as the fraction of binding retained after chaotrope treatment relative to the matched no-chaotrope control at the same antibody concentration, or alternatively as the chaotrope concentration that reduced binding by 50% (AI50) derived from a 4-parameter logistic fit. Results were reported as mean avidity index (AI50) across technical replicates for both SAB-142 and rATG, as shown in TABLE 7.

TABLE 7
shows the measured avidity index compared
between SAB-142 and rATG.

| CD3 Avidity Index | | CD8 Avidity Index | | CD4 Avidity Index | |
|---|---|---|---|---|---|
| SAB-142 | 0.69 | SAB-142 | 3.28 | SAB-142 | 0.97 |
| rATG | 0.23 | rATG | 4.23 | rATG | 0.69 |

TABLE 8
describes product impurities of purified human ATG.

| | | | PD2501059TT |
|---|---|---|---|
| Test | | Units | (Lot 4) |
| Anti-A, Anti-B Hemagglutinins | A | mg/mL | 0.125 |
| | B | mg/mL | 0.125 |
| | O | mg/mL | 0.125 |
| Anti-CD3 Inhibition Activity | | μg/mL | 170 |
| Anti-D Hemagglutinins | O+ | mg/mL | 0.125 |
| | O− | mg/mL | 0.125 |

| | | |
|---|---|---|
| Anti-Human Fibroblast CDC | % CDC | <2 |
| Anti-Human PBMC CDC | μg/mL | 18.3 |
| Anti-GBM | U/mL | <10 |
| Anti-Human Platelet Binding | μg/mL | >52.5 |
| Activity | | |
| Anti-Human Serum Protein | μg/mL | 0.26 |
| Antibodies (at 100 μg/mL) | | |
| Hemagglutination Assay for ATG | μg/mL | 10000 |

The purified human ATG provided herein was developed to have low antibodies to human serum proteins and low binding to human platelets, as shown in TABLE 9.

TABLE 9
describes the quality aspects of the purified human ATG.

| Test | Quality Attribute | Acceptance Criteria | |
|---|---|---|---|
| Anti-Complement Activity (ACA) | Process Related Impurity (Safety) | ACA % ≤ Complement | |
| (Ph. Eur. 2.6.17) | | Control (100%) | |
| Anti-A, Anti-B Hemagglutinins | Process Related Impurity (Safety) | ≥7.813 | μg/mL |
| (Ph. Eur. 2.6.20) | | | |
| Anti-CD3 Antibody Activity | Potency | EC₅₀ 75-300 | μg/mL |
| Anti-D Hemagglutinins | Process Related Impurity (Safety) | ≥15.625 | μg/mL |
| (Ph. Eur. 2.6.26) | | | |
| Anti-GBM | Process Related Impurity (Safety) | ≤20 | U/mL |
| Anti-Human Fibroblast CDC | Process Related Impurity (Safety) | PL₂₀₀ ≤ 10% | |
| Anti-Human PBMC CDC* | Potency/Identity | LT₅₀: 4.4-27.2 μg/mL | |
| Anti-Human Platelet Binding | Process Related Impurity (Safety) | BC₅₀ ≥ 1.0 | μg/mL |
| Anti-Human Serum Protein | Process Related Impurity (Safety) | ≤1.2 | μg/mL |
| Antibodies | | | |
| Hemagglutination Assay for ATG | Process Related Impurity (Safety) | ≥50 | μg/mL |

In some embodiments, the fully human or substantially human immunoglobulins bind to an immune cell surface protein with an avidity (dissociation rate constant) of a population of immunoglobulins that bind to an immune cell surface protein, with an avidity of at least 1×10⁻⁵, at least 1×10⁻⁶ or at least 1×10⁻⁷ at a concentration of 0.1 mg/ml of the composition. In some embodiments, the cell surface protein may be CD2, CD3, CD4 and/or CD8. In some embodiments, the avidity can be at least 1×10⁻⁷ to 1×10⁻¹ at a concentration of 0.1 mg/ml of the composition. In some embodiments, the avidity can be from about 1×10⁻² to 1×10⁻⁵ at a concentration of 0.1 mg/ml of the composition. In some embodiments, the compositions described herein can include immunoglobulins with high avidity (e.g., avidity (dissociation rate constant) of 1×10⁻⁵), low avidity (e.g., avidity (dissociation rate constant) of 1×10⁻¹) or a combination thereof.

In some embodiments, the polyclonal immunoglobulins of the disclosure are more potent in a complement-dependent cytotoxicity (CDC) assay than a reference product (e.g. Thymoglobulin® or ATGAM®). In some embodiments, the polyclonal immunoglobulins of the disclosure are at least about 5%, at least about 10%, at least about 25%, at least about 50%, at least about 100%, at least about 150%, or more at least about 200% potent in a complement-dependent cytotoxicity (CDC) assay than a reference product (e.g. Thymoglobulin® or ATGAM®).

In some embodiments, the polyclonal immunoglobulins of the disclosure generate higher toxicity towards CD8+ cells than a reference product (e.g., Thymoglobulin® or ATGAM®). In some embodiments, the polyclonal immunoglobulins of the disclosure are at least about 5%, at least about 10%, at least about 25%, at least about 50%, at least about 100%, at least about 150%, or at least about 200% more potent in CD8+ cell killing assay than a reference product (e.g. Thymoglobulin® or ATGAM®).

In some embodiments, the polyclonal immunoglobulins of the disclosure generate lower rates of CD4+ T cell apoptosis than a reference product (e.g., Thymoglobulin® or ATGAM®. In some embodiments, the polyclonal immunoglobulins of the disclosure are at least about 5%, at least about 10%, at least about 25%, at least about 50%, at least about 100%, at least about 150%, or at least about 200% less toxic in a CD4+ cell apoptosis assay than a reference product (e.g. Thymoglobulin® or ATGAM®).

In some embodiments, the polyclonal immunoglobulins of the disclosure better preserve T_{reg} to conventional T cell ratios than a reference product (e.g., Thymoglobulin® or ATGAM®). In some embodiments, the polyclonal immunoglobulins of the disclosure are at least about 5%, at least about 10%, at least about 25%, at least about 50%, at least about 100%, at least about 150%, or at least about 200% less toxic to T_{reg} cells than a reference product (e.g., Thymoglobulin® or ATGAM®).

### Binding Avidity of SAB-142

In some embodiments of the methods and compositions of the disclosure, the population of fully human immunoglobulins (or substantially human) specifically binds human thymocytes, T cells, B cells, and/or monocytes. In some embodiments, the population of fully human (or substantially human) immunoglobulins specifically binds human thymocytes.

In some embodiments, the fully human or substantially human immunoglobulins can bind to an immune cell surface protein with a resonance unit of at least 5, at a concentration of about 0.1 mg/ml. The resonance unit can be measured by using surface plasmon resonance. In some embodiments, the immune cell surface protein can be CD2, CD3, CD4, and/or CD8.

In some embodiments, the resonance unit can be about 5 to 350, 10 to 350, 20 to 350, 30 to 350, 40 to 350, 50 to 350, 60 to 350, 70 to 350, 80 to 350, 90 to 350, 100 to 350, 110 to 350, 120 to 350, 130 to 350, 140 to 350, 150 to 350, 160 to 350, 170 to 350, 180 to 350, 190 to 350, 200 to 350, 210 to 350, 220 to 350, 230 to 350, 240 to 350, 250 to 350, 260 to 350, 270 to 350, 280 to 350, 290 to 350, 300 to 350, 310 to 350, 320 to 350, 330 to 350, 340 to 350,5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, or 5 to 340, at a concentration of 0.1 mg/mL of the composition.

Treatment of Diseases with SAB-142

In some embodiments, the compositions (eg., SAB-142) and methods of the disclosure may be used for treating conditions beyond type 1 diabetes, including organ transplantation, and islet cell transplantation. In some embodiments, the compositions (eg., SAB-142) and methods of the disclosure may be used for treating various autoimmune diseases, such as celiac disease, systemic lupus erythematosus (SLE), scleroderma, polymyositis, dermatomyositis, etc. These conditions share a common underlying mechanism involving dysregulated or undesirable T-cell activity and may benefit from immunomodulatory approaches that target T-cell populations.

In the context of organ transplantation, T-cell-mediated rejection represents a barrier to graft survival. Following transplantation of solid organs such as kidney, heart, liver, or lung, recipient T-cells may recognize donor antigens as foreign and mount an immune response against the transplanted tissue. CD4+ helper T-cells and CD8+ cytotoxic T-cells may infiltrate the graft and contribute to acute and chronic rejection through direct cytotoxicity and inflammatory cytokine release. Suppressing the T-cell response may reduce the risk of rejection episodes and support long-term graft function. In some embodiments, administration of SAB-142 may provide immunosuppression in organ transplant recipients while preserving regulatory T-cell populations that may promote tolerance to the transplanted organ.

Islet cell transplantation presents similar immunological challenges, where transplanted insulin-producing beta cells may be targeted by recipient T-cells. In addition to alloimmune rejection, patients with type 1 diabetes who receive islet cell transplants may also experience recurrent autoimmunity, wherein the same autoreactive T-cells that originally destroyed native beta cells may attack the transplanted islets. Suppressing T-cell activity may protect transplanted islets from both allorejection and autoimmune destruction, thereby supporting transplant maintenance and sustained insulin production. In some embodiments, SAB-142 may be administered to islet cell transplant recipients to modulate T-cell responses and promote graft survival.

Autoimmune diseases such as celiac disease, systemic lupus erythematosus (SLE), scleroderma, polymyositis, and dermatomyositis share pathological mechanisms involving dysregulated T-cell activity directed against self-antigens. In celiac disease, gluten-reactive CD4+ T-cells in the intestinal mucosa influence inflammation and villous atrophy through the release of pro-inflammatory cytokines and activation of cytotoxic intraepithelial lymphocytes. In SLE, autoreactive T-cells contribute to the production of autoantibodies and immune complex deposition affecting multiple organ systems including the kidneys, skin, and joints. Scleroderma involves T-cell infiltration into skin and internal organs, where CD4+ T-cells may promote fibroblast activation and excessive collagen deposition leading to tissue fibrosis. Polymyositis and dermatomyositis are inflammatory myopathies characterized by T-cell-mediated muscle damage, with CD8+ cytotoxic T-cells infiltrating muscle fibers in polymyositis and CD4+ T-cells contributing to inflammation in dermatomyositis.

In these autoimmune conditions, the therapeutic approach may involve selectively depleting pathogenic T-cell populations while preserving regulatory T-cells that help maintain immune tolerance. Regulatory T-cells play a role in suppressing autoreactive immune responses, and their preservation or enhancement may contribute to sustained disease control. In some embodiments, SAB-142 may modulate T-cell populations in autoimmune diseases by inducing T-cell exhaustion, reducing effector T-cell activity, and maintaining regulatory T-cell function.

The methods and compositions of the disclosure may also be used for treating patients at various stages of type 1 diabetes progression. In patients with Stage 3 new onset type 1 diabetes, autoreactive T-cells continue to destroy remaining beta cells, and early intervention may preserve residual beta cell function. In patients with recent onset type 1 diabetes, defined as greater than 100 days but less than one year from diagnosis, immunomodulation may slow the rate of beta cell decline. In patients with established type 1 diabetes, defined as greater than one year but less than two years from diagnosis, treatment may help maintain any remaining endogenous insulin production. In some embodiments, SAB-142 may be administered to patients across these stages of type 1 diabetes to delay disease progression and preserve beta cell function through modulation of autoreactive T-cell responses.

The common rationale underlying these therapeutic applications is that suppressing or modulating the T-cell response may be beneficial across conditions where T-cell activity contributes to tissue damage, whether through autoimmunity, alloimmunity, or both. By targeting T-cell populations while preserving regulatory immune function, the compositions and methods of the disclosure may provide therapeutic benefit in organ transplantation, islet cell transplantation, autoimmune diseases, and various stages of type 1 diabetes.

### Administration of SAB-142 in Type 1 Diabetes

SAB-142 is formulated for intravenous administration at doses of 1.5 mg/kg or 2.5 mg/kg, delivered as split doses over two consecutive days, with treatment periods occurring at baseline and Month 6, and optionally at Month 12 and Month 18 for extended treatment in patients with newly diagnosed type 1 diabetes. As a fully human ATG IgG, SAB-142 is expected to combine the beneficial immunomodulatory effects of existing anti-thymocyte globulin therapies with a more favorable safety profile, including reduced incidence of serum sickness and anti-drug antibody formation.

In some embodiments, a therapeutically effective amount of SAB-142 is administered to a type 1 diabetes patient, wherein the type 1 diabetes is treated. The SAB-142 may be administered at doses of 1.5 mg/kg or 2.5 mg/kg, delivered as split doses over two consecutive days, with treatment periods occurring at baseline and Month 6, and optionally at Month 12 and Month 18 for extended treatment.

In some embodiments, type 1 diabetes treatment success is measured by one or more endpoints selected from: preservation of p cell function as measured by C-peptide AUC during 2-hour MMTT, improvement in time in tight range (TITR) defined as percentage of time glucose is >70 but ≤140 mg/dL, reduction in HbA1c levels, improvement in time in range (TIR) defined as percentage of time glucose is >70 but 180 mg/dL, reduction in exogenous insulin use, reduction in clinically important hypoglycemic episodes, achievement of partial clinical remission defined as insulin requirement of <0.25 units per kg per day and HbA1c <6.5%, achievement of partial remission defined as insulin dose-adjusted A1c (IDAA1c)≤9, improvement in total BETA-2 score, with minimal to no serum sickness and minimal to no anti-drug antibodies (ADA) observed.

In some embodiments, treatment success is defined as a reduction in the decline of C-peptide AUC of at least 25%, 30%, 40%, or 50% compared to placebo at 12 months or 24 months. In some embodiments, treatment success is defined as HbA1c reduction of at least 0.2%, 0.3%, 0.4%, or 0.5% at 6 months, 12 months, or 24 months compared to baseline or placebo. In some embodiments, treatment success is defined as a reduction in daily insulin use of at least 10%, 15%, 20%, or 25% at 6 months, 12 months, or 24 months compared to baseline or placebo.

In some embodiments, SAB-142 treatment results in minimal to no serum sickness, defined as less than 5%, 3%, 2%, or 1% incidence of serum sickness compared to historical controls with rabbit anti-thymocyte globulin. In some embodiments, SAB-142 treatment results in minimal to no ADA formation, defined as less than 10%, 8%, 5%, or 3% incidence of anti-SAB-142 antibodies at 12 months or 24 months post-treatment initiation.

CP_{EST} will be determined at the specified timepoints using the following equation:

$${log}_{e} ( C {P}_{E S T} + 1 ) = 0.317 + 0.00956 \times BMI ( kg / {m}^{2} ) - 0.0 \; 00159 \times duration ( days ) + 0.71 \times fasting C - peptide ( nmol / L ) - 0.0117 \times fasting plasma glucose ( mmol / l ) - 0.0186 \times HbA 1 c ( \% ) - 0.0665 \times insulin ( U / kg ) .$$

The basis of the quantitative response (QR) is an analysis of covariance (ANCOVA) model of C-peptide that adjusts for baseline C-peptide and age. The QR is a standardized measure of the difference between an individual's observed and predicted C-peptide AUC mean one year after study entry. Values above zero indicate a better—than-expected outcome, and values below zero indicate a worse-than-expected outcome. QR is an exploratory endpoint, and will be calculated at Months 3, 6, and 12 (Parts A and B) and Months 18 and 24 (Part C). The following formula will be used for calculation of QR:

$$QR = ln ( C {p}_{1 year} + 1 ) - 0 \text{.812} \cdot ln ( {Cp}_{0} + 1 ) - 0.00638 \cdot Age + 0.191$$

where: Cp₀ and Cp_{1year} represent 2-hour C-peptide AUCₘₑₐₙ (AUC divided by 120 min, in nanomoles per liter) at baseline and one year post treatment, respectively. Age is the age at randomization, in years.

### Production of SAB-142 Transchromosomic Bovine Anti-Thymocyte Immunoglobulin

The composition is produced through immunization of transchromosomic bovines with fresh human thymocytes administered subcutaneously at multiple injection sites, followed by plasma collection and purification using protein affinity chromatography and red blood cell adsorption.

Provided are methods of producing transchromosomic bovine anti-thymocyte globulin, herein referred to as “SAB-142”, comprising administering human thymocytes to a transchromosomic ungulate. Thymocytes are hematopoietic progenitor cells present in the thymus. They are available from various sources, including pediatric and young adult cardiac surgeries, where thymus tissue must be removed from the patient and would normally be discarded. The thymocytes may be fresh human thymocytes, as fresh human thymocytes better preserve the conformation of surface antigens.

The disclosure further provides a method of producing a human antibody, comprising: (a) administering human thymocytes, or other target antigen of the disclosure, to the transchromosomic ungulate of any embodiment or combination of embodiments of the disclosure to produce and accumulate a population of human immunoglobulins specific to human thymocytes (or to T cells, B cells, and/or monocytes) in the serum or plasma of the ungulate; and optionally (b) isolating, recovering, and/or purifying the population of human immunoglobulins specific to the human thymocytes (or to T cells, B cells, and/or monocytes) from the serum or plasma of the ungulate.

In some embodiments, the antigen used to generate an ATG product may be, rather than a thymocyte, a cell sharing one or more endogenous protein markers with thymocytes, a cell recombinantly expressing one or more thymocyte proteins, recombinant thymocyte proteins, or nucleic acids that encode thymocyte proteins (e.g., RNA, linear DNA, or plasmid DNA).

### Appropriate Antigens for Production of SAB-142

In some embodiments, the method comprises administering an effective amount of human thymocytes. The fresh thymocytes are preferably administered within 24 hours of isolation, more preferably within 12 hours of isolation, and most preferably within 6 hours of isolation to maintain optimal viability and antigenicity. In embodiments, the effective amount is at least about 1×10⁸, at least about 5×10⁸, at least about 1×10⁹, at least about 5×10⁹, at least about 1×10¹⁰, or at least about 5×10¹¹ thymocytes.

The disclosure further provides compositions produced by immunizing a transchromosomic ungulate with human thymocytes, wherein the composition comprises a population of fully human or substantially human immunoglobulins and wherein the population of fully human or substantially human immunoglobulins specifically binds human thymocytes, T cells, B cells, and/or monocytes.

In a variation, non-human thymocytes are used (e.g., thymocytes of a domesticated animal such as a dog, cat, sheep, etc.). The transchromosomic ungulate may, in such cases, comprise an artificial chromosome encoding an Ig locus of the non-human species, such that antibodies of that species are generated.

The disclosure also provides a method of recovering the protein sequence of a human antibody that comprises: (i) isolating lymphocytes from the transchromosomic ungulate; (ii) generating a human monoclonal antibody-producing hybridoma from the lymphocytes; and (iii) recovering a human monoclonal antibody specific to the human thymocytes from the hybridoma.

In another embodiment, the lymphocytes from the transchromosomic ungulate are isolated from the lymph nodes of the transchromosomic ungulate. In a further embodiment, the transchromosomic ungulate is hyperimmunized with the human thymocytes or other target antigen of the disclosure.

The disclosure provides a “vaccine formulation” used to immunize transchromosomic bovines during production of SAB-142. This material is used to generate the hyperimmune plasma, not as part of the patient-facing drug substance formulation.

Illustrative adjuvants suitable for the thymocytes include an aluminum salt adjuvant, an oil-in-water emulsion (e.g., an oil-in-water emulsion comprising squalene, such as MF59®, an oil-in-water emulsion composed of squalene and two surfactants, Tween 80 and Span 85; or AS03®, an oil-in-water emulsion composed of squalene, polysorbate 80, and α-tocopherol (vitamin E)), a TLR7 agonist (such as imidazoquinoline or imiquimod), or a combination thereof. Suitable aluminum salts include hydroxides (e.g. oxyhydroxides), phosphates (e.g., hydroxyphosphates, orthophosphates), (e.g. see chapters 8 & 9 of Vaccine Design. (1995) eds. Powell & Newman. ISBN: 030644867X. Plenum), or mixtures thereof. Further illustrative adjuvants include, but are not limited to, Adju-Phos®, Adjumerlm, albumin-heparin microparticles, algal glucan, Algammulin™ (alum embedded in inulin particles converted to gamma inulin), alum, antigen formulation, AS02 adjuvant (monophosphoryl lipid A and QS21 (saponin from Quillaja saponaria Molina tree) in an oil in water emulsion), autologous dendritic cells, autologous PBMC, Avridine™, B7-2, benzalkonium chloride (BAK), BAY R1005 (a synthetic lipopeptide), bupivacaine, bupivacaine-HCl, calcitriol, calcium phosphate gel, CCR5 peptides, complete Freund's adjuvant (CFA), cholera holotoxin (CT) and cholera toxin B subunit (CTB), cholera toxin Al-subunit-protein A D-fragment fusion protein, CpG, CRL1005 (a copolymer based on a 12 kDa polyoxypropylene core with 5% polyoxyethylene), cytokine-containing liposomes, D-murapalmitine, dimethyl dioctadecyl ammonium bromide (DDA), dehydroepiandrosterone (DHEA), diphtheria toxoid, poly(DL-lactide-co-glycolide (DL-PGL), 1,2-dimyristoyl-sn-glycero-3-phosphocholine (DMPC), dimyristoylphosphatidylglycerol (DMPG), deoxycholate (DOC)/Alum complex, Fowlpox, Freund's complete adjuvant, gamma inulin, Gerbu Adjuvant, GM-CSF, glucosaminyl muramyl dipeptide (GMDP), hGM-CSF, hIL-12 (N222L), hTNF-alpha, IFA, IFN-7 in pcDNA3, IL-12 DNA, IL-12 plasmid, IL-12/GMCSF plasmid (Sykes®), IL-2 in pcDNA3, IL-2/Ig plasmid, IL-2/Ig protein, IL-4, IL-4 in pcDNA3, imiquimod, ImmTher™ immunoliposomes containing antibodies to costimulatory molecules, interferon-gamma, interleukin-1 beta, interleukin-12, interleukin-2, interleukin-7, ISCOM(s)™, Iscoprep™ 7.0.3, Montanide™ ISA-25, keyhole limpet hemocyanin, lipid-based adjuvant, liposomes, loxoribine, LT(R192G), LT-OA or LT oral adjuvant, LT-R192G, LTK63, LTK72, MF59, Montanide® ISA 51, Montanide® ISA 720, monophosphoryl lipid A (MPL), MPL-SE (monophosphoryl lipid A and squalene emulsion), muramyl tripeptide-phosphatidylethanolamine (MTP-PE), MTP-PE liposomes, murametide, murapalmitine, NAGO (neuraminidase and galactose oxidase), nCT native cholera toxin, non-ionic surfactant vesicles, non-toxic mutant El 12K of cholera toxin mCT-E112K, p-hydroxybenzoique acid methyl ester, pCIL-10, pCIL12, pCMVmCAT1, pCMVN, peptomer-NP, pleuran, poly-α-1-glutamine (PLG), poly(lactic-co-glycolic) acid (PLGA), poly-glutamic acid (PGA), and poly(lactic acid) (PLA), Pluronic® L121, poly(methyl methacrylate) (PMMA), PODDS™, poly(A:U), polysorbate 80, protein cochleates, QS-21, Quadri A saponin, Quil-A®, ISA-25/Quil-A®, Rehydragel® HPA, Rehydragel® LV, Ribi adjuvant system, Ribilike adjuvant system (MPL, TMD, CWS), S-28463, SAB-adj-1, SAB-adj-2, SAF-1, Sclavo® peptide, Sendai® proteoliposomes, Sendai®-containing lipid matrices, Span® 85, specol, squalane 1, squalene 2, stearyl tyrosine, tetanus toxoid (TT), Theramide™, threonyl muramyl dipeptide (TMDP), Ty Particles, and Walter Reed® liposomes.

A thymocyte-specific human immunoglobulin may be produced by immunizing the transchromosomic ungulate having the HAC vector with human thymocytes, or other target antigen of the disclosure, to produce the thymocyte-specific human immunoglobulin in the serum or plasma of the transchromosomic ungulate and recovering the thymocyte-specific human immunoglobulin from the serum or plasma of the transchromosomic ungulate.

In some embodiments, the thymocytes are administered before, during, or after administration of one or more adjuvants. In some embodiments, the thymocytes and one or more adjuvants are administered together in a single composition, comprising optionally one or more pharmaceutically acceptable excipients. In some embodiments, the thymocytes and one or more adjuvants are administered separately. In some aspects, the thymocytes and one or more adjuvants are administered at different locations, at different times, or a combination thereof.

Using at least 90% viable fresh human thymocytes, with intact membranes, is important for successful production of human ATG in transchromosomic ungulates, as it prevents off-target antibody effects, internalization, and elicits better CD protein presentation. The viability of fresh human thymocytes can be assessed by staining with a viability dye, such as fixable viability dye FVD-660, propidium iodide, or trypan blue. The number of viable thymocytes can be determined by manually counting a sample pool of thymocytes using a hemocytometer or by using an automatic counting apparatus, such as a flow cytometer or automated cell counter. Other methods for measuring cell viability include luminescent or fluorescent ATP quantification assays, metabolic activity assays such as tetrazolium-based assays (MTT, XTT) or resazurin reduction assays, and impedance-based or label-free viability measurements using electronic sensors. The proportion of viable cells can be determined by dividing the number of viable cells by the total number of cells. Viability of thymocytes is calculated as the mean of each single-stained well (n=10) of thymocytes.

Using at least 70% intact fresh human thymocytes is important for successful immunization and production of SAB-142 in transchromosomic ungulates. Intact cells can be measured by gating for SC/SSC morphology, singlets, or the size of thymocytes. Mean viability strongly correlates with mean intact cells, in a test where fresh thymocytes were stained with fixable viability dye (FVD-660). FVD-660 labels cells with compromised membranes, so viable cells are gated as FVD-negative. Counts of intact cells correlated strongly with viability as measured by FVD-660.

In some embodiments, the fresh thymocytes administered to the transchromosomic ungulate comprise at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% viable fresh human thymocytes at the time of administration. In some embodiments, the fresh thymocytes comprise about 80% to about 100%, about 80% to about 90%, about 85% to about 95%, about 91% to about 99%, about 92% to about 98%, about 93% to about 97%, about 94% to about 96%, about 90% to about 95%, about 95% to about 100%, about 92% to about 100%, about 94% to about 100%, about 96% to about 100%, about 98% to about 100%, about 90% to about 98%, about 90% to about 96%, about 90% to about 94%, about 92% to about 96%, about 94% to about 98%, or about 96% to about 99% viable fresh human thymocytes at the time of administration.

Thymocytes used for immunization are characterized by staining for surface protein CD thymocyte markers and non-thymocyte surface proteins, such as CD markers. Thymocyte CD surface markers tested include CD2, CD3, CD4, CD8, CD28. Non-thymocyte CD surface markers tested include CD14, CD19, CD41, CD61, and CD235a. A high percentage of thymocytes used for immunization are positive for thymocyte-specific surface markers, while less than about 10% of thymocytes used for immunization are positive for non-thymocyte-specific markers, reflecting the expected binding trends, with high staining for thymocyte markers and low or background-only staining for non-thymocyte markers. Variability observed among and within thymocyte-specific positivity measurements aligns with anticipated outcomes for heterogeneous thymocyte populations at distinct maturation stages. For instance, expression of the pro-thymocyte marker CD2 is substantially uniform across the population. In contrast, CD3 positivity exhibits a relative reduction compared to CD4/CD8 markers, which is consistent with CD3 being predominantly localized within the cytoplasm during the CD4/CD8 Double Positive (DP) developmental stage. T cell characterization results indicate that the thymocytes collected are primarily composed of immature T lymphocytes. In some embodiments, the thymocytes are immature T lymphocytes. In some embodiments, the thymocytes include less than about 10% of thymocytes that are positive for non-thymocyte-specific markers such as CD14, CD19, CD41, CD61, and CD235a. In some embodiments, the thymocytes include less than about 15%, about 10%, about 8%, about 6%, about 4%, or about 2% of thymocytes that are positive for non-thymocyte-specific markers such as CD14, CD19, CD41, CD61, and CD235a. In some embodiments, the thymocytes include less than about 8% of thymocytes that are positive for non-thymocyte-specific markers such as CD14, CD19, CD41, CD61, and CD235a. In some embodiments, the thymocytes include less than about 5% of thymocytes that are positive for non-thymocyte-specific markers such as CD14, CD19, CD41, CD61, and CD235a. In some embodiments, the thymocytes include less than about 1% of thymocytes that are positive for non-thymocyte-specific markers such as CD14, CD19, CD41, CD61, and CD235a.

The immunization may be carried out by administering human thymocytes with, for example, a complete Freund's adjuvant, Quila A, or an appropriate adjuvant such as an aluminum hydroxide gel, and pertussis bacteria vaccine, administered subcutaneously, intravenously, intramuscularly, or intraperitoneally into a transchromosomic ungulate.

In one embodiment, the immunization includes hyperimmunization. In various embodiments, the human thymocytes are administered once to 10 times every 1 to 4 weeks after a first administration. After 1 to 14 days following each administration, blood is collected from the animal.

In some embodiments, the fresh thymocytes are administered 3, 4, 5, 6 or more times. Administration of the fresh thymocytes may be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma may be harvested from the transchromosomic ungulate one or more times. For example, the method may include performing controls and blood collections two or three times at intervals of about 7-14 days.

### Methods of Administration of Thymocytes

Human thymocytes are administered to transchromosomic ungulate for immunization The volume of thymocyte injected in a single site ranges from 1 to 10 mL. In some embodiments, the human thymocytes are administered into 1, 2, 3, 4, 5, 6, 7, 8, 9, or 10 different locations. In some embodiments, the human thymocytes are administered into 2 different locations. In some embodiments, the human thymocytes are administered at 3 different locations. In some embodiments, the human thymocytes are administered at 4 different locations. In some embodiments, the human thymocytes are administered at 5 different locations. In some embodiments, the human thymocytes are administered at 6 different locations. In some embodiments, the human thymocytes are administered at 7 different locations. In some embodiments, the human thymocytes are administered at 8 different locations. In a preferred embodiment, the human thymocytes are administered into 6 different locations.

In other embodiments, 2 mL of fresh human thymocytes is administered and 2 mL of adjuvant is administered. In further embodiments, 3 mL of fresh human thymocytes is administered, and 3 mL of adjuvant is administered. In alternative embodiments, 4 mL of fresh human thymocytes is administered, and 3 mL of adjuvant is administered. In yet other embodiments, 5 mL of fresh human thymocytes is administered, and 4 mL of adjuvant is administered.

### Immunization Schedule and Plasma Collection

Immunization of transchromosomic ungulates is performed in multiple administrations, typically separated by an interval of approximately three to four weeks (see TABLE 10 for vaccination/immunization schedule). Each immunization includes administration of at least 2×109 human thymocytes. For effective hyperimmunization, it is important to increase the quantity of human thymocytes administered after completion of the initial two immunizations. In some embodiments, the first and second immunizations include about 2 to 6×10⁹ human thymocytes. In some embodiments, the first and second immunizations include more than 2 to 6×10⁹ human thymocytes. In some embodiments, the first and second immunizations include about 2×10⁹ human thymocytes

Plasma collection may commence seven days following each immunization, with up to four collections permissible per immunization cycle. A minimum interval of seventy-two hours is maintained between successive plasma collections, with longer intervals used as required by the physiological condition of the transchromosomic ungulate. For each plasma collection, up to 2.4% of the transchromosomic ungulate body weight can be collected. For example, for a 400 kg transchromosomic ungulate, up to 9.5L of plasma can be collected in each collection session. To increase the amount of plasma that can be collected, the feasibility of a fourth plasma collection after V3 and up (to be determined on a case-by-case basis) will be explored. This collection would occur at approximately 17 days after target antigen vaccination from V3 and up. When a fourth plasma collection occurs, there must be at least 4 weeks between the fourth plasma collection and the first plasma collection after the next vaccination.

In some embodiments, plasma up to about 1% to about 3% of transchromosomic ungulate body weight is collected in each collection session. In some embodiments, plasma up to about 3% of transchromosomic ungulate body weight is collected in each collection session. In some embodiments, plasma up to about 2.5% of transchromosomic ungulate body weight is collected in each collection session. In some embodiments, plasma up to about 2.3% of transchromosomic ungulate body weight is collected in each collection session. In some embodiments, plasma up to about 2.1% of transchromosomic ungulate body weight is collected in each collection session. In some embodiments, plasma up to about 1.8% of transchromosomic ungulate body weight is collected in each collection session. In some embodiments, plasma up to about 1.5% of transchromosomic ungulate body weight is collected in each collection session. In some embodiments, plasma up to about 1.2% of transchromosomic ungulate body weight is collected in each collection session. In some embodiments, plasma up to about 1% of transchromosomic ungulate body weight is collected in each collection session.

### Plasma Pooling Methods to Increase Efficacy

SAB-142 is generated by pooling plasma produced from two or more transchromosomic ungulates at different immunization points after V3 immunization (see TABLE 10 for immunization timings). Pooling plasma is essential in generating efficacious SAB-142 lots with consistency, as the process allows controlling the titer/avidity to target proteins. Non-clinical lots can be generated by pooling plasma from immunizations V3-V7, while clinical lots are generated by pooling plasma from immunizations V3-V5. This plasma selection strategy for clinical lot generation was chosen because in vitro studies demonstrated an improved safety-to-efficacy benefit ratio. Metrics considered when determining pooling strategy include RBC, platelet, glomerular basement membrane, serum protein binding or activity, anti-human PBMC CDC activity, and anti-CD binding. Lots made with pooled plasma collected in immunizations V6 onwards may change IgG titer/avidity against target antigens, decrease epitope coverage, and increase off-target risk to host proteins.

In some embodiments, plasma is pooled from 2 to 7 transchromosomic ungulates. In some embodiments, plasma is pooled from 2 transchromosomic ungulates. In some embodiments, plasma is pooled from 3 transchromosomic ungulates. In some embodiments, plasma is pooled from 4 transchromosomic ungulates. In some embodiments, plasma is pooled from 5 transchromosomic ungulates. In some embodiments, plasma is pooled from 6 transchromosomic ungulates. In some embodiments, plasma is pooled from 7 transchromosomic ungulates.

### Plasma Purification and SAB-142 Manufacturing Methods

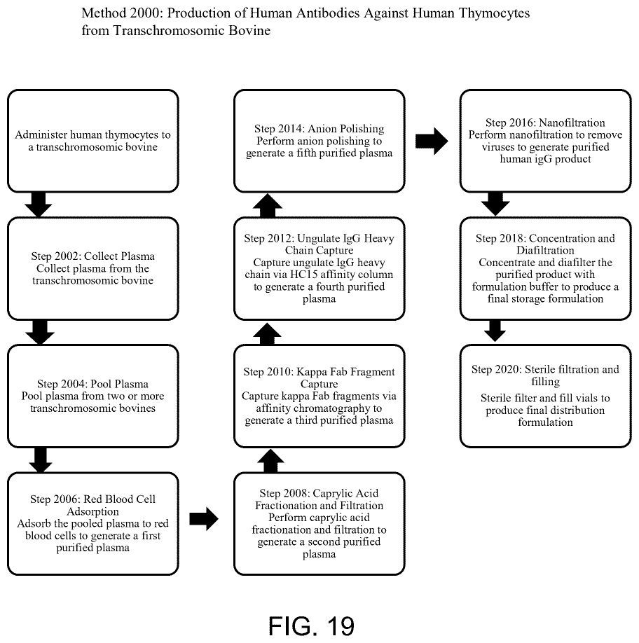

SAB-142 lots undergo multiple steps of plasma purification. FIG. 19 illustrates a manufacturing method 2000 for producing human antibodies against human thymocytes from transchromosomic bovine. The method 2000 begins with administering human thymocytes to a transchromosomic bovine. In a step 2002, plasma is collected from the transchromosomic bovine. In a step 2004, plasma from two or more transchromosomic bovines is pooled. In a step 2006, the pooled plasma is adsorbed to red blood cells to generate a first purified plasma. In a step 2008, caprylic acid fractionation and filtration are performed to generate a second purified plasma. In a step 2010, kappa Fab fragments are captured via affinity chromatography to generate a third purified plasma. In a step 2012, ungulate IgG heavy chain is captured via HC15 affinity column to generate a fourth purified plasma. In a step 2014, anion polishing is performed to generate a fifth purified plasma. In a step 2016, nanofiltration is performed to remove viruses and create the purified human igG product. In a step 2018, the purified human igG product is concentrated and diafiltered with the formulation buffer to produce a final storage formulation. In a step 2020, the final storage formulation is sterile filtered and filled into vials to produce a final distribution formulation.

Pooled plasma first undergoes red blood cell (RBC) adsorption to remove anti-RBC antibodies or fragments thereof and generate a first purified plasma sample. Pooled plasma is contacted with washed pooled RBC from different donors, which comprises a blood type selected from the group consisting of A, B, AB, O, Rh-positive, or Rh-negative, and a combination thereof. The RBC adsorbed plasma is centrifuged, and the supernatant is collected for the next steps. Next, viruses, pathogens, and/or non-IgG bovine plasma proteins are removed from the first purified plasma sample to generate a second purified plasma sample. The removing comprises using caprylic acid fractionation, filter purification, or a combination thereof. The pH of the first purified plasma sample is adjusted to about 4 to 6, preferably about 4.5 to 4.9, using 20% acetic acid, and caprylic acid is then added in a weight amount equal to a weight of total protein plasma content. Supernatant neutralized to pH 7.3-8.0 is depth filtered with a Celpure® P1000 filter aid and a 0.22 m filter washed with phosphate-buffered saline at least 100 LMH flux. The second purified plasma sample is then added to an affinity purification column to capture and elute kappa Fab fragments to generate a third purified plasma sample. The elute is collected starting at 0.9 column volumes and ending at 4.9 column volumes. Captured kappa Fab fragments are incubated at a low pH between about 3 to 4, preferably between about 3.6-3.8, for viral inactivation. The third purified plasma sample is then added to an affinity column designed for binding ungulate IgG heavy chain proteins and eluting the third purified plasma sample to generate a fourth purified plasma sample. The purified IgG is subjected to a Q Sepharose® fast flow anion polishing step to remove additional impurities selected from the group consisting of IgA, IgM, host cell proteins, DNA, endotoxin, and a combination thereof, generating a fifth purified plasma sample. The fifth purified plasma sample is subjected to nanofiltration to remove viruses, producing a sixth purified plasma sample that is then concentrated to produce purified human ATG, then diafiltrated with a formulation buffer to produce a human ATG formulation.

In some embodiments, the RBC adsorption step may be performed at a temperature ranging from about 2° C. to about 37° C. In some embodiments, the RBC adsorption is performed at about 4° C., about 15° C., about 20° C., about 25° C., or about 37° C. In some embodiments, the RBC adsorption is performed at room temperature.

In some embodiments, the pooled plasma is contacted with RBC at a ratio of about 1:1 to about 10:1 (v/v) plasma to packed RBC. In some embodiments, the ratio is about 2:1, about 3:1, about 4:1, about 5:1, about 6:1, about 7:1, about 8:1, about 9:1, or about 10:1. In some embodiments, the ratio is about 5:1 plasma to packed RBC.

In some embodiments, the pooled plasma and RBC mixture is incubated for a period ranging from about 10 minutes to about 120 minutes. In some embodiments, the incubation period is about 15 minutes, about 30 minutes, about 45 minutes, about 60 minutes, about 90 minutes, or about 120 minutes. In some embodiments, the incubation period is about 30 minutes to about 60 minutes.

In some embodiments, the RBC adsorption step is performed with gentle mixing or agitation. In some embodiments, the mixing is performed by end-over-end rotation, orbital shaking, or gentle stirring. In some embodiments, the mixing is performed at about 5 to about 30 rotations per minute.

In some embodiments, the RBCs used for adsorption are washed one, two, three, or more times with a physiologically compatible buffer prior to contact with the pooled plasma. In some embodiments, the washing buffer comprises phosphate-buffered saline, normal saline, or Tris-buffered saline. In some embodiments, the RBCs are washed three times with phosphate-buffered saline.

In some embodiments, the RBCs are obtained from human donors and may include a mixture of different blood types. In some embodiments, the RBC mixture comprises cells from at least two different blood types. In some embodiments, the RBC mixture comprises cells from at least three different blood types. In some embodiments, the RBC mixture comprises cells from blood types A, B, AB, and O. In some embodiments, the RBC mixture comprises both Rh-positive and Rh-negative cells.

In some embodiments, the RBC adsorption is performed one or more times to enhance the removal of anti-RBC antibodies or fragments thereof. In some embodiments, the RBC adsorption is performed twice using fresh RBC for each adsorption cycle. In some embodiments, the RBC adsorption is performed three times.

In some embodiments, following RBC adsorption and centrifugation, the supernatant is carefully separated from the RBC pellet to minimize carryover of cellular material. In some embodiments, the supernatant is filtered through a depth filter or membrane filter to remove residual RBC or cellular debris. In some embodiments, the filtration is performed using a filter with a pore size of about 0.2 μm to about 5 μm.

In some embodiments, the RBC adsorption step removes immunoglobulins that bind to blood group antigens, including anti-A, anti-B, and anti-Rh antibodies. In some embodiments, the RBC adsorption reduces anti-RBC antibody content by at least about 50%, at least about 60%, at least about 70%, at least about 80%, or at least about 90%.

In some embodiments, the effectiveness of RBC adsorption may be monitored by measuring the reduction in binding to RBC antigens using flow cytometry or hemagglutination assays. In some embodiments, the adsorbed plasma is tested for residual anti-RBC antibodies before proceeding to subsequent purification steps.

In some embodiments, the first purified plasma sample is subjected to caprylic acid fractionation. In some embodiments, the caprylic acid fractionation is performed at a temperature ranging from about 2° C. to about 37° C. In some embodiments, the caprylic acid fractionation is performed at about 4° C., about 15° C., about 20° C., about 25° C., or about 37° C. In some embodiments, the caprylic acid fractionation is performed at room temperature.

In some embodiments, a pH adjustment to about 4 to 6 is performed gradually over a period of about 5 minutes to about 60 minutes. In some embodiments, the pH adjustment is performed over about 10 minutes, about 15 minutes, about 20 minutes, about 30 minutes, or about 45 minutes.

In some embodiments, the pH is monitored continuously during adjustment to ensure accurate control.

In some embodiments, the caprylic acid is added at a concentration ranging from about 0.5% to about 2% (v/v) relative to the plasma volume. In some embodiments, the caprylic acid is added at about 0.75%, about 1%, about 1.25%, about 1.5%, or about 1.75% (v/v). In some embodiments, the caprylic acid is added dropwise or in a controlled manner to ensure uniform distribution.

In some embodiments, following caprylic acid addition, the mixture is incubated for a period ranging from about 30 minutes to about 180 minutes. In some embodiments, the incubation period is about 45 minutes, about 60 minutes, about 90 minutes, about 120 minutes, or about 150 minutes. In some embodiments, the incubation is performed with gentle stirring or mixing.

In some embodiments, the caprylic acid-treated plasma is centrifuged at about 1500 to about 5000×g for about 10 to about 60 minutes. In some embodiments, the centrifugation is performed at about 2000×g, about 2500×g, about 3000×g, about 3500×g, or about 4000×g. In some embodiments, the centrifugation is performed for about 15 minutes, about 20 minutes, about 30 minutes, or about 45 minutes.

In some embodiments, the supernatant is neutralized using a buffer selected from the group consisting of sodium hydroxide, Tris base, sodium phosphate, and combinations thereof. In some embodiments, the neutralization is performed to achieve a pH of about 7.0 to about 8.5. In some embodiments, the neutralized supernatant has a pH of about 7.3, about 7.5, about 7.7, about 8.0, or about 8.3.

In some embodiments, the depth filtration is performed using a filter aid comprising diatomaceous earth, perlite, cellulose, or combinations thereof. In some embodiments, the filter aid is Celpure® P65, Celpure® P100, Celpure® P300, or Celpure® P1000. In some embodiments, the filter aid is pre-coated onto a filtration surface before the neutralized supernatant is applied.

In some embodiments, the depth filtration is performed at a flux rate ranging from about 50 to about 200 liters per square meter per hour (LMH). In some embodiments, the depth filtration is performed at a flux rate ranging from about 100 to about 240 liters per square meter per hour (LMH). In some embodiments, the flux rate is about 75 LMH, about 100 LMH, about 125 LMH, about 150 LMH, about 175 LMH, about 200 LMH, about 240 LMH, about 280 LMH, or about 300 LMH. In some embodiments, the flux rate is maintained at about 100 LMH or greater.

In some embodiments, the first purified plasma sample is further filtered through a membrane filter with a pore size of about 0.1 μm to about 0.5 μm. In some embodiments, the membrane filter has a pore size of about 0.15 μm, about 0.2 μm, about 0.22 μm, about 0.3 μm, or about 0.45 μm. In some embodiments, the membrane filter is a polyethersulfone, polyvinylidene fluoride, or cellulose acetate membrane.

In some embodiments, the caprylic acid fractionation removes at least about 50%, at least about 60%, at least about 70%, at least about 80%, at least about 90%, or at least about 95% of non-IgG proteins. In some embodiments, the caprylic acid fractionation substantially removes albumin, transferrin, and other serum proteins while preserving IgG content. In some embodiments, the caprylic acid fractionation generates a second purified plasma sample that is at least 90% IgG.

In some embodiments, the affinity purification column comprises a resin that specifically binds kappa light chains, and processing the second purified plasma sample by binding the kappa chain generates a third purified plasma sample. In some embodiments, the resin is KappaSelect™ CaptureSelect™ Kappa, or a similar kappa-specific affinity resin. In some embodiments, the column is equilibrated with a buffer at pH 6.0 to pH 8.0 prior to sample loading.

In some embodiments, the second purified plasma sample is loaded onto the affinity column at a flow rate of about 50 to about 500 cm/hour. In some embodiments, the flow rate is about 100 cm/hour, about 150 cm/hour, about 200 cm/hour, about 250 cm/hour, about 300 cm/hour, or about 400 cm/hour. In some embodiments, the loading is performed at about 4° C. to about 25° C.

In some embodiments, the affinity column is washed with a wash buffer after sample loading to remove unbound material. In some embodiments, the wash buffer comprises phosphate-buffered saline, Tris-buffered saline, or a similar physiologically compatible buffer. In some embodiments, the wash is performed for about 3 to about 10 column volumes. In some embodiments, the wash is performed for about 4, about 5, about 6, about 7, or about 8 column volumes.

In some embodiments, the kappa Fab fragments are eluted using an elution buffer with a pH ranging from about 2.5 to about 4.5. In some embodiments, the elution buffer has a pH of about 3.0, about 3.2, about 3.4, about 3.6, about 3.8, about 4.0, or about 4.2. In some embodiments, the elution buffer comprises glycine-HCl, citrate, or acetate buffer.

In some embodiments, the elution is performed at a flow rate of about 50 to about 300 cm/hour. In some embodiments, the elution flow rate is about 75 cm/hour, about 100 cm/hour, about 150 cm/hour, about 200 cm/hour, or about 250 cm/hour. In some embodiments, the elution is collected in fractions of about 0.1 to about 1.0 column volumes.

In some embodiments, the elute fractions are monitored by measuring absorbance at 280 nm to determine protein content. In some embodiments, fractions with absorbance above a predetermined threshold are pooled. In some embodiments, the predetermined threshold is about 0.1, about 0.2, about 0.3, about 0.5, or about 1.0 absorbance units.

In some embodiments, capture of the kappa Fab fragments is followed by viral inactivation at a low pH hold at room temperature to inactivate enveloped viruses. In some embodiments, the virus reduction is measured using model viruses such as minute virus of mice (MVM) or xenotropic murine leukemia virus (X-MuLV). In some embodiments, the viral inactivation at low pH results in an average log reduction of XMuLV of at least about 3.0, at least about 3.5, at least about 4.0, at least about 4.11, at least about 4.5, or at least about 5.0. In some embodiments, the viral inactivation at low pH results in an average log reduction of XMuLV of about 3.0 to about 6.0, about 3.5 to about 5.5, or about 4.0 to about 5.0. In some embodiments, the viral inactivation at low pH results in an average log reduction of PRV of at least about 5.0, at least about 5.5, at least about 6.0, at least about 6.46, at least about 7.0, or at least about 7.5. In some embodiments, the viral inactivation at low pH results in an average log reduction of PRV of about 5.0 to about 8.0, about 5.5 to about 7.5, or about 6.0 to about 7.0.

In some embodiments, following viral inactivation, the pH of the third purified plasma sample is adjusted to about 6.0 to about 8.0. In some embodiments, following viral inactivation, the pH of the third purified plasma sample is adjusted to about 7.8 to about 8.0. In some embodiments, the pH is adjusted to about 6.5, about 7.0, about 7.5, or about 8.0. In some embodiments, the pH adjustment is performed using Tris base, sodium hydroxide, or sodium phosphate.

In some embodiments, the third purified plasma sample is subjected to an affinity column for binding ungulate IgG heavy chain proteins, generating a fourth purified plasma sample. In some embodiments, the affinity column for binding ungulate IgG heavy chain comprises Protein A, Protein G, Protein A/G, or an ungulate IgG heavy chain-specific resin. In some embodiments, the resin is HC15, which specifically binds bovine IgG heavy chains. In some embodiments, the column is equilibrated with a buffer at pH 6.5 to pH 8.0 prior to sample loading.

In some embodiments, the third purified plasma sample is loaded onto the ungulate IgG heavy chain affinity column at a flow rate of about 50 to about 400 cm/hour. In some embodiments, the third purified plasma sample is loaded onto the ungulate IgG heavy chain affinity column at a flow rate of about 90 to about 110 cm/hour. In some embodiments, the flow rate is about 50 cm/hour, about 90 cm/hour, about 110 cm/hour, or about 150 cm/hour.

In some embodiments, the column is washed after sample loading to remove unbound material and contaminants. In some embodiments, the wash is performed for about 3 to about 10 column volumes. In some embodiments, the wash buffer comprises phosphate-buffered saline or Tris-buffered saline at pH 7.0 to pH 8.0.

In some embodiments, the third purified plasma sample is eluted from the ungulate IgG heavy chain affinity column using an elution buffer with a pH ranging from about 2.5 to about 4.5. In some embodiments, the elution buffer has a pH of about 3.0, about 3.5, about 4.0, or about 4.5. In some embodiments, the elution buffer comprises glycine-HCl, citrate, or acetate buffer. In some embodiments, processing the third purified plasma sample with the ungulate IgG heavy chain affinity column generates a fourth purified plasma sample.

In some embodiments, the fourth purified plasma sample is immediately neutralized to prevent protein degradation. In some embodiments, the neutralization is performed by adding a neutralization buffer to achieve a pH of about 6.0 to about 8.0. In some embodiments, the neutralization buffer comprises Tris-HCl, sodium phosphate, or HEPES.

In some embodiments, the fourth purified plasma sample is concentrated and buffer-exchanged prior to the Q Sepharose® anion polishing step. In some embodiments, the concentration and buffer exchange are performed using tangential flow filtration or ultrafiltration.

In some embodiments, the buffer exchange is performed into a low ionic strength buffer at pH 7.0 to pH 8.5.

In some embodiments, the Q Sepharose® fast flow resin is equilibrated with a buffer at pH 7.0 to pH 9.0 prior to sample loading. In some embodiments, the equilibration buffer has a pH of about 7.5, about 7.9, about 8.0, about 8.5, or about 9.0. In some embodiments, the equilibration buffer comprises Tris-HCl, sodium phosphate, or HEPES.

In some embodiments, the fourth purified plasma sample is loaded onto the Q Sepharose® column at a flow rate of about 50 to about 500 cm/hour. In some embodiments, the flow rate is about 100 cm/hour, about 150 cm/hour, about 200 cm/hour, about 300 cm/hour, or about 400 cm/hour.

In some embodiments, the Q Sepharose® column is operated in flow-through mode, where the desired IgG product flows through the column while impurities bind to the resin. In some embodiments, the flow-through is collected and represents the fifth purified plasma sample. In some embodiments, the column is washed with equilibration buffer, and the wash is combined with the flow-through.

In some embodiments, the Q Sepharose® polishing step removes at least about 50%, at least about 60%, at least about 70%, at least about 80%, at least about 90%, or at least about 95% of residual IgA. In some embodiments, the Q Sepharose® polishing step removes at least about 50%, at least about 60%, at least about 70%, at least about 80%, at least about 90%, or at least about 95% of residual IgM.

In some embodiments, the Q Sepharose® polishing step removes at least about 70%, at least about 80%, at least about 90%, at least about 95%, or at least about 99% of host cell proteins. In some embodiments, the Q Sepharose® polishing step removes at least about 80%, at least about 90%, at least about 95%, or at least about 99% of residual DNA.

In some embodiments, the Q Sepharose® polishing step reduces endotoxin levels by at least about 1-log, at least about 2-log, at least about 3-log, or at least about 4-log. In some embodiments, the endotoxin level in the fifth purified plasma sample is less than about 1 EU/mg, less than about 0.5 EU/mg, less than about 0.2 EU/mg, or less than about 0.1 EU/mg.

In some embodiments, the fifth purified plasma sample is subjected to nanofiltration to produce a sixth purified plasma sample. In some embodiments, the nanofiltration is performed using a membrane with a pore size of about 15 nm to about 50 nm. In some embodiments, the membrane has a pore size of about 20 nm, about 25 nm, about 30 nm, about 35 nm, or about 40 nm. In some embodiments, the nanofiltration membrane is a Planova™ 15N, Planova™ 20N, Planova™ 35N, or similar virus removal filter. In some embodiments, the filtration solution is Viresolve® Pro, or similar virus filtration solution.

In some embodiments, the nanofiltration is performed at a transmembrane pressure of about 5 to about 50 psi. In some embodiments, the nanofiltration is performed at a transmembrane pressure of about 20 to about 30 psi. In some embodiments, the transmembrane pressure is about 10 psi, about 15 psi, about 20 psi, about 25 psi, about 30 psi, or about 40 psi. In some embodiments, the nanofiltration is performed at a temperature of about 18° C. to about 30° C. In some embodiments, the nanofiltration achieves at least about 4-log reduction of viruses. In some embodiments, the nanofiltration achieves at least about 5-log, at least about 6-log, or at least about 7-log reduction of viruses

In some embodiments, the sixth purified plasma sample is concentrated after nanofiltration to produce purified human ATG. In some embodiments, the concentration is performed to achieve a protein concentration of about 10 mg/mL to about 100 mg/mL. In some embodiments, the concentration is performed to achieve a protein concentration of about 21 mg/mL to about 31 mg/mL. In some embodiments, the protein concentration is about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL.

In some embodiments, the purified human ATG is subjected to a final formulation buffer exchange. In some embodiments, the buffer exchange is performed using tangential flow filtration or diafiltration. In some embodiments, the final formulation buffer comprises L-glutamic acid monosodium salt, D-sorbitol, and polysorbate 80 at pH 5.5.

In some embodiments, the RBC adsorption step is performed with gentle mixing or agitation. In some embodiments, the mixing is performed by end-over-end rotation, orbital shaking, or gentle stirring. In some embodiments, the mixing is performed at about 5 to about 30 rotations per minute.

In some embodiments, the RBC used for adsorption are washed one, two, three, or more times with a physiologically compatible buffer prior to contact with the pooled plasma. In some embodiments, the washing buffer comprises phosphate-buffered saline, normal saline, or Tris-buffered saline. In some embodiments, the RBC are washed three times with phosphate-buffered saline.

In some embodiments, the RBC are obtained from human donors and may include a mixture of different blood types. In some embodiments, the RBC mixture comprises cells from at least two different blood types. In some embodiments, the RBC mixture comprises cells from at least three different blood types. In some embodiments, the RBC mixture comprises cells from blood types A, B, AB, and O. In some embodiments, the RBC mixture comprises both Rh-positive and Rh-negative cells.

In some embodiments, the RBC adsorption step may be repeated one or more times to enhance removal of off-target immunoglobulins. In some embodiments, the RBC adsorption is performed twice using fresh RBC for each adsorption cycle. In some embodiments, the RBC adsorption is performed three times.

In some embodiments, following RBC adsorption and centrifugation, the supernatant is carefully separated from the RBC pellet to minimize carryover of cellular material. In some embodiments, the supernatant is filtered through a depth filter or membrane filter to remove residual RBC or cellular debris. In some embodiments, the filtration is performed using a filter with a pore size of about 0.2 μm to about 5 μm.

In some embodiments, the RBC adsorption step removes immunoglobulins that bind to blood group antigens, including anti-A, anti-B, and anti-Rh antibodies. In some embodiments, the RBC adsorption reduces off-target immunoglobulin content by at least about 50%, at least about 60%, at least about 70%, at least about 80%, or at least about 90%.

In some embodiments, the effectiveness of RBC adsorption may be monitored by measuring the reduction in binding to RBC antigens using flow cytometry or hemagglutination assays. In some embodiments, the adsorbed plasma is tested for residual anti-RBC antibodies before proceeding to subsequent purification steps.

### Human Artificial Chromosome

In embodiments of the methods of the disclosure, the genome of the transchromosomic ungulate comprises a human immunoglobulin locus. Illustrative methods are provided in U.S. Pat. Nos. 9,902,970; 9,315,824; 7,652,192; and 7,429,690; and 7,253,334, the disclosures of which are each incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699.

The disclosure provides a human artificial chromosome (HAC) vector comprising genes encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.
   Construction of the isHAC and isKcHACΔ Vectors in Chicken DT40 Cells.

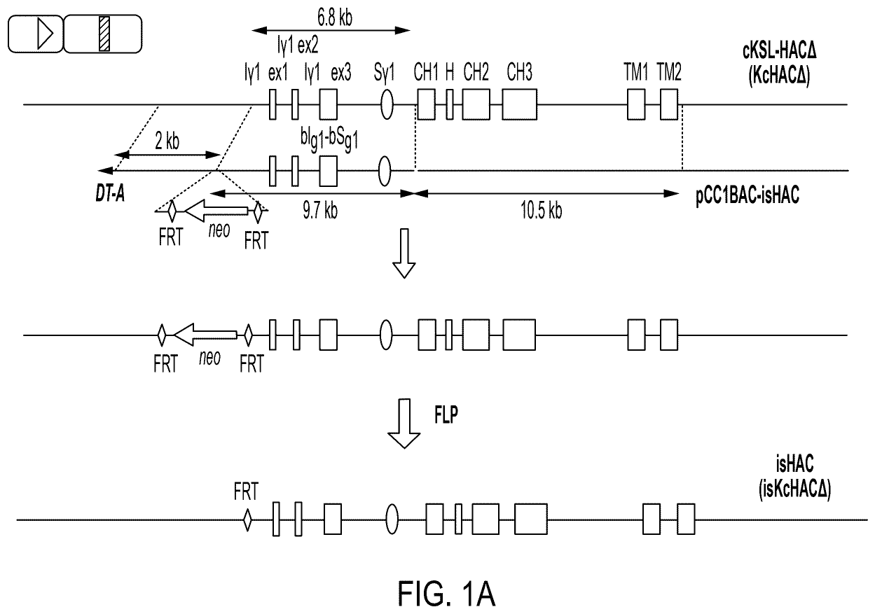

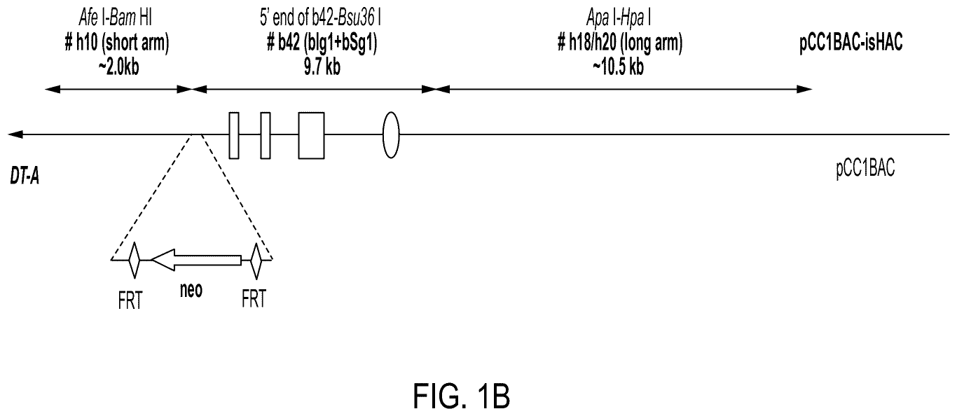

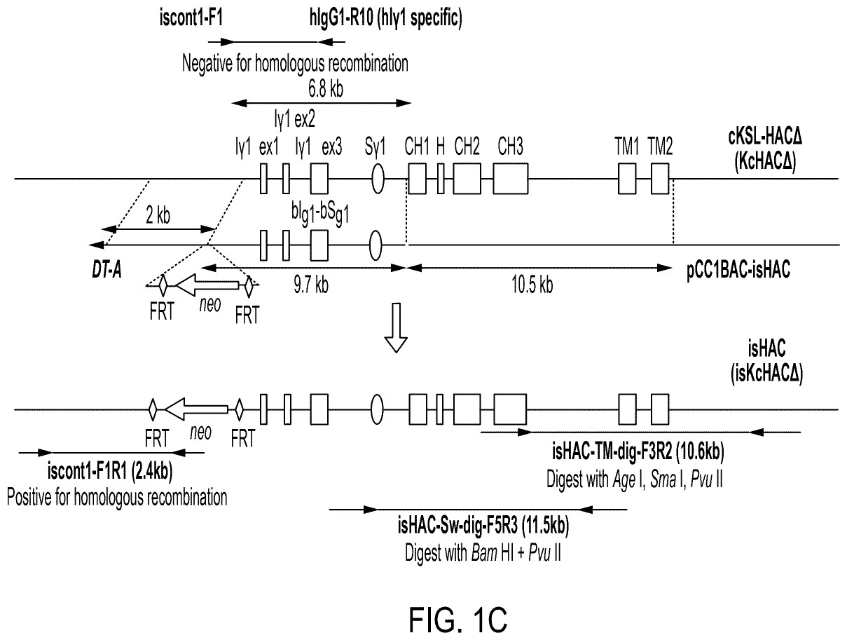

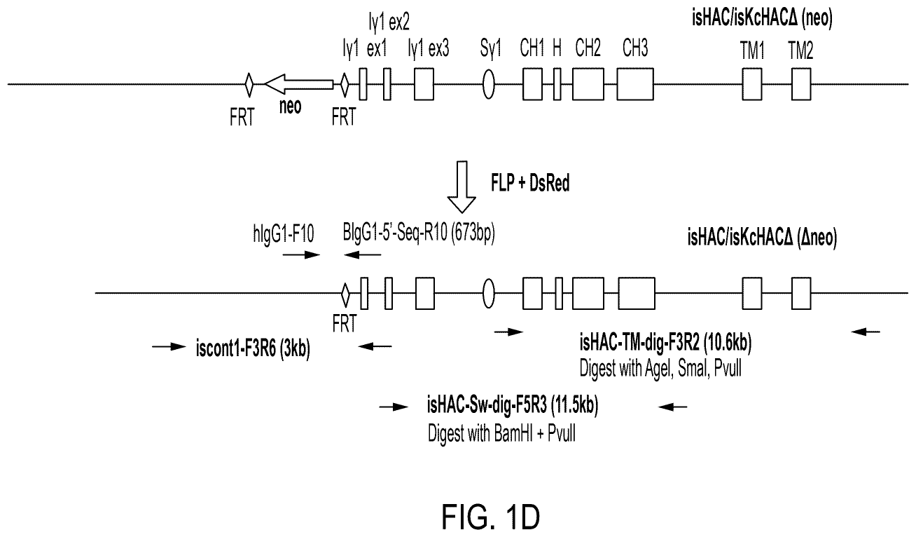

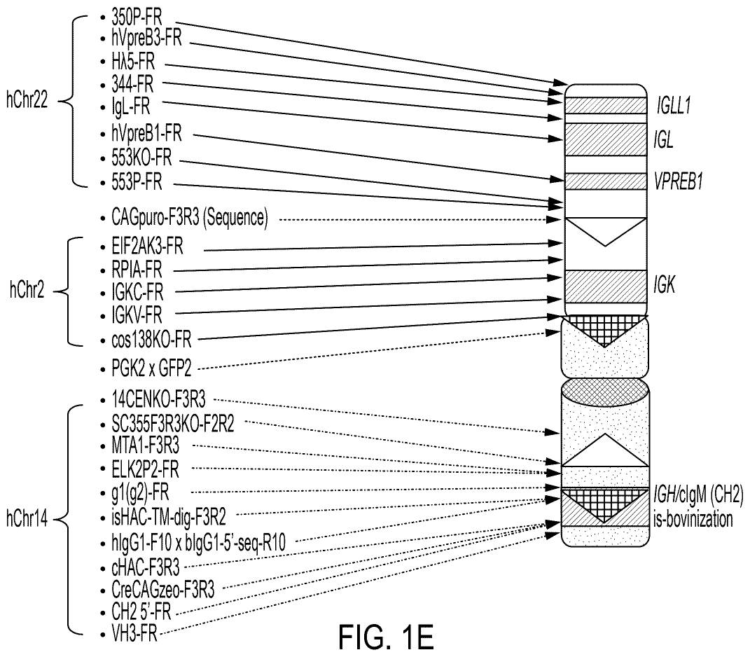

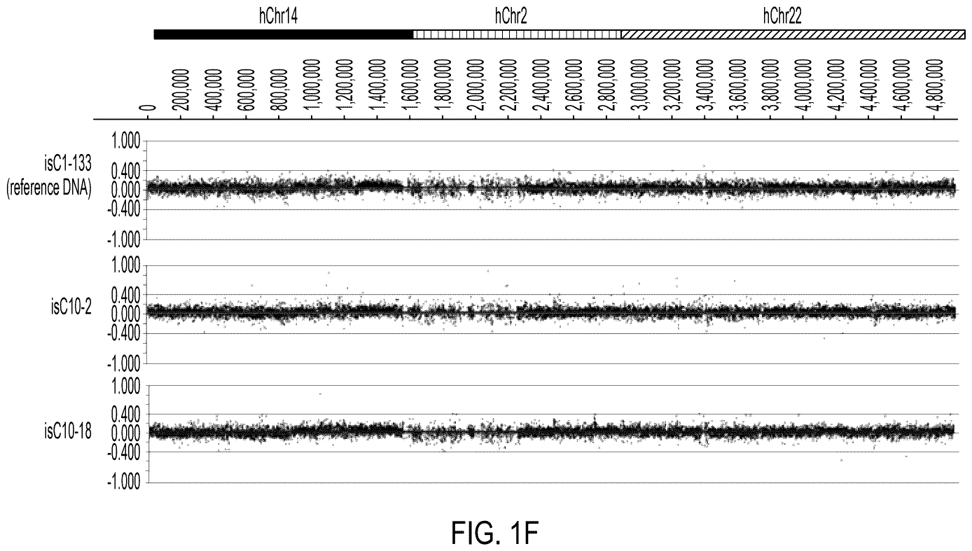

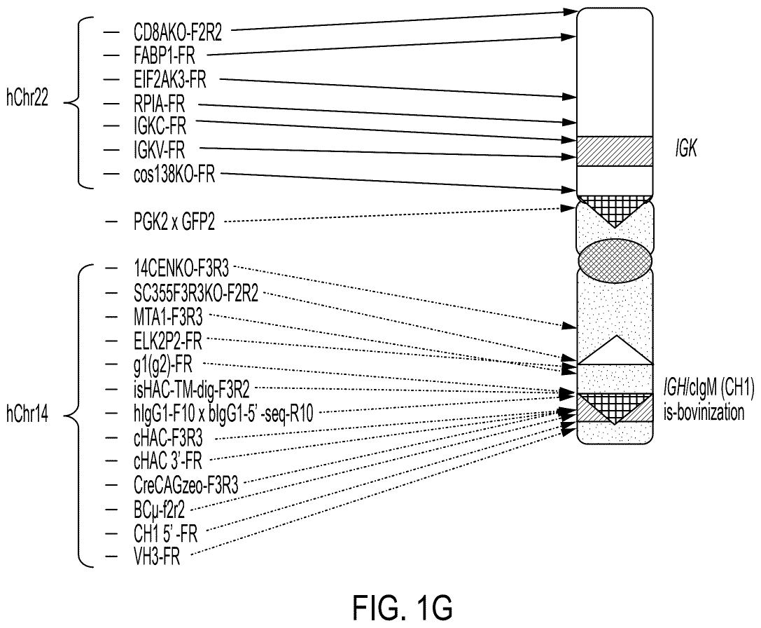

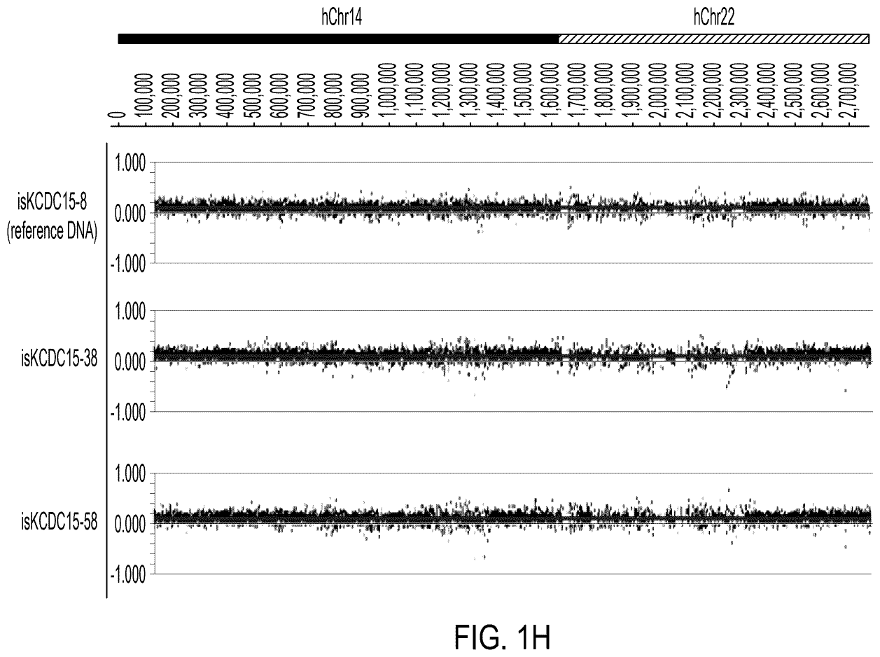

Outline of the isHAC (isKcHACA) construction is depicted in FIG. 1A. The targeting vector pCC1BAC-isHAC was constructed (FIG. 1B) and used to bovinize the I_{γ1}-S_{γ1} region on the cKSL-HACΔ or KcHACΔ. Clone cKSLDD1, a chicken DT40 cell line retaining the cKSL-HACΔ obtained by MMCT from cKSLDH22 (2L), was electroporated with the targeting vector pCC1BAC-isHAC. Colonies were selected with G418 and their genomic DNA was subjected to PCR screening with primers, iscont1-F1R1, to identify the occurrence of the homologous recombination. Furthermore, additional diagnostic PCRs were also performed to check structural integrity (FIG. 1C). One clone is 1-11 was selected for the subsequent neo cassette deletion by introduction of the FLP-expression plasmid. The is 1-11 was co-transfected with the FLP-expressing plasmid and the DsRed-expressing plasmid. DsRed-positive cells were sorted and subjected to single colony isolation. G418-sensitive colonies where the neo cassette was deleted by the FRT-FLP recombination were tested for the genomic PCRs including iscont1-F3/R6 (FIG. 1D). Finally, isH1 1-S2 and isH9-3 were selected, and then they were transferred to CHO cells to establish master cell banks, isCl-133, isC10-2 and isC10-18, respectively, for which the extensive genomic PCR and CGH were performed to check structural integrity (FIG. 1E, FIG. 1F). The isKcHACΔ was constructed in DT40 cells, similarly to the isHAC, and two clones, isKCDH17, isKCDH30, were selected and then were transferred to CHO cells to establish master cell banks, isKCDC8 and isKCDC38, respectively, for which the extensive genomic PCR and CGH were performed to check structural integrity (FIG. 1G, FIG. 1H).

The HAC vectors of the disclosure can be used, for example, for large-scale production of fully human antibodies by transchromosomic animals, as described for the methods of the invention. The HAC vector of the present disclosure comprises one or more genes encoding a human antibody heavy chain. Any human antibody heavy chain or combinations of human antibody heavy chains in combination may be encoded by one or more nucleic acids on the HAC. In various embodiments, 1, 2, 3, 4, 5, 6, 7, 8, or all 9 of human antibody heavy chains IgM, IgG1, IgG2, IgG3, IgG4, IgAQ1, IgA2, IgE, and IgD may be encoded on the HAC in one or more copies. In one embodiment, the HAC comprises a human IgM antibody heavy chain encoding gene, alone or in combination with 1, 2, 3, 4, 5, 6, 7, or the other 8 human antibody chain encoding genes. In one preferred embodiment, the HAC comprises a gene encoding at least a human IgG1 antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, the HAC comprises a gene encoding at least a human IgA antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another preferred embodiment, the HAC comprises genes encoding all 9 antibody heavy chains, and more preferably, where the gene encoding a human IgM antibody heavy chain has been chimerized to encode an ungulate-derived IgM heavy chain constant region. In another embodiment, the HAC may comprise a portion of human chromosome 14 that encodes the human antibody heavy chains. The variable region genes and the constant region genes of the human antibody heavy chain form a cluster, and the human heavy chain locus is positioned at 14q32 on human chromosome 14. In one embodiment, the region of human chromosome 14 inserted in the HAC comprises the variable region and the constant region of the human antibody heavy chains from the 14q32 region of human chromosome 14.

In some embodiments of the HAC vectors of the present disclosure, at least one class switch regulatory element of the human antibody heavy chain encoding nucleic acid is replaced with an ungulate-derived class switch regulatory element. The class switch regulatory element refers to nucleic acid that is 5′ to an antibody heavy chain constant region. Each heavy chain constant region gene is operatively linked with (i.e., under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. As each heavy chain constant region gene is linked with its own switch region.

Any suitable ungulate-derived class switch regulatory element can be used. For example, the human heavy chain gene isotypes listed below have the following class switch regulatory elements:
IgM: Iμ-Sμ,
IgG1: Iγ1-Sγ1,
IgG2: Iγ2-Sγ2,
IgG3: Iγ3-Sγ3,
IgG4: Iγ4-Sγ4,
IgAQ1: Iα1-Sα1,
IgA2: Iα2-Sα2, and
IgE: Iε-Sε.

In various embodiments, 1, more than 1, or all of the human antibody heavy chain genes on the HAC have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid on the HAC (such as that in SEQ ID NO: 1 of International Patent Publication WO2021163035; the contents of which are herein incorporated by reference in its entirety) is replaced with an ungulate I_{γ1}-S_{γ1} class switch regulatory element. Exemplary ungulate Iγ1-Sγ1 class regulatory switch elements include a bovine IgG1 Iγ1-Sγ1 class switch regulatory element (SEQ ID NO: 2 of International Patent Publication), a horse Iγ1-Sγ1 class switch regulatory element (SEQ ID NO: 3 of International Patent Publication WO2021163035), and a pig Iγ1-Sγ1 class switch regulatory element (SEQ ID: 4). However, it is not necessary to replace the human class switch regulatory element with an ungulate class switch regulatory element from the corresponding heavy chain isotype. Thus, for example, an Iγ3-Sγ3 human class switch regulatory element for human IgG3 heavy chain encoding nucleic acid on the HAC can be replaced with an ungulate Iγ1-Sγ1 class switch regulatory element. As will be apparent to those of skill in the art based on the teachings herein, any such combination can be used in the HACs of the disclosure.

In another embodiment, the HAC comprises at least one ungulate enhancer element to replace an enhancer element associated with one or more human antibody heavy chain constant region encoding nucleic acids on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′ Ea enhancers that can be used include 3′Eα, 3′Eα1, and 3′Eα2. Exemplary 3′Eα enhancer elements from bovine that can be used in the HACs and replace the human enhancer include, but are not limited to, bovine HS3 enhancer (SEQ ID NO: 5 of International Patent Publication WO2021163035), bovine HS12 enhancer (SEQ ID NO: 6 of International Patent Publication WO2021163035), and bovine enhancer HS4. This embodiment is particularly preferred in embodiments wherein the HAC comprises the variable region and the constant region of the human antibody heavy chains from the 14q32 region of human chromosome 14.

The HAC vectors of the present disclosure may comprise one or more genes encoding a human antibody light chain. Any suitable human antibody light chain-encoding genes can be used in the HAC vectors of the invention. The human antibody light chain includes two types of genes, i.e., the kappa/K chain gene and the lambda/L chain gene. In one embodiment, the HAC comprises genes encoding both kappa and lambda, in one or more copies. The variable region and constant region of the kappa chain are positioned at 2p 11.2-2p12 of the human chromosome 2, and the lambda chain forms a cluster positioned at 22q1 1.2 of the human chromosome 22. Therefore, in one embodiment, the HAC vectors of the invention comprise a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q1 1.2 region.

In another embodiment, the HAC vector comprises at least one gene encoding a human antibody surrogate light chain. The gene encoding a human antibody surrogate light chain refers to a gene encoding a transient antibody light chain, which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (SEQ ID NO: 7 of International Patent Publication WO2021163035), VpreB3 (SEQ ID NO: 8 of International Patent Publication WO2021163035) and X5 (also known as IgLL1, SEQ ID NO: 9 of International Patent Publication WO2021163035) human antibody surrogate light chains, and combinations thereof. The VpreB gene and the X5 gene are positioned within the human antibody lambda chain gene locus at 22q11.2 of the human chromosome 22. Therefore, in one embodiment, the HAC may comprise the 22q11.2 region of human chromosome 22 containing the VpreB gene and the X5 gene. The human VpreB gene of the present invention provides either or both of the VpreB1 gene (SEQ ID NO: 7 of International Patent Publication WO2021163035) and the VpreB3 (SEQ ID NO: 8 of International Patent Publication WO2021163035) gene, and in one embodiment, provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid can be used, including but not limited to bovine IgM, (SEQ ID NO: 10 of International Patent Publication WO2021163035), horse IgM, (SEQ ID NO: 11 of International Patent Publication WO2021163035), sheep IgM, (SEQ ID NO: 12 of International Patent Publication WO2021163035), and pig IgM, (SEQ ID NO: 13 of International Patent Publication WO2021163035). In one embodiment, the chimeric IgM comprises the sequence in SEQ ID NO: 14 of International Patent Publication WO2021163035. Pre-BCR/BCR signaling through the IgM heavy chain molecule promotes proliferation and development of the B cell by interacting with the B cell membrane molecule Ig-alpha/Ig-beta to cause a signal transduction in cells. The transmembrane region and/or other constant region of IgM are considered to have important roles in the interaction with Ig-alpha/Ig-beta for signal transduction. Examples of the IgM heavy chain constant regions include nucleic acids encoding constant region domains such as CH1, CH2, CH3, and CH4, and the B-cell transmembrane and cytoplasmic domains such as TM1 and TM2. The nucleic acid encoding an ungulate-derived IgM heavy chain constant region which is comprised in the human artificial chromosome vector of the invention is not particularly limited so long as the region is in a range which may sufficiently induce the signal of the B-cell receptor or B-cell proliferation/development in the above-described IgM heavy chain constant region. In one embodiment, the nucleic acid encoding an ungulate-derived IgM heavy chain constant region provides a transmembrane and cytoplasmic TM1 domain and TM2 domain derived from an ungulate, and in other embodiments encodes the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, the gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, the gene encoding a bovine IgM heavy chain constant region is included in the IGHM region.

In a further embodiment, the HAC comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, a gene encoding the transmembrane domain and the intracellular domain of an ungulate-derived (such as bovine) IgG (such as IgG1) heavy chain constant region is used to replace the corresponding regions of the human IgG heavy chain gene. In another embodiment, the gene encoding the TM1 and TM2 domains of an ungulate-derived (such as bovine) IgG (such as IgG1) heavy chain constant region is used to replace the corresponding regions of the human IgG heavy chain gene. In another embodiment, the gene encoding one or more of the CH1-CH4 domains and/or the TM1 and TM2 domains of an ungulate-derived (such as bovine) IgG (such as IgG1) heavy chain constant region is used to replace the corresponding regions of the human IgG heavy chain gene.

The disclosure further provides transchromosomic ungulates comprising an HAC vector according to any embodiment or combination of embodiments of the disclosure. The transchromosomic ungulate comprising the HAC vector of the present invention refers to an animal into which the human artificial chromosome vector of the present invention is introduced. The transchromosomic ungulate having the HAC of the present invention is not particularly limited so long as the animal is a transchromosomic ungulate in which the human artificial chromosome fragment may be introduced into a cell thereof, and any non-human animals, for example, ungulates such as cows, horses, goats, sheep, pigs, and the like may be used. In one aspect, the transchromosomic ungulate is a bovine. A transchromosomic ungulate having the HAC vector of the present invention may be constructed, for example, by introducing the HAC vector of the present disclosure into an oocyte of a host animal using any suitable technique, such as those described herein. The HAC vector of the present invention may, for example, be introduced into a somatic cell derived from a host ungulate by a microcell fusion method. Thereafter, an animal with the HAC vector may be constructed by transplanting a nucleus or chromatin agglomerate from the cell into an oocyte, and then transplanting the oocyte or the embryo formed from it into the uterus of a host animal, allowing it to give birth. The confirmation of this can be achieved using the method developed by Kuroiwa et al. (Kuroiwa et al., Nature Biotechnology, 18, 1086-1090, 2000, and Kuroiwa et al., Nature Biotechnology, 20, 889-894).

### Transchromosomic Ungulates Including a Human Artificial Chromosome

The disclosure further provides transchromosomic ungulates comprising genes integrated into their genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

In such embodiments, the transchromosomic ungulate may comprise any embodiment or combination of embodiments of the nucleic acids as described herein for the HAC, but rather than being present in a HAC, they are integrated into a chromosome of the ungulate.

### SAB-142 Pharmacodynamics and Mechanisms of Action

Pharmacodynamic data show that both 1.5 mg/kg and 2.5 mg/kg doses of SAB-142 demonstrated sustained pharmacodynamic effects for parameters statistically correlated with C-peptide preservation. A sustained exhaustion profile was observed over Day 7 up to Day 120 following a single administration of these two dose levels, indicating durable immunomodulatory effects. Equally important, T-regulatory cell preservation and sustained activation/proliferation were demonstrated for both doses, which supports the intended mechanism of action. Finally, T-conventional differentiation into memory phenotypes was observed for the 1.5 mg/kg and 2.5 mg/kg dose levels, indicating that these doses successfully drive immune adaptation. Given these positive pharmacodynamic responses, the favorable safety and tolerability profile of SAB-142 that demonstrated no serum sickness, no sustained lymphodepletion or depletion of neutrophils, platelets, and red blood cells, and dose-proportional pharmacokinetics, these two dose levels were selected as optimal candidates for further clinical development.

The disclosure demonstrates that the pharmacodynamic effects of SAB-142 are characterized by sustained immunomodulation without sustained lymphodepletion. Following administration, SAB-142 induces transient peripheral lymphopenia within the first 48-72 hours, which resolves by Day 7 as lymphocytes return to baseline levels. This transient reduction in circulating lymphocytes may be attributed to margination rather than cytotoxic depletion.

Circulating lymphocytes are likely not killed or depleted during the transient reduction period; rather, they likely temporarily leave the bloodstream by adhering to vessel walls or redistributing into tissues.

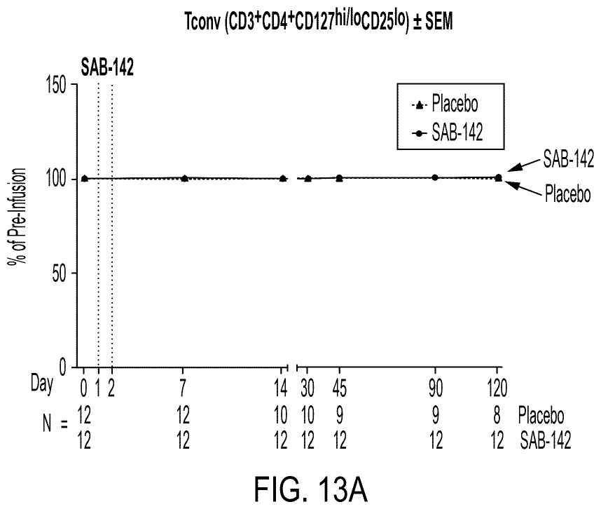

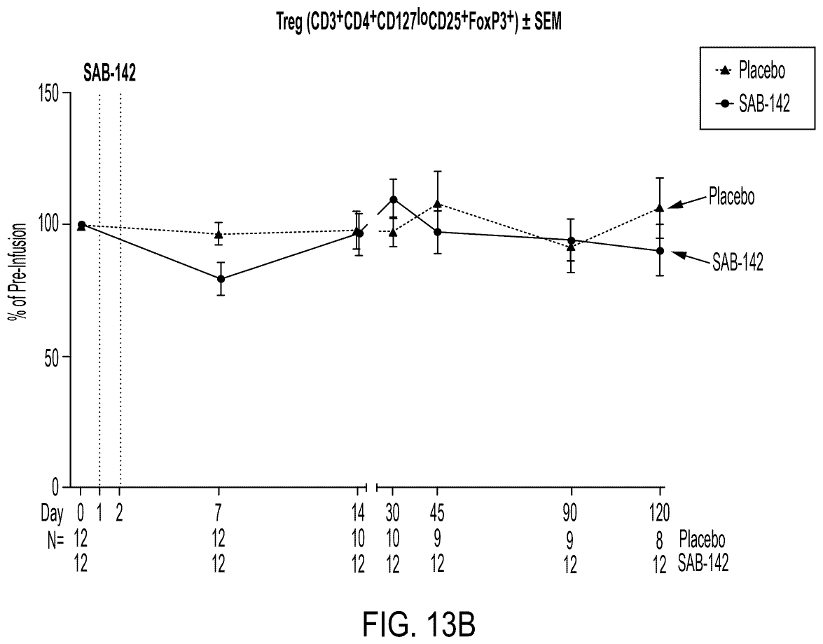

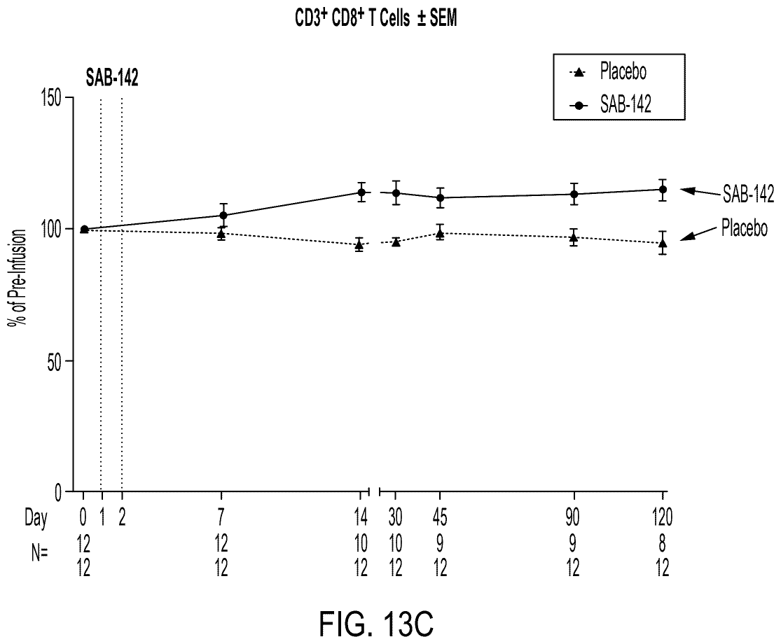

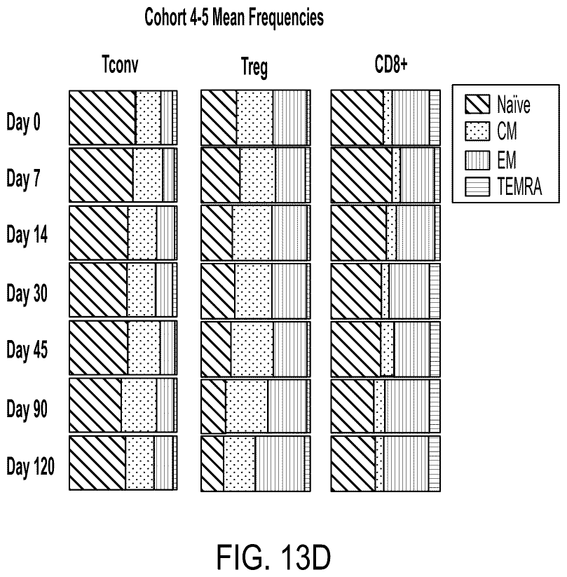

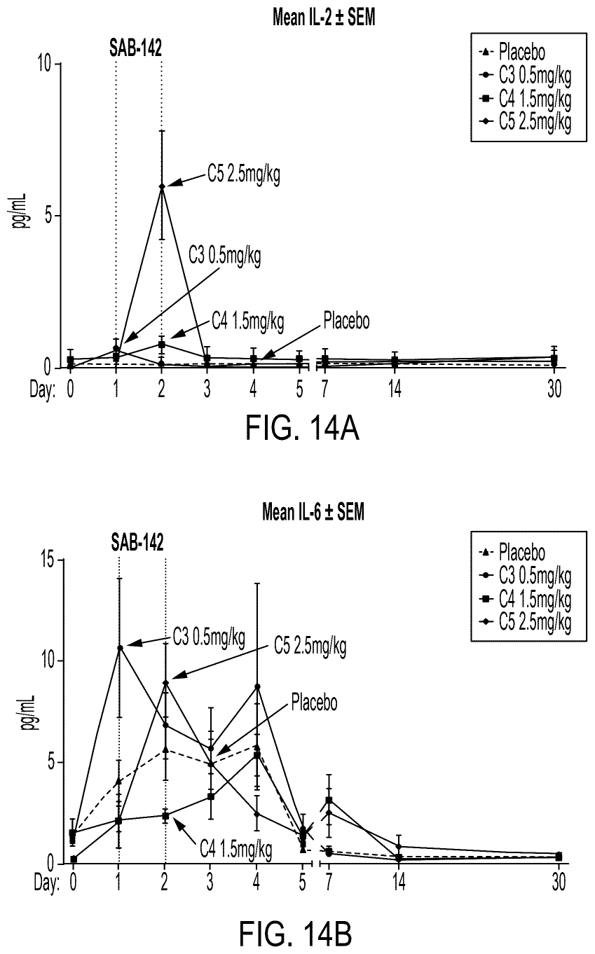

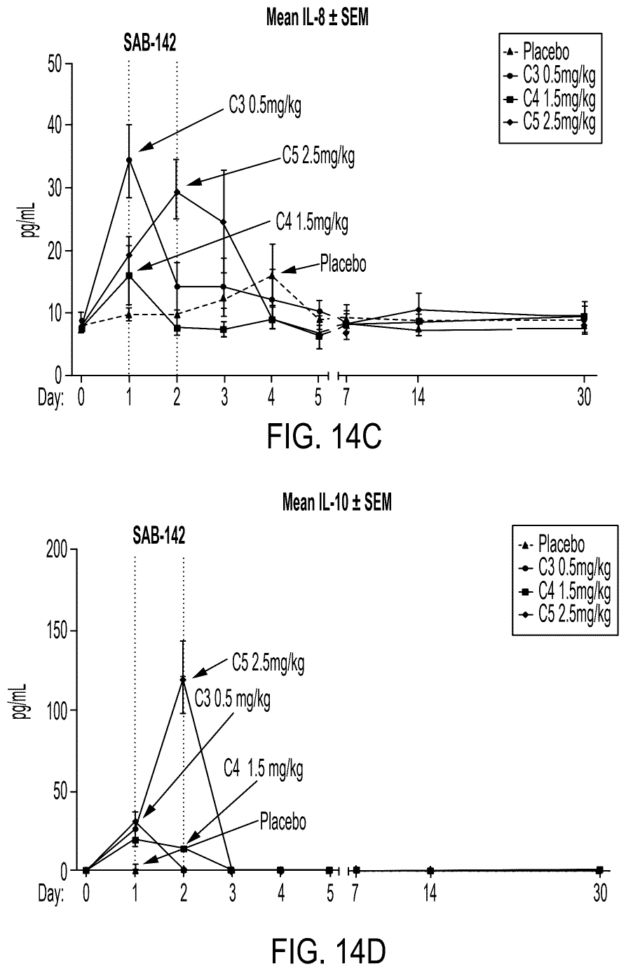

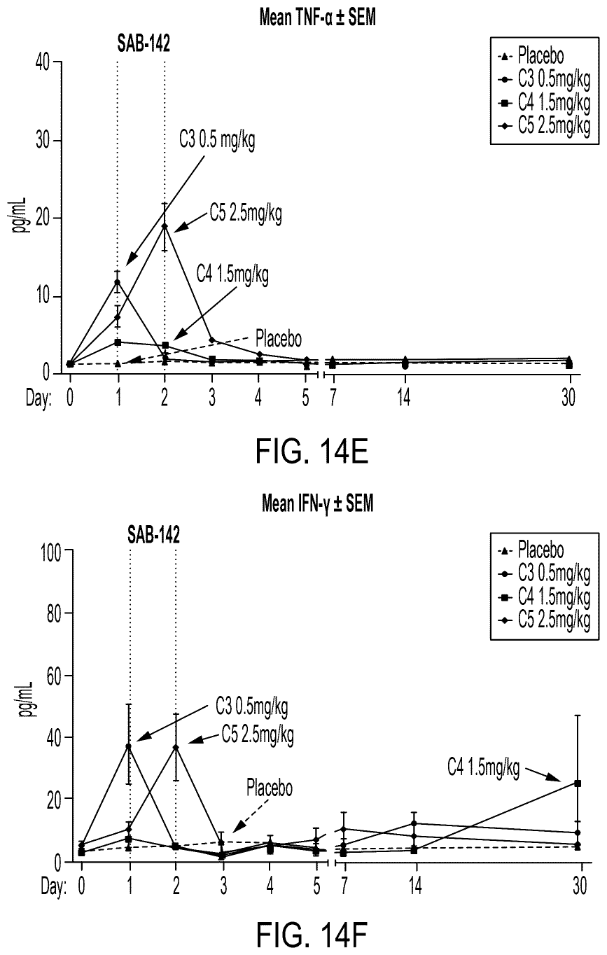

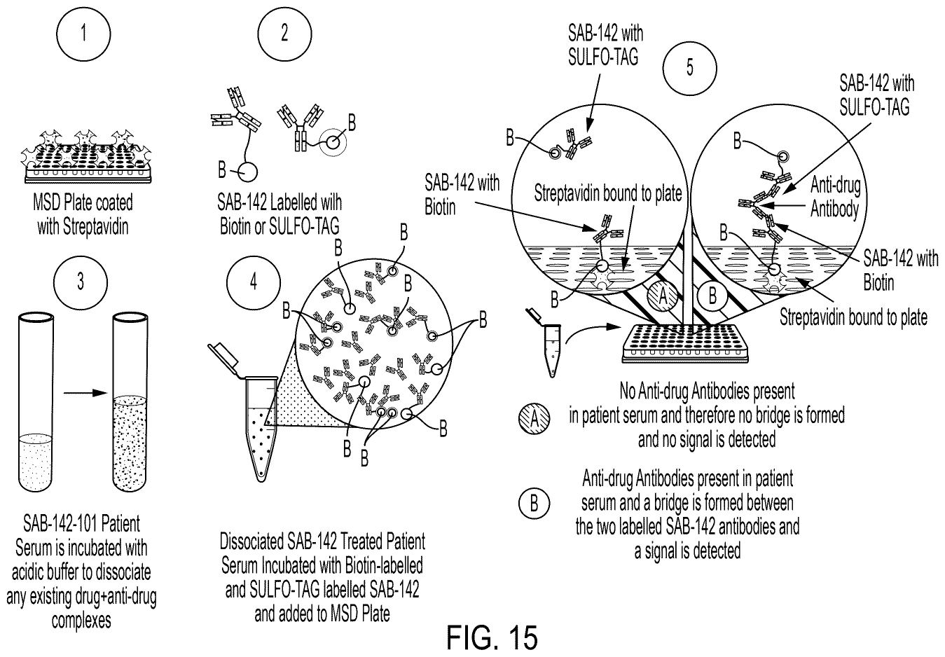

In contrast to the transient reduction in lymphocytes, SAB-142 induces a sustained T-cell exhaustion signature, characterized by increased expression of co-inhibitory receptors, including PD-1, TIGIT, and KLRG1, on both CD4+ and CD8+ T cells. This exhaustion phenotype persists through at least 120 days post-administration and has been correlated with C-peptide preservation in type 1 diabetes patients. SAB-142 preserves regulatory T cell (Treg) populations, with CD3⁺ CD4⁺ CD127^{lo}CD25⁺ FoxP3+ cells remaining stable throughout the observation period (e.g., FIG. 13B). Additionally, treatment with SAB-142 may result in increased expression of TIGIT on Treg cells, potentially enhancing their suppressive function. SAB-142 induces phenotypic shifts in T cell populations, with a notable shift toward central memory (CM) phenotypes in both CD4+ and CD8+ T cells. The pharmacodynamic profile of SAB-142 also includes transient elevation of Th1 and innate inflammatory cytokines, particularly IL-6 (FIG. 14B), IL-10 (FIG. 14D), TNF-α (FIG. 14E), and IFN-7 (FIG. 14F), which peak within the first week following administration and return toward baseline levels by Week 4. Unlike rabbit anti-thymocyte globulin, SAB-142 does not cause sustained depletion of CD4+ T cells, red blood cells, neutrophils, or platelets, with all hematologic parameters returning to baseline by Day 7 post-treatment.

In some embodiments, SAB-142 induces transient peripheral lymphopenia within about 24 to about 96 hours following administration. In some aspects, the transient peripheral lymphopenia may occur within about 36 to about 84 hours, about 40 to about 80 hours, or about 48 to about 72 hours following administration. In some cases, the lymphopenia may resolve by about Day 3 to about Day 14, by about Day 5 to about Day 10, or by about Day 7 following the last infusion.

In some embodiments, SAB-142 induces T-cell exhaustion characterized by increased expression of co-inhibitory receptors on CD4+ and CD8+ T cells. In some aspects, T-cell exhaustion is characterized by altered expression compared to baseline for biomarkers including PD-1, TIGIT, KLRG1, LAG-3, TIM-3, BTLA, CD160, CD57, CD244/2B4, CTLA-4, Interferon-α, interferon-β, IL-2, interferon-γ, TNF-α, perforin, and granzyme. In some aspects, the expression of a biomarker may increase by about 10% to about 200%, about 20% to about 150%, about 30% to about 120%, or about 40% to about 100% relative to baseline levels. In some cases, the expression of a biomarker may decrease by about 15% to about 80%, about 25% to about 75%, or about 35% to about 65% relative to baseline levels.

In some embodiments, SAB-142 induces T-conventional (Tconv) cell exhaustion characterized by increased expression of co-inhibitory receptors on CD4+ T cells. In some aspects, Tconv exhaustion is characterized by altered expression compared to baseline for biomarkers including PD-1, TIGIT, KLRG1, LAG-3, TIM-3, BTLA, CD160, CD57, CD244/2B4, CTLA-4, Interferon-α, interferon-β, IL-2, interferon-γ, TNF-α, perforin, and granzyme. In some aspects, the expression of a biomarker may increase by about 10% to about 200%, about 20% to about 150%, about 30% to about 120%, or about 40% to about 100% relative to baseline levels. In some cases, the expression of a biomarker may decrease by about 15% to about 80%, about 25% to about 75%, or about 35% to about 65% relative to baseline levels.

In some aspects, the expression of PD-1 may increase by about 10% to about 200%, about 20% to about 150%, about 30% to about 120%, or about 40% to about 100% relative to baseline levels. In some cases, the expression of TIGIT may increase by about 15% to about 180%, about 25% to about 140%, or about 35% to about 110% relative to baseline levels. In some embodiments, the expression of KLRG1 may increase by about 10% to about 150%, about 20% to about 120%, or about 30% to about 100% relative to baseline levels.

In some embodiments, the ratio of PD-1 to KLRG1 expression on T cells may increase following SAB-142 administration. In some aspects, the PD-1:KLRG1 ratio may increase by about 10% to about 200%, about 20% to about 120%, about 30% to about 100%, or about 40% to about 80% relative to baseline levels at Day 30, Day 45, Day 90, or Day 120 post-administration. In some cases, the median percent change in PD-1:KLRG1 ratio from pre-infusion may range from about 0% to about 250%, about 10% to about 80%, or about 20% to about 60% at Day 90 or Day 120.

In some embodiments, the ratio of PD-1 to TIGIT expression on T cells may increase following SAB-142 administration. In some aspects, the PD-1:TIGIT ratio may increase by about 5% to about 100%, about 10% to about 80%, about 15% to about 60%, or about 20% to about 50% relative to baseline levels at Day 30, Day 45, Day 90, or Day 120 post-administration. In some cases, the median percent change in PD-1:TIGIT ratio from pre-infusion may range from about −20% to about 180%, about −10% to about 60%, or about 0% to about 50% at Day 90 or Day 120.

In some embodiments, the ratio of KLRG1 to TIGIT expression on T cells may increase following SAB-142 administration. In some aspects, the KLRG1:TIGIT ratio may increase by about 10% to about 200%, about 20% to about 150%, about 25% to about 100%, or about 30% to about 80% relative to baseline levels at Day 30, Day 45, Day 90, or Day 120 post-administration. In some cases, the median percent change in KLRG1:TIGIT ratio from pre-infusion may range from about 0% to about 250%, about 10% to about 80%, or about 20% to about 60% at Day 90 or Day 120.

In some embodiments, the sustained increase in exhaustion marker ratios may distinguish SAB-142-treated subjects from placebo-treated subjects. In some aspects, placebo-treated subjects may exhibit minimal changes or decreases in PD-1:KLRG1, PD-1:TIGIT, and KLRG1:TIGIT ratios over the same observation period. In some cases, the difference in median percent change between SAB-142 and placebo groups for these ratios may range from about 20% to about 150%, about 30% to about 120%, or about 40% to about 100% at Day 90 or Day 120.

In some embodiments, the T-cell exhaustion phenotype persists for at least about 30 days, at least about 60 days, at least about 90 days, at least about 120 days, at least 2 months, at least 3 months, at least 4 months, at least 5 months, at least 6 months, or at least 7 months post-administration. In some aspects, the exhaustion phenotype may persist for about 1 month to about 7 months, about 4 months to about 6 months, about 30 days to about 180 days, about 60 to about 160 days, or about 90 to about 120 days. In some cases, sustained expression of co-inhibitory receptors may be observed at about Day 30, about Day 60, about Day 90, about Day 120, about 5 months, about 6 months, or about 7 months following treatment.

In some embodiments, SAB-142 treatment results in C-peptide preservation ranging from about 5% to about 50%, about 10% to about 45%, about 15% to about 40%, or about 20% to about 35% improvement relative to baseline or placebo at from about 1 to 12 months post-administration. In some embodiments, SAB-142 treatment results in C-peptide preservation ranging from about 5% to about 50%, about 10% to about 45%, about 15% to about 40%, or about 20% to about 35% improvement relative to baseline or placebo at 6 months post-administration. In some aspects, the change from baseline or placebo in C-peptide area under the curve may range from about −30% to about +10%, about −25% to about +5%, about −20% to about 0%, or about −15% to about −5% in SAB-142-treated patients relative to baseline or placebo at 6 months post-administration. In some cases, patients receiving SAB-142 may maintain C-peptide levels at about 60% to about 100%, about 70% to about 95%, about 75% to about 90%, or about 80% to about 85% of baseline or placebo values at 6 months post-administration.

In some embodiments, SAB-142 induces transient elevation of Th1 and innate inflammatory cytokines. In some aspects, IFN-7 levels may increase by about 2-fold to about 100-fold, about 5-fold to about 80-fold, about 10-fold to about 60-fold, or about 15-fold to about 50-fold relative to baseline within the first week following administration. In some cases, IL-6 levels may increase by about 2-fold to about 50-fold, about 3-fold to about 40-fold, about 5-fold to about 30-fold, or about 8-fold to about 25-fold relative to baseline. In some embodiments, TNF-α levels may increase by about 2-fold to about 40-fold, about 3-fold to about 30-fold, about 5-fold to about 25-fold, or about 8-fold to about 20-fold relative to baseline. In some aspects, IP-10 levels may increase by about 2-fold to about 60-fold, about 5-fold to about 50-fold, about 10-fold to about 40-fold, or about 15-fold to about 35-fold relative to baseline.

In some embodiments, the peak elevation of Th1 and innate inflammatory cytokines occurs within about 12 hours to about 7 days, about 24 hours to about 5 days, about 36 hours to about 4 days, or about 48 hours to about 3 days following administration. In some aspects, cytokine levels may return to within about 1.5-fold to about 3-fold, about 1.5-fold to about 2.5-fold, or about 1.5-fold to about 2-fold of baseline levels by about Week 2 to about Week 6, about Week 3 to about Week 5, or about Week 4 following treatment.

In some embodiments, the transient reduction in circulating lymphocytes may be characterized by a decrease to about 20% to about 80%, about 30% to about 70%, about 40% to about 60%, or about 45% to about 55% of baseline levels. In some aspects, lymphocyte counts may return to about 70% to about 130%, about 80% to about 120%, about 85% to about 115%, or about 90% to about 110% of baseline levels by Day 7.

In some embodiments, SAB-142 preserves Treg populations at about 80% to about 120%, about 85% to about 115%, about 90% to about 110%, or about 95% to about 105% of baseline levels throughout the observation period. In some aspects, the ratio of Tregs to conventional T cells may increase by about 5% to about 100%, about 10% to about 80%, about 15% to about 60%, or about 20% to about 50% relative to baseline following SAB-142 administration.

### SAB-142 Composition: Initial Safety Results in Non-Human Primates

SAB-142 shows binding specific to human T-cells, B-cells, and monocytes, which were isolated from human PBMCs, which in a broad sense is somewhat comparable to rabbit- or horse-derived ATG antibodies, Thymoglobulin® and ATGAM®, respectively. SAB-142 is also able to influence the survival and activation of T-cell subsets, such as T-regulatory and T-conventional cells, and exhibits potency against CD8+ T-cells, similar to Thymoglobulin®. SAB-142 has specific binding to a number of human immune system cell types and mimics the mechanism of action of Thymoglobulin®, as was suggested by lymphocyte modulation in cynomolgus monkeys in a GLP toxicology study.

Results from these studies suggest that the mechanism of action of SAB-142 is analogous to Thymoglobulin® and may be beneficial as an immunomodulatory therapeutic agent in T1D to delay its onset and progression.

Cynomolgus monkeys were selected as the relevant species for a single-dose 1-month GLP toxicology study. SAB-142 was well tolerated following a single intravenous (IV) infusion of 1, 5, or 10 mg/kg in male and female cynomolgus monkeys up to the maximum dose tested, 10 mg/kg (no observed adverse effect level [NOAEL] dose). There was no evidence of treatment-related AEs, and these results supported a first-in-human (FIH) study of SAB-142 in healthy volunteers (clinical trial SAB-142-101).

A GLP study in juvenile cynomolgus monkeys was also conducted to support dosing in children and adolescents. This study aimed to evaluate the toxicity, toxicokinetics, and immunologic effects of SAB-142 when administered via IV infusion to juvenile cynomolgus monkeys (equivalent to a human age of 5 to <8 years), followed by at least 26 weeks of observation. After at least 26 weeks of observation, animals received a second dose, administered via IV infusion, followed by at least 12 weeks of observation. Additionally, the toxicity, toxicokinetics, and immunologic effects of SAB-142 were compared to the active comparator (Thymoglobulin®) administered via IV infusion at 25 mg/kg, as the first and the second dose after approximately 24 weeks of observation following the initial dosing. For this report, Groups are referred to by the initial and second dose levels administered, i.e., 5/50, 10/10, and 25/25 mg/kg of SAB-142 compared to 25/25 mg/kg of the active comparator Thymoglobulin®.

No SAB-142 or Thymoglobulin®-related mortality was observed. No SAB-142- or Thymoglobulin®-related alterations in body weight or overall body weight change occurred, and no changes in qualitative food consumption were observed. No SAB-142 or Thymoglobulin®-related neurobehavioral, electrocardiogram (ECG), or urinalysis effects were observed. No organ weight differences, macroscopic observations, or microscopic findings were noted at the terminal sacrifice. The T-cell Mediated Antibody Response (TDAR) assessment showed no negative impact on cellular and humoral immunity measured by anti-KLH IgM and IgG titers; as such, no negative effects indicate that SAB-142 and Thymoglobulin®, administered at doses up to 50 mg/kg at initial dose or redosing don't impact immune response to neo- or recall vaccination. This immunotoxicology assessment indicates no immunosuppression is induced by SAB-142 in juvenile monkeys. No SAB142-related hematology, coagulation, or clinical chemistry effects were noted for animals administered 5/50, 10/10 or 25/25 mg/kg SAB-142 (initial/re-dose). The NOAEL was declared at 50 mg/kg, the highest dose level tested.

### SAB-142 Pharmacokinetic Profiles in Humans

SAB-142 in human serum samples is quantitatively measured using the validated SAB-142 PK multiple target assay. This is a flow cytometry-based PK assay that utilizes pooled human PBMCs, gated for lymphocytes, as the targeted cell populations to measure the concentration of unbound free SAB-142 in the serum of patients infused with SAB-142. Following an IV infusion of SAB-142, SAB-142 binds to the lymphocytes in the blood, resulting in lower concentration of unbound free SAB-142 circulating in the bloodstream.

The maximum observed concentration (Cₘₐₓ) and area under the concentration-time curve (AUC) values of SAB-142 in human serum increased in a dose-proportional manner across the 0.5, 1.5, and 2.5 mg/kg doses. Systemic exposure to SAB-142 was limited at the 0.5 mg/kg dose. The Cₘₐₓ values ranged from 0.679 to 1.01 and 1.04 to 3.00 μg/mL at 1.5 and 2.5 mg/kg, respectively, and AUC from time 0 to the last measurable concentration (AUCₗₐₛₜ) values ranged from 9.15 to 36.0 and 31.7 to 105 h*μg/mL at 1.5 and 2.5 mg/kg, respectively. The half-life (t_{1/2}) from Cohort 5 at 2.5 mg/kg was between 26.4 and 61.6 hours.

A meso scale discovery electro-chemiluminescent-based bridging assay, with the multi-tiered approach recommended by the FDA, has been developed and validated to measure anti-SAB-142 antibodies in human serum. This approach includes screening, confirmatory, and titre (US FDA: Immunogenicity Testing of Therapeutic Protein Products—Developing and Validating Assays for Anti-Drug Antibody Detection, 2019). Among 24 subjects dosed with SAB-142 at 0.5, 1.5, and 2.5 mg/kg, respectively, only one subject in Cohort 3, who was dosed at 0.5 mg/kg, tested positive for anti-drug antibodies (ADA), but the titre was negligible, with a dilution of 1:10, which is the minimal required dilution for this ADA assay and is equivalent to the background level. At the target doses of 1.5 and 2.5 mg/kg, Cohorts 4 and 5 did not generate anti-SAB-142 antibodies in healthy volunteers. This demonstrated that SAB-142, fully human IgG polyclonal antibodies, is less immunogenic than Thymoglobulin®, rabbit IgG polyclonal antibodies.

### SAB-142 Induces Balanced Immunomodulation in Humans Transient Lymphocyte Reduction Without Lymphodepletion

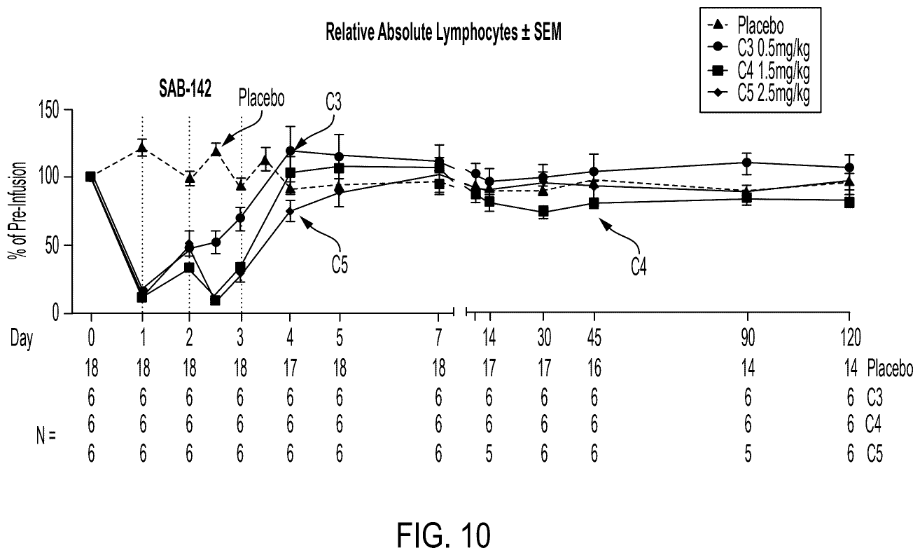

SAB-142 induces a transient decrease in circulating lymphocytes that resolves rapidly without sustained lymphodepletion. In some embodiments, the transient nature of lymphocyte reduction may be characterized by a return to baseline levels within approximately 48 to 72 hours following the last infusion. In some aspects, the transient lymphocyte decrease may be evidenced by recovery to at least 80%, at least 85%, at least 90%, or at least 95% of pre-infusion lymphocyte counts within 7 days of treatment (e.g., FIG. 10). In some cases, the transient nature may be demonstrated by the absence of sustained reductions in absolute lymphocyte counts beyond 14 days post-administration. In some embodiments, the transient lymphocyte decrease may be distinguished from lymphodepletion by the preservation of lymphocyte populations at Day 7, Day 14, or Day 30 post-treatment. In some aspects, the transient decrease may be characterized by a low point occurring within 24 to 60 hours post-infusion, followed by rapid recovery. In some cases, the transient nature may be indicated by the maintenance of lymphocyte counts above a threshold of 200 cells/μL 500 cells/μL, 750 cells/μL, or 1000 cells/μL throughout the observation period. In some embodiments, the transient lymphocyte decrease may be evidenced by flow cytometry analysis showing redistribution rather than depletion of lymphocyte populations. In some aspects, the transient nature may be confirmed by the absence of prolonged lymphopenia extending beyond one week post-administration.

In some embodiments, the compositions of the disclosure induce sustained lymphocyte exhaustion, e.g., T cell exhaustion. In some embodiments, the T cell exhaustion is sustained for at least 10 days, 20 days, 30 days, 2 months, 3 months, 4 months, 5 months, 6 months or more after a treatment with compositions disclosed herein. In some embodiments, the composition induces sustained T cell exhaustion, which persists for at least 30 days or longer. In some embodiments, the T cell exhaustion comprises an increase in the expression of PD-1, TIGIT, and KLRG1, or a combination thereof. In some embodiments, T cell exhaustion is characterized by an increase in the ratio of PD-1 to KLRG1, PD-1 to TIGIT, and/or KLRG1 to TIGIT, compared to a baseline or reference level.

In some embodiments, the composition does not induce a sustained reduction in immune cell numbers. In some embodiments, the immune cells can be NK cells, B cells, or T cells. In some embodiments, the composition can induce a transient but not sustained reduction in the number of immune cells.

### Sustained T-Cell Exhaustion Following SAB-142 Administration

In some embodiments, T cell exhaustion may be detected by measuring the expression of inhibitory receptors on T cell surfaces. In some aspects, exhaustion may be characterized by increased expression of PD-1 on CD4+ T cells, CD8+ T cells, or both. In some cases, exhaustion may be identified by elevated expression of TIGIT on conventional T cells (Tconv) or regulatory T cells (Tregs). In some embodiments, exhaustion may be evidenced by increased expression of KLRG1 on T cell subsets. In some aspects, exhaustion may be detected by measuring co-expression of multiple inhibitory receptors.

In some embodiments, T cell exhaustion may be measured by flow cytometry analysis of peripheral blood mononuclear cells. In some aspects, exhaustion may be quantified by determining the percentage of T cells expressing one or more exhaustion markers. In some cases, exhaustion may be assessed by measuring median fluorescence intensity (MFI) of inhibitory receptor expression. In some embodiments, exhaustion may be evaluated by comparing the frequency of exhaustion marker-positive cells at baseline versus post-treatment timepoints.

In some embodiments, T cell exhaustion may be detected by analyzing changes in T cell phenotype. In some embodiments, T cell exhaustion may be measured by assessing functional capacity of T cells. In some aspects, exhaustion may be characterized by reduced proliferative capacity in response to stimulation. In some embodiments, exhaustion may be detected by measuring reduced cytotoxic activity of CD8+ T cells.

In some embodiments, sustained exhaustion may be confirmed by longitudinal measurements at multiple timepoints. In some aspects, sustained exhaustion may be evidenced by persistent elevation of exhaustion markers at 30 days, 60 days, 90 days, 120 days, 5 months, 6 months, or 7 months post-treatment. In some cases, sustained exhaustion may be characterized by maintenance of elevated inhibitory receptor expression throughout the observation period. In some embodiments, sustained exhaustion may be distinguished from transient activation by the durability of the exhaustion phenotype beyond acute treatment effects.

### Preservation of Regulatory T Cell Populations

In some embodiments, SAB-142 demonstrates preservation of regulatory T cell (Treg) populations following administration. In some aspects, Treg preservation may be characterized by maintenance of Treg cell frequencies at or near baseline levels throughout the treatment period. In some cases, Treg populations may remain stable at Day 7, Day 30, Day 60, Day 90, Day 120, 5 months, 6 months, or 7 months post-administration. In some embodiments, Treg preservation may be evidenced by the absence of sustained reductions in absolute Treg counts following treatment. In some embodiments, Treg preservation is measured using levels of CD25, CD127, FoxP3, TIGIT, and a combination thereof.

In some embodiments, SAB-142 may induce activation of Treg populations. In some aspects, Treg activation may be characterized by increased expression of inhibitory receptors on Treg cells. In some cases, Treg activation may be evidenced by elevated TIGIT expression on Treg cells. In some aspects, Treg activation may be characterized by upregulation of co-inhibitory molecules that enhance suppressive function.

In some embodiments, Treg activation may be measured by flow cytometry analysis. In some aspects, activation may be quantified by determining the percentage of TIGIT+ cells within the Treg population. In some cases, activation may be assessed by measuring median fluorescence intensity of activation markers on Tregs. In some embodiments, activation may be evaluated by comparing marker expression at baseline versus post-treatment timepoints.

In some embodiments, Treg preservation may be distinguished from conventional T cell responses. In some aspects, while conventional T cells may exhibit transient reductions in circulating numbers, Treg populations may remain stable. In some cases, the ratio of Tregs to conventional T cells may increase following SAB-142 administration. In some embodiments, selective preservation of Tregs may contribute to immunomodulatory effects without sustained immunosuppression.

In some embodiments, Treg preservation and activation may persist throughout the observation period. In some aspects, sustained Treg activation may be evidenced by persistent elevation of TIGIT expression at 30 days, 60 days, 90 days, 120 days, 5 months, 6 months, or 7 months post-treatment. In some cases, maintained Treg populations may support long-term immunomodulatory effects. In some embodiments, the combination of Treg preservation and activation may distinguish SAB-142 from other immunomodulatory agents that deplete or impair Treg function.

In some embodiments, Treg sparing may be measured by comparing Treg frequencies before and after treatment. In some aspects, preservation may be defined as maintenance of Treg frequencies within 80%, 85%, 90%, 95%, or 100% of baseline values. In some cases, Treg preservation may be assessed by measuring absolute Treg counts rather than percentages. In some embodiments, preservation may be confirmed by the absence of significant reductions in Treg populations at any measured timepoint.

In some embodiments, the Treg-sparing effect may contribute to the safety profile of SAB-142. In some aspects, preservation of regulatory immune function may reduce the risk of opportunistic infections. In some cases, maintained Treg populations may prevent excessive immune activation. In some embodiments, Treg preservation may support the potential for repeat dosing without cumulative immunosuppressive effects.

### Sustained T-Cell Exhaustion Signature

A sustained T-cell exhaustion signature across key immune cell populations has been identified as an important factor associated with C-peptide preservation in type 1 diabetes. Treatment with SAB-142 results in a decrease in CXCR3+ CD4+ and CXCR3+ conventional T cells (Tconv) and shifts the T cell profile toward a central memory (CM) phenotype, particularly in cohorts receiving 1.5 to 2.5 mg/kg (see FIG. 13D). Additionally, SAB-142 reduces the population of CD4+ TEMRA cells, a reduction that is associated with decreased cytotoxic effects on residual beta cells. A multi-target T-cell exhaustion profile, as measured by PD-1 expression on both CD4+ and CD8+ cells, has been shown to correlate with C-peptide preservation in type 1 diabetes, underscoring the therapeutic potential of SAB-142 in promoting durable immunomodulation and preservation of endogenous insulin production.

In some embodiments, SAB-142 administration may result in decreased frequencies of CXCR3+CD4+ T cells. In some aspects, the reduction in CXCR3+CD4+ cells may be observed within the first weeks following treatment. In some cases, decreased CXCR3+expression may be detected on non-naive CD4+ T cell populations. In some embodiments, the reduction in CXCR3+CD4+ cells may persist for at least 30 days, 60 days, 90 days, 120 days, 5 months, 6 months, or 7 months post-administration.

In some embodiments, SAB-142 may reduce the frequency of CXCR3+ conventional T cells (Tconv). In some aspects, the decrease in CXCR3+ Tconv cells may be characterized by reduced expression of this chemokine receptor on CD4+ effector populations. In some aspects, the reduction may be measured as a percentage of total Tconv cells expressing CXCR3. In some embodiments, decreased CXCR3+ Tconv frequencies may be accompanied by phenotypic shifts in T cell memory subsets.

In some embodiments, the reduction in CXCR3+ cells may be measured by flow cytometry analysis. CXCR3 expression may be quantified on gated CD4+ T cell populations. Changes in CXCR3 expression may be assessed by comparing baseline values to post-treatment timepoints.

In some embodiments, the reduction in CXCR3+CD4+ cells may be sustained throughout the observation period. In some aspects, decreased frequencies may be maintained at 3 months, 6 months, 9 months, or 12 months post-treatment. In some cases, the durability of CXCR3 reduction may support long-term disease modification. In some embodiments, sustained decreases in CXCR3+ cells may enable extended intervals between treatment administrations.

A sustained T-cell exhaustion signature across key immune cell populations has been identified as an important factor associated with C-peptide preservation in type 1 diabetes. Treatment with SAB-142 results in a decrease in CXCR3+CD4+ and CXCR3+ conventional T cells (Tconv) and shifts the T cell profile toward a central memory (CM) phenotype, particularly in cohorts receiving 1.5 to 2.5 mg/kg (see FIG. 13D). Additionally, SAB-142 reduces the population of CD4+ TEMRA cells, a reduction that is associated with decreased cytotoxic effects on residual beta cells. A multi-target T-cell exhaustion profile, as measured by PD-1 expression on both CD4+ and CD8+ cells, has been shown to correlate with C-peptide preservation in type 1 diabetes, underscoring the therapeutic potential of SAB-142 in promoting durable immunomodulation and preservation of endogenous insulin production.

In some embodiments, SAB-142 administration may result in decreased frequencies of CXCR3+CD4+ T cells. In some aspects, the reduction in CXCR3+CD4+ cells may be observed within the first weeks following treatment. In some cases, decreased CXCR3+expression may be detected on non-naive CD4+ T cell populations. In some embodiments, the reduction in CXCR3+CD4+ cells may persist for at least 30 days, 60 days, 90 days, 120 days, 5 months, 6 months, or 7 months post-administration.

In some embodiments, SAB-142 may reduce the frequency of CXCR3+ conventional T cells (Tconv). In some aspects, the decrease in CXCR3+ Tconv cells may be characterized by reduced expression of this chemokine receptor on CD4+ effector populations. In some aspects, the reduction may be measured as a percentage of total Tconv cells expressing CXCR3. In some embodiments, decreased CXCR3+ Tconv frequencies may be accompanied by phenotypic shifts in T cell memory subsets.

In some embodiments, the reduction in CXCR3+ cells may be measured by flow cytometry analysis. CXCR3 expression may be quantified on gated CD4+ T cell populations. Changes in CXCR3 expression may be assessed by comparing baseline values to post-treatment timepoints.

In some embodiments, the reduction in CXCR3+ CD4+ cells may be sustained throughout the observation period. In some aspects, decreased frequencies may be maintained at 3 months, 6 months, 9 months, or 12 months post-treatment. In some cases, the durability of CXCR3 reduction may support long-term disease modification. In some embodiments, sustained decreases in CXCR3+ cells may enable extended intervals between treatment administrations.

### Superior Cytokine Release Profiles of SAB-142 Composition

SAB-142 and rATG induced cytokine secretion in similar patterns across a selected subset of cytokines. SAB-142 induced release of pro-inflammatory cytokines including IL-6, IL-8, TNF-α, and IFN-γ. In some cases, SAB-142 may also induce secretion of regulatory cytokines including IL-2 and IL-10. In some embodiments, the cytokine release profile of SAB-142 may be characterized by transient elevation of Th1 and innate inflammatory cytokines.

Cytokine release may be observed following SAB-142 administration. In some aspects, notable elevations may be observed in IFN-γ, IL-6, IP-10, and TNF-α in SAB-142-treated groups compared to placebo. In some cases, cytokine concentrations may return toward baseline levels by Week 12 and Week 24. In some embodiments, the transient surge in cytokines may be particularly evident at Week 2 following treatment initiation.

In some embodiments, cytokine release may be measured using a validated multiplex cytokine assay. In some aspects, the assay may measure a panel of cytokines including IL-1β, IL-2, IL-4, IL-6, IL-8, TNF-α, IFN-γ, IL-10, IL-12p70, and IL-13. In some cases, peripheral blood mononuclear cells (PBMCs) may be incubated with SAB-142 and cytokine concentrations in the culture supernatant may be quantified using a multiplexed cytokine bead array platform. In some embodiments, quantitation of cytokine concentrations may be performed by interpolation from standard curves of known values.

In some embodiments, the cytokine release pattern may be consistent with immune activation following anti-thymocyte globulin administration. In some aspects, the transient nature of cytokine elevation may distinguish SAB-142 from agents causing sustained inflammatory responses. In some cases, the return of cytokine levels to baseline may indicate resolution of acute immune activation. The cytokine release profile may provide mechanistic insight into the immunomodulatory effects of SAB-142 in type 1 diabetes patients.

In some embodiments, responders to SAB-142 therapy may exhibit a distinct cytokine signature. In some aspects, responders may show transient Th1 cytokine elevation. In some cases, the cytokine release pattern may correlate with therapeutic response. In some embodiments, the cytokine profile may serve as a potential biomarker for predicting therapeutic response to SAB-142 in type 1 diabetes.

Following single-dose administration of SAB-142 in juvenile cynomolgus monkeys, no SAB-142- or Thymoglobulin®-related changes in TNF-α, IL-8, GM CSF, IL-10, IFN-γ, IL-17a, IL-10, IL-5, or IL-4 were noted. Both agents elicited increases in pro-inflammatory cytokines such as IL-6, IL-8, TNF-α, and IFN-γ, as well as regulatory cytokines IL-2 and IL-10. No long-term SAB-142 or rATG-related changes in pro-inflammatory cytokines (TNF-α, IL-8, etc.) were noted (see FIG. 14A-FIG. 14F). The overall cytokine release profiles were highly analogous, indicating that SAB-142 recapitulates the immunomodulatory effects of rATG at the level of cytokine induction.

C-Peptide Preservation After Treatment with SAB-142

Pharmacodynamic data show that both 1.5 mg/kg and 2.5 mg/kg doses of SAB-142 demonstrated sustained pharmacodynamic effects for parameters statistically correlated with C-peptide preservation. C-peptide preservation has been indicated as a measure of endogenous insulin production and beta-cell function in patients with type 1 diabetes. A T-regulatory sparing effect, transient cytokine induction, and CD4⁺ cell exhaustion were observed following a single administration of these two dose levels, indicating durable immunomodulatory effects. Additionally, T-conventional differentiation into a memory phenotype was observed for the 1.5 mg/kg and 2.5 mg/kg dose levels, indicating that these 2 doses successfully drive immune adaptation.

C-peptide levels may be measured using a 2-hour mixed meal tolerance test (MMTT). The area under the concentration-time curve (AUC) of C-peptide following the MMTT may provide a quantitative measure of beta-cell function. In some cases, C-peptide AUC may be calculated using the trapezoidal rule from samples collected at multiple timepoints during the MMTT.

In some embodiments, SAB-142 treatment may result in a significantly reduced decline in C-peptide levels compared to placebo. In some aspects, the change from baseline in C-peptide ln(AUC+1) at 12 months may serve as a primary endpoint for evaluating therapeutic efficacy. In some cases, smaller decreases in C-peptide levels may indicate better preservation of beta-cell function. In some embodiments, maintained C-peptide production may enable patients to achieve better glycemic control with lower exogenous insulin doses.

In some embodiments, SAB-142 treatment may result in C-peptide preservation ranging from about 5% to about 50% improvement relative to baseline at 12 months. In some aspects, the change from baseline in C-peptide levels may range from about −10% to about +10% in SAB-142-treated patients. In some cases, patients receiving SAB-142 may maintain C-peptide levels at about 70% to about 95% of baseline values at 12 months. In some aspects, individual patient responses may vary, with some patients maintaining C-peptide levels at about 80% to about 100% of baseline, while others may experience declines to about 60% to about 80% of baseline values. In some cases, the degree of C-peptide preservation may correlate with baseline C-peptide levels, with patients having higher baseline values potentially showing greater absolute preservation.

In some aspects, fasting C-peptide levels may range from about 0.05 nmol/L to about 0.8 nmol/L at 12 months following SAB-142 administration. In some cases, stimulated peak C-peptide levels during the MMTT at 12 months following SAB-142 administration may be at least about 0.2 nmol/L, at least about 0.3 nmol/L, at least about 0.4 nmol/L, at least about 0.5 nmol/L, at least about 0.6 nmol/L, at least about 0.7 nmol/L, at least about 0.8 nmol/L, at least about 0.9 nmol/L, or at least about 1.0 nmol/L.

In some embodiments, the proportion of patients maintaining a clinically significant stimulated peak C-peptide of at least 0.2 nmol/L during the 2-hour MMTT may be about 40%, about 50%, about 60%, about 70%, about 80%, or about 90% at 12 months post-treatment.

In some embodiments, the proportion of participants maintaining clinically significant stimulated peak C-peptide levels may be assessed. In some aspects, a clinically significant C-peptide level may be defined as a stimulated peak C-peptide of at least 0.2 nmol/L during the 2-hour MMTT. In some cases, maintenance of C-peptide levels above this threshold may be associated with meaningful clinical benefits. In some embodiments, higher proportions of participants maintaining clinically significant C-peptide levels in SAB-142 treatment groups compared to placebo may indicate therapeutic efficacy.

In some embodiments, C-peptide preservation may correlate with the immunomodulatory effects of SAB-142. In some aspects, participants exhibiting sustained T-cell exhaustion markers may demonstrate better C-peptide preservation. In some cases, the multi-target T-cell exhaustion profile, as measured by PD-1 expression on CD4+ and CD8+ cells, may correlate with maintained C-peptide production.

In some embodiments, responders to SAB-142 therapy may be characterized by a maintained or slower decline in C-peptide levels compared to placebo. In some aspects, responders may exhibit distinct immunological signatures, including sustained expression of inhibitory receptors on T cells, such as PD-1, TIGIT, KLRG. In some cases, a combination of preserved C-peptide and specific immune markers may identify participants most likely to benefit from SAB-142 treatment. In some embodiments, C-peptide response patterns may inform decisions regarding repeat dosing or treatment duration.

In some embodiments, C-peptide preservation may be evaluated over extended periods beyond 12 months. In some aspects, long-term follow-up at 18 months and 24 months may assess the durability of beta-cell preservation. In some cases, sustained C-peptide production over 24 months may indicate disease modification rather than temporary stabilization. In some embodiments, participants receiving repeat doses of SAB-142 may demonstrate enhanced or prolonged C-peptide preservation compared to single-dose treatment.

In some embodiments, the fasting proinsulin-to-C-peptide ratio may provide additional information about beta-cell function. In some aspects, this ratio may serve as a measure of beta-cell endoplasmic reticulum stress and dysfunction. In some cases, lower proinsulin-to-C-peptide ratios, compared to baseline or a placebo control, may indicate healthier beta-cell function. In some embodiments, SAB-142 treatment may be associated with favorable changes in the proinsulin-to-C-peptide ratio.

In some embodiments, calculated estimated residual stimulated C-peptide (CP_{EST}) may be used to estimate beta-cell function. In some aspects, CP_{EST} may be calculated from fasting C-peptide and HbA1c values. In some cases, CP_{EST} may provide an estimate of stimulated C-peptide without requiring an MMTT. In some embodiments, CP_{EST} values may be monitored at timepoints between scheduled MMTT assessments.

In some embodiments, C-peptide preservation may translate into clinical benefits for patients. In some aspects, maintained endogenous insulin production may reduce the risk of severe hypoglycemia. In some cases, preserved C-peptide may facilitate achievement of glycemic targets with lower exogenous insulin doses. In some embodiments, participants with better C-peptide preservation may experience improved time in range and reduced glycemic variability. In some aspects, the clinical benefits of C-peptide preservation may extend beyond glycemic control to include reduced risk of long-term diabetes complications.

### Low Risk of Serum Sickness With SAB-142

Serum sickness may represent a significant adverse event associated with the administration of heterologous immunoglobulin preparations. In some embodiments, SAB-142 does not cause serum sickness, due to its fully human nature. This is a substantial improvement compared to rATG or horse ATG.

Serum sickness may occur when the recipient's immune system recognizes and responds to xenogeneic proteins present in animal-derived immunoglobulin preparations. This immune response may manifest as fever, rash, arthralgia, lymphadenopathy, and other systemic symptoms.

In some cases, serum sickness may develop approximately 7 to 14 days following administration of heterologous immunoglobulin. The fully human composition of SAB-142 eliminates or reduces the antigenic stimulus that triggers serum sickness.

In some embodiments, clinical studies of SAB-142 may demonstrate an absence of serum sickness across all dose levels tested. In some aspects, no cases of serum sickness may be observed in participants receiving doses ranging from about 0.5 mg/kg to about 2.5 mg/kg. In some cases, the incidence of serum sickness in SAB-142-treated participants may be about 0% across all cohorts. In some embodiments, this may contrast with reported serum sickness rates of about 5% to about 40% in patients receiving rabbit anti-thymocyte globulin.

In some embodiments, the absence of serum sickness may enable repeat dosing of SAB-142. In some aspects, participants may receive multiple doses of SAB-142 at intervals of about 6 months without developing serum sickness. In some cases, the ability to administer repeat doses may allow for sustained immunomodulation over extended periods. In some embodiments, repeat dosing may be performed at intervals ranging from about 3 months to about 12 months, from about 4 months to about 9 months, or from about 5 months to about 7 months.

In some embodiments, monitoring for serum sickness may include assessment of clinical symptoms and laboratory parameters. In some aspects, participants may be monitored for fever, defined as body temperature exceeding about 38° C. or about 100.4° F. In some embodiments, inflammatory markers may remain within normal ranges in SAB-142-treated participants. In some embodiments, transient elevations in inflammatory markers may occur during the first week following administration but may resolve without progression to serum sickness.

In some embodiments, the absence of serum sickness may improve the safety profile of SAB-142 compared to heterologous anti-thymocyte globulin preparations. In some aspects, participants may not require prophylactic or therapeutic corticosteroids to prevent or manage serum sickness. In some cases, the elimination of corticosteroid use may be particularly beneficial in type 1 diabetes patients, as corticosteroids may impair beta-cell function. In some embodiments, avoiding corticosteroid administration may preserve the therapeutic benefits of SAB-142 on C-peptide levels.

In some embodiments, the lack of serum sickness may contribute to improved treatment adherence and patient acceptance. In some aspects, participants may be more willing to receive repeat doses when serum sickness risk is eliminated. In some cases, the absence of serum sickness may reduce treatment-related anxiety and improve quality of life during the treatment period.

In some embodiments, long-term follow-up may confirm the sustained absence of serum sickness. In some aspects, participants may be monitored for delayed-onset serum sickness symptoms for periods extending to about 30 days, about 60 days, or about 90 days following each dose. In some cases, no delayed cases of serum sickness may be observed even with extended monitoring periods. In some embodiments, the consistent absence of serum sickness across multiple doses and extended observation periods may support the safety of chronic SAB-142 administration.

In some cases, the lack of anti-drug antibodies may prevent the formation of immune complexes that trigger serum sickness. In some embodiments, the fully human nature of SAB-142 may result in immunogenicity rates of about 0% to about 5% at doses ranging from about 1.5 mg/kg to about 2.5 mg/kg.

### Fc Binding Profile of SAB-142 Contributes to Reduced Antibody-Dependent Cellular Cytotoxicity

SAB-142 may exhibit distinct Fc receptor binding characteristics compared to rabbit anti-thymocyte globulin. These binding characteristics may contribute to the favorable safety profile and immunomodulatory effects of SAB-142. Differential Fc receptor engagement may influence effector functions, including antibody-dependent cellular cytotoxicity (ADCC) and antibody-dependent cellular phagocytosis (ADCP).

In some embodiments, SAB-142 may demonstrate enhanced binding to the neonatal Fc receptor (FcRn) compared to rabbit anti-thymocyte globulin. In some aspects, the IC50 for FcRn binding may be about 1.0 to about 5.0 for SAB-142. In some cases, the IC50 for FcRn binding may be about 1.5 to about 3.5 for SAB-142. In some embodiments, the IC50 for FcRn binding may be about 2.0 to about 2.5 for SAB-142. In some aspects, SAB-142 may exhibit an IC50 for FcRn binding of about 2.16. In some cases, this may represent about 5-fold to about 10-fold higher binding affinity compared to rabbit anti-thymocyte globulin, which may exhibit an IC50 of about 15 to about 25, or about 20.11.

Enhanced FcRn binding may contribute to improved pharmacokinetic properties of SAB-142. Increased FcRn binding may result in enhanced antibody recycling and prolonged serum half-life. In some embodiments, the serum half-life of SAB-142 may range from about 20 hours to about 70 hours, from about 25 hours to about 65 hours, or from about 26.4 hours to about 61.6 hours. Enhanced FcRn binding may facilitate improved tissue distribution and access to lymphoid organs where target T cells reside.

In some embodiments, SAB-142 may exhibit binding to FcγRI comparable to rabbit anti-thymocyte globulin. In some aspects, the IC50 for FcγRI binding may be similar between SAB-142 and rabbit anti-thymocyte globulin. Comparable FcγRI binding may indicate similar potential for immune activation through this receptor pathway.

Reduced FcγRIIIa binding may result in decreased antibody-dependent cellular cytotoxicity (ADCC). ADCC activity may be measured using reporter cell lines expressing human FcγRIIIα. In some embodiments, SAB-142 may demonstrate EC50 values for ADCC activation that are about 5-fold to about 15-fold higher than those of rabbit anti-thymocyte globulin. In some embodiments, SAB-142 may exhibit EC50 values for ADCC activation ranging from about 2 μg/mL to about 6 μg/mL. In some aspects, SAB-142 may exhibit EC50 values for ADCC activation of about 3 μg/mL to about 5 μg/mL, or about 3.7 μg/mL. Rabbit anti-thymocyte globulin may exhibit EC50 values for ADCC activation of about 0.2 μg/mL to about 0.5 μg/mL, or about 0.3 μg/mL.

Reduced ADCC activity may contribute to the lymphocyte-sparing properties of SAB-142. Lower ADCC activity may result in immunomodulation without sustained lymphodepletion. In some embodiments, participants receiving SAB-142 may experience transient peripheral lymphopenia that resolves within about 48 hours to about 72 hours, rather than sustained lymphocyte depletion lasting weeks to months. The absence of sustained lymphodepletion may reduce the risk of opportunistic infections and other complications associated with prolonged immunosuppression.

In some embodiments, the Fc binding profile of SAB-142 may be characterized by a combination of enhanced FcRn binding, increased FcγRIIa binding, and reduced FcγRIIIa binding compared to rATG. This combination may provide a balance between effective immunomodulation and acceptable safety. Enhanced FcRn binding may improve pharmacokinetics and tissue distribution, increased FcγRIIa binding may promote ADCP-mediated immune modulation, and reduced FcγRIIIa binding may increase induction of ADCP, minimizing cytotoxic effects.

ADCC and ADCP activity may be measured using cell-based reporter assays. Reporter cell lines may express human Fc receptors coupled to luciferase or other reporter genes. Activation of downstream signaling pathways may result in measurable reporter expression. In some embodiments, EC50 values may represent the concentration required to achieve 50% of maximal pathway activation.

The Fc binding profile of SAB-142 may support its use in chronic dosing regimens. Reduced ADCC activity may minimize cumulative cytotoxic effects with repeat dosing. In some embodiments, participants may receive multiple doses at intervals of about 6 months without experiencing progressive lymphodepletion. Maintaining Fc receptor binding characteristics across repeat doses may ensure consistent immunomodulatory effects.

### Design of Phase 3b Clinical Trial for Efficacy of SAB-142 in Type 1 Diabetes

The SAB-142-301 clinical trial represents a comprehensive Phase 3 study designed to evaluate the efficacy and safety of SAB-142 in participants with Stage 3 new-onset type 1 diabetes (NOT1D). The trial incorporates a domized, double-blind, placebo-controlled design that enables systematic evaluation of safety and efficacy across different disease stages. Data from 36 participants with Stage 3 NOT1D enrolled in the SAFEGUARD study will constitute the synthetic Cohort 1, while approximately 72 participants (36 with ROT1D and 36 with EOT1D) will be enrolled in the PRISE study and randomized 2:1 to receive SAB-142 at 2.5 mg/kg or placebo. The study includes age step-downs to enable enrollment of younger adolescent participants (12-14 years) and pediatric participants (5-11 years) following safety review by the Data Monitoring Committee.

The timing of the second treatment period at Month 6 reflects consideration of immune reconstitution kinetics and may reinforce initial immunomodulatory effects to extend C-peptide preservation. The selection of C-peptide area under the curve during a 2-hour mixed meal tolerance test as an endpoint provides a direct measure of endogenous insulin secretion, with comprehensive secondary endpoints including time in tight range, HbA1c, exogenous insulin use, and partial clinical remission rates.

The trial includes extensive mechanistic assessments that may provide insights into SAB-142's mechanism of action and identify biomarkers predictive of treatment response.

Immunophenotyping assessments will evaluate T cell exhaustion markers, including PD-1, TIGIT, and KLRG1, regulatory T cell populations, and samples for DNA methylation, RNA sequencing, and microRNA analysis to identify epigenetic and transcriptional signatures associated with treatment response. An independent program-level Data Monitoring Committee provides critical oversight of participant safety and study conduct, with authority to make recommendations regarding study continuation, modification, or termination based on emerging safety or efficacy data. The statistical analysis plan incorporates ed-model repeated-measures (MMRM) analysis with a two-sided alpha of 0.05 for the primary comparison combining data from cohorts 1, 2, and 3.

### Dosing Schedule of SAB-142 During Phase 3 Clinical Trial

The selection of appropriate dosage levels allows optimization of the therapeutic efficacy of SAB-142 while minimizing potential adverse effects. The Phase 3 clinical trial evaluates SAB-142 at a dose of 2.5 mg/kg in participants with Stage 3 New Onset T1D (NOT1D), Recent Onset T1D (ROT1D), and Established Onset T1D (EOT1D). The split-dose regimen, with 0.5 mg/kg administered on the first day followed by 2.0 mg/kg on the second day, is designed to mitigate infusion-related reactions and cytokine release while maintaining therapeutic drug exposure. A second treatment period at Month 6 provides an additional dose of SAB-142 or placebo, also delivered as a split dose over 2 days, to evaluate the effects of repeat dosing on β-cell function preservation and immunomodulation.

Subjects in the Phase 3 clinical trial will be randomized to receive SAB-142 formulation at 2.5 mg/kg or placebo in a 2:1 allocation ratio. In some embodiments, the subjects are administered a dose of 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, or 5.0 mg/kg. In some embodiments, the dosage is about 0.5 mg/kg to about 5.0 mg/kg. In some embodiments, the dosage is about 1.0 mg/kg to about 4.0 mg/kg. In some embodiments, the dosage is about 1.5 mg/kg to about 3.0 mg/kg. In some embodiments, the dosage is 0.5 mg/kg, 1.5 mg/kg, or 2.5 mg/kg. In some embodiments, the dosage is administered as a split dose over two consecutive days, with 0.5 mg/kg administered on the first day and the remainder of the dose administered on the second day. In some embodiments, the dosage is administered during two treatment periods, with the first treatment period occurring at baseline and the second treatment period occurring at approximately Month 6.

In some embodiments, the dosage of SAB-142 is administered intravenously.

### Objectives and Endpoints for Phase 3 Clinical Trial

The SAB-142-301 clinical trial encompasses multiple objectives designed to comprehensively evaluate the therapeutic potential of SAB-142 in participants with Stage 3 new-onset type 1 diabetes (NOT1D), recent onset type 1 diabetes (ROT1D), and established type 1 diabetes (EOT1D). The primary objective is to determine whether SAB-142 slows the loss of 0-cells and preserves 3-cell function over 12 months in participants with Stage 3 NOT1D, ROT1D, and EOT1D as measured by the area under the concentration-time curve (AUC) of C-peptide after a 2-hour mixed meal tolerance test (change from baseline in C-peptide ln(AUC+1) at 12 months) in hATG compared to placebo. Secondary objectives include identifying a personalized immunotherapy approach based on previously identified baseline pre-treatment in vitro testing to identify responders versus non-responders to SAB-142; evaluating participant improvements in key clinical parameters of diabetes management, such as time in tight range, hemoglobin A1c levels, time in range, time above range, time below range, exogenous insulin use, clinically important hypoglycemic episodes, proportion of participants achieving partial clinical remission, total BETA-2 score, and insulin-dose adjusted A1c; determining the safety and tolerability of SAB-142 through assessment of treatment-emergent adverse events, adverse events of special interest, and serious adverse events; evaluating the pharmacokinetics and immunogenicity of SAB-142 through measurement of serum concentrations and anti-SAB-142 antibodies including neutralizing antibodies if indicated; and evaluating changes in immune cell populations following administration of SAB-142 through comprehensive immunophenotyping. Exploratory objectives aim to determine the effects of SAB-142 on type 1 diabetes clinical parameters, C-peptide quantitative response metric, 2-hour MMTT C-peptide AUC, fasting C-peptide, proportion of participants maintaining clinically significant stimulated peak C-peptide, fasting proinsulin-to-C-peptide ratio, calculated estimated residual stimulated C-peptide, proportion of participants with poor glycemic control, incidence of hypoglycemia and diabetic ketoacidosis, patient- and parent-reported outcomes including age-appropriate Diabetes Treatment Satisfaction Questionnaire and PedsQL Diabetes Module, and mechanistic, molecular, and genetic markers including serum cytokine panels, safety immune functional assays, functional exhaustion assays, activation induced marker assays, neutrophil assays, ex vivo treatment of PBMCs, single nucleotide polymorphism analysis, DNA methylation, RNA-sequencing, and microRNA expression that may provide insights into treatment response and disease progression.

In some embodiments, treatment with SAB-142 may result in a change from baseline in C-peptide ln(AUC+1) at 12 months ranging from about −0.1 to about 0.3, from about −0.05 to about 0.25, or from about 0 to about 0.2, wherein C-peptide ln(AUC+1) is measured using a 2-hour mixed meal tolerance test with blood samples collected at multiple timepoints (approximately −10 minutes, 0 minutes, 15 minutes, 30 minutes, 60 minutes, 90 minutes, and 120 minutes relative to meal consumption) and C-peptide concentrations quantified using immunoassay methods, with the area under the concentration-time curve calculated using the trapezoidal rule and natural logarithm transformation applied to normalize the distribution. In some aspects, participants receiving SAB-142 may demonstrate a smaller decline in C-peptide levels compared to placebo, with differences ranging from about 0.1 to about 0.4 ln(AUC+1) units.

In some embodiments, the change from baseline in C-peptide AUC may range from about −20% to about +10%, from about −15% to about +5%, or from about −10% to about 0%. In some cases, participants receiving SAB-142 may maintain C-peptide levels within about 80% to about 110% of baseline values at 12 months, while placebo-treated participants may experience declines to about 50% to about 80% of baseline values.

In some embodiments, the change from baseline in C-peptide AUC may range from about −20% to about +10%, from about −15% to about +5%, from about −10% to about 0%, from about −5% to about +5%, or from about −25% to about +15%. In some cases, participants receiving SAB-142 may maintain within about 80% to about 110% of baseline values at 12 months, within about 85% to about 105% of baseline values, within about 90% to about 100% of baseline values, or within about 75% to about 115% of baseline values, while placebo-treated participants may experience declines to about 50% to about 60% of baseline values, to about 55% to about 65% of baseline values, to about 60% to about 70% of baseline values, to about 65% to about 75% of baseline values, to about 70% to about 80% of baseline values, or to about 45% to about 55% of baseline values.

In some embodiments, time in tight range (glucose >70 but ≤140 mg/dL) may increase from baseline by about 5% to about 30%, from about 10% to about 25%, or from about 15% to about 20% in participants receiving SAB-142, wherein time in tight range is measured using continuous glucose monitoring devices worn by participants for specified periods of at least 10 days, with glucose readings recorded at regular intervals and the percentage of time spent within the defined glucose range calculated from the continuous data stream. In some aspects, participants may achieve time in tight range values of about 40% to about 70%, from about 45% to about 65%, or from about 50% to about 60% at 12 months. In some cases, the difference in time in tight range between SAB-142 and placebo may range from about 5% to about 20%, from about 8% to about 15%, or from about 10% to about 12%.

In some embodiments, hemoglobin A1c levels may be maintained at about 5.5% to about 7.5%, from about 6.0% to about 7.0%, or from about 6.2% to about 6.8% in participants receiving SAB-142, wherein hemoglobin A1c levels are measured from venous blood samples using high-performance liquid chromatography or immunoassay methods that quantify the percentage of hemoglobin molecules with glucose attached, providing an integrated measure of average glucose levels over approximately the preceding 2-3 months. In some aspects, the change from baseline in HbA1c may range from about −0.5% to about +1.0%, from about −0.3% to about +0.8%, or from about 0% to about +0.5%. In some cases, participants receiving SAB-142 may demonstrate HbA1c values that are about 0.2% to about 1.0% lower than placebo-treated participants at 12 months, from about 0.3% to about 0.8% lower, or from about 0.4% to about 0.6% lower.

In some embodiments, time in range (glucose >70 but 180 mg/dL) may be maintained at about 60% to about 85%, from about 65% to about 80%, or from about 70% to about 75% in participants receiving SAB-142, wherein time in range is measured using continuous glucose monitoring devices worn by participants for specified periods of at least 10 days, with glucose readings recorded at regular intervals and the percentage of time spent within the defined glucose range calculated from the continuous data stream. In some aspects, the change from baseline in time in range may range from about −5% to about +15%, from about 0% to about +12%, or from about +2% to about +10%. In some cases, participants receiving SAB-142 may achieve time in range values that are about 5% to about 15% higher than placebo-treated participants.

In some embodiments, time above range (glucose >180 mg/dL) may be reduced to about 10% to about 35%, from about 15% to about 30%, or from about 18% to about 25% in participants receiving SAB-142, wherein time above range is measured using continuous glucose monitoring devices worn by participants for specified periods of at least 10 days, with glucose readings recorded at regular intervals and the percentage of time spent above the defined glucose threshold calculated from the continuous data stream. In some aspects, the change from baseline in time above range may range from about −20% to about +5%, from about −15% to about 0%, or from about −12% to about −5%. In some cases, participants receiving SAB-142 may demonstrate time above range values that are about 5% to about 15% lower than placebo-treated participants.

In some embodiments, time below range (glucose <70 mg/dL) may be maintained at about 1% to about 5%, from about 1.5% to about 4%, or from about 2% to about 3% in participants receiving SAB-142, wherein time below range is measured using continuous glucose monitoring devices worn by participants for specified periods of at least 10 days, with glucose readings recorded at regular intervals and the percentage of time spent below the defined glucose threshold calculated from the continuous data stream. In some aspects, the change from baseline in time below range may range from about −2% to about +3%, from about −1% to about +2%, or from about 0% to about +1%. In some cases, participants receiving SAB-142 may demonstrate time below range values similar to placebo-treated participants, with differences of less than about 2%, less than about 1.5%, or less than about 1%.

In some embodiments, exogenous insulin use may range from about 0.05 to about 0.8 U/kg/day, from about 0.08 to about 0.7 U/kg/day, from about 0.1 to about 0.6 U/kg/day, from about 0.12 to about 0.55 U/kg/day, from about 0.15 to about 0.5 U/kg/day, from about 0.18 to about 0.45 U/kg/day, from about 0.2 to about 0.4 U/kg/day, from about 0.22 to about 0.38 U/kg/day, from about 0.25 to about 0.35 U/kg/day, from about 0.1 to about 0.45 U/kg/day, from about 0.15 to about 0.4 U/kg/day, from about 0.2 to about 0.5 U/kg/day, from about 0.12 to about 0.48 U/kg/day, from about 0.18 to about 0.52 U/kg/day, or from about 0.22 to about 0.42 U/kg/day in participants receiving SAB-142 at 12 months, wherein exogenous insulin use is measured through participant or caregiver recording of all insulin doses administered via injection or insulin pump in study diaries during specified monitoring periods, with daily insulin requirements calculated as total units per kilogram of body weight per day. In some aspects, the change from baseline in insulin use may range from about −0.2 to about +0.5 U/kg/day, from about −0.15 to about +0.45 U/kg/day, from about −0.1 to about +0.4 U/kg/day, from about −0.05 to about +0.38 U/kg/day, from about 0 to about +0.35 U/kg/day, from about +0.02 to about +0.32 U/kg/day, from about +0.05 to about +0.3 U/kg/day, from about +0.08 to about +0.28 U/kg/day, from about +0.1 to about +0.25 U/kg/day, from about −0.08 to about +0.42 U/kg/day, from about −0.02 to about +0.36 U/kg/day, from about +0.03 to about +0.33 U/kg/day, from about +0.06 to about +0.29 U/kg/day, from about +0.04 to about +0.31 U/kg/day, or from about +0.07 to about +0.27 U/kg/day. In some cases, participants receiving SAB-142 may require about 0.02 to about 0.25 U/kg/day less insulin than placebo-treated participants, from about 0.04 to about 0.22 U/kg/day less, from about 0.05 to about 0.2 U/kg/day less, from about 0.06 to about 0.18 U/kg/day less, from about 0.08 to about 0.15 U/kg/day less, from about 0.09 to about 0.14 U/kg/day less, from about 0.1 to about 0.12 U/kg/day less, from about 0.03 to about 0.23 U/kg/day less, from about 0.05 to about 0.19 U/kg/day less, from about 0.07 to about 0.17 U/kg/day less, from about 0.08 to about 0.16 U/kg/day less, from about 0.09 to about 0.15 U/kg/day less, from about 0.1 to about 0.14 U/kg/day less, from about 0.11 to about 0.13 U/kg/day less, or from about 0.06 to about 0.21 U/kg/day less.

In some embodiments, the proportion of participants achieving partial clinical remission (insulin requirement <0.25 U/kg/day and HbA1c <6.5%) may range from about 20% to about 60%, from about 22% to about 58%, from about 25% to about 55%, from about 28% to about 52%, from about 30% to about 50%, from about 32% to about 48%, from about 35% to about 45%, from about 37% to about 43%, or from about 38% to about 42% in participants receiving SAB-142, wherein partial clinical remission is assessed by combining insulin dose data from participant diaries with hemoglobin A1c measurements from blood samples, with participants classified as achieving remission when insulin requirements fall below 0.25 U/kg/day and HbA1c values remain below 6.5%. In some aspects, the proportion of participants achieving partial clinical remission may be at least about 20%, at least about 25%, at least about 30%, at least about 35%, at least about 40%, at least about 45%, at least about 50%, at least about 55%, or at least about 60%. In some aspects, the difference in partial clinical remission rates between SAB-142 and placebo may range from about 10% to about 40%, from about 12% to about 38%, from about 15% to about 35%, from about 17% to about 33%, from about 20% to about 30%, from about 22% to about 28%, or from about 24% to about 26%. In some aspects, the difference in partial clinical remission rates between SAB-142 and placebo may be at least about 10%, at least about 12%, at least about 15%, at least about 18%, at least about 20%, at least about 22%, at least about 25%, at least about 28%, at least about 30%, at least about 35%, or at least about 40%.

In some embodiments, the proportion of participants achieving partial remission as defined by IDAA1c <9 may range from about 20% to about 70%, from about 25% to about 65%, from about 28% to about 62%, from about 30% to about 60%, from about 32% to about 58%, from about 35% to about 55%, from about 38% to about 52%, from about 40% to about 50%, or from about 42% to about 48% in participants receiving SAB-142, wherein IDAA1c-defined partial remission is calculated using the formula IDAA1c=HbA1c (%)+[4×insulin dose (U/kg/24h)], with values <9 indicating remission status. In some embodiments, the proportion of participants achieving partial remission as defined by IDAA1c <9 may be at least about 25%, at least about 30%, at least about 35%, at least about 40%, at least about 45%, at least about 50%, at least about 55%, at least about 60%, or at least about 65% in participants receiving SAB-142. In some aspects, the difference in IDAA1c-defined remission rates between SAB-142 and placebo may range from about 10% to about 50%, from about 15% to about 45%, from about 18% to about 42%, from about 20% to about 40%, from about 22% to about 38%, from about 25% to about 35%, from about 27% to about 33%, or from about 28% to about 32%. In some embodiments, the difference in IDAA1c-defined remission rates between SAB-142 and placebo may be at least about 15%, at least about 18%, at least about 20%, at least about 22%, at least about 25%, at least about 28%, at least about 30%, at least about 35%, at least about 40%, or at least about 45%.

In some embodiments, the total BETA-2 score may range from about 4 to about 20, from about 5 to about 18, from about 5 to about 15, from about 6 to about 14, from about 6 to about 12, from about 7 to about 11, from about 7 to about 10, from about 7.5 to about 9.5, or from about 8 to about 9 in participants receiving SAB-142 at 12 months, wherein the BETA-2 score is calculated as a composite measure incorporating fasting plasma glucose (mmol/L), HbA1c (%), daily insulin dose (U/kg), and fasting C-peptide (nmol/L), with higher scores indicating poorer metabolic control. In some embodiments, the total BETA-2 score may be less than about 15, less than about 14, less than about 13, less than about 12, less than about 11, less than about 10, less than about 9, less than about 8, less than about 7, or less than about 6. In some aspects, the change from baseline in BETA-2 score may range from about −4 to about +10, from about −3 to about +9, from about −2 to about +8, from about −1 to about +7, from about 0 to about +6, from about +0.5 to about +5.5, from about +1 to about +5, from about +1.5 to about +4.5, from about +2 to about +4, or from about +2.5 to about +3.5. In some embodiments, the change from baseline in BETA-2 score may be less than about +8, less than about +7, less than about +6, less than about +5, less than about +4, less than about +3, less than about +2, or less than about +1. In some cases, participants receiving SAB-142 may demonstrate BETA-2 scores that are about 0.5 to about 8 points lower than placebo-treated participants, from about 1 to about 7 points lower, from about 1 to about 5 points lower, from about 1.5 to about 4.5 points lower, from about 1.5 to about 4 points lower, from about 2 to about 3.5 points lower, from about 2 to about 3 points lower, or from about 2.5 to about 3 points lower. In some embodiments, participants receiving SAB-142 may demonstrate BETA-2 scores that are at least about 0.5 points lower, at least about 1 point lower, at least about 1.5 points lower, at least about 2 points lower, at least about 2.5 points lower, at least about 3 points lower, at least about 3.5 points lower, at least about 4 points lower, at least about 4.5 points lower, or at least about 5 points lower than placebo-treated participants.

In some embodiments, fasting C-peptide levels may be maintained at about 0.2 to about 0.8 nmol/L, from about 0.3 to about 0.7 nmol/L, from about 0.35 to about 0.65 nmol/L, from about 0.4 to about 0.6 nmol/L, from about 0.45 to about 0.55 nmol/L, or about 0.5 nmol/L in participants receiving SAB-142 at 12 months, wherein fasting C-peptide levels are measured from blood samples collected after participants have fasted for at least 8-10 hours, with C-peptide concentrations quantified using immunoassay methods. In some aspects, the change from baseline in fasting C-peptide may range from about −0.3 to about +0.1 nmol/L, from about −0.2 to about 0 nmol/L, from about −0.15 to about −0.05 nmol/L, from about −0.12 to about −0.08 nmol/L, or about −0.1 nmol/L. In some cases, participants receiving SAB-142 may maintain fasting C-peptide levels that are about 0.05 to about 0.3 nmol/L, from about 0.1 to about 0.25 nmol/L, from about 0.12 to about 0.2 nmol/L, from about 0.14 to about 0.18 nmol/L, or about 0.15 to about 0.17 nmol/L higher than placebo-treated participants.

In some embodiments, the proportion of participants maintaining stimulated peak C-peptide >0.2 nmol/L may range from about 70% to about 95%, from about 72% to about 93%, from about 74% to about 91%, from about 75% to about 92%, from about 76% to about 90%, from about 78% to about 89%, from about 80% to about 90%, from about 80% to about 88%, from about 82% to about 88%, from about 82% to about 86%, from about 83% to about 87%, from about 84% to about 88%, from about 84% to about 86%, from about 85% to about 90%, or from about 85% to about 87% in participants receiving SAB-142 at 12 months, wherein stimulated peak C-peptide is determined from the highest C-peptide value obtained during the 2-hour mixed meal tolerance test, with values >0.2 nmol/L considered clinically significant for indicating residual 0-cell function. In some aspects, the difference in the proportion maintaining adequate C-peptide between SAB-142 and placebo may range from about 15% to about 45%, from about 20% to about 40%, or from about 25% to about 35%.

In some embodiments, the incidence of clinically important hypoglycemic episodes (Level 2 and Level 3) may range from about 0.5 to about 5 events per patient-year, from about 1 to about 4 events per patient-year, or from about 1.5 to about 3 events per patient-year in participants receiving SAB-142, wherein clinically important hypoglycemic episodes are documented through participant or caregiver reporting in study diaries and verified using continuous glucose monitoring data, with Level 2 hypoglycemia defined as blood glucose <54 mg/dL (3.0 mmol/L) and Level 3 hypoglycemia defined as severe events requiring external assistance for recovery. In some aspects, the difference in hypoglycemic event rates between SAB-142 and placebo may range from about −1 to about +2 events per patient-year, from about −0.5 to about +1.5 events per patient-year, or from about 0 to about +1 event per patient-year.

In some embodiments, the proportion of participants with poor glycemic control (HbA1c >9%) may range from about 2% to about 15%, from about 3% to about 12%, from about 5% to about 10%, from about 2% to about 5%, from about 3% to about 6%, from about 4% to about 8%, from about 5% to about 8%, from about 6% to about 10%, from about 7% to about 12%, from about 8% to about 12%, from about 10% to about 15%, from about 3% to about 8%, or from about 4% to about 9% in participants receiving SAB-142 at 12 months, wherein poor glycemic control is assessed by determining whether hemoglobin A1c values meet or exceed 9% at specified timepoints. In some aspects, the difference in poor glycemic control rates between SAB-142 and placebo may range from about −10% to about −2%, from about −8% to about −3%, from about −6% to about 4%, from about −10% to about −5%, from about −8% to about −5%, from about −7% to about −4%, from about −6% to about −2%, from about −5% to about −2%, from about −4% to about −1%, from about −9% to about −6%, or from about −7% to about −3%.

In some embodiments, calculated estimated residual stimulated C-peptide may range from about 0.3 to about 1.2 nmol/L, from about 0.4 to about 1.0 nmol/L, from about 0.5 to about 0.8 nmol/L, from about 0.3 to about 0.6 nmol/L, from about 0.4 to about 0.7 nmol/L, from about 0.5 to about 0.9 nmol/L, from about 0.6 to about 1.0 nmol/L, from about 0.7 to about 1.1 nmol/L, from about 0.8 to about 1.2 nmol/L, from about 0.3 to about 0.5 nmol/L, from about 0.4 to about 0.6 nmol/L, from about 0.5 to about 0.7 nmol/L, from about 0.6 to about 0.8 nmol/L, from about 0.7 to about 0.9 nmol/L, from about 0.8 to about 1.0 nmol/L, from about 0.9 to about 1.1 nmol/L, or from about 1.0 to about 1.2 nmol/L in participants receiving SAB-142 at 12 months, wherein calculated estimated residual stimulated C-peptide is derived using mathematical models that incorporate multiple C-peptide measurements from the mixed meal tolerance test along with participant characteristics such as age, diabetes duration, and baseline C-peptide levels. In some aspects, the change from baseline in estimated residual C-peptide may range from about −0.4 to about +0.2 nmol/L, from about −0.3 to about 0 nmol/L, from about −0.2 to about −0.1 nmol/L, from about −0.4 to about −0.1 nmol/L, from about −0.3 to about +0.1 nmol/L, from about −0.2 to about +0.1 nmol/L, from about −0.1 to about +0.2 nmol/L, from about −0.4 to about −0.2 nmol/L, from about −0.3 to about −0.1 nmol/L, from about −0.2 to about 0 nmol/L, from about −0.1 to about +0.1 nmol/L, or from about 0 to about +0.2 nmol/L.

In some embodiments, the fasting proinsulin-to-C-peptide ratio may range from about 0.01 to about 0.08, from about 0.02 to about 0.06, from about 0.03 to about 0.05, from about 0.01 to about 0.04, from about 0.02 to about 0.05, from about 0.03 to about 0.06, from about 0.04 to about 0.07, from about 0.05 to about 0.08, from about 0.01 to about 0.03, from about 0.02 to about 0.04, from about 0.03 to about 0.05, from about 0.04 to about 0.06, from about 0.05 to about 0.07, or from about 0.06 to about 0.08 in participants receiving SAB-142 at 12 months, wherein the fasting proinsulin-to-C-peptide ratio is calculated by dividing fasting proinsulin concentration by fasting C-peptide concentration, both measured from the same blood sample collected after an overnight fast, with elevated ratios indicating increased p-cell endoplasmic reticulum stress and impaired proinsulin processing. In some aspects, the change from baseline in proinsulin-to-C-peptide ratio may range from about −0.02 to about +0.05, from about −0.01 to about +0.04, from about 0 to about +0.03, from about −0.02 to about +0.01, from about −0.01 to about +0.02, from about 0 to about +0.02, from about +0.01 to about +0.04, from about +0.02 to about +0.05, from about −0.02 to about 0, from about −0.01 to about +0.01, from about 0 to about +0.04, or from about +0.01 to about +0.03. In some cases, lower proinsulin-to-C-peptide ratios may indicate reduced p-cell endoplasmic reticulum stress.

In some embodiments, DNA methylation patterns and RNA sequencing may be performed on blood samples collected at baseline and at specified follow-up timepoints to assess epigenetic and transcriptomic changes associated with treatment response. In some aspects, single nucleotide polymorphism analysis may be conducted to identify genetic variants that may influence treatment outcomes. In some cases, microRNA expression profiling may provide additional molecular markers associated with therapeutic response.

In some embodiments, functional immune assays may be performed to assess T cell activation, proliferation, and cytotoxic capacity, wherein peripheral blood mononuclear cells are isolated and subjected to ex vivo stimulation protocols. In some aspects, activation-induced marker expression may be evaluated to characterize the functional state of T cells following SAB-142 treatment. In some cases, neutrophil function assays may be conducted at selected timepoints to assess innate immune responses.

In some embodiments, treatment with SAB-142 may result in reduced incidence of serum sickness compared to rabbit anti-thymocyte globulin, wherein serum sickness is characterized by fever, rash, arthralgia, and immune complex deposition occurring days to weeks after administration of heterologous immunoglobulin preparations. In some aspects, the incidence of serum sickness in participants receiving SAB-142 may range from about 0% to about 5%, from about 0% to about 3%, from about 0% to about 2%, or from about 0% to about 1%, whereas rabbit anti-thymocyte globulin has been associated with serum sickness rates ranging from about 30% to about 70% in type 1 diabetes populations. In some cases, the fully human nature of SAB-142 may reduce recognition of the therapeutic antibody as foreign by the recipient's immune system.

In some embodiments, treatment with SAB-142 may result in reduced immunogenicity compared to rabbit anti-thymocyte globulin, wherein immunogenicity is assessed by measuring anti-drug antibodies in serum samples collected at baseline and at specified follow-up timepoints. In some aspects, the incidence of anti-drug antibodies in participants receiving SAB-142 at target doses may range from about 0% to about 10%, from about 0% to about 5%, or from about 0% to about 2%. In some cases, the absence or low incidence of anti-drug antibodies may enable safe redosing of SAB-142 at subsequent timepoints without increased risk of hypersensitivity reactions.

In some embodiments, treatment with SAB-142 may result in reduced incidence of anaphylaxis or severe allergic reactions compared to rabbit anti-thymocyte globulin, wherein anaphylaxis is defined as a severe, potentially life-threatening systemic hypersensitivity reaction characterized by rapid onset of symptoms affecting multiple organ systems. In some aspects, the incidence of Grade 4 allergic reactions including anaphylaxis in participants receiving SAB-142 may range from about 0% to about 2%, from about 0% to about 1%, or may be approximately 0%. In some cases, the reduced allergic potential may be attributed to the fully human immunoglobulin structure of SAB-142.

In some embodiments, treatment with SAB-142 may result in the absence of sustained lymphodepletion, wherein sustained lymphodepletion is defined as a prolonged reduction in peripheral lymphocyte counts extending beyond 7 days following treatment administration. In some aspects, participants receiving SAB-142 may experience transient lymphopenia during the initial 2-3 days following administration, with lymphocyte counts returning to baseline or near-baseline levels by Day 7. In some cases, the absence of sustained lymphodepletion may reduce the risk of opportunistic infections and may preserve protective immunity.

In some embodiments, treatment with SAB-142 may result in reduced incidence of cytokine release syndrome compared to higher doses of anti-thymocyte globulin preparations, wherein cytokine release syndrome is characterized by systemic inflammatory response with fever, hypotension, and elevated pro-inflammatory cytokines. In some aspects, the incidence of Grade 3 or higher cytokine release syndrome in participants receiving SAB-142 may range from about 0% to about 5%, from about 0% to about 3%, or from about 0% to about 1%. In some cases, mild Grade 1 cytokine release syndrome presenting as flu-like symptoms may occur during the first 1-2 days of treatment and may resolve without intervention.

In some embodiments, treatment with SAB-142 may result in reduced incidence of infusion-related reactions compared to rabbit anti-thymocyte globulin, wherein infusion-related reactions include symptoms such as fever, chills, headache, nausea, and local reactions at the infusion site. In some aspects, the majority of infusion-related reactions observed with SAB-142 may be mild (Grade 1) and may resolve within the first week following treatment. In some cases, infusion-related reactions may be managed with standard supportive care measures without requiring treatment discontinuation.

In some embodiments, treatment with SAB-142 may result in absence of sustained neutropenia, wherein sustained neutropenia is defined as absolute neutrophil count below the lower limit of normal persisting beyond 14 days following treatment administration. In some aspects, neutrophil counts in participants receiving SAB-142 may remain within normal ranges throughout the study period. In some cases, the preservation of neutrophil populations may reduce the risk of bacterial infections.

In some embodiments, treatment with SAB-142 may result in absence of sustained thrombocytopenia, wherein sustained thrombocytopenia is defined as platelet count below the lower limit of normal persisting beyond 14 days following treatment administration. In some aspects, platelet counts in participants receiving SAB-142 may remain within normal ranges throughout the study period. In some cases, the preservation of platelet populations may reduce the risk of bleeding complications.

In some embodiments, treatment with SAB-142 may result in absence of sustained anemia, wherein sustained anemia is defined as hemoglobin or red blood cell count below the lower limit of normal persisting beyond 14 days following treatment administration. In some aspects, red blood cell counts and hemoglobin levels in participants receiving SAB-142 may remain within normal ranges throughout the study period.

In some embodiments, treatment with SAB-142 may result in reduced incidence of opportunistic infections compared to lymphodepleting regimens, wherein opportunistic infections include viral, fungal, or bacterial infections that occur in the setting of immunosuppression. In some aspects, the incidence of Grade 3 or higher infections in participants receiving SAB-142 may range from about 0% to about 5%, from about 0% to about 3%, or from about 0% to about 1%. In some cases, the preservation of regulatory T cell populations and absence of sustained lymphodepletion may maintain protective immunity against pathogens.

In some embodiments, treatment with SAB-142 may result in absence of clinically significant coagulation abnormalities, wherein coagulation parameters including prothrombin time, activated partial thromboplastin time, and fibrinogen levels remain within normal ranges throughout the study period. In some aspects, no clinically significant changes in coagulation markers may be observed at doses up to 2.5 mg/kg.

In some embodiments, treatment with SAB-142 may enable safe administration in ambulatory settings without requirement for intensive monitoring, wherein the favorable safety profile characterized by absence of serum sickness, low immunogenicity, and transient rather than sustained effects on lymphocyte populations may permit outpatient administration. In some aspects, the reduced burden of adverse events may improve treatment accessibility and participant quality of life compared to therapies requiring prolonged hospitalization or intensive medical supervision.

In some embodiments, participants identified as responders based on early immunological markers such as SNP, microRNA, and expression markers, may demonstrate enhanced preservation of 3-cell function compared to the overall treated population, wherein responders may maintain C-peptide levels at 12 months that are about 15% to about 40%, about 20% to about 35%, or about 25% to about 30% higher than non-responders receiving the same SAB-142 dose. In some aspects, participants selected from a responder group characterized by sustained CD4+ T cell exhaustion markers (PD-1+ TIGIT+) at Week 4 may exhibit C-peptide preservation that exceeds the overall population mean by about 0.1 to about 0.4 nmol/L, about 0.15 to about 0.35 nmol/L, or about 0.2 to about 0.3 nmol/L at 12 months. In some cases, responders may demonstrate insulin dose requirements that are about 0.1 to about 0.3 U/kg/day, about 0.12 to about 0.25 U/kg/day, or about 0.15 to about 0.2 U/kg/day lower than non-responders at 12 months. In some embodiments, the proportion of participants achieving partial clinical remission may range from about 40% to about 70%, about 45% to about 65%, or about 50% to about 60% among responders, compared to about 10% to about 30%, about 15% to about 25%, or about 18% to about 22% in the overall SAB-142 treated population.

### ALTERNATIVE EMBODIMENTS

Clause 1. A method for producing a plurality of human antibodies or fragments thereof against human thymocytes, comprising:
   a) administering a plurality of human thymocytes to a transchromosomic ungulate, wherein a viability of the plurality of human thymocytes is at least about 80%;
   b) collecting plasma from the transchromosomic ungulate; and
   c) purifying the plurality of human antibodies from the plasma,
   wherein the viability can be measured by dividing a number of viable cells over a number of total cells, and
   wherein the method produces a purified human immunoglobulin (IgG) product.
Clause 2. The method of clause 1, further comprising administering to the transchromosomic ungulate an adjuvant.
Clause 3. The method of clause 2, wherein the adjuvant is administered at the substantially same time as the plurality of human thymocytes.
Clause 4. The method of clause 2 or clause 3, wherein the adjuvant is administered prior to the administration of the plurality of human thymocytes.
Clause 5. The method of any one of clauses 2-4, wherein the adjuvant is administered subsequent to the administration of the plurality of human thymocytes.
Clause 6. The method of any of clauses 1-5, wherein the viability is at least about 90%.
Clause 7. The method of any of clauses 1-5, wherein the viability is at least about 95%.
Clause 8. The method of any of clauses 1-5, wherein the plurality of human thymocytes comprises at least 75% intact cells.
Clause 9. The method of any of clauses 1-5, wherein the plurality of human thymocytes comprises at least 90% intact cells.
Clause 10. The method of any of clauses 1-5, wherein the plurality of human thymocytes comprises at least 95% intact cells.
Clause 11. The method of any of clauses 1-5, wherein the viability is measured by counting intact cells.
Clause 12. The method of clause 11, wherein the intact cells are stained with a viability dye, and wherein the viability dye is propidium iodide, or Trypan blue.
Clause 13. The method of any of the preceding clauses, wherein the administering the plurality of human thymocytes is administered via a subcutaneous injection.
Clause 14. The method of clause 13, wherein the subcutaneous injection is administered at one or more different locations of the transchromosomic ungulate.
Clause 15. The method of clause 14, wherein the one or more different locations comprise at least 2, 3, 4, 5, 6, 7, 8, 9, or 10 locations.
Clause 16. The method of clause 1, wherein the administering step is followed by a collecting step comprising collecting a plasma sample from the transchromosomic ungulate.
Clause 17. The method of clause 16, wherein the plasma sample is about 4 L to about 10 L for a 400 kg transchromosomic ungulate per collection.
Clause 18. The method of clause 17, wherein the plasma sample collected is about 9.5 L per 400 kb animal per collection.
Clause 19. The method of clause 17 or 18, wherein the plasma sample comprises about 1% to 3% of a body weight of the transchromosomic ungulate.
Clause 20. The method of clause 19, wherein the plasma sample comprises about 2.4% of the body weight.
Clause 21. The method of clause 1, wherein a plasma sample is collected from each of two or more transchromosomic ungulates.
Clause 22. The method of clause 21, further comprising pooling the plasma sample collected from each of the two or more transchromosomic ungulates to produce a pooled plasma.
Clause 23. The method of clause 22, further comprising purifying the pooled plasma.
Clause 24. The method of clause 23, wherein the purifying comprises adsorbing the pooled plasma to red blood cells (RBCs) to remove an anti-RBC antibody or fragment thereof and generate a first purified plasma sample.
Clause 25. The method of clause 24, wherein the RBCs comprises a blood type, and wherein the blood type is selected from the group consisting of A, B, AB, O, Rh-positive, or Rh-negative, and a combination thereof.
Clause 26. The method of clause 24, wherein the purifying comprises removing viruses, pathogens, and/or non-IgG bovine plasma proteins from the first purified plasma sample to generate a second purified plasma sample.
Clause 27. The method of clause 26, wherein the removing viruses, pathogens, and non-IgG bovine plasma proteins comprises using caprylic acid fractionation, filter purification, or a combination thereof.
Clause 28. The method of clause 27, wherein the caprylic acid fractionation is followed by the filter purification to produce the second purified plasma sample.
Clause 29. The method of clause 27, wherein the filter purification comprises depth filtration of the fractionated plasma, followed by a washing step.
Clause 30. The method of clause 29, wherein the washing comprises phosphate-buffered saline. Clause 31. The method of any one of clauses 24-29, further comprising adjusting a pH value of the first purified plasma sample to about 4 to 6.
Clause 32. The method of clause 31, wherein the pH value is adjusted to about 4.5 to 4.9.
Clause 33. The method of clause 26, wherein the purifying comprises adding the second purified plasma sample to an affinity purification column to capture and elute kappa Fab fragments to generate a third purified plasma sample.
Clause 34. The method of clause 33, further comprising incubating captured kappa Fab fragments at a low pH for viral inactivation.
Clause 35. The method of clause 34, wherein the low pH is between about 3 to 4.
Clause 36. The method of clause 35, wherein the low pH is between about 3.6 to 3.8.
Clause 37. The method of clause 33, wherein the purifying further comprises removing chimeric IgG (cIgG), transchromosomic bovine IgG (t-bIgG), or a combination thereof.
Clause 38. The method of clause 33, wherein the purifying further comprises removing host cell proteins, non-immunoglobulin bovine plasma proteins, or a combination thereof.
Clause 39. The method of clause 33, wherein the purifying comprises adding the third purified plasma sample to an affinity column designed for binding ungulate IgG heavy chain proteins and eluting the third purified plasma sample to generate a fourth purified plasma sample.
Clause 40. The method of clause 39, further comprising subjecting the fourth purified plasma sample to an anion polishing step to generate a fifth purified plasma sample.
Clause 41. The method of clause 40, wherein the purifying further comprises removing additional impurities from the fourth purified plasma sample.
Clause 42. The method of clause 41, wherein the additional impurities are selected from the group consisting of IgA, IgM, host cell proteins, DNA, endotoxin, and a combination thereof.
Clause 43. The method of clause 41, wherein the additional impurities comprise residual immunoglobulin M (IgM), immunoglobulin A (IgA), DNA, or endotoxin.
Clause 44. The method of any one of clauses 40-43, further comprising subjecting the fifth purified plasma sample to nanofiltration to generate a purified human IgG product.
Clause 45. The method of clause 44, wherein the nanofiltration removes viruses.
Clause 46. The method of clause 44 or 45, further comprising subjecting the purified human IgG product to at least one of ultrafiltration, diafiltration, bioburden reduction, sterile filtration, or a combination thereof, to generate a storage/final distribution formulation.
Clause 47. The method of clause 46, wherein the storage/final distribution formulation comprises about 1 to 100 mM L-glutamic acid monosodium salt; about 50 to 500 mM D-sorbitol; and about 0.01 to 2 mg/mL Tween 80, and about 50 to 100 mg/mL purified human IgG.
Clause 48. The method of clause 47, wherein a pH is between about 3 to 8, about 4 to 7, or about 5 to 6.
Clause 49. The method of clause 46, wherein the bioburden reduction comprises using a 0.1 to 0.3 μm filter.
Clause 50. The method of any one of clauses 23-29, wherein a total human immunoglobulin content in the pooled plasma is greater than 2.0 mg/mL.
Clause 51. The method of clauses 1-46 wherein the ungulate is a bovine.
Clause 52. The method of any one of clauses 1-47, wherein a concentration of human IgG in the storage/final distribution formulation is between 20 mg/mL and 32 mg/mL.
Clause 53. A pharmaceutical composition, comprising:
   a) a plurality of fully human or substantially human antibodies or fragments thereof against human thymocytes, wherein the plurality of fully human or substantially human antibodies comprises an immunoglobulin (IgG) profile that is about 80% IgG1, optionally, wherein the IgG1 is about 87% or 88%; and
   b) one or more pharmaceutically acceptable excipients.
Clause 54. The pharmaceutical composition of clause 53, wherein the IgG profile further comprises IgG2; optionally, wherein the IgG2 is less than 13%; further optionally, wherein the IgG profile further comprises about 12% IgG2.
Clause 55. The pharmaceutical composition of clause 53, wherein the IgG profile comprises substantially zero IgG3.
Clause 56. The pharmaceutical composition of clause 53, wherein the IgG profile comprises substantially zero IgG4.
Clause 57. A pharmaceutical composition, comprising:
   a) a plurality of fully human or substantially human antibodies or fragment thereof against human thymocytes, wherein the plurality of fully human or substantially human antibodies comprises a glycosylation profile having detectable alpha-galactose in released N-linked glycans and the released N-linked glycans can be measured by mass spectrometry; and
   b) one or more pharmaceutically acceptable excipients.
Clause 58. The pharmaceutical composition of clause 57, wherein the released N-linked glycans comprises about 5.7% detectable alpha-galactose.
Clause 59. The pharmaceutical composition of clause 57, wherein the released N-linked glycans comprises mono-sialylated or di-sialylated glycans.
Clause 60. The pharmaceutical composition of clause 59, wherein the sialylated glycans comprises detectable N-glycolylneuraminic acid (NGNA/Neu5Gc).
Clause 61. The pharmaceutical composition of clause 59, wherein the sialylated glycans comprises detectable N-Acetylneuraminic acid (NANA/Neu5Ac).
Clause 62. The pharmaceutical composition of any of clauses 57 to 61, wherein the released N-linked glycans comprises about 8% to about 15% of bisecting N-acetylglucosamine (GlcNAc).
Clause 63. The pharmaceutical composition of any of clauses 57 to 62, wherein the released N-linked glycans comprises about more than 85% fucosylated glycans.
Clause 64. The pharmaceutical composition of any of clauses 57 to 63 wherein the plurality of fully human or substantially human antibodies or fragments thereof are multi-specific.
Clause 65. The pharmaceutical composition of any of clauses 57 to 64, wherein the plurality of fully human or substantially human antibodies or fragments thereof bind to one or more T-cell surface markers selected from the group consisting of CD2, CD3, CD4, CD8, CD20, and CD28.
Clause 66. The pharmaceutical composition of any of clauses 57 to 65, wherein the plurality of fully human or substantially human antibodies or fragments thereof has a titer activity ranging from 2000 to 50000 U/mg against an immune cell surface protein.
Clause 67. The pharmaceutical composition of clause 66, wherein the plurality of fully human or substantially human antibodies or fragments thereof, has an avidity to the immune cell surface protein of at least 1000 seconds k_off residence time.
Clause 68. The pharmaceutical composition of clause 67, wherein the k_off residence time is determined by a surface plasmon resonance assay or a chaotropic enzyme-linked immunosorbent assay (ELISA).
Clause 69. The pharmaceutical composition of clause 68, wherein the k_off residence time is greater than a k_off residence time for a rabbit antithymocyte globulin.
Clause 70. The pharmaceutical composition of clause 65, wherein the plurality of fully human or substantially human antibodies or fragments thereof has an avidity index to an immune cell surface protein of about 0.5 to 3.5.
Clause 71. The pharmaceutical composition of clause 68, wherein the plurality of fully human or substantially human antibodies or fragments thereof has an avidity index to an immune cell surface protein of about 0.69 to 3.28.
Clause 72. A composition, produced by immunizing a transchromosomic ungulate with a plurality of human thymocytes, wherein a viability of the plurality of human thymocytes is at least about 80%, and wherein the composition comprises a plurality of fully human or substantially human antibodies or fragments thereof, wherein the viability can be measured by dividing a number of viable cells over a number of total cells wherein the plurality of fully human or substantially human antibodies or fragments thereof is a purified human immunoglobulin (IgG) product.
Clause 73. The composition of clause 72, wherein a viability of the plurality of human thymocytes is measured by counting intact cells.
Clause 74. The composition of clause 72, wherein an intact cell is stained with a viability dye, and wherein the viability dye is propidium iodide, or Trypan blue.
Clause 75. The composition of clause 72, wherein the immunizing comprises administering a second immunization to the transchromosomic ungulate 1, 2, 3, 4, 5, 6, 7, or 8 times after a first immunization.
Clause 76. The composition of clause 75, wherein the second immunization is performed at about 3 weeks, 6 weeks, 9 weeks, 12 weeks after the first immunization.
Clause 77. The composition of clause 75, wherein the first immunization comprises at least 2×10⁹ of human thymocytes.
Clause 78. The composition of clause 75, wherein an immunization at about 3 weeks comprises at least 2×10⁹, an immunization at about 6 weeks comprises at least 2×10⁹, an immunization at about 9 weeks comprises at least 5.5×10⁹, and an immunization at about 12 weeks comprises at least 2.5×109 of human thymocytes.
Clause 79. A composition of clause 72 produced by collecting plasma from an immunized transchromosomic ungulate 1 to 3 times after each immunization, and/or after a 3^{rd} to 5^{th} immunization.
Clause 80. A composition, comprising a plurality of fully human or substantially human antibodies or fragments thereof against human thymocytes, produced by pooling plasma collected from two or more transchromosomic ungulates subsequent to immunizing the two or more transchromosomic ungulates with a plurality of human thymocytes.
Clause 81. The composition of clause 80, wherein the pooling plasma is pooled from samples collected after a third, fourth, fifth, sixth, or seventh immunization.
Clause 82. The composition of clause 80, wherein the pooled plasma contains human anti-thymocyte immunoglobulin (ATG), and the pooled plasma is further purified to produce a purified human IgG product.
Clause 83. The composition of clause 82, wherein:
   a. any anti-red blood cell antibody is substantially removed in a first purified plasma sample;
   b. any viruses, pathogens, and/or non-IgG bovine plasma proteins are removed in a second purified plasma sample;
   c. transchromosomic IgG is isolated from the pooled plasma by capturing a kappa Fab fragment in a third purified plasma sample;
   d. transchromosomic IgG is further captured in a fourth purified plasma sample;
   e. IgA, IgM, host cell proteins, DNA, endotoxin, or a combination thereof are substantially removed in a fifth purified plasma sample;
   f. viruses are substantially removed; or
   g. the human ATG is concentrated, diafiltrated, or a combination thereof as a purified human IgG product.
Clause 84. The composition of clause 83, wherein the purified human IgG product is formulated with excipients in a storage formulation.
Clause 85. The composition of clause 84, wherein the purified human IgG product is filtered to reduce bioburden.
Clause 86. The composition of clause 84, wherein the purified human IgG product is sterile-filtered.
Clause 87. The composition of clause 83, wherein a pooled RBC comprises a blood type, wherein the blood type is selected from the group consisting of A, B, AB, O, Rh-positive, or Rh-negative, and a combination thereof.
Clause 88. The composition of clause 83, wherein an anti-RBC antibody or fragment thereof is removed via adsorption to RBCs.
Clause 89. The composition of clause 83, wherein the viruses, pathogens, and/or non-IgG bovine plasma proteins are removed by caprylic acid fractionation, filter purification, or a combination thereof.
Clause 90. The composition of clause 89, wherein the second purified plasma sample is purified via a filter.
Clause 91. The composition of any one of clauses 83-87, wherein the first purified plasma sample is adjusted to have a pH of about 4 to 6.
Clause 92. The composition of clause 91, wherein the pH of the first purified plasma sample is about 4.5 to 4.9.
Clause 93. The composition of any one of clauses 88 to 92, wherein the first purified plasma sample is subjected to caprylic acid fractionation for removal of non-IgG proteins and virus inactivation.
Clause 94. The composition of clause 87, wherein a kappa Fab fragment of the second purified sample is further captured and eluted.
Clause 95. The composition of clause 94, wherein a captured kappa Fab fragment is incubated at a pH that is sufficiently low for viral inactivation.
Clause 96. The composition of clause 95, wherein the pH is between about 3 to 4.
Clause 97. The composition of clause 96, wherein the pH is between 3.6-3.8.
Clause 98. The composition of any one of clauses 95 to 97, wherein the third purified plasma sample has reduced levels of cIgG, t-bIgG, or a combination thereof.
Clause 99. The composition of clause 83, wherein a transchromosomic IgG of the third plasma sample is captured using an affinity column specific for binding an ungulate IgG heavy chain protein.
Clause 100. The composition of clause 83, wherein the fourth purified plasma sample is further purified via an anion polishing step.
Clause 101. The composition of clause 100, wherein residual IgM, IgA, DNA, and endotoxin in the fourth purified plasma sample is substantially removed.
Clause 102. The composition of clause 83, wherein the fifth purified plasma sample is further purified via a nanofiltration step to generate a purified human IgG product.
Clause 103. The composition of clause 83, wherein viruses in the fifth purified plasma sample are substantially removed.
Clause 104. The composition of clause 83, wherein the purified human IgG product is concentrated via an ultrafiltration step.
Clause 105. The composition of clause 104, wherein the purified human IgG product has a concentration of human IgG of no less than 10 mg/mL.
Clause 106. The composition of clause 84, wherein the human IgG product is formulated with an excipient buffer via diafiltration to generate a storage formulation.
Clause 107. The composition of clause 106, wherein the excipient buffer comprises 50 mM L-glutamic acid monosodium salt; optionally 262 mM D-sorbitol; further optionally 0.05 mg/ml Tween 80; and further optionally pH 5.5±0.1.
Clause 108. The composition of clause 107, wherein the storage formulation is further purified via an 0.1 to 0.3p m filter, a sterile filter, or a combination thereof to produce a storage formulation or a final distribution formulation.
Clause 109. A method for treating an autoimmune disease or disorder in a subject in need thereof, comprising administering to the subject an effective amount of the pharmaceutical composition of claims 52-71 or the composition of clauses 72-108, wherein the T1D in the subject is treated.
Clause 110. The method of clause 109, wherein the subject has Stage 3 New Onset of T1D (NOT1D).
Clause 111. The method of clause 109 or clause 92, wherein the effective amount comprises between about 0.5 mg/kg to 4 mg/kg of a purified human IgG product.
Clause 112. The method of clause 111, wherein the effective amount comprises about 1.5 mg/kg of the purified human IgG product.
Clause 113. The method of clause 111, wherein the effective amount comprises about 2.5 mg/kg of the purified human IgG product.
Clause 114. The method of any one of clauses 109-113, wherein the composition is administered intravenously.
Clause 115. The method of any one of clauses 109 to 114, wherein the composition is administered as a first single dose or as a split dose over two consecutive days.
Clause 116. The method of any one of clauses 109-115, wherein the administering comprises:
   a first dose of about 0.5 mg/kg; and
   a second dose of about 1 mg/kg or 2 mg/kg,
   wherein the second dose is administered the day after the first dose.
Clause 117. The method of clause 115, wherein the composition is administered about every 6 months after the first single dose or the split dose.
Clause 118. The method of any one of clauses 109-116, wherein treatment of the T1D in the subject produces an effect of partial clinical remission or stabilization of type 1 diabetes.
Clause 119. The method of clause 109, wherein treatment of the T 1D in the subject produces of p-cell function.
Clause 120. The method of clause 119, wherein the preserving of 3-cell function is measured by an amount of C-peptide.
Clause 121. The method of clause 113, wherein treatment of the T1D in the subject produces an effect of an insulin requirement of <0.25 units per kg of body weight per day in the subject.
Clause 122. The method of clause 113, wherein treatment of the T1D in the subject produces an effect of an HbA1c <6.5% in the subject.
Clause 123. The method of clause 118, wherein the partial clinical remission results in an insulin-dose adjusted A1C of <9.
Clause 124. The method of clause 113, wherein treatment of the T1D in the subject produces an effect of reduction in exogenous insulin use compared to a baseline or reference level.
Clause 125. The method of clause 113, wherein treatment of the T1D in the subject produces an effect of a peak C-peptide of≥2nmol/L.
Clause 126. The method of clause 113, wherein treatment of the T1D in the subject produces an effect of an HbA1c <7.5% and an exogenous insulin dose of <0.25, 0.25 to <0.50, 0.50 to <0.75, 0.75 to <1.0, 1.0 to <1.25, or >1.25 U/kg/day.
Clause 127. The method of any one of clauses 109-117, wherein treatment of the T1D in the subject produces substantially no or reduced adverse events compared to administering of a rabbit anti-thymocyte globulin.
Clause 128. The method of clause 127, wherein the adverse events are selected from the group consisting of serum sickness, immunogenicity, anaphylaxis, cytokine release syndrome, infusion-related reactions, sustained lymphodepletion, sustained lymphopenia, neutropenia, thrombocytopenia, anemia, hypoglycemia, diabetic ketoacidosis, hyperglycemia, and increased coagulation markers.
Clause 129. The method of clause 120, wherein treatment of the T1D in the subject produces no sustained depletion of T regulatory cells at 2, 4, 6, 8, 10, or 12 months post-treatment.
Clause 130. The method of any one of clauses 109-117, wherein treatment of the T1D in the subject produces no clinically significant changes in neutrophil counts, red blood cell counts, or platelet counts at 2, 4, 6, 8, 10, or 12 months post-treatment.
Clause 131. The method of any one of clauses 109-117, wherein treatment of the T1D in the subject produces no negative impact on cellular or humoral immunity.
Clause 132. The method of any one of clauses 109-117, wherein treatment of the T1D in the subject produces no clinically significant changes in hematology parameters, coagulation parameters, or clinical chemistry parameters at doses up to 5 mg/kg.
Clause 133. The method of any one of clauses 109-117, wherein treatment of the T1D in the subject produces a sustained CD4+ T-cell exhaustion.
Clause 134. The method of clause 133, wherein the exhaustion is measured by any marker selected from the group consisting of PD-1, TIGIT, KLRG1, LAG-3, TIM-3, BTLA, CD160, CD57, CD244/2B4, CTLA-4, Interferon-α, interferon-β, IL-2, interferon-γ, TNF-α, perforin, and granzyme.
Clause 135. The method of clause 133, wherein the sustained exhaustion persists for at least 90 to 120 days following treatment.
Clause 136. The method of any one of clauses 109-117, wherein treatment of the T1D in the subject produces a sustained activation and/or proliferation of T-regulatory cells.
Clause 137. The method of clause 136, wherein the sustained activation and/or proliferation of T-regulatory cells persists for at least 6 months.
Clause 138. The method of any one of clauses 109-117, wherein treatment of the T1D in the subject produces an immune adaptation to preserve protective and regulatory immunity while avoiding broad immunosuppression.
Clause 139. The method of clause 109, wherein the subject has Stage 2 T1D.

### EXAMPLES

The following specific examples are to be construed as merely illustrative, and not limitative of the remainder of the disclosure in any way whatsoever.

### Example 1

### Production of Human Polyclonal ATG in Transchromosomic Bovine (TcB) System

This Example describes the development of a novel human polyclonal ATG product (termed herein the “TcB product” or “SAB-142”) that overcomes known limitations of animal ATGs. The diversitAb™ technology, a transchromosomic bovine (TcB) system, in which cows with a bovine Ig locus replaced by a human artificial chromosome express fully human polyclonal antibodies was utilized.

A TcB subject was immunized with fresh human thymocytes and adjuvant at 3-5 week intervals. Hyperimmune plasma was collected after the 3rd-5th vaccinations (V3-V5). Immunization study design is summarized in TABLE 10. The amount of hyperimmune plasma collected from the subject animals at days 7, 11, and 14 after vaccination 5 (V5) was 2.100 of plasma by weight of animal (BW). Additionally, 6^{th} and 7^{th} vaccinations were also performed (V6 and V7).

TABLE 10
shows vaccine formulations and vaccination
schedule in the immunization study.

| Vaccination | | Blood Collection |
|---|---|---|
| (3-6 week interval) | Vaccine Formulation | (D = day) |
| V1 | 2 × 10⁹ fresh thymocytes + | D 0, D 4, D 20 |
| | adjuvant | |
| V2 | 2 × 10⁹ fresh thymocytes + | D 11, D 14, D 21 |
| | adjuvant | |
| V3 | 2 × 10⁹ fresh thymocytes + | D 7, D 11, D 14 |
| | adjuvant | |
| V4 | 5.5 × 10⁹ fresh thymocytes + | D 7, D 11, D 14 |
| | adjuvant | |
| V5 | 4.12 × 10⁹ fresh thymocytes + | D 7, D 11, D 14 |
| | adjuvant | |
| V6 | 4.75 × 10⁹ fresh thymocytes + | D 7, D 11, D 14 |
| | adjuvant | |
| V7 | 5.5 × 10⁹ fresh thymocytes + | D 7, D 11, D 14 |
| | adjuvant | |

Different lots of SAB-142 were generated by pooling the plasma produced from different immunizations. For example, SAB-142 Lot 1 was generated by pooling immunizations V3 and V4. SAB-142 Lot 3 was generated by pooling plasma from immunizations V5-V7 and SAB-142 Non-clinical Lot was generated by pooling plasma from immunizations V3-V7. Subsequent experiments described herein used these lots to characterize SAB-142.

### Example 2

Binding Profile of SAB-142 to T cell Surface Markers

The objective of this study is to determine and compare the binding specificity profile of SAB-142 and Thymoglobulin® against T cell surface markers by using an in vitro expression system that allows transmembrane surface proteins to be presented to the antibodies in their native conformation.

The selected human CD markers were stably expressed in BW5147 cells (mouse T cell line). Methods related to BW5147 are disclosed in I. Popow, et al. Am J Transplant 2013 Vol. 13 Issue 12 Pages 3103-13 (the contents of which are herein incorporated by reference in its entirety.) Antigen binding was assessed by flow cytometry analysis using species-specific secondary antibodies. Due to the differences in the secondary antibodies, a direct comparison of Thymoglobulin® and SAB-142 was not possible. Using CD2, CD4, or CD8 expressing T cell lines, the binding of Thymoglobulin® and SAB-142 to the overexpressed target antigens was determined.

### Methods

BW5147 cell lines were engineered to stably express human T cell surface antigens known to be targets of ATG products. SAB-142 and rATG were fluorescently labeled, and flow cytometry analysis was used to measure binding to the expressed human antigens.

### Results

SAB-142 and rATG were directly labeled with a fluorescent tag to allow for direct comparison of binding. Flow cytometry analysis showed that SAB-142 and rATG bound to human T cell associated surface proteins, including CD2, TCR-CD3, CD4, CD8, CD45, and HLA-DR (FIG. 2A- FIG. 2F). Of the studied antigens, human ATG and rATG had the highest relative binding to HLA-DR followed by CD2. All ATGs bound to CD8 at a higher level than CD4. Generally, the binding specificity profiles of human ATG and rATG strongly overlapped.

### Conclusions

Here, the first data on the characterization of human ATG binding to T cell associated CD proteins is presented. Human surface proteins expressed on mouse T cells serve as a conformational system to study antibody targeting. In vitro studies indicate a strong similarity in the specificity binding profile of human ATG and rATG, which is consistent with both products being generated using fresh human thymocytes as the antigen for immunization. These results highlight the therapeutic potential of human ATG for T1D treatment, which avoids the risks of immune reactions that can occur with rabbit immunoglobulins.

SAB-142 binds to Receptors

SAB-142 demonstrates a binding profile that closely mirrors that of rabbit antithymocyte globulin (rATG), targeting the same key T cell surface antigens implicated in immunomodulation. This similarity in receptor engagement supports the potential of SAB-142 to achieve comparable immunological effects as rATG, while offering the added advantages of a fully human antibody composition. These results highlight the therapeutic potential of human ATG for T1D treatment, which avoids the risks of immune reactions that can occur with rabbit immunoglobulins. As a result, SAB-142 may provide a safer and more tolerable alternative for clinical applications requiring targeted T cell modulation, with the benefits of reduced immunogenicity and improved patient outcomes.

### Example 3

### Resonance Units of SAB-142, Thymoglobulin®, and ATGAM(R) Binding to T Cell CD Markers

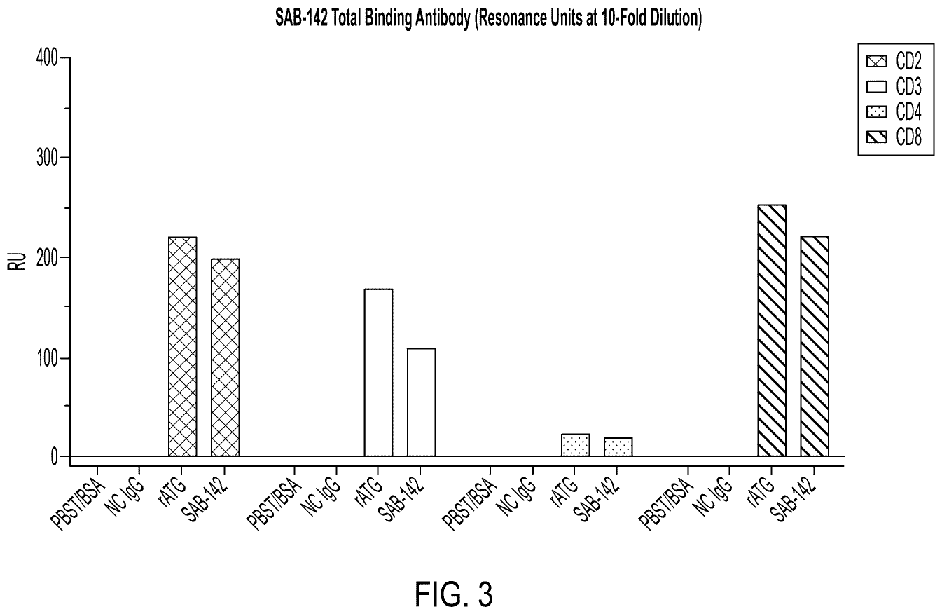

As used herein, a resonance unit (RU) is defined as a unit used to measure changes in mass on a gold-coated sensor chip in Surface Plasmon Resonance (SPR). A shift of 10⁻⁴ in the resonance angle is equal to 1 RU, which corresponds to an increase of 1 μg per square millimeter of surface mass. RU is a quantitative measure that can be used to determine the relative amount of different polyclonal antibody products that bind to a particular antigen of interest. To determine and compare the RUs SAB-142 and Thymoglobulin® against CD2, CD3, CD4, and CD8, each of the antibody's RUs was analyzed as a quantitative assessment against purified CD recombinant proteins by Surface Plasmon Resonance (SPR)-based antibody kinetics assays. The purified recombinant CD proteins were captured to a Ni-NTA sensor chip. Purified SAB-142 and Thymoglobulin® (rATG) were diluted and injected onto the chip for association, and then resonance units for each CD2, CD3, CD4, and CD8 were determined. SAB-142 and Thymoglobulin® both have lower relative binding to CD4 and the highest binding to CD8 (FIG. 3).

### Example 4

### Avidity of SAB-142 Against T Cell CD Markers

The binding strength of an antibody to a monovalent epitope is referred to as affinity. Polyclonal antibodies interact with multiple epitopes on one or more antigens. Avidity entails the affinity of each antibody for its epitope, the number of antibody binding sites on multivalent antigens, and the geometry of the resulting antibody-antigen complexes. The binding strength of a polyclonal antibody population is therefore defined by measuring avidity.

To characterize the avidity of SAB-142 against T cell surface antigens, the avidities of SAB-142 and of individual plasma immunization timepoints were studied.

The antibody avidity was analyzed against purified CD recombinant proteins by Surface Plasmon Resonance (SPR)-based antibody kinetics assays. The purified recombinant CD proteins were captured to a Ni-NTA sensor chip. Purified SAB-142, Thymoglobulin® (rATG), and Tc Bovine plasma were diluted and injected onto the chip for association, and disassociation was performed over time. Antibody off-rate constants were determined directly from the antibody interaction with recombinant purified CD proteins.

To characterize the individual timepoint plasma samples, the avidity to CD proteins was examined with SPR analysis. The measured avidity inverse is graphed such that the higher the amount of residence time, the greater the avidity.

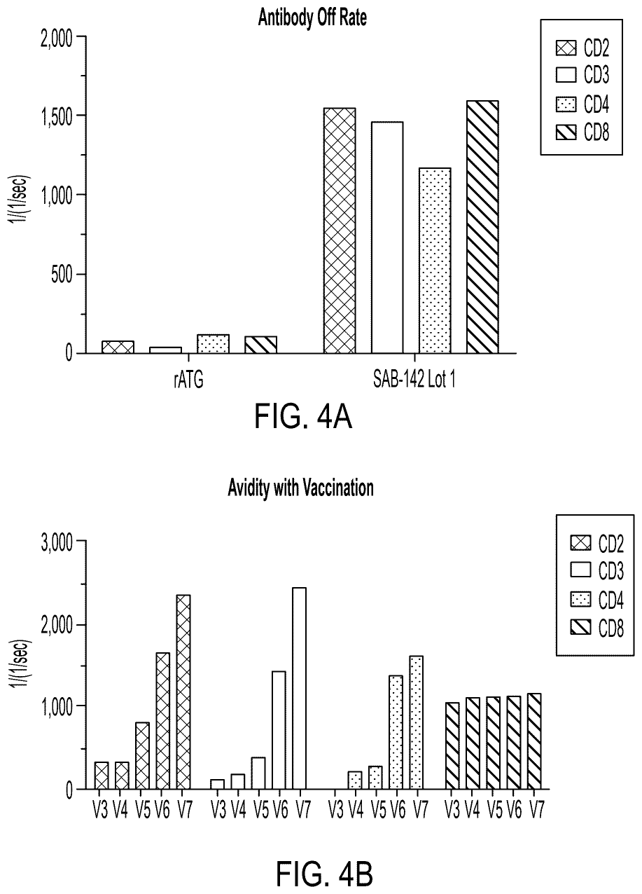

Plasma was collected from Tc Bovines after each immunization with fresh human thymocytes (samples V3-V7) and partially purified over an anti-human IgG column. Since the Tc Bovine is hyperimmunized, affinity maturation can occur, generating high-affinity antibodies at later vaccination time points. When the avidity of these samples was examined, avidity to the CD proteins also increased over time (FIG. 4B).

Interestingly, rATG had very low avidity to all CD proteins tested compared to SAB-142 (FIG. 4A). The avidity of Tc Bovine plasma to CD2, CD3, CD4, and CD8 increased with each thymocyte vaccination, indicating the impact of repeated immunization on avidity and antibody affinity maturation (FIG. 4B).

### Example 5

### Characterization of SAB-142 & Thymoglobulin® Fc Receptor Function

Antibody fragment crystallizable (Fc) regions mediate functional antibody outcomes as well as antibody homeostasis in the unbound state.

Antibody Recycling and Transport into Tissues:

IgG homeostasis is controlled by antibody Fc binding to the FcRn. Increased binding leads to higher levels of recycling, extended presence in the plasma, and movement from the vasculature into tissues.

The objective was to define the binding of SAB-142 to the human Fc neonatal receptor (FcRn). The FcRn controls antibody recycling in the endocytic compartment following antibody internalization. Increased binding leads to higher antibody recycling and increased antibody concentrations in the plasma and tissues. The Promega Lumit® Assay, a competitive binding inhibition assay, where left-shifted curves indicate higher binding, was used. Native human IgG was used a standard control (see FIG. 8A).

SAB-142 Lots have similar FcRn binding compared to native human IgG but have higher binding than Thymoglobulin®. The binding differences to the FcRn suggest higher levels of SAB-142 recycling relative to Thymoglobulin®.

A negative competition assay where increased binding to human FcγRI from test article results in decreased luminescent signal was performed. The standard control was native human immunoglobulin. As shown in FIG. 8B, binding to FcγRI was similar for all SAB-142 lots tested and Thymoglobulin®.

To investigate binding of the antibodies to the human FcγRII, which is the initial step in activating cellular pathways leading to antibody-dependent cellular phagocytosis (ADCP), a negative competition assay where increased binding to human FcγRII by the test article results in decreased luminescent signal was performed. The standard control was native human immunoglobulin.

SAB-142 Lots and control human IgG show similar binding affinity to human FcγRII. SAB-142 curves had a lower IC₅₀ compared to rATG, indicating that SAB-142 has a higher binding affinity for FcγRII than Thymoglobulin® (see FIG. 8C). These data show that SAB-142 may have increased potential to activate ADCP in vivo.

Next, FcγR downstream signaling, which is controlled by immune complex Fc/FcR binding, followed by receptor clustering, was analyzed. Activation of downstream pathways provides additional evidence for ADCP. Genetically engineered reporter cell lines expressing human FcγRII utilize native downstream signaling pathways to drive reporter expression indicative of ADCP activation and were used in these experiments, where increased luminescence signifies increased activation.

Overall, SAB-142 appeared to have slightly increased FcγRIIa pathway activation relative to rATG, consistent with increased FcγRII binding. This may suggest SAB-142 could have higher ADCP in vivo.

To investigate binding to the human FcγRIII, which activates cellular pathways leading to antibody-dependent cellular cytotoxicity (ADCC), assays similar to those described above were performed. SAB-142 shows lower binding to FcγRIIIa and increased IC₅₀ pathway activation values compared to Thymoglobulin®, suggestive of decreased ADCC for SAB-142 in vivo.

FcγR downstream signaling is controlled by immune complex Fc/FcR binding, followed by receptor clustering. Activation of downstream pathways provides additional evidence of ADCC. Genetically engineered reporter cell lines expressing human FcγRIII utilize native downstream signaling pathways to drive reporter expression and indicate ADCC activation. Based on EC₅₀ values, all SAB-142 lots had significantly lower pathway activity compared to Thymoglobulin® (FIG. 8D). This data is consistent with the ADCC binding data, suggesting that SAB-142 could have notably decreased ADCC activity in vivo relative to rATG.

In conclusion, SAB-142 and Thymoglobulin® have similar binding to human FcγRI, suggesting comparable levels of overall immune activation. SAB-142 has higher binding and pathway activation of FcγRIIa, suggesting increased induction of ADCP. SAB-142 has lower FcγRIIIa binding and pathway activation, suggesting reduced ADCC compared to Thymoglobulin®.

### Example 6

### SAB-142 Preserves and Activates T Regulatory Cells

In clinical evaluation, SAB-142 has demonstrated a great ability to preserve and activate regulatory T cells (Tregs), which maintain immune homeostasis and self-tolerance. Data from the SAB-142-101 Phase I study indicate that administration of SAB-142 results in the preservation of Treg populations, thereby supporting the maintenance of regulatory immune function. Specifically, analysis of healthy volunteer cohorts receiving doses of 1.5 mg/kg or 2.5 mg/kg SAB-142 revealed that Treg cell numbers remained stable throughout the observation period. . Mean subject normalized plots for CD4+ Tconv, Treg, and CD8+ T-cells show generally stable populations among treated subjects and a lack of depletion (FIG. 13A, FIG. 13B, and FIG. 13C). This preservation of Tregs distinguishes SAB-142 from other immunomodulatory agents that may cause depletion of these critical cells.

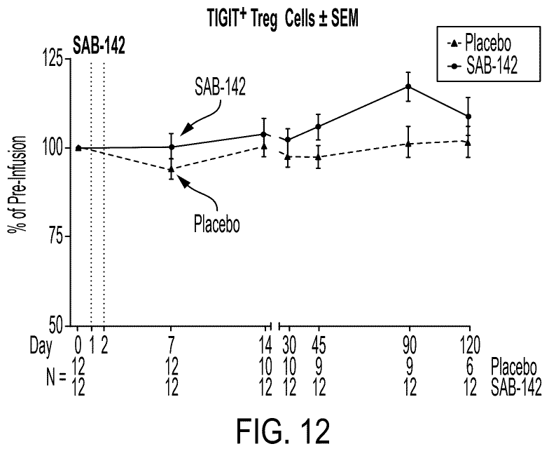

Furthermore, SAB-142 treatment was associated with the induction of the inhibitory receptor TIGIT on Treg cells (FIG. 12). The increased expression of TIGIT is indicative of potential Treg activation, which may contribute to the sustained immunomodulatory effects observed with SAB-142 administration. The activation and preservation of Tregs are believed to play a large role in promoting long-term immune tolerance and reducing the risk of autoimmune responses.

Collectively, these results highlight the potential of SAB-142 to provide targeted immunomodulation while maintaining the integrity and function of the regulatory T cell compartment.

### Example 7

### Phase I Clinical Results

This Example discloses the result of the Phase I clinical trial in a single-ascending dose among healthy volunteers.

SAB-142 was well-tolerated and demonstrated a favorable safety profile that positions SAB-142 for chronic dosing in an ambulatory setting.

The SAB-142 Phase I dose range was between 0.03 mg/kg up to 2.5 mg/kg, which demonstrated favorable safety profile based on the 0% reported serum sickness and low/no immunogenicity. The majority of adverse events (AEs) reported were mild, associated with day 1-2 infusions & resolved by the end of the first week.

SAB-142 demonstrated sustained immunomodulation with a CD4+ and CD8+, T-cell exhaustion marker signature (PD-1, KLRG1, TIGIT) known to correlate with C-peptide preservation

Lymphocyte-sparing and Treg activation effect of SAB-142 was observed in clinical studies. Consistent with CBC data, both CD4+ and CD8+ cells, expressed as a percent of mature (CD3⁺) T cells, are largely flat throughout the monitoring period, even during transient loss from the periphery. The observed consistency in primary lymphocyte subsets supports a lack of cytotoxic targeting of either compartment. The noncytotoxic effects of SAB-142 were extended to B lymphocytes and Treg cells, as evidenced by their stability relative to pre-infusion values. In addition to its absence of cytotoxicity, SAB-142 treatment results in increased positivity of the co-inhibitory receptor TIGIT on Treg cells, potentially enhancing their suppressive function.

Inhibitory co-receptor expression of PD-1, KLRG1, and TIGIT was also observed on CD4+ and CD8+ T-cells. Normalized PD-1 and TIGIT positivity from pooled cohorts 4-5N (1.5-2.5 mg/kg) are presented for CD4+ and CD8+ cells. Increasing positivity for both co-inhibitory receptors at later time points is evident in both compartments and is more consistent with exhaustion than activation, which typically features an early, acute spike and subsequent resolution.

SAB-142 showed a transient reduction in circulating lymphocytes followed by rapid recovery to baseline within approximately 48 hours from the last infusion, consistent with ephemeral margination (FIG. 10). Thus, SAB-142 showed no sustained reduction in peripheral immune cell numbers, including CD4+, CD8+, or B-cell number reduction, an advantage over rabbit ATG, which causes a decrease in CD4+ T-cells for up to 2 years.

Cohorts of SAB-142 did not generate anti-SAB-142 antibodies in healthy volunteers at target doses (2.5 mg/kg), indicating that SAB-142, as a fully human IgG pAb, is significantly less immunogenic compared to Thymoglobulin®, rabbit IgG pAb.

SAB-142 is a Fully Human, Multi-Specific, Targeted Anti-Thymocyte Globulin (hATG) for Delaying Onset and Progression of T1D

### Potential Disease Modification

SAB-142 is a lead candidate with the potential to deliver meaningful disease modification in patients with newly diagnosed Stage 3 type 1 diabetes (T1D). The therapeutic regimen is designed for convenient twice-yearly dosing, which is supported by robust clinical data and a de-risked mechanism of action. This approach aims to alter the course of T1D by intervening early in the disease process, improving long-term outcomes.

### Positive Phase I Data Supports De-Risked Method of Action

Results from the Phase I clinical trial demonstrate that SAB-142 exhibits a mechanism of action (MOA) comparable to rabbit anti-thymocyte globulin (ATG), a well-established immunomodulatory agent. Importantly, SAB-142 offers improved safety and tolerability, with the potential for repeat dosing—an advantage over rabbit ATG, which is limited by immunogenicity and adverse reactions. These findings support the advancement of SAB-142 into the Phase 2b SAFEGUARD study, where its efficacy and safety will be further evaluated in a larger patient population.

The unique multi-specific antibody platform sab-142 was developed using a first-of-its-kind platform capable of generating a diverse repertoire of multi-specific, targeted, anti-thymocyte human IgG antibodies. This innovative technology enables precise targeting of immune cell populations implicated in T1D pathogenesis, while minimizing off-target effects and immunogenicity. The SAB-142-101 Phase I study was conducted as a randomized, double-blind, placebo-controlled, single- and multiple ascending dose, adaptive design clinical trial in healthy volunteers and patients with established T1D (total n=68; healthy volunteers n=62, T1D patients n=6). The study design and results provide a strong foundation for the continued clinical development of SAB-142 as a disease-modifying therapy for T1D.

TABLE 11
shows the dosing for each cohort of subjects tested.

| | |
|---|---|
| Repeat Dose | n = 8 |
| 1.5 (mg/kg) | |
| 4.5 mg/kg | n = 8 |
| 2.5 mg/kg | n = 22 |
| 1.5 mg/kg | n = 8 |
| 0.5 mg/kg | n = 16 |
| 0.1 mg/kg | n = 8 |

SAB-142 has demonstrated a clinically validated, multi-specific mechanism of action (MOA) characterized by sustained immunomodulation. This therapeutic profile is supported by comprehensive clinical data, positioning SAB-142 as a lead candidate for safe and reliable chronic dosing in patients requiring long-term immune intervention. Importantly, SAB-142 does not cause lymphodepletion, nor does it induce neutropenia or sustained decreases in red blood cells or thrombocytes. This safety profile distinguishes SAB-142 from other immunomodulatory therapies that are often associated with significant cytopenias and related complications.

Pharmacokinetic and pharmacodynamic (PK/PD) data further demonstrate that SAB-142 induces a sustained “T-cell exhaustion” signature, a hallmark of effective immunomodulation in autoimmune conditions. This signature has been clinically validated by rabbit ATG and other T-cell targeting immunomodulatory drugs used in type 1 diabetes (T1D), and is proven to correlate with C-peptide preservation in clinical studies involving patients with new onset T1D.

AB-142 also offers significant advantages in terms of tolerability and immunogenicity. The therapy does not cause serum sickness and exhibits low or no immunogenicity, as evidenced by the absence of anti-drug antibody formation in clinical studies. This enables the potential for repeat dosing and long-term use without the risk of hypersensitivity reactions or loss of efficacy.

SAB-142 shares some similarities with rabbit ATG, binding to the same key T cell surface receptors (see Example 2). Functionally, SAB-142 demonstrates sustained exhaustion of CD4+conventional T cells, analogous to the effects observed with rATG. Specifically, SAB-142 induces persistent expression of inhibitory receptors such as PD-1 and TIGIT on CD4+ T cells. This multi-target T-cell exhaustion profile, as measured by PD-1 mean fluorescence intensity (MFI), is strongly associated with C-peptide preservation in T1D.

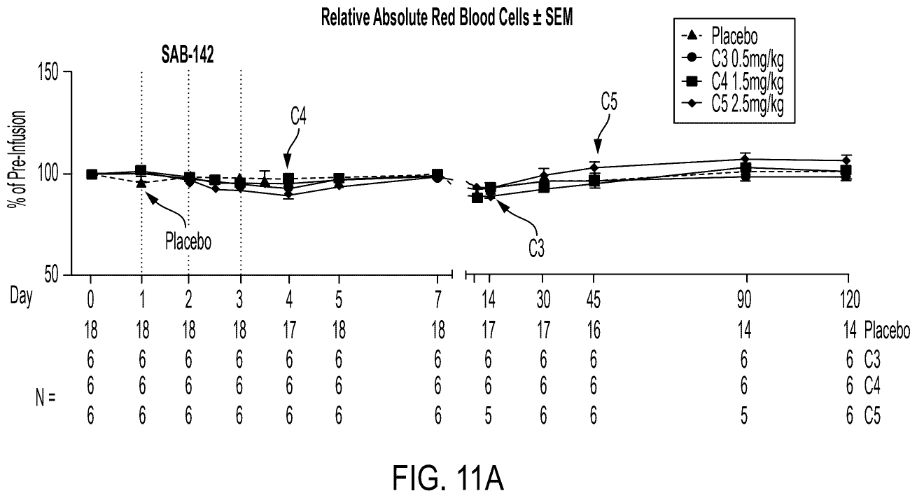

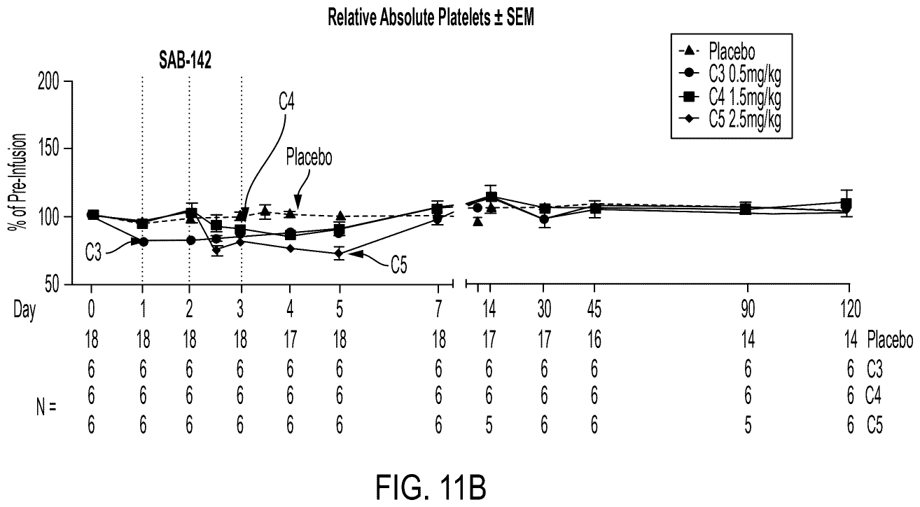

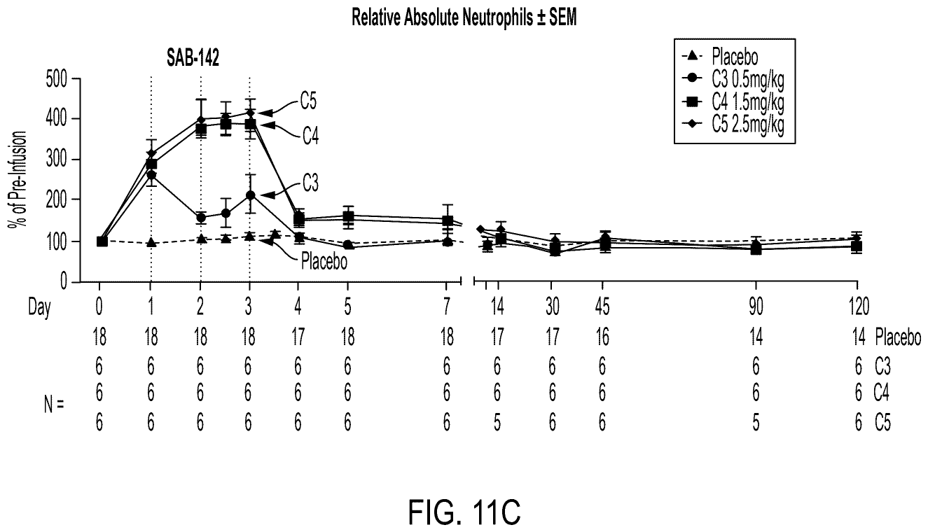

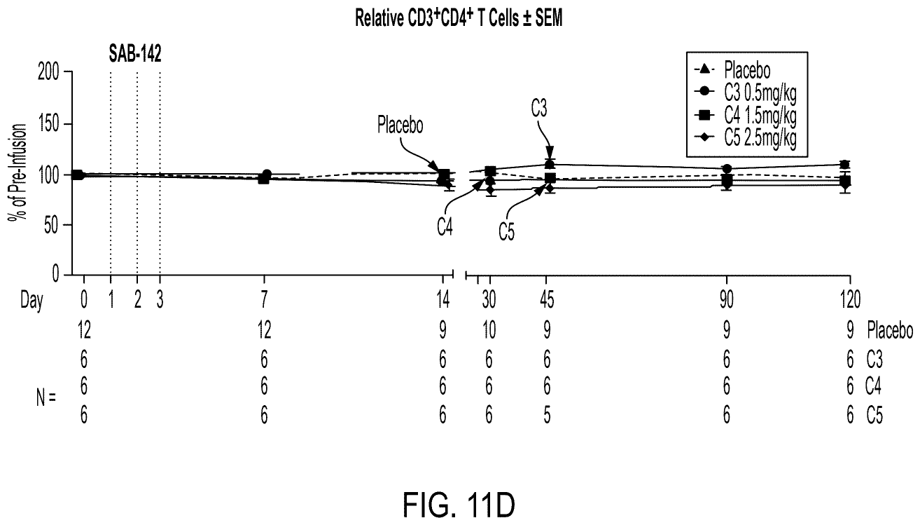

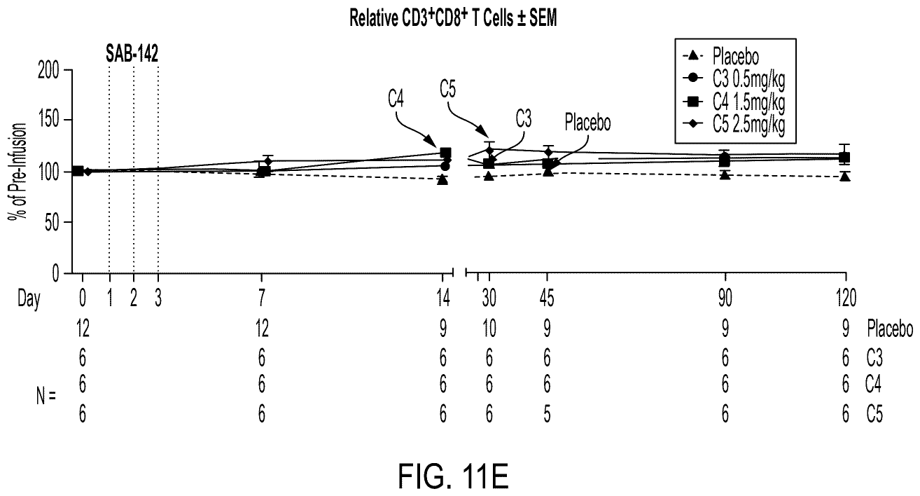

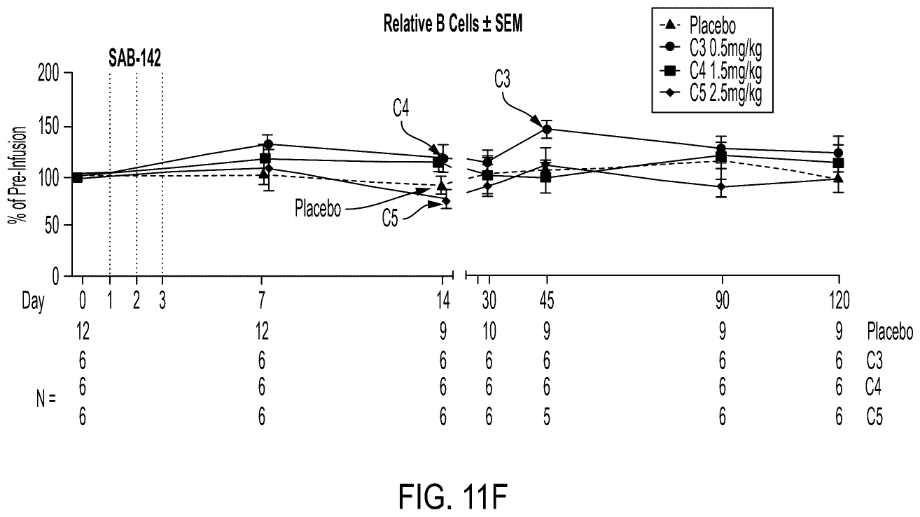

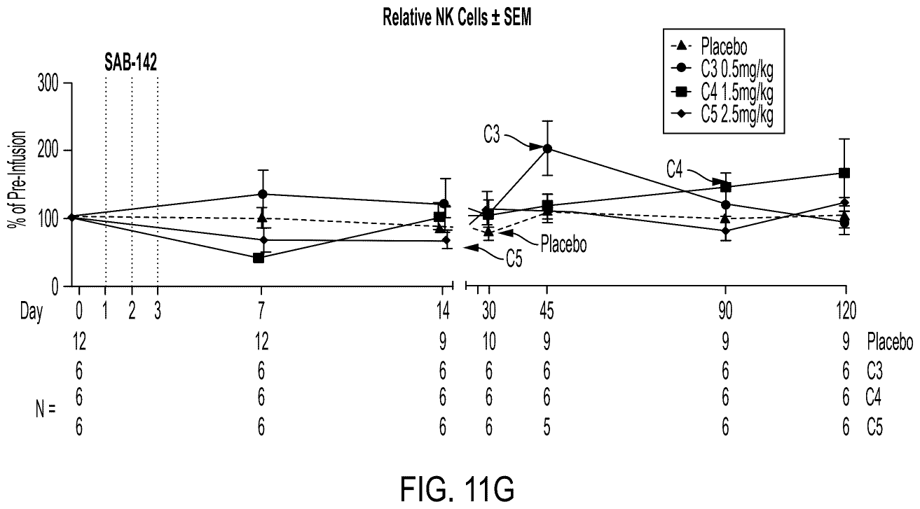

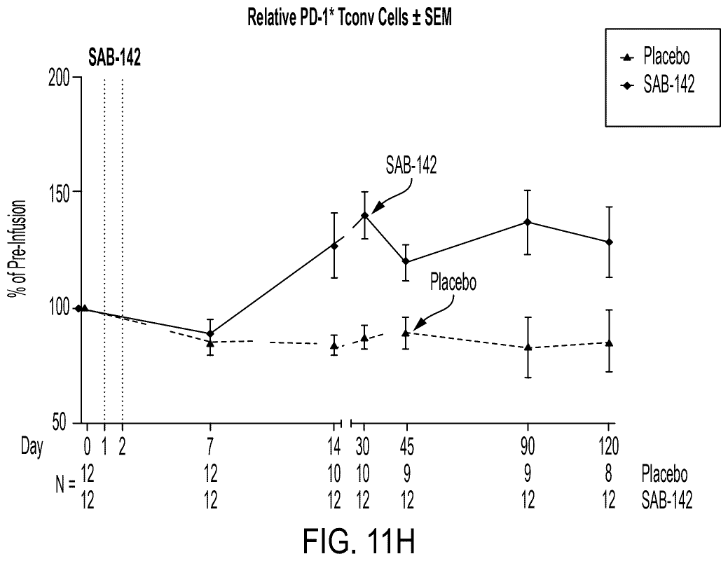

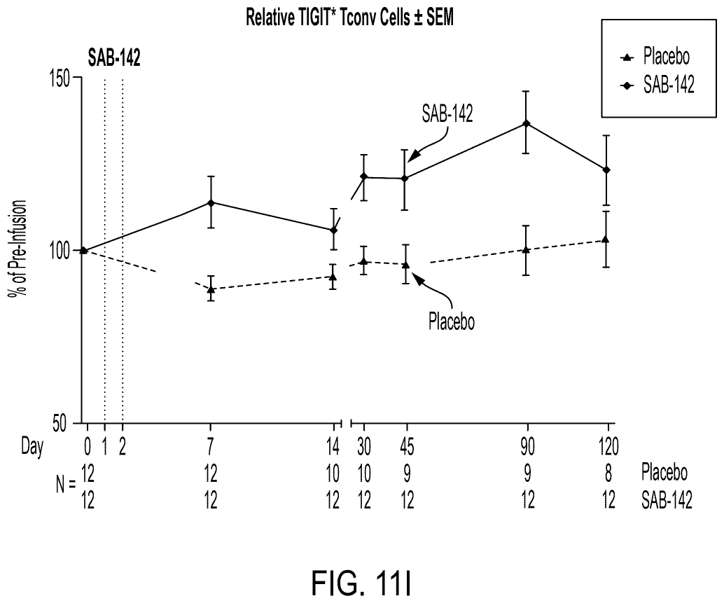

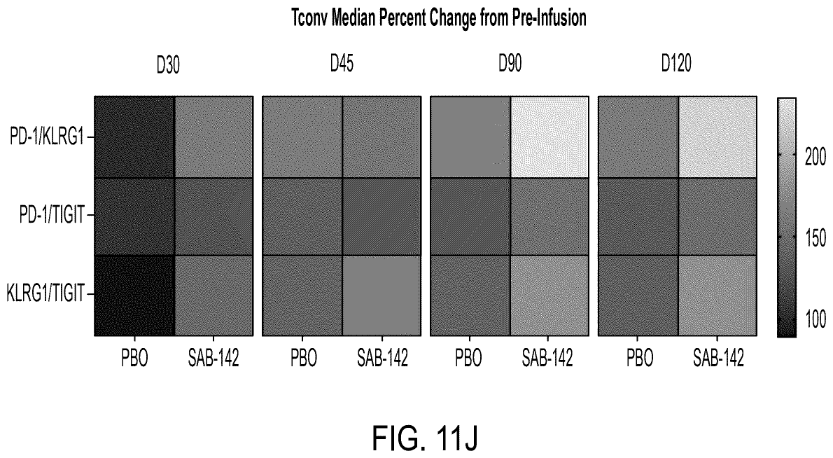

SAB-142 has been shown to induce persistent expression of co-inhibitory receptors on CD4+ conventional T cells, as illustrated in FIG. 11I. This effect mirrors the mechanism observed with low-dose anti-thymocyte globulin (ATG), which also induces sustained expression of co-inhibitory receptors such as PD-1 and KLRG1 on CD4+ cells. The resulting exhaustion-like phenotype has been correlated with preservation of C-peptide in patients with type 1 diabetes, as described by Jacobsen et al. (JCI Insight 8.16 (2023): e161812, incorporated herein by reference in its entirety).

Unlike traditional rabbit ATG, SAB-142 does not cause sustained depletion of immune cells. Clinical data demonstrate that all cell populations, including lymphocytes, recover to baseline levels by Day 7 following administration (FIG. 10, FIG. 11B, FIG. 11C, FIG. 11D, FIG. 11E, FIG. 11F, and FIG. 11G. There is no evidence of sustained lymphodepletion, a significant safety advantage over rabbit ATG, which is known to cause prolonged decreases in CD4+ T-cell counts for up to two years.

FIG. 11H shows an increase in PD-1+ Tconv cells after treatment with SAB-142, supporting an extended Tconv exhaustion phenotype.

SAB-142 does not deplete red blood cells, neutrophils, or platelets, with all hematologic cell types returning to baseline by Day 7 post-treatment (FIG. 11A, FIG. 11B, FIG. 11C, and FIG. 11G). The median percentage change for multiple targets including PD-1:KLRG1, PD-1:TIGIT, and KLRG1:TIGIT at day 30, day 45, day 90, and day 120 are shown in FIG. 11J. The ratio of PD-1:KLRG1 is increased over time after administration of SAB-142, in addition to the ratio of KLRG1:TIGIT and the ratio of PD-1:TIGIT (at a lesser extent). This favorable safety profile, combined with its validated mechanism of action, positions SAB-142 as a potentially best-in-class immunotherapy for type 1 diabetes. The agent achieves effective immunomodulation without the risks associated with sustained cytopenias, supporting its use in chronic or repeat dosing regimens.

Collectively, these findings highlight SAB-142's potential as a best-in-class immunotherapy, offering durable immunomodulation, a favorable safety profile, and the ability to preserve endogenous insulin production in patients with T1D.

### Example 8

### Detection of SAB-142 is in Cynomolgus Lymph Nodes

The purpose of the experiment was to evaluate whether SAB-142 was present in lymphocytes isolated from lymph nodes of SAB-142 treated juvenile cynomolgus monkeys.

Lymph nodes were surgically removed from monkeys re-dosed at 6 months with doses of 10, 25 or 50 mg/kg of SAB-142 or placebo (n=6; 3m:3f).

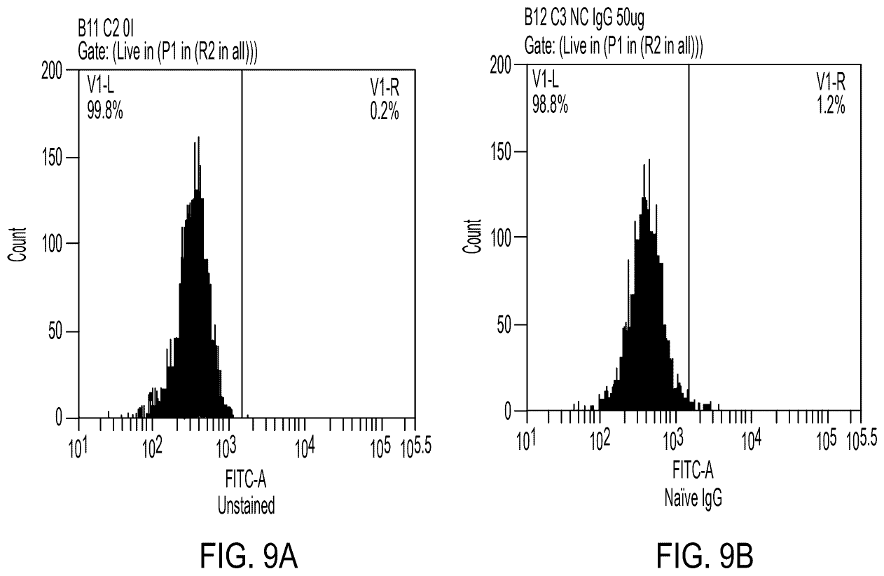

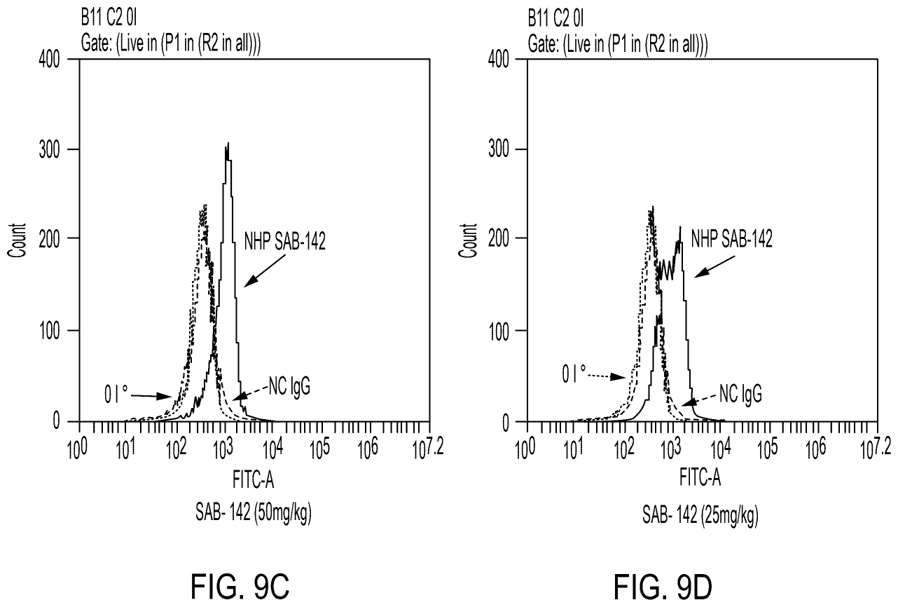

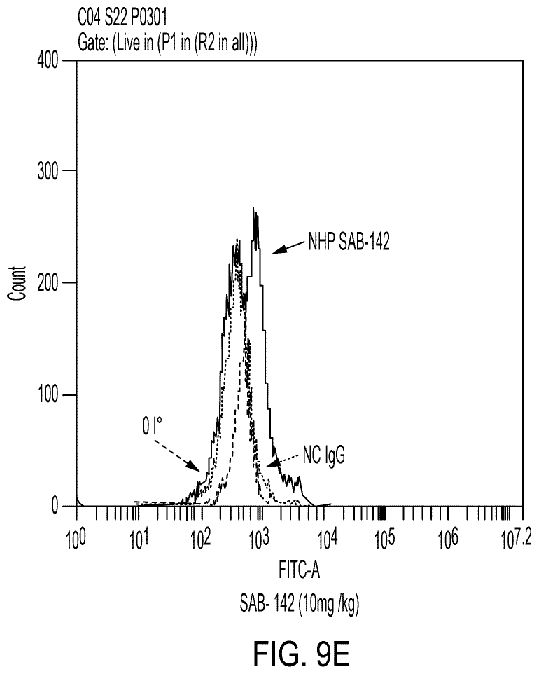

Single cell suspensions from lymph node tissue were generated, and cells were analyzed by flow cytometry using an anti-human IgG detection antibody. A shift in fluorescence intensity from SAB-142 in re-dosed Cynomolgus was present compared to no treatment and the negative control antibody, indicating that SAB-142 is present in the lymph nodes of Cynomolgus (FIG. 9A, FIG. 9B, FIG. 9C, and FIG. 9D). The 10 mg/kg dose is predicted to be in the range of 2.5-3.5 mg/kg for human subjects based on surface area conversion (FIG. 9E).

### Example 9

Characterization of Induced T Cell Cytokine Release after Treatment with SAB-142 & Thymoglobulin®

Therapies targeting lymphocytes often lead to the release of cytokines from targeted cells. It has been demonstrated that both SAB-142 and Thymoglobulin® bind to lymphocytes. To further investigate this effect, a cytokine release assay was developed to interrogate in vitro cytokine release profiles and to evaluate potential consistencies or differences between various lots of SAB-142 and Thymoglobulin®. In vitro analysis of cytokine release from peripheral blood mononuclear cells (PBMCs) following administration of anti-thymocyte globulin (ATG) was conducted using a cytometric bead array. This multiplexed assay format enables the simultaneous interrogation of seven different cytokines, with cytokine levels interpolated from standards of known concentration to ensure quantitative accuracy.

SAB-142 lots and Thymoglobulin® exhibited similar cytokine release profiles across six commonly affected cytokines when compared to control treatments. This finding shows a comparable immunomodulatory effect between the two agents in terms of cytokine induction.

### SAB-142 and Thymoglobulin® In Vitro Cytokine Release Assay

An in vitro cytokine release assay was conducted to evaluate the effects of SAB-142 and Thymoglobulin® on lymphocyte cytokine secretion.

Cytokine release from lymphocytes is a well-recognized biological outcome of therapies that target these cells. To assess this, a multiplexed assay was developed to stimulate peripheral blood mononuclear cells (PBMCs) and measure cytokine release. The methods involved the use of a multiplexed cytokine bead array to evaluate cytokine levels secreted from PBMCs incubated with anti-thymocyte globulins (ATGs). Quantitation of cytokine concentrations was performed by interpolation from standard curves of known values. The outcome of the assay showed that fluorescence shifts between bead populations labeled with allophycocyanin (APC) were distinct and well delineated. Additionally, fluorescence shifts in the detection antibody (phycoerythrin, PE) increased proportionally with cytokine concentration, indicating that the assay performed as intended and was suitable for quantitative cytokine measurement.

To accurately assess this response, a multiplexed assay was developed to stimulate peripheral blood mononuclear cells (PBMCs) and measure the resulting cytokine release.

### Methods:

A multiplexed cytometric bead array was employed to evaluate cytokine levels secreted from PBMCs incubated with anti-thymocyte globulins (ATGs), SAB-142 and Thymoglobulin®. Quantitative analysis was performed by interpolating results from standard curves generated with known cytokine concentrations.

### Outcome

SAB-142 and Thymoglobulin® (rATG) have been observed to induce cytokine secretion in similar patterns across all tested cytokines.

### Example 10

### Assessing SAB-142 Cytotoxicity and Mechanisms of Cell Death

This example describes a dynamic assay assessment of SAB-142 & rATG Treatment of Human T cells. The purpose of this example was to evaluate the kinetics of regulated cell death in T cells, following exposure to SAB-142.

### Methods

A kinetic apoptosis and necrosis assay was employed to monitor cell death in real time. This approach involved the use of reagents that detect phosphatidylserine (PS) exposure as an early marker of apoptosis, measured by luminescence. Additionally, fluorescence was used to indicate loss of membrane integrity, which is consistent with necrotic cell death. The combination of these real-time kinetic measurements allowed for detailed characterization of the temporal sequence and mechanisms of cell death induced by SAB-142 in human T cells. Outcome

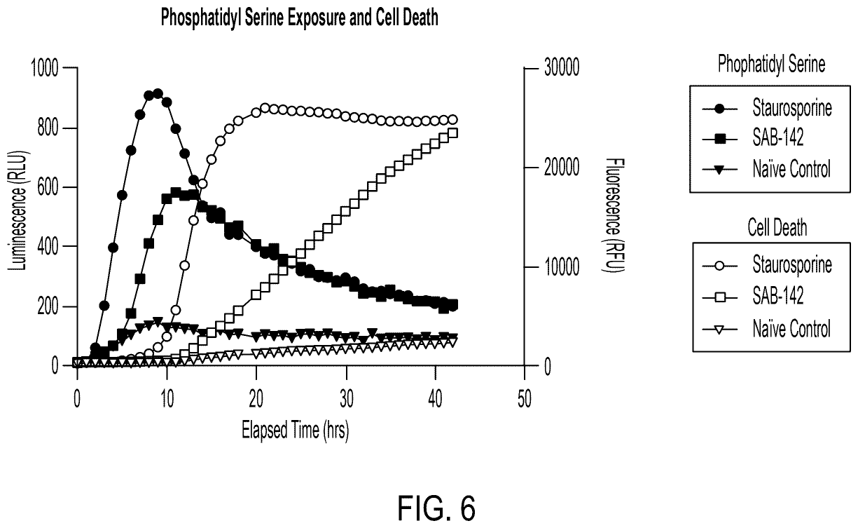

SAB-142 was found to induce delayed phosphatidylserine (PS) exposure on T cells within 5 to 7 hours post-administration (FIG. 6). This was followed by cell membrane permeabilization, a marker of late-stage cell death, which began at approximately 12 to 13 hours after treatment.

The extent of PS exposure observed with SAB-142 was delayed and less compared to what was observed in staurosporine. Furthermore, SAB-142 did not reach the level of cell death observed with positive control, struaoporine.

These findings are inconsistent with the regulation of cell death by SAB- through an apoptotic mechanism. However, as PS exposure occurs in other forms of regulated cell death, assessment of caspase activation can further support the primary pathway involved.

### Caspase Activation Triggering Apoptosis

The objective of this experiment was to investigate caspase activation, a process required for the execution of apoptosis.

### Methods

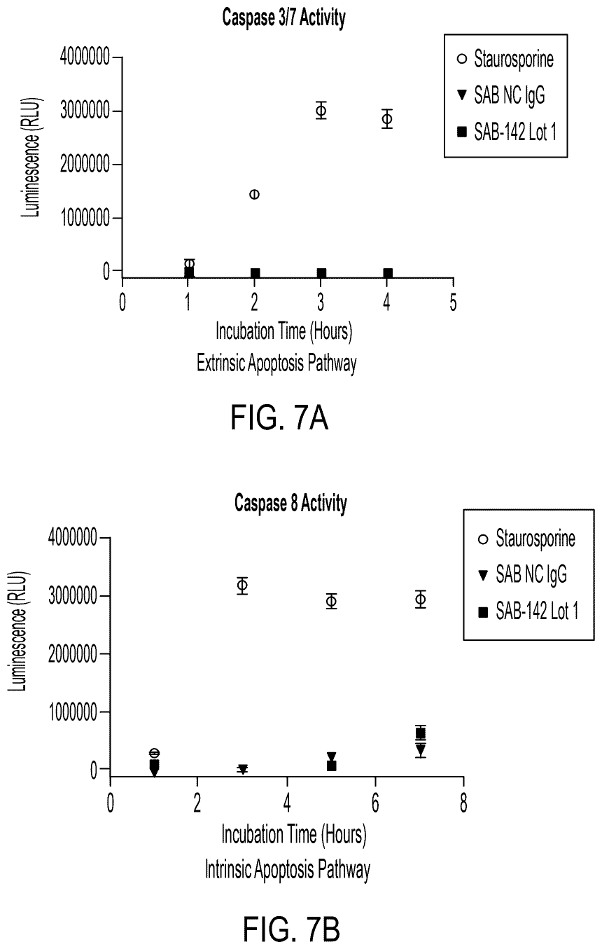

Caspase activation in Jurkat cells treated with SAB-142 and rATG was assessed using a Caspase Glow Assay. This assay employs a reagent that emits luminescence upon cleavage by active caspases, with the intensity of the luminescent signal being directly proportional to the level of caspase activity present in the sample (FIG. 7A and FIG. 7B). Staurosporine was included as a positive control to validate the assay's ability to detect caspase-mediated apoptosis.

### Outcome

Minimal activation of Caspase 3/7 and Caspase 8 was observed in Jurkat cells treated with SAB-142 (FIG. 7A and FIG. 7B). This finding further suggests that SAB-142 does not induce cell death primarily through apoptotic pathways, as caspase activation is a hallmark of apoptosis.

Instead, alternative mechanisms of regulated cell death are likely involved in the cytotoxic effects observed with these agents.

### Evaluation of SAB-142 and Thymoglobulin® Cytotoxicity

The purpose of this experiment was to extend the analysis of regulated cell death beyond the Jurkat cell line by evaluating the effects of SAB-142 on human peripheral blood mononuclear cells (PBMCs).

PBMCs were thawed and cultured overnight to allow for cellular recovery. On the following day, the cells were treated with control reagents, SAB-142, or rATG at concentrations ranging from 12.5 to 100 μg/mL. After incubation at 37° C., cells were harvested at designated time points for analysis.

To assess cell death and phosphatidylserine (PS) exposure, cells were double-stained with FVD-660 and ApoTracker Green. ApoTracker Green is a fluorogenic probe that specifically binds to PS, serving as a marker for early stages of cell death. Fixable Viability Dyes (FVD-660) irreversibly label dead cells with a fluorescent dye that cannot penetrate live cell membranes, thereby distinguishing live from dead cells. Cell viability and PS exposure were subsequently examined by flow cytometry.

After 24 hours of treatment, cells that received no treatment or were exposed to negative control IgG showed approximately one-third dead cells. In contrast, treatment with staurosporine resulted in 98% cell death, while SAB-142 and rATG treatments led to 78% and 86% dead cells, respectively.

SAB-142 and rATG Treatment of PBMCs

Cell viability was monitored at 3, 7, and 24 hours post-treatment to evaluate the effects of SAB-142 and rATG on PBMCs over time. For untreated cells and those treated with negative control IgG, viability remained steady throughout the observation period, indicating minimal spontaneous cell death. In contrast, cells treated with SAB-142 or rATG exhibited a decrease in viability, as reflected by a reduced percentage of intact cells. Notably, a substantial portion of cell death occurred rapidly, with a 30-60% reduction in viable cells already observed at the 3-hour time point, suggesting that much of the cytotoxic effect takes place early after exposure.

Given the rapid onset of cell death, it was determined that future experiments should include earlier time points to more precisely capture the kinetics of cell death induction. In these studies, cells were tested at 0.5, 2.5, and 6.5 hours post-treatment.

The data collected revealed a rapid transition from phosphatidylserine (PS) exposure to FVD-positive dead cell staining. This observation suggests that the predominant mode of killing may involve a mechanism that does not primarily expose PS, indicating the involvement of non-classical apoptotic pathways in the cytotoxic effects of SAB-142 and rATG on PBMCs.

### Summary of SAB-142 Cytotoxicity and Mechanisms of Cell Death

SAB-142 and Thymoglobulin® have been shown to induce phosphatidylserine (PS) exposure on T cells, a hallmark of early cell death processes. Throughout the experimental time course, the viability of untreated cells or those treated with negative control naive IgG remained consistent, indicating minimal spontaneous cell death under these conditions.

At the 30-minute time point, only a small proportion of PBMCs were single positive for ApoTracker, suggesting that few cells exhibited PS exposure while maintaining intact membranes. In contrast, the majority of cells were double positive for both ApoTracker and the viability stain, which is indicative of dead cells.

These findings suggest a rapid transition from PS exposure to loss of membrane integrity, highlighting the swift progression of cell death following treatment. Although caspase activation is a requirement for classical apoptosis, the data indicate that neither Caspase 3/7 nor Caspase 8 is activated in response to SAB-142 or Thymoglobulin®. This observation implies that cell death induced by these agents does not occur via classical apoptotic pathways. Instead, alternative mechanisms of regulated cell death that also involve PS exposure may be responsible and warrant further investigation.

### Example 11

Immunomodulation of Adult Cynomolgus Treated with SAB-142 or Thymoglobulin®

In this example, the immunomodulatory effects and safety profile of SAB-142 were evaluated in a preclinical study using adult Cynomolgus primates as a model system.

### BACKGROUND

A Good Laboratory Practice (GLP) toxicity study was conducted in adult Cynomolgus primates to evaluate the safety and immunomodulatory effects of SAB-142. Four groups, each consisting of six animals (three males and three females), received either 1, 5, or 10 mg/kg of SAB-142, or 5 mg/kg of the active comparator Thymoglobulin® (rATG). The primates were observed over a 28-day period, with safety designated as the primary endpoint. Immunomodulation was assessed as a secondary endpoint through immunophenotyping of lymphocytes, natural killer (NK) cells, and monocytes. SAB-142 successfully met its primary safety endpoint in this study and was then further evaluated in human clinical trials.

### Methods

Immunophenotype data were generated for each animal and treatment group throughout the study using flow cytometry analysis of immune cell marker antigens. These data were compared to pre-study baseline values, with any values above or below baseline considered as increased or decreased, respectively, regardless of the magnitude of change. Both the extent and frequency of these increases or decreases were evaluated to assess the potential biological significance of any observed alterations in immune cell populations.

### Results

Cynomolgus monkeys treated with SAB-142 had decreased whole blood absolute counts including total T (CD3+), T-helper (CD3+/CD4+), T-cytotoxic (CD3+/CD8+), and B (CD20+) cells beginning at Day 2. There were SAB-142 related decreases in naive, central and effector memory, and NK cells beginning at Day 2 but not monocytes.

Animals administered 10 mg/kg SAB-142 tended to have counts similar in magnitude and time compared to the active comparator (5 mg/kg rATG) beginning at Day 2 for total T, Th, and Tc cells (including central memory, effector memory, and naive subsets), and NK cells. The cell counts generally returned to pretreatment range by Day 8. Mean frequencies for naive, CM, EM, and TEMRA demonstrated differentiated states with SAB-142 treatment in each cell type. Compared to rATG, SAB-142 had a similar magnitude of decrease in B cell counts for animals administered≥1 mg/kg SAB-142 beginning at Day 2 through Day 8. B cell counts for animals administered SAB 142 or rATG generally returned to pretreatment values at Day 13.

### Conclusion

SAB-142 and rATG (Thymoglobulin®) exhibited similar immunophenotyping profiles in Cynomolgus monkeys, demonstrating comparable immunomodulatory effects on peripheral lymphocytes, B cells, monocytes, and natural killer (NK) cells. These findings were derived from the secondary endpoints; it should be noted, however, that the study was not specifically powered to statistically confirm similarities or differences in T cell phenotypes between the treatment groups.

### Example 12

Immunomodulation Data of Juvenile Cynomolgus Treated with SAB-142 or Thymoglobulin® (with Comparison to Selected Adult Study Results)

A 9-month, two-dose chronic GLP toxicology study in juvenile cynomolgus monkeys was conducted in which 2 dose administrations of SAB-142, Thymoglobulin®, or placebo were administered via 2-hour IV infusion with the initial dose over one or two days with at least 24 weeks of observation, and redosing occurring at ˜6 months, followed by at least 12 weeks of observation. The objective of the study was to evaluate the toxicity, TK, and immunologic effects of SAB-142 compared to placebo and Thymoglobulin® when administered via IV infusion. The dosing regimen in this study was designed to mimic the anticipated SAB 142 human dosing regimen of every 6 months dosing. Dose levels were selected to provide up to 20-fold safety margin of anticipated human dose levels.

Animals were administered the first dose of SAB-142 at 5, 10, and 25 mg/kg, Thymoglobulin® at 25 mg/kg as a positive control or saline solution as placebo (PBO)/negative control. Following the initial observation period, animals were re-dosed with SAB-142 (10, 25, and 50 mg/kg) or Thymoglobulin® (25 mg/kg), with at least 12-weeks of observation prior to necropsy. Through the entire study, no SAB-142 or Thymoglobulin® related mortality was observed. During the observation periods following both the first dose and re-dose, no SAB-142 related changes were reported for ophthalmic observations, ECG changes, urinalysis tests, heamatology, coagulation, or clinical chemistry. Notably, no SAB-142 specific changes in liver function tests were observed. Finally, no SAB 142 related changes were noted in the T cell dependent antibody response (TDAR) as measured by anti-KLH IgM or IgG titers following both the initial dose and re-dose of SAB-142 and Thymoglobulin®, a positive safety finding indicating no negative impact on the T cell dependent immune response to neo- and recall antigens as a part of the vaccination. Overall, the data for this 9-month chronic GLP toxicology study of SAB-142 and Thymoglobulin® in juvenile cynomolgus monkeys suggest both to be safe and well tolerated. A NOAEL was determined to be 50 mg/kg SAB-142, the highest dose level evaluated in the completed 9-month two-dose chronic GLP toxicology study.

In contrast, Thymoglobulin® at the 25 mg/kg dose in the 9-month study showed a sustained and greater lymphodepletion profile of T lymphocytes.

Assessment of T cell antibody dependent immune response (TDAR) assay on cellular and humoral immunity was conducted using Keyhole Limpet Hemocyanin (KLH) as a neo-antigen following administration of SAB-142 and Thymoglobulin® and later as a recall antigen. Following administration SAB-142 and Thymoglobulin® to juvenile animals, no effects were observed indicating that SAB-142 and Thymoglobulin®, administered at doses up to 50 mg/kg at initial or redosing, have no immunosuppression in juvenile monkeys of approximately 2 years of age, equivalent to approximately 5 years of human age. No SAB-142- or Thymoglobulin® related changes were noted in anti-KLH IgM or IgG titers or in the AUC calculations.

Results of the 9-month juvenile toxicology study indicated that SAB-142 was well tolerated with no mortality or weight alterations in doses of SAB-142 ranging from 5 mg/kg to 50 mg/kg following an initial dose and re-dose at ˜6 months. No accumulation of SAB-142 was observed after multiple doses.

In the 28-day acute toxicology in young adult NHPs, SAB-142 was well tolerated without unexpected or concerning findings on clinical or veterinary observations, body weight, qualitative or quantitative ECG parameters, haematology, coagulation, clinical chemistry, urinalysis, cytokine, blood or lymph node immunophenotyping parameters, organ weights, macroscopic or microscopic pathology in cynomolgus monkeys at any dose level. A NOAEL of 10 mg/kg, the highest dose administered in this study, was determined and was used to guide dose selection and monitoring for SAB-142-101.

In both GLP studies, transient decreases in peripheral lymphocytes (returning to pre-dose levels by Day 7), indicative of margination, were observed after administration of SAB-142 in both GLP studies, with no sustained lymphodepletion up to the highest dose tested at 50 mg/kg. The results of both studies demonstrated that SAB-142, administered at doses from 1-50 mg/kg, was safe and well tolerated in young adult/adolescent and juvenile NHPs, equivalent to a human age of 5 to <8 years of age.

### Example 13

Immunomodulation without Sustained Lymphodepletion: SAB-142, a Fully Human Anti-Thymocyte Globulin

This example describes a Phase I study (SAB-142-101) evaluating the safety and tolerability of SAB-142 in healthy volunteers.

Lymphodepleting regimens have been explored in type 1 diabetes (T1D) but pose significant challenges. Current strategies in T1D represent a paradigm shift, focusing on targeted immune modulation to achieve a state of immunotolerance to beta cells. Rabbit Anti-Thymocyte Globulin (rATG) has previously been used to treat T1D patients, but immunogenicity prevents repeat dosing. To overcome this immunogenicity, a fully human anti-thymocyte polyclonal antibody (SAB-142) was generated and is currently in a Phase I clinical trial.

### Materials and Methods

SAB-142-101 is a randomized, double-blind, placebo-controlled Phase I study evaluating the safety, tolerability, PK, and PD of SAB-142 in healthy volunteers. SAB-142 was administered intravenously (IV) as an ascending dose trial of up to 0.5 mg/kg on Day 1 and up to 2 mg/kg on Day 2. To investigate SAB-142 and rATG effector function, a commercial neonatal Fc receptor (FcRn) binding immunoassay was utilized. For measuring antibody dependent cellular cytotoxicity (ADCC) activation, Jurkat cells that contain stably expressed FcRs with a luciferase bioreporter downstream of the endogenous signaling pathways were used.

### Results

Following a single IV infusion, only a transient lymphopenia following administration of SAB-142 was observed, characterized by abnormal clinically significant (low) lymphocyte levels in all participants who received a total dose of 0.5, 1.5, and 2.5 mg/kg (FIG. 10). However, by Day 3, only 19% of participants who received 1.5 or 2.5 mg/kg had abnormal lymphocyte levels, and no abnormal values were observed from Day 4 to the end of the study at Day 120. No sustained lymphodepletion was observed. On the contrary, in clinical trials with low dose rATG at 2.5 mg/kg, a profound and long-lasting lymphopenia of predominantly CD4+ T cells was observed, lasting beyond 2 years from the dose administration.

To gain insight into T cell lymphodepletion, in vitro studies characterizing the Fc effector functions of SAB-142 and rATG were performed. The human FcRn transports IgG across endothelial barriers and facilitates the movement of IgG in both directions. For FcRn binding, results showed that SAB-142 had 5 to 7 times higher binding to the FcRn in comparison to rATG (FIG. 8A). For FcγRI binding, SAB-142 had comparable binding to FcγRI compared to rATG (FIG. 8B). Additionally, activation of ADCC via Fc binding to the FcγRIIIA was examined. The EC50 of rATG for ADCC activation was 0.3 μg/mL compared to EC50 of 3.7 μg/mL for SAB-142 (FIG. 8D). Binding to the FcγRIII on natural killer cells activated cellular pathways leading to antibody dependent cellular cytotoxicity (ADCC) (FIG. 8C). No ADCC was observed at therapeutic doses of SAB-142, showing that there was no lymphodepletion.

### Conclusion

For the SAB-142-101 trial, only transient peripheral lymphopenia was observed over the initial days of dosing. The safety profile of SAB-142, with a lack of sustained lymphopenia, is a major safety advantage of SAB-142. No sustained depletion of major blood cells, coupled with the lack of serum sickness and immunogenicity, creates a favorable SAB-142 safety profile for the target patient population across adult, adolescent, and pediatric age groups. The binding differences of SAB-142 and rATG to the FcRn in vitro suggest that SAB-142 may have higher levels of in vivo endothelial transport and recycling into lymph nodes or other highly perfused tissues. The measured ADCC EC50 levels demonstrate that rATG has 10 times greater ADCC activity compared to SAB-142. This result indicates that a significantly lower concentration of rATG is required for ADCC pathway activation, which is consistent with clinical lymphodepletion by rATG in T1D clinical studies, even at low doses.

SAB-142: Transient lymphopenia due to lymphocyte margination. Lymphocytes recover back to baseline by Day 7 (see FIG. 10 and FIG. 11D). SAB-142 results in immunomodulation with no depletion of CD8+ or CD4+ T cells, including T regulatory cells. SAB-142 demonstrated validated MOA to deliver potentially Best-in-Class T1D immunotherapy. No sustained lymphodepletion, unlike rabbit ATG, which causes a decrease in CD4+ T-cells for up to 2 years.

### Example 14

Sustained T-Cell Exhaustion Signature Across Key Cells Associated with C-Peptide Preservation

A sustained T-cell exhaustion signature across key immune cell populations has been identified as an important factor associated with C-peptide preservation in type 1 diabetes. Treatment with SAB-142 results in a decrease in CXCR3+CD4+ and CXCR3+ conventional T cells (Tconv) and shifts the T cell profile toward a central memory (CM) phenotype, particularly in cohorts receiving 1.5 to 2.5 mg/kg (see FIG. 13D). Additionally, SAB-142 reduces the population of CD4+ TEMRA cells, a reduction that is associated with decreased cytotoxic effects on residual beta cells. No significant difference was observed in CD8+ cells, which likely contributes to the differences in the magnitude of PD-1 response between these studies. The multi-target T-cell exhaustion profile, as measured by PD-1 expression on both CD4+ and CD8+ cells, has been shown to correlate with C-peptide preservation in T1D, underscoring the therapeutic potential of SAB-142 in promoting durable immunomodulation and preservation of endogenous insulin production.

### Example 15

SAB-142 Cytokine Profile is Analogous to rATG

A validated multiplex cytokine assay was employed to comprehensively characterize the cytokine release profile induced by SAB-142. The assay measured a panel of key cytokines, including IFN-γ, TNF, IL-10, IL-6, IL-4, and IL-2. Peripheral blood mononuclear cells (PBMCs) were incubated with SAB-142, and cytokine concentrations in the culture supernatant were quantified using the multiplex bead array platform.

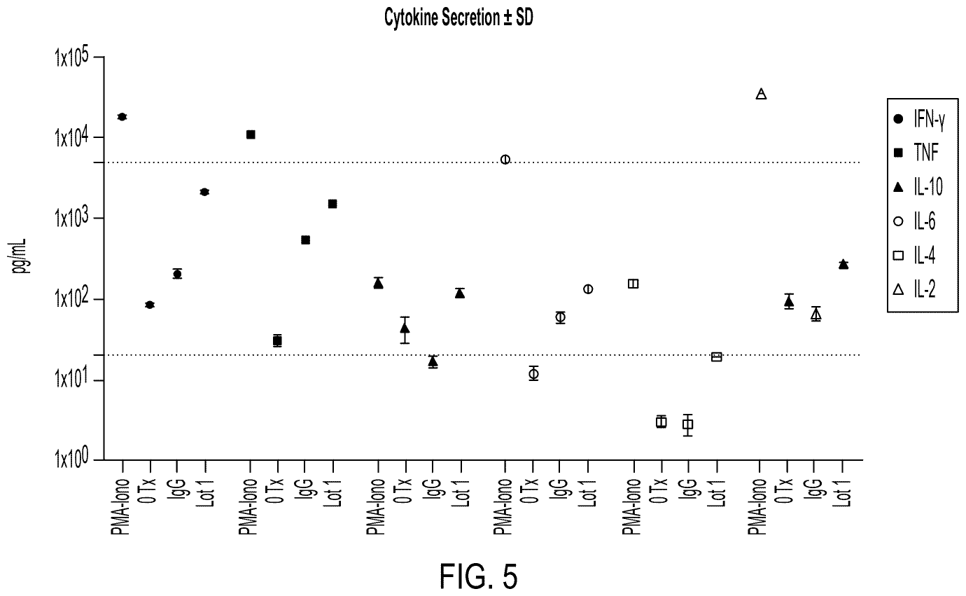

The results demonstrated that SAB-142 and rATG induced secretion of these cytokines in similar patterns and magnitudes across the tested panel (FIG. 5). Both agents elicited transient increases in pro-inflammatory cytokines such as IL-6, IL-8, TNF-α, and IFN-γ, as well as regulatory cytokines IL-2 and IL-10. The overall cytokine release profiles were highly analogous, indicating that SAB-142 recapitulates the immunomodulatory effects of rATG at the level of cytokine induction.

### Example 16

### Mechanism of Action of a Fully Human Anti-Thymocyte Globulin, SAB-142, for the Treatment of Type 1 Diabetes

This example describes the mechanisms of action of SAB-142 from data gathered in a Phase I clinical trial.

SAB-142 is a fully human Anti-Thymocyte Globulin (hATG) developed as a multi-target immunomodulating therapy intended to change the treatment paradigm of T1D by delaying its onset and inhibiting disease progression. Recent clinical studies utilizing Rabbit Anti-Thymocyte Globulin (rATG) have demonstrated C-peptide preservation in patients with T1D and delayed disease progression. Unfortunately, due to its heterologous nature, rATG has caused serum sickness in the majority of treated patients and cannot safely be re-dosed to maintain C-peptide preservation. The objective of this study was to characterize the pharmacologic mechanism of action of SAB-142 through immunoprofiling in both in vitro experiments and a first-in-human clinical study.

Pre-clinical studies were performed using human PBMCs isolated from fresh whole blood and incubated with fluorescently labeled antibodies prior to analysis via flow cytometry. The SAB-142 Phase I trial was a randomized, double-blind, placebo-controlled, single-ascending dose, adaptive design clinical study among healthy volunteers and one cohort of participants with T1D. Clinical samples were collected and PBMCs isolated and analyzed by cytometry.

### Results:

Pre-clinical data with SAB-142 and rATG showed comparable cytotoxic effects, largely sparing Treg cells while reducing CD8+ T cells. In addition, both ATG products promoted activation of naive Tconv cells and their subsequent proliferation, albeit SAB-142 did so to a greater extent. In vivo, SAB-142 induced sustained exhaustion in both CD4+ and CD8+ T-cell subsets as evidenced by increased PD-1 and TIGIT positivity by Day 30 that continued through the end of the study, Day 120. CD4+ and CD8+ T-cell populations were preserved at baseline levels, and there was a notable shift to a memory phenotype in CD4+ and CD8+ cells (FIG. 14D). Importantly, Tregs also exhibited increased inhibitory receptor expression, which potentially enhanced their suppressive functions.

### Conclusion:

These data suggest the mechanism of action of SAB-142 involves the activation of CD4+ and CD8+ T-cells, promoting their differentiation and increased inhibitory receptor expression in the absence of cytotoxicity. Activation of Tregs may contribute to sustained suppression of CD4+ and CD8+ T-cells involved in antigen specific killing of beta islet cells. Similarities to rATG include Treg preservation and inhibitory receptor induction in CD4+ T-cells. However, SAB-142 is distinct in its pharmacodynamic profile compared to rATG in that SAB-142 promotes additional modulation of CD8+ T cells and there was no observed CD4+ or CD8+ cytotoxicity. These findings support a muti-target mechanism of action that sustainably modulates CD4+ and CD8+ T-cell populations and unlike rATG does not have sustained lymphodepletion, which has not been correlated to C-peptide preservation in previous T1D clinical studies. In addition, SAB-142 does not cause serum sickness or generate anti-drug antibodies allowing for re-dosing. The pharmacologic profile of SAB-142 suggests a mechanism of action that has the potential to safely and sustainably restore immunotolerance, significantly improving the treatment and prognosis of T1D.

### Example 17

### Novel Pharmacokinetic (PK) Assay for Measuring SAB-142, a Fully Human Anti-Thymocyte Globulin

This example describes a novel pharmacokinetic assay to measure parameters related to treatment with SAB-142.

Background and Aims: In developing a pharmacokinetic (PK) assay for monitoring the fully human anti-thymocyte globulin (hATG), SAB-142, in clinical trials, several challenges arise due to its fully human nature and the difficulty in distinguishing it from native polyclonal human immunoglobulins. Here, a novel assay is presented that was developed and validated for the quantitative measurement of the multi-targeted SAB-142 in human serum samples from an SAB-142 Phase I clinical study with low-dose SAB-142 administration. This assay effectively overcomes these challenges through innovative approaches.

### Material and Methods

This is a flow cytometry-based PK assay that utilizes pooled human peripheral blood mononuclear cells (PBMCs), specifically gated for lymphocytes as the targeted cell populations, to measure the concentration of unbound free SAB-142 in the serum of study participants infused with SAB-142. The assay was validated for suitability and performance characteristics, including accuracy, precision, selectivity, and range, to ensure accurate quantification of SAB-142 in human serum. The validated assay has been employed to quantitatively measure the concentration of SAB-142 in human serum samples from participants in SAB-142 clinical trials (see TABLE 13).

TABLE 13 describes results showing selectivity in type 1
diabetes serum spiked with SAB-142 at HCQ and LLOQ concentrations.
Type 1 Diabetes

| | | | | | | | | Mean | | |
|---|---|---|---|---|---|---|---|---|---|---|
| | | | | | | | Mean | Recovery² | | Passed/ |
| Sample¹ | 1 | 2 | 3 | 4 | 5 | 6 | (pg/mL) | (%) | CV %³ | Failed⁴ |
| HQC | 14.7 | 16.3 | 13.1 | 15.1 | 16.3 | 12.6 | 14.7 | 98% | 11% | Passed |
| LLOQ | 0.32 | 0.20 | 0.26 | 0.32 | 0.27 | 0.20 | 0.26 | 104% | 24% | Passed |

¹HQC = High Quality Control; Lower Limit of Quantitation (0.25 μg/mL); Individual samples ran in triplicate
²(Actual concentration/theoretical concentration)*100
³Coefficient of Variation; (Standard Deviation/Mean)*100
⁴Accpetance criteria: HQC % Recovery = 80-120% and CV % ≤ 25%; LLOQ % Recovery = 70-130% and CV % ≤ 30%

### Results

The novel PK assay was validated with a linear range between 80 μg/mL and 0.25 μg/mL and R2 value of 0.9998. In the Phase I study (SAB-142-101, see Example 13), the Cmax and AUClast (Area Under the Curve from time zero to the last time point with measurable concentration) values of SAB-142 in human serum increased in a dose proportional manner across the 0.5 (Cohort 3), 1.5 (Cohort 4), and 2.5 (Cohort 5) mg/kg doses (Cohorts 5, 5N, and 5P (TiD patients)) (FIG. 16). Although systemic exposure to SAB-142 was limited for Cohort 3 at 0.5 mg/kg, the Cmax values ranged from 0.679 to 1.01, 1.04 to 3.00 and 0.99 to 4.32 μg/mL for Cohort 4 at 1.5 mg/kg and Cohorts 5 and 5N at 2.5 mg/kg, respectively, and AUClast values ranged from 9.15 to 36.0 and 31.7 to 105 h* g/mL at 1.5 mg/kg (Cohort 4) and 2.5 mg/kg (Cohort 5), respectively. The half-life from Cohort 5 at 2.5 mg/kg was between 26.4 and 61.6 hours.

### Conclusion

The novel SAB-142 PK assay was developed and validated with a Lower Limit of Quantification (LLOQ) in human serum of 0.25 μg/mL. This assay has been utilized for monitoring PK in a first-in-man, randomized, double-blind, placebo-controlled Phase I study (SAB-142-101). Results from this study demonstrated that the overall drug exposure of free SAB-142, as assessed by Cmax and AUClast, increased in a dose-proportional manner as the dose was increased from 1.5 to 2.5 mg/kg.

### Example 18

### Safety Profile of SAB-142: A Fully Human Anti-Thymocyte Globulin

This example shows the results of a Phase I study evaluating the safety profile and demonstrating the groundbreaking lack of immunogenicity for SAB-142.

There is no licensed treatment to halt or reverse the progression of new-onset type 1 diabetes (NOT1D). Rabbit anti-thymocyte globulin (rATG) has been clinically evaluated in NOT1D; however, the recipients develop neutralizing antibodies and hypersensitivity to rATG that limit its therapeutic utility. Outcomes of humans and primates that received an anti-thymocyte immunoglobulin composed of fully human polyclonal IgG antibodies (SAB-142) purified from the hyperimmune plasma of transchromosomic cattle immunized with human thymocytes are reported.

### Materials and Methods

SAB-142-101 is a randomized, double-blind, placebo-controlled Phase I study evaluating the safety, tolerability, PK, and PD of SAB-142 in healthy participants and a cohort of patients with type 1 diabetes (T 1D). SAB-142 was administered intravenously (IV) as a single dose ranging from 0.03 mg/kg to 2.5 mg/kg. A GLP study evaluated the safety, toxicokinetics, and immunologic effects of SAB-142 in juvenile cynomolgus monkeys. It was administered via IV infusion as an initial single or two-day dose, followed by 24 weeks of observation, then a re-dose over one to three days with 12 weeks of observation. The effects of SAB-142 (up to 50 mg/kg) were also compared to rATG (up to 25 mg/kg).

### Results:

There were no deaths or drug-related Serious Adverse Events (SAEs) in the first human (FIH) study. There were no participants in Treatment-Emergent Adverse Events (TEAEs) leading to study withdrawal. No abnormal (low value) findings were reported for neutrophils, erythrocytes, or platelets. Changes in hematology parameters of lymphocytes, neutrophils, and platelets demonstrate a lack of sustained lymphodepletion (transient peripheral lymphopenia only indicative of lymphocyte margination rather than cell death), neutropenia, thrombocytopenia, or anemia. There were no abnormal clinically significant assessments for any coagulation parameters.

In the non-human primates study, no SAB-142 or rATG-related mortality was observed. No SAB-142- or rATG-related alterations in body weight or overall body weight change occurred, and no changes in qualitative food consumption were observed. No SAB-142 or rATG-related neurobehavioral, ECG, or urinalysis effects were observed. No long-term SAB-142 or rATG-related changes in pro-inflammatory cytokines (TNF-α, IL-8, etc.) were noted (FIG. 14A, FIG. 14B, FIG. 14C, FIG. 14E, FIG. 14F). No SAB-142 or rATG-related mortality or organ weight differences occurred. No unexpected SAB-142 related hematology, coagulation, or clinical chemistry effects were noted for animals administered an initial dose or re-dose of up to 50 mg/kg. Only incidental macroscopic and microscopic observations were noted across all groups, including control groups at the terminal sacrifice.

### Conclusion:

SAB-142 exhibited a favorable safety profile in healthy volunteers when administered as an IV infusion of single ascending doses (0.03-2.5 mg/kg). No deaths, drug-related (SAEs, or TEAE-related study discontinuations occurred. Among Adverse Event of Special Interest (AESIs), no serum sickness or Anti-Drug Antibodies (ADAs) were observed. The juvenile non-human primate study safety findings were mild or transient. Following an initial 5 mg/kg SAB-142 (lowest) dose, ˜6 months of observation, and a 50 mg/kg (highest) re-dose with −3 months of monitoring, the doses were well tolerated. The No Observed Adverse Effect Level (NOAEL) is 50 mg/kg with up to a 20× safety margin based on human dosing, supporting safe dosing and re-dosing at six months.

### SAB-142 Shown to be Not Immunogenic

SAB-142 is a Fully Human, Multi-Specific, Targeted Anti-Thymocyte Globulin (hATG) for Delaying Onset and Progression of type 1 diabetes (T1D). Challenges arise in developing a pharmacokinetic (PK) assay for monitoring SAB-142 exposure in clinical trials. The difficulty in distinguishing SAB-142 from native human immunoglobulins due to its fully human nature. High-sensitivity assay needed.

### Low Dose Level of SAB-142

An even lower concentration of unbound free active SAB-142 circulating in the bloodstream. After infusion, the active components of SAB-142 bind to the lymphocytes in the patient's blood. To address these challenges, a novel high-sensitivity PK assay was developed and validated for the quantitative measurement of the SAB-142 in human serum samples from SAB-142 clinical trials.

Anti-SAB-142 antibodies were assessed in the Phase 1 trial using a validated ADA assay. This ADA assay is a meso-scale discovery (MSD) ECL-based bridging assay that utilizes a multi-tiered approach, including screening, confirmatory, and titer stages. The Minimum Required Dilution (MRD) was 1:10. Titration assays were run with two-fold dilutions. Immunogenicity was evaluated in the participants enrolled in the Phase 1 trial. Forty-one Subjects received single doses of SAB-142 at 0.5, 1.5, 2.5, 4.5 mg/kg, or were re-dosed at 2.5 mg/kg followed by 1.5 mg/kg.

The ADA incidence in this SAB-142 Phase 1 study was low, with no treatment-emergent ADAs above background detected in any subject, including both healthy volunteers and participants with stable Type 1 Diabetes (T1D). Notably, all subjects from the re-dosing cohort (2.5 mg/kg followed by 1.5 mg/kg) tested negative for ADA. This consistent finding across populations indicates that ADA development in humans is minimal and not a safety concern for SAB-142.

### Methods

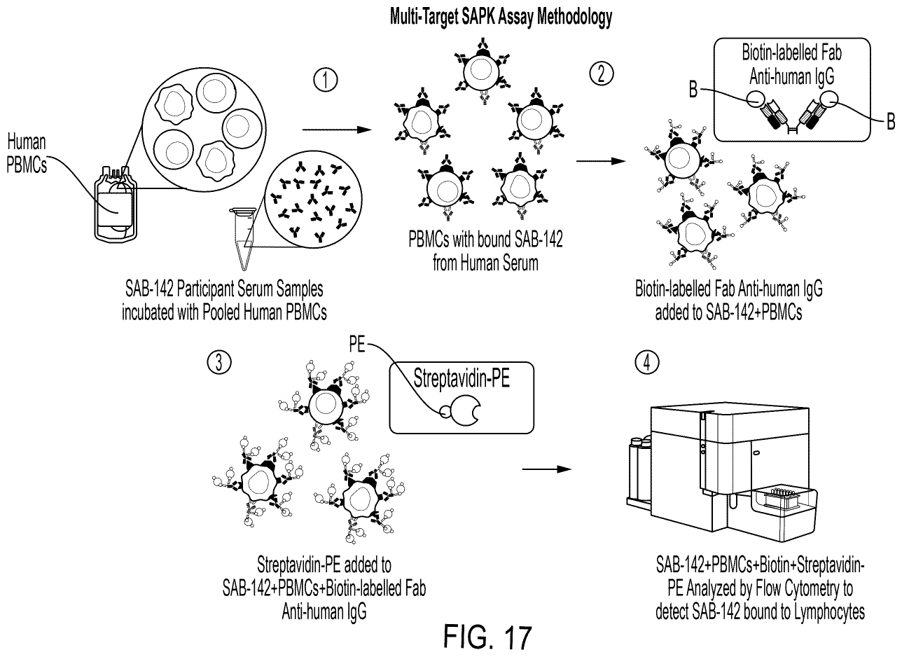

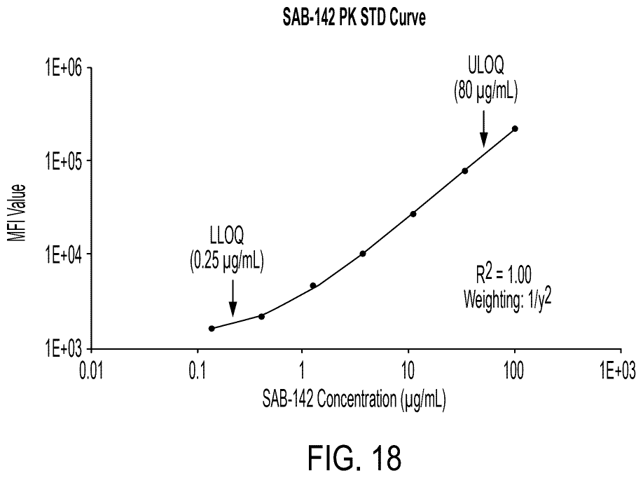

Pooled human PBMCs (5×10⁵ cells) were incubated with human serum samples for 1 hr at RT. Cells were washed, incubated with biotin F(ab′)₂ goat anti-human IgG-Fc (30 mins at RT), washed, and finally incubated with streptavidin-PE (15 mins at RT). Fluorescently-labeled PBMCs were gated for lymphocytes and analyzed via flow cytometry. SAB-142 concentration is determined by interpolating Median Fluorescence Intensity (MFI) against the standard. The assay was validated for accuracy, precision, selectivity and range (FIG. 17 and FIG. 18).

The design of the anti-drug antibody (ADA) assay is illustrated in FIG. 15. At the target dose of 2.5 mg/kg, neither cohort of healthy volunteers (HVs) treated with SAB-142 developed anti-SAB-142 antibodies, indicating no immunogenicity at this dose level. This finding demonstrates that SAB-142, a fully human IgG polyclonal antibody (pAb), is less immunogenic than Thymoglobulin®, a rabbit-derived IgG pAb, as evidenced by a minimum required dilution (MRD) of 1:10. In conclusion, following a single intravenous infusion of SAB-142, no ADAs were detected at target doses. The competitive safety profile of SAB-142 supports its use in ambulatory dosing settings. In contrast, teplizumab is associated with a high percentage of adverse events, as detailed in TABLE 14.

TABLE 14
lists adverse events and occurrence rates in the clinical trial
for SAB-142, where N = 54 for Placebo vs. Cohorts 5 & 5N.

| | Day 1 to 7 | | | Day 8-180 | | | |
|---|---|---|---|---|---|---|---|
| | Pooled | Pooled | | Pooled | | Pooled | |
| | Placebo | SAB-142 | | Placebo | | SAB-142 | |
| | HV | HV | | HV | | HV | |
| | (N = 14) | (N = 40) | | (N = 14) | | (N = 40) | |
| Category | n (%) | n (%) | | n (%) | | n (%) | |
| Number of | | | | | | | |
| participants | | | | | | | |
| with any: | | | | | | | |
| TEAEs | 8 (57.1%) | 36 | (90.0%) | 6 | (42.9%) | 18 | (45.0%) |
| TEAEs by | | | | | | | |
| Severity: | | | | | | | |
| Grade 1 | 6 (42.9%) | 2 | (5.0%) | 2 | (14.3%) | 13 | (32.5%) |
| Grade 2 | 2 (14.3%) | 3 | (7.5%) | 4 | (28.6%) | 4 | (10.0%) |
| Grade 3 | 0 | 14 | (35.0%) * | 0 | | 1 | (2.5%) |
| Grade 4 | 0 | 17 | (42.5%) * | 0 | | 0 | |
| Grade 5 | 0 | 0 | | 0 | | 0 | |
| Treatment- | | | | | | | |
| related TEAEs | | | | | | | |
| by Severity: | | | | | | | |
| Grade 1 | 3 (21.4%) | 1 | (2.5%) | 0 | | 4 | (10.0%) |
| Grade 2 | 2 (14.3%) | 3 | (7.5%) | 1 | (7.1%) | 1 | (2.5%) |

| | | | | | |
|---|---|---|---|---|---|
| Grade 3 | 0 | 14 | (35.0%) | 0 | 0 |
| Grade 4 | 0 | 17 | (42.5%) | 0 | 0 |

| | | | | |
|---|---|---|---|---|
| Grade 5 | 0 | 0 | 0 | 0 |

TABLE 15
is modified from a presentation at the International Society for
Pediatric and Adolescent Diabetes (ISPAD) conference in 2024.

| | Teplizumab AE |
|---|---|
| Symptom | (N = 791) |
| Lymphopenia | 80% |
| Nausea | 20% |
| Leukopenia | 63% |
| Rash | 35% |
| Headache | 27% |
| AST increased | 28% |
| ALT increased | 27% |
| Neutropenia | 40% |
| Thrombocytopenia | 22% |
| Cytokine Release Syndrome (CTCAE Version 5.0 grade | 5.80% |
| 2, with hypotension responsive to IV fluids) | |
| Anemia | 29% |

The majority of adverse events (AEs) observed were mild, typically associated with infusions administered on Days 1 and 2, and resolved by the end of the first week. This favorable safety profile supports the feasibility of ambulatory dosing for SAB-142. The clinical evaluation included seven cohorts of healthy volunteers (HVs), with a total of 54 participants, 40 receiving SAB-142 and 12 receiving a placebo. Dosing was conducted via intravenous infusion on Day 1 and Day 2. The most frequently reported AEs included headaches, which are common with all T-cell engaging therapies and were primarily observed during the first two days of treatment. Lymphopenia, an anticipated pharmacodynamic effect, was also noted but resolved rapidly within one to three days. Other reported events included infusion-related reactions (IRRs) such as mild, grade 1 cytokine release syndrome (CRS) presenting as flu-like symptoms on Days 1 and 2, as well as infusion-site reactions (ISRs) like erythema, tenderness, and phlebitis. Importantly, there were no drug-related serious adverse events (SAEs), no cases of serum sickness, and no adverse events associated with the development of anti-drug antibodies (ADAs).

The novel PK assay developed and validated for accuracy, precision, selectivity, and range. SAB-142 had a dose-proportional PK profile in the Phase I Study. No major differences in systemic exposure to SAB-142 between HVs and T1D patients dosed at 2.5 mg/kg.

In summary, the safety data from the clinical evaluation of SAB-142 demonstrated a favorable tolerability profile, with the majority of adverse events being mild, transient, and resolving within the first week of treatment.

### Example 19

### Positive Clinical Translation Of A Juvenile Non-Human Primate Study For Evaluating Sab-142, A Multi-Specific Tcell Targeting T1 D Therapy, In Humans

This example describes the study design, methods and results used in a non-human primate pre-clinical study of SAB-142. SAB-142 is a fully human, multi-specific, targeted Anti-Thyrnmyte Globulin (hATG) developed for delaying the onset and progression of T1D with the capability for safe and effective redosing. A 9-month, two-dose chronic GLP toxicology study in 30 juvenile cynomolgus monkeys was conducted to evaluate the toxicity, toxicokinetic, and immunologic effects of SAB-142. Pharmacological outcomes of this study were compared to outcomes from a first-in-human Phase I clinical study of SAB-142 to assess clinical translatability.

### Methods

In this positively predictive GLP toxicology study, three dose levels of SAB-142 were administered at study initiation and redosed at 6 months. This study was designed to mimic the regimen of every 6-months dosing and provided up to a 20-fold safety margin of target dose levels in a Phase I and an ongoing Phase 2b clinical trial.

TABLE 16
describes the non-human primate study design.

| Duration of | | 9 months | | |
|---|---|---|---|---|
| Observation | | ~6 months | ~3 months | |
| Group | Test | Initial dose | Re-dose at ~6 mo | Number of |
| No. | Material | (mg/kg) | (mg/kg) | Animals |
| 1 | Placebo | 0 | 0 | 3 monkeys/ |
| | Control | | | |
| 2 | Rabbit Anti- | 25 (Split | 25 (Split | sex per |
| | Thymocyte | over 2 days: | over 2 days: | treatment arm |
| | Globulin | 10/15) | 10/15) | 30 monkeys |
| 3 | SAB-142 | 5 | 50 (Split | total |
| | | | over 3 days: | |
| | | | 20/20/10) | |
| 4 | SAB-142 | 10 | 10 | |
| 5 | SAB-142 | 25 (Split | 25 (Split | |
| | | over 2 days: | over 2 days: | |
| | | 10/15) | 10/15) | |

Test material, active comparator or placebo were administered in a volume of 10 mL/kg infused over 2 hours via intravenous infusion.

### Results

The 9-month chronic GLP toxicology study demonstrated that SAB-142 was safe and well tolerated in juvenile cynomolgus monkeys. Consistent with its expected pharmacological activity, SAB-142 induced a transient, dose-proportional reduction in total peripheral lymphocyte counts. This effect was short-lived and aligned with the anticipated mechanism of lymphocyte margination.

### Conclusions

The positive translational value of this 9-month, two-dose chronic GLP toxicology study was supported by Phase I clinical data, with both studies demonstrating no sustained lymphocyte depletion across all tested dose levels of SAB-142. In the Phase I clinical study, peripheral lymphocyte counts returned to baseline by Day 7, in both healthy volunteers and patients with stable T1D, confirming the clinical translation of the transient pharmacodynamic effect observed in this preclinical toxicology study in cynomolgus monkeys.

### Example 20

### Phase 2b Clinical Trial Evaluating SAB-142 for Delaying Progression of Type 1 Diabetes (SAFEGUARD Study)

This example describes a Phase 2b clinical trial evaluating the efficacy and safety of SAB-142 in participants with Stage 3 New Onset Type 1 Diabetes (NOTID).

### Study Design

The SAFEGUARD study (SAFety and Efficacy of Human Anti-thymocyte ImmunoGlobUlin SAB-142 ARresting Progression of Type 1 Diabetes) is a Phase 2b, randomized, double-blind, placebo-controlled, parallel-arm dose finding study. The study is designed to evaluate the efficacy, safety and tolerability of SAB-142 in patients with Stage 3 NOT1D.

### Study Population

Male and female participants aged 5-40 years old (inclusive) at the time of randomization are eligible for enrollment. Part A of the study enrolls participants aged 15-40 years old, while Part B may enroll participants aged 5-40 years old following safety review. Participants must have a weight of≥16.0 kg at time of randomization.

Participants must have received a diagnosis of T1D according to American Diabetes Association criteria within 100 days of randomization. For participants who were initially misdiagnosed with Type 2 diabetes, time from misdiagnosis with Type 2 diabetes to randomization is 100 days. The date of diagnosis is defined as the date of the first insulin dose or any other glucose lowering medication. An extension of no more than 14 days is permitted if a participant has planned and/or is required to receive a vaccination within 30 days prior to randomization or is completing the 10 day CGM period.

Participants must have random C-peptide levels of >0.2 nmol/L, measured during Screening. One random C-peptide retest during screening period is allowed. Participants must have completed all scheduled samples for C-peptide collected during the MMTT test during Screening.

Participants must have a positive result on testing for at least one of the following T1D-related autoantibodies during screening: Glutamic acid decarboxylase 65 (GAD65), Islet antigen 2 (IA-2), Zinc transporter 8 (ZnT8), or Insulin autoantibodies (if testing within the first 14 days of insulin treatment).

### Planned Number of Participants

Approximately 159 participants are planned to be enrolled in the study.

### Study Arms and Interventions

The study includes three arms: High Dose SAB-142, Low Dose SAB-142, and Placebo. Part A is an open-label, parallel arm study. Part B is a double-blind, placebo-controlled, parallel-arm study. Enrollment into Part B may commence once all participants in Part A have been randomized.

### Primary Objectives and Endpoints

For Part A, the primary objective is to evaluate the safety of SAB-142. The primary endpoint for Part A is the incidence of treatment-emergent adverse events (TEAEs), adverse events of special interest (AESIs), and serious adverse events (SAEs) from dose administration through Week 4.

For Part B, the primary objective is to determine whether SAB-142 slows the loss of 3 cells and preserves β cell function over 12 months in participants with Stage 3 NOT1D. The primary endpoint for Part B is the area under the concentration-time curve (AUC) of C-peptide after a 2-hour mixed meal tolerance test (MMTT), a measure of endogenous insulin production and β cell function (change from baseline in C-peptide ln [AUC+1] at 12 months).

### Secondary Objectives and Endpoints

Secondary endpoints for Part B include time in tight range (TITR) expressed as a daily average of the percentage of time in a 24-hour day a participant's glucose is >70 but ≤140 mg/dL (>3.9 to 7.8 mmol/L), assessed using continuous glucose monitoring (CGM), evaluated at baseline, Months 3, 6, 9 and 12.

Hemoglobin A1c (HbA1c) levels expressed in % and mmol/mol are evaluated at baseline, Months 3, 6, 9 and 12.

Time in range (TIR) expressed as a daily average of the percentage of time in a 24-hour day a participant's CGM reading is >70 but ≤180 mg/dL (>3.9 to 10.0 mmol/L), assessed by CGM, is evaluated at baseline, Months 3, 6, 9 and 12.

Time above range, assessed by CGM, is evaluated at baseline, Months 3, 6, 9 and 12.

Time below range, assessed by CGM, is evaluated at baseline, Months 3, 6, 9 and 12.

Exogenous insulin use defined as a daily average in units per kilogram per day (U/kg/day) (total daily insulin based on participant's diary at predefined study periods) is evaluated at baseline, Months 3, 6, 9 and 12.

Number of clinically important episodes defined as the total number of Level 2 and 3 hypoglycemic events and/or episodes of cognitive impairment requiring external assistance for recovery (participant's diary and CGM-based) is evaluated at baseline, Months 3, 6, 9 and 12.

Proportion of participants with partial clinical remission defined as an insulin requirement of <0.25 units per kg of body weight per day and HbA1c <6.5% (47 mmol/mol) is evaluated at baseline, Months 3, 6, 9 and 12.

Proportion of participants with partial remission defined as insulin-dose adjusted A1c (IDAA1c)+[4×insulin dose (units per kilogram per 24 hours)]≤9 is evaluated at baseline, Months 3, 6, 9 and 12.

Total BETA-2 score, comprised of fasting plasma glucose (mmol/L), HbA1c (%), daily insulin (U/kg), and fasting C-peptide (nmol/L), is evaluated at baseline, Months 3, 6, and 12.

Insulin dose-adjusted A1c (IDAA1C) is evaluated at baseline, Months 3, 6, 9 and 12.

Incidence of TEAEs, AESIs, and SAEs is evaluated from dose administration through Month 12.

SAB-142 serum concentrations are evaluated on Days 1 and 2 of each treatment period (pre- and post-dose/end of infusion [EOI]), plus Weeks 1, 4, and Months 3, 6, and 7.

Incidence and titers of anti-SAB-142 antibodies including optional neutralizing antibodies (nAbs) in serum are evaluated at baseline, Week 4, Months 3, 6, 7, 9 and 12.

Immunophenotyping (IPT) is evaluated at baseline (Day 1, pre-dose) and at subsequent timepoints throughout the study.

### Conclusion

Based on the study design and endpoints described, the SAFEGUARD study is designed to evaluate whether SAB-142 demonstrates dose-dependent preservation of j-cell function as measured by C-peptide levels, along with improvements in glycemic control parameters and favorable immunomodulatory effects. The results may demonstrate sustained T-cell exhaustion markers and preservation of regulatory T-cell populations without sustained lymphodepletion, consistent with the mechanism of action observed in the Phase I study.

### Example 21

A Phase 3, Randomized, Double-Blind, Placebo-Controlled, Study Evaluating the Efficacy and Safety of SAB-142 for Delaying the Progression of Type 1 Diabetes (TID) in Patients with Stage 3 New Onset of Type 1 Diabetes (NOTID), Recent Onset TID and Established TID
PRISE (Personalized Response and Immunologic Surveillance of Endogenous C-Peptide Preservation in New, Recent, and Established Onset Type 1 Diabetes Treated with Human Anti-Thymocyte Globulin [h-ATG]) Study

This Example Describes a Phase 3 Study Evaluating the Safety and Efficacy of SAB-142 for Type 1 Diabetes Patients.

### Study Rationale

Type 1 diabetes occurs due to the autoimmune destruction of pancreatic β cells. Preclinical research and clinical trials demonstrate that rabbit anti-thymocyte globulin (rATG), Thymoglobulin®, reduces this autoimmune response by acting on T-lymphocytes while preserving the T-regulatory cells. However, undesirable adverse events (AEs) such as serum sickness and high immunogenicity have been observed due to the heterologous nature of rabbit-derived immunoglobulins. SAB-142 is a preparation of purified fully human polyclonal anti-thymocyte globulin (ATG) immunoglobulin G (IgG) and is being developed as a disease-modifying therapeutic agent to delay the onset and progression of T1D. As a fully human ATG IgG, SAB-142 is expected to combine the beneficial effects of Thymoglobulin® with a more favorable safety profile. This study will evaluate the efficacy, safety and tolerability of SAB-142 in patients with New Onset (NOT1D,<100 days post diagnosis), Recent Onset T1D (ROT1D, >100 days <1 year) and Established Onset T1D (EOT1D, >1 year to <2 years).

### Study Population

Male and female participants with Stage 3 NOT1D, ROT1D and EOT1D aged 5-40 years old (inclusive) at the time of randomization.

### Study Design

This study is a randomized, double-blind, placebo-controlled study. The study will have 3 Cohorts. Cohort 1 is a synthetic cohort consisting of Stage 3 NOT1D participants from the SAFEGUARD study randomly selected in 2:1 allocation ratio for SAB-142, 2.5 mg/kg and placebo. Cohort 1 will match cohorts 2 and 3 participants age distribution. Cohort 2 consists of ROT1D participants recruited for the PRISE study enrolled and randomized to receive SAB-142, 2.5 mg/kg or placebo in a 2:1 allocation ratio. Cohort 3 consists of EOT1D participants recruited for the PRISE study enrolled and randomized to receive SAB-142, 2.5 mg/kg or placebo in a 2:1 allocation ratio. For cohort 2 and 3, approximately 72 participants will be recruited in a 1:1 ratio (36 participants with ROT1D and 36 participants with EOT1D).

### Planned Number of Participants

A total of up to 72 participants are planned to be enrolled across 2 cohorts in the PRISE study: Cohort 2 (ROT1D) and Cohort 3 (EOT1D). Each cohort will enroll approximately 36 participants randomized at a ratio of 2:1 to receive SAB-142, 2.5 mg/kg or placebo. Data from 36 participants with Stage 3 NOT1D enrolled, randomized and treated with SAB-142, 2.5 mg/kg or placebo in the SAFEGUARD study will constitute the synthetic Cohort 1. Together, a total of 108 participants will be included and evaluated in this study.

### Dosing Schedule

There will be two treatment periods (TPs) during this study for Cohort 2 and 3. For TP1, SAB-142 or placebo will be split into two separate part-doses for IV administration over two days (Study Day 1 and Study Day 2). Day 1 dosing consists of 0.5 mg/kg SAB-142 or equivalent volume of placebo, and Day 2 dosing consists of 2.0 mg/kg SAB-142 or equivalent volume of placebo. For TP2, participants will receive a second dose of SAB-142 or placebo at Month 6. The dose at Month 6 will also be delivered as a split dose over 2 days, as per TP1.

### Study Objectives and Endpoints

The primary objective is to determine whether SAB-142 slows the loss of β cells and preserves β cell function over 12 months in participants with Stage 3 NOT1D, ROT1D and EOT1D. The primary endpoint will be to calculate area under the concentration-time curve (AUC) of C-peptide after a 2-hour mixed meal tolerance test (MMTT), a measure of endogenous insulin production and β cell function (change from baseline in C-peptide ln [AUC+1] at 12 months) in hATG compared to placebo.

The secondary objectives will be to identify a personalized immunotherapy approach based on previously identified baseline pre-treatment in vitro testing to identify responders vs non-responders to SAB-142; to evaluate participant improvements in key clinical parameters of diabetes management following administration of SAB-142; to determine the safety and tolerability of SAB-142; to evaluate the pharmacokinetics (PK) and immunogenicity SAB-142; and to evaluate changes in immune cell populations following administration with SAB-142. The secondary endpoints will include: AUC of C-peptide after a 2-hour MMTT (change from baseline in C-peptide ln [AUC+1] at 12 months) in a priori identified “responders” and “non-responders” to hATG and compared to placebo; hemoglobin A1c levels expressed in % and mmol/mol; time in tight range expressed as a daily average of the percentage of time in a 24-hour day a participant's glucose is >70 but ≤140 mg/dL (>3.9 to ≤7.8 mmol/L), assessed using continuous glucose monitoring (CGM); time in range expressed as a daily average of the percentage of time in a 24-hour day a participant's glucose is >70 but ≤180 mg/dL (>3.9 to 10.0 mmol/L), assessed by CGM; time above range, assessed by CGM; time below range, assessed by CGM; exogenous insulin use defined as a daily average in units per kilogram per day (U/kg/day) (total daily insulin based on participant's diary at predefined study periods); clinically important episodes defined as the total number of Level 2 and 3 hypoglycemic events and/or episodes of cognitive impairment requiring external assistance for recovery (participant's diary and CGM-based); proportion of participants with partial clinical remission defined as an insulin requirement of <0.25 units per kg of body weight per day and HbA1c <6.5% (47 mmol/mol); proportion of participants with partial remission defined as insulin-dose adjusted A1c (IDAA1c)+[4×insulin dose (units per kilogram per 24 hours) ≤9; total BETA-2 score, comprised of fasting plasma glucose (mmol/L), HbA1c (%), daily insulin (U/kg), and fasting C-peptide (nmol/L); and IDAA1C. Endpoints related to safety and tolerability include the incidence of treatment-emergent adverse events, incidence of serious adverse event, and incidence of adverse event of special interest. Endpoints related to PK and immunogenicity of SAB-142 include SAB-142 serum concentrations, incidence and titers of anti-SAB-142 antibodies in serum (neutralizing antibodies if indicated), and immunophenotyping.

Exploratory objectives are to determine the effects of SAB-142 on type 1 diabetes clinical parameters, including composite clinical end points and end points assessing j-cell function, and on patient- and parent-reported outcomes; and to evaluate mechanistic, molecular, and genetic markers. Endpoints for these objectives include C-peptide quantitative response (baseline, Month 3, 6, and 12), 2-hour MMTT C-peptide AUC (baseline, Month 3, 6, and 12), fasting C-peptide (baseline, Month 3, 6, 9, and 12), the proportion of participants maintaining a clinically significant stimulated peak C-peptide of >2 nmol/L during the 2-hour MMTTs (baseline, Month 3, 6, and 12), and fasting proinsulin-to-C-peptide ratio, a measure of p-cell endoplasmic reticulum stress and dysfunction (baseline, Month 3, 6, 9, and 12).

Exploratory endpoints also include calculated estimated residual stimulated C-peptide, the proportion of participants with poor glycemic control defined as HbA1c of >9%, the number of participants who do not require exogenous insulin because they are able to achieve local, regional, or national age-based glycemic management goals for HbA1c and/or routine blood glucose levels, the incidence of Level 1, 2, and Level 3 hypoglycemia during the CGM reporting periods; incidence of diabetic ketoacidosis requiring medical attention defined as a hyperglycemic episode with serum or urine ketones elevated beyond upper limit of normal along with serum bicarbonate <18 mmol/L or blood pH<7.3, or both, and resulting in outpatient, emergency room visit or hospitalization; and the number of participants with both HbA1c in the American Diabetes Association target range (i.e., ≤7.5%) and exogenous insulin dose in specific ranges (<0.25, 0.25 to ≤0.50, 0.50 to ≤0.75, 0.75 to ≤1.0, 1.0 to ≤1.25, and >1.25 U/kg/day. Patient-reported outcomes include the Age-appropriate Diabetes Treatment Satisfaction Questionnaire (DTSQ), the age-appropriate PedsQL™ Diabetes Module Version 3.2, and parent reports for DTSQ if participants are under the age of 18 years old.

Mechanistic, molecular and genetic endpoints include a serum cytokine panel, a safety immune functional assay, a functional exhaustion immune assay, an activation induced marker immune assay (evaluated at week 4, month 6 and month 12), a neutrophil immune assay (evaluated at month 6 and month 9), ex vivo treatment of pre-dose PBMCs (at baseline), a whole blood sample for IPT (evaluated at baseline, week 1, week 4, month 3, month 6, month 7, month 9, and month 12), a single nucleotide polymorphism analysis at baseline, DNA methylation and RNA sequencing (evaluated at baseline, month 6 and month 9), and microRNA expression (evaluated at month 3, month 6, and month 9).

### Adverse Events

### Hypoglycemia

This study will consider clinically important hypoglycemia Level 2 and/or Level 3 using the definitions below. Clinically important hypoglycemia is defined as BG values of <54 mg/dL (3.0 mmol/L) and termed “Level 2 Hypoglycemia”. Significant impairment consistent with hypoglycemia that requires assistance, often referred to as “severe” hypoglycemia, even in the absence of a BG reading, is termed “Level 3 Hypoglycemia”. Level 3 events may include cognitive impairment, altered/loss of consciousness, confusion, seizure, syncope/fainting, or coma, and support may be general assistance, glucagon, or oral carbohydrate (i.e., fruit juice or glucose tablets). These events may or may not require medical attention or hospitalization. These events will constitute one of the key secondary endpoints of the study:

Level 2: BG concentration <54 mg/dL (3.0 mmol/L). It is noted that this is the threshold at which neuroglycopaenic symptoms begin to occur and requires immediate action to resolve the hypoglycemic event. If a participant has Level 2 hypoglycemia without adrenergic or neuroglycopaenic symptoms, they likely have hypoglycemia unawareness.

Level 3: A severe event characterized by altered mental and/or physical functioning that requires assistance from another person for recovery.

Blood glucose values of >54 but ≤70 mg/dL (3.0-3.9 mmol/L) (level 1 hypoglycemia) and other milder symptoms should also be recorded during the designated diary periods; diary and CGM-based Level 1 hypoglycemic events will be part of the exploratory analyses.

### Diabetic Ketoacidosis

In this study, hyperglycemia will be reported as a disease-specific adverse event if it meets Grade 4 severity and/or is associated with clinically significant diabetic ketoacidosis. Clinically significant diabetic ketoacidosis is defined as current or very recent hyperglycemia, for example, a blood glucose level of greater than 250 milligrams per deciliter (13.9 millimoles per liter), combined with acidemia, for example arterial pH less than or equal to 7.3, bicarbonate level of less than or equal to 18 milliequivalents per liter adjusted for albumin gap of 10-12.3, and ketonemia or ketonuria, for example serum or urine ketones elevated beyond the upper limit of normal, and requiring medical attention such as unplanned outpatient care, emergency room care, or hospitalization.

### Anticipated Events

For the purposes of this study, the following will be considered anticipated events: hypoglycemia (a T1D-related anticipated event), diabetic ketoacidosis or DKA (a T1D-related anticipated event), and transient peripheral lymphopenia (a treatment-related, anticipated pharmacodynamic effect of SAB-142). In the SAB-142-101 Phase I study, 100% of transient lymphopenia self-resolved within 48-72 hours after drug administration; it is a desired and anticipated PD effect.

### Assessment of Severity

The severity of each AE and SAE will be assessed by the Investigator according to the National Cancer Institute Common Terminology Criteria for Adverse Events (NCI-CTCAE) Version 5.0 grading scale. The scale for grading the intensity of each AE and SAE includes Grade 1 events which are mild, asymptomatic or have mild symptoms with clinical or diagnostic observations only and intervention not indicated. Grade 2 events are moderate with minimal, local or noninvasive intervention indicated and limiting age-appropriate instrumental activities of daily living (ADL). Grade 3 events are severe or medically significant but not immediately life-threatening, with hospitalization or prolongation of hospitalization indicated, disabling, and limiting self-care ADL. Grade 4 events have life-threatening consequences with urgent intervention indicated. Grade 5 events result in death related to the AE.

Based on the study design and endpoints described, dose-dependent preservation of p-cell function is observed, as measured by C-peptide levels, along with improvements in glycemic control parameters and favorable immunomodulatory effects. The results may demonstrate sustained T-cell exhaustion markers and preservation of regulatory T-cell populations without sustained lymphodepletion.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for producing a plurality of human antibodies or fragments thereof against human thymocytes, comprising:
a) administering a plurality of human thymocytes to a transchromosomic ungulate, wherein a viability of the plurality of human thymocytes is at least about 70%;
b) collecting plasma from the transchromosomic ungulate; and
c) purifying the plurality of human antibodies from the plasma,
wherein the viability can be measured by dividing a number of viable cells over a number of total cells,
wherein the method produces a purified human immunoglobulin (IgG) product.

2. The method of claim 1, further comprising administering to the transchromosomic ungulate an adjuvant.

3. The method of claim 1, wherein the plurality of human thymocytes comprises at least 75%, 80%, 85%, 90%, or 95% intact cells.

4. The method of claim 1, wherein the viability is measured by counting intact cells.

5. The method of claim 1, wherein the administering the plurality of human thymocytes is administered via a subcutaneous injection.

6. The method of claim 5, wherein the subcutaneous injection is administered at one or more different locations of the transchromosomic ungulate.

7. The method of claim 1, wherein the administering step is followed by collecting a plasma sample from the transchromosomic ungulate.

8. The method of claim 7, wherein the plasma sample is about 4 L to about 10 L for a 400 kg transchromosomic ungulate per collection.

9. The method of claim 8, wherein the plasma sample comprises about 1% to 3% of a body weight of the transchromosomic ungulate.

10. The method of claim 1, wherein a plasma sample is collected from each of two or more transchromosomic ungulates.

11. The method of claim 10, further comprising pooling the plasma sample collected from each of the two or more transchromosomic ungulates to produce a pooled plasma.

12. The method of claim 11, further comprising purifying the pooled plasma.

13. The method of claim 12, further comprising subjecting a purified human IgG product to at least one of ultrafiltration, diafiltration, bioburden reduction, sterile filtration, or a combination thereof, to generate a storage formulation.

14. The method of claim 13, wherein the storage formulation comprises:
a) about 1 to 100 mM L-glutamic acid monosodium salt;
b) about 50 to 500 mM D-sorbitol;
c) about 0.01 to 2 mg/mL Tween 80; and
d) about 21 to 31 mg/mL human IgG.

15. The method of claim 12, wherein a total human IgG content in the pooled plasma is greater than 2.0 mg/mL.

16. The method of claim 1, wherein the ungulate is a bovine.

17. A pharmaceutical composition, comprising:
a) a plurality of fully human or substantially human antibodies or fragments thereof against human thymocytes, wherein the plurality of fully human or substantially human antibodies comprises an immunoglobulin (IgG) profile that is about 70% IgG1; and
b) and one or more pharmaceutically acceptable excipients.

18. A pharmaceutical composition, comprising:
a) a plurality of fully human or substantially human antibodies or fragment thereof against human thymocytes, wherein the plurality of fully human or substantially human antibodies comprises a glycosylation profile having detectable alpha-galactose in released N-linked glycans and the released N-linked glycans can be measured by mass spectrometry; and
b) and one or more pharmaceutically acceptable excipients.

19. The pharmaceutical composition of claim 18 wherein the plurality of fully human or substantially human antibodies or fragments thereof are multi-specific.

20. The pharmaceutical composition of claim 19, wherein the plurality of fully human or substantially human antibodies or fragments thereof, has an avidity to an immune cell surface protein of at least 1000 seconds k_off residence time.

21. A composition, produced by immunizing a transchromosomic ungulate with a plurality of human thymocytes, wherein a viability of the plurality of human thymocytes is at least about 70%, and
wherein the composition comprises a plurality of fully human or substantially human antibodies or fragments thereof,
wherein the viability can be measured by dividing a number of viable cells over a number of total cells,
wherein the plurality of fully human or substantially human antibodies or fragments thereof is a purified human IgG product.

22. The composition of claim 21, wherein a viability of the plurality of human thymocytes is measured by counting intact cells.

23. The composition of claim 22, wherein the immunizing comprises administering a second immunization to the transchromosomic ungulate 1, 2, 3, 4, 5, 6, 7, or 8 times after a first immunization.

24. A composition, comprising a plurality of fully human or substantially human antibodies or fragments thereof against human thymocytes, produced by pooling plasma collected from two or more transchromosomic ungulates subsequent to immunizing the two or more transchromosomic ungulates with a plurality of human thymocytes.

25. The composition of claim 24, wherein the pooled plasma contains human anti-thymocyte immunoglobulin (ATG), and the pooled plasma is further purified to produce a purified human IgG product.

26. The composition of claim 25, wherein:
a. any anti-red blood cell antibody is substantially removed in a first purified plasma sample;
b. any viruses, pathogens, and/or non-IgG bovine plasma proteins are removed in a second purified plasma sample;
c. transchromosomic IgG is isolated from the pooled plasma by capturing a kappa Fab fragment in a third purified plasma sample;
d. transchromosomic IgG is further captured in a fourth purified plasma sample;
e. IgA, IgM, host cell proteins, DNA, endotoxin, or a combination thereof are substantially removed in a fifth purified plasma sample;
f. viruses are substantially removed; or
g. the human ATG is concentrated, diafiltrated, or a combination thereof as a purified human IgG product.

27. The composition of claim 26, wherein the human IgG product is formulated with an excipient buffer via diafiltration to form a storage formulation.

28. The composition of claim 27, wherein the excipient buffer comprises 50 mM L-glutamic acid monosodium salt; 262 mM D-sorbitol; 0.05 mg/ml Tween 80; and pH 5.5±0.1.

29. The composition of claim 27, wherein the storage formulation is further purified via an 0.1 to 0.3p m filter, a sterile filter, or a combination thereof to produce a final distribution formulation.

30. A method for treating Type 1 Diabetes (T1D) in a subject in need thereof, comprising administering to the subject an effective amount of the composition of claims 21-29, wherein the T1D in the subject is treated.

31. The method of claim 30, wherein the subject has Stage 3 New Onset of T1D (NOT1D).

32. The method of claim 31, wherein the effective amount for patient body weight comprises between about 0.5 mg/kg to 4.5 mg/kg of a purified human IgG product formulated with a buffer.

33. The method of claim 30, wherein the administering comprises:
a first dose of about 0.5 mg/kg; and
a second dose of about 1 mg/kg or 2 mg/kg,
wherein the second dose is administered the day after the first dose.

34. The method of claim 30, wherein the composition is administered about every 6 months after a first single dose or a split dose.

35. The method of claim 30, wherein treatment of the T1D in the subject produces an effect of partial clinical remission or stabilization of type 1 diabetes.

36. The method of claim 30, wherein treatment of the T1D in the subject produces substantially no or reduced adverse events compared to administering of a rabbit anti-thymocyte globulin.
